(19) United States
(12) Patent Application Publication
(10) Pub. No.: US 2026/0315372 A1
(43) Pub. Date: Oct. 15, 2026

(54) INJECTABLE PHARMACEUTICAL FORMULATIONS

(71) Applicant: Cybin IRL Limited, Dublin (IE)

(72) Inventors: Alex Nivorozhkin, West Roxbury, MA (US); Mohammed I. Shukoor, Lexington, MA (US); Ju Feng, Groton, MA (US); Kenneth L. Avery, Merrimack, NH (US); Pradip M. Pathare, Lexington, MA (US); Geoffrey B. Varty, Annandale, NJ (US); Michael E. Morgan, San Diego, CA (US); Michael Palfreyman, St. Petersburg, FL (US)

(73) Assignee: Cybin IRL Limited, Dublin (IE)

(21) Appl. No.: 19/481,242

(22) PCT Filed: May 6, 2024
(86) PCT No.: PCT/EP2024/062406

§ 371 Date: Nov. 4, 2025

Related U.S. Application Data
(60) Provisional application No. 63/464,265, filed on May 5, 2023.
(60) Provisional application No. 63/507,062, filed on Jun. 8, 2023.
(60) Provisional application No. 63/599,483, filed on Nov. 15, 2023.

(51) Int. Cl. A61K 9/00; A61K 9/08; A61K 31/135; A61K 31/4045; A61K 47/36

(52) U.S. Cl. CPC A61K 9/0019; A61K 9/08; A61K 31/135; A61K 31/4045; A61K 47/36

(57) ABSTRACT

Provided herein are injectable pharmaceutical formulations, methods for their production, kits, and uses thereof in the treatment of diseases or disorders. The injectable pharmaceutical formulations include a psychopharmaceutical agent, a release modifier, and an aqueous vehicle. The pharmaceutical formulations allow for a time-restricted temporal controlled-release of the psychopharmaceutical agent upon administration by injection such as subcutaneous injection. The diseases or disorders treatable with the pharmaceutical formulations include, for example, neuropsychiatric diseases or disorders or inflammatory diseases or disorders, such as a central nervous system (CNS) disorder and/or psychological disorder, including those associated with a 5-HT₂ receptor.

### CROSS-REFERENCE

This application claims priority to U.S. Provisional Application No. 63/464,265, filed on May 5, 2023, U.S. Provisional Application No. 63/507,062, filed on Jun. 8, 2023, and U.S. Provisional Application No. 63/599,483, filed on Nov. 15, 2023, each incorporated herein by reference in their entireties.

### FIELD

The present disclosure relates generally to injectable pharmaceutical formulations comprising a psychopharmaceutical agent and uses in the treatment of neuropsychiatric diseases or disorders or inflammatory diseases or disorders, such as a central nervous system (CNS) disorder and/or psychological disorder, including those associated with a 5-HT₂ receptor.

### BACKGROUND

The pharmacological treatment of mood, anxiety, trauma-related, and substance use disorders with currently available drugs is characterized by (partial) ineffectiveness. A substantial proportion of patients fail to recover despite treatment with therapeutic doses and various augmentation agents, and patients who do achieve symptomatic relief often experience burdensome adverse effects and run a serious risk of suicidal behavior or completed suicide.

In the last three decades, there has been a resurgence of interest in psychedelic, dissociative, and entactogenic drug research. Various such drugs when combined with psychotherapy, have shown early, but robust effectiveness following a limited number of administrations (typically 1-3 drug assisted psychotherapy sessions). For instance, the entactogen methylenedioxymethamphetamine (MDMA) has recently passed the first phase-III trial in patients with PTSD [Mitchell J M, et al. MDMA-assisted therapy for severe PTSD: a randomized, double-blind, placebo-controlled phase 3 study. Nat Med. 2021; 27: 1025-1033]. Lysergic acid diethylamide (LSD) has shown clinical promise in alcohol use disorder [Chi T, Gold J A. A review of emerging therapeutic potential of psychedelic drugs in the treatment of psychiatric illnesses. J Neurol Sci. 2020, 411: 116715; Fuentes J J, et al. Therapeutic Use of LSD in Psychiatry: A Systematic Review of Randomized-Controlled Clinical Trials. Front Psychiatry. 2020; 10: 1-14]. In the tryptamine class of serotonergic psychedelics, psilocybin (the active ingredient in ‘magic mushrooms’) has shown promise in a phase II trial for MDD, end of life anxiety [Griffiths R R, et al. Psilocybin produces substantial and sustained decreases in depression and anxiety in patients with life-threatening cancer: A randomized double-blind trial. J Psychopharmacol. 2016; 30: 1181-1197], and smoking cessation [Garcia-Romeu A, Griffiths R R, Johnson M W. Psilocybin-occasioned mystical experiences in the treatment of tobacco addiction. Curr Drug Abuse Rev. 2014; 7: 157-164; de Veen B T H, et al. Psilocybin for treating substance use disorders? Expert Rev Neurother. 2017; 17: 203-212; Johnson M W, et al. Pilot study of the 5-HT2AR agonist psilocybin in the treatment of tobacco addiction. J Psychopharmacol. 2014; 28: 983-992; and Johnson M W, Garcia-Romeu A, Griffiths R R. Long-term follow-up of psilocybin-facilitated smoking cessation. Am J Drug Alcohol Abuse. 2017; 43: 55-60]; and N,N-dimethyltryptamine (DMT; also referred to as 2-(1H-indol-3-yl)-N,N-dimethylethan-1-amine) (usually as an active ingredient in ayahuasca) has shown promising effects on mood [Vollenweider F X, Preller K H. Psychedelic drugs: neurobiology and potential for treatment of psychiatric disorders. Nat Rev Neurosci. 2020; 21: 611-624; Barker S A. N,N-dimethyltryptamine (DMT), an endogenous hallucinogen: Past, present, and future research to determine its role and function. Front Neurosci. 2018; 12: 1-17; and Strassman R J, et al. Dose-response study of N,N-Dimethyltryptamine in humans II: Subjective effects and preliminary results of a new rating scale. Arch Gen Psychiatry. 1994; 51: 98-108] and potentially, addictions [Thomas G, et al. Ayahuasca-assisted therapy for addiction: Results from a preliminary observational study in Canada. Curr Drug Abuse Rev. 2013; Oliveira-Lima A J, et al. Effects of ayahuasca on the development of ethanol-induced behavioral sensitization and on a post-sensitization treatment in mice. Physiol Behav. 2015; 142: 28-36; Nolli L M, et al. Effects of the hallucinogenic beverage ayahuasca on voluntary ethanol intake by rats and on cFos expression in brain areas relevant to drug addiction. Alcohol. 2019; Fibregas J M, et al. Assessment of addiction severity among ritual users of ayahuasca. Drug Alcohol Depend. 2010; 111: 257-261; Argento E, et al. Exploring ayahuasca-assisted therapy for addiction: A qualitative analysis of preliminary findings among an Indigenous community in Canada. Drug Alcohol Rev. 2019; 38: 781-789; and Noorani T, et al. Psychedelic therapy for smoking cessation: Qualitative analysis of participant accounts. J Psychopharmacol. 2018; 32: 756-769].

DMT's binding profile is well characterized. DMT acts on numerous ionotropic and metabotropic receptors. It binds with affinity to 5-hydroxytryptamine-(5-HT)_{1A}, 5-HT_{2A}, 5-HT_{2C}, and serotonin transporter (SERT) receptors from the 5-HT/serotonin family [Cameron L P, Olson D E. Dark Classics in Chemical Neuroscience: N,N-Dimethyltryptamine (DMT). ACS Chem Neurosci. 2018; 9: 2344-2357]. DMT is a potent agonist at the 5-HT_{2A} receptor, through which (not unlike other serotonergic psychedelics) it exerts many subjective, visual, and potentially therapeutic effects [Cameron L P, Olson D E. Dark Classics in Chemical Neuroscience: N,N-Dimethyltryptamine (DMT). ACS Chem Neurosci. 2018; 9: 2344-2357; Carbonaro T M, Gatch M B. Neuropharmacology of N,N-dimethyltryptamine. Brain Res Bull. 2016; 126: 74-88]. 5-HT_{2A} activation has also been linked with increased synaptic plasticity [Ly C, et al. Psychedelics Promote Structural and Functional Neural Plasticity. Cell Rep. 2018; 23: 3170-3182; Ly C, et al. Transient Stimulation with Psychoplastogens Is Sufficient to Initiate Neuronal Growth. ACS Pharmacol Transl Sci. 2020; and Inserra A, De Gregorio D, Gobbi G. Psychedelics in Psychiatry: Neuroplastic, Immunomodulatory, and Neurotransmitter Mechanisms. Pharmacol Rev. 2021; 73: 202-277]. Additionally, DMT binds to other receptors, including trace amine-associated receptors (TAARs) and the sigma-1 receptor, potentially contributing to its neuroprotective plasticity enhancing effects [Carbonaro T M, Gatch M B. Neuropharmacology of N,N-dimethyltryptamine. Brain Res Bull. 2016; 126: 74-88; Barker S A. N,N-dimethyltryptamine (DMT), an endogenous hallucinogen: Past, present, and future research to determine its role and function. Front Neurosci. 2018; 12: 1-17].

Despite high potencies at serotonin 5-HT₂Rs, the therapeutic value of certain tryptamine psychedelics has been hampered by their accelerated metabolism in the liver and gastrointestinal tract most notably by monoamine oxidase (MAO) enzymes. For example, DMT is not orally active—it is converted to inactive metabolites before sufficient brain penetration can occur. For this reason, DMT is typically dosed with a monoamine oxidase inhibitor (as is the case in ayahuasca) to prolong its duration of action. Likewise, 5-methoxy-N,N-dimethyltryptamine (5-MeO-DMT) lacks oral bioavailability and is instead ordinarily vaporized and inhaled to produce psychedelic effects. Such MAO-mediated metabolism is also believed to contribute to high variability in the pharmacokinetic (PK) profiles of various tryptamines in humans, including significant patient-to-patient pharmacokinetic variability after oral psilocybin administration and intravenous (IV) administration of DMT.

Additionally, in marked contrast to the hours-long subjective effects of other serotonergic psychedelics such as LSD (8 to 20 hour duration of action) and psilocybin (6 to 8 hour duration of action), the duration of action time course of short-acting tryptamine psychedelics such as DMT and 5-MeO-DMT administered as a bolus to humans via inhalation or intravenous or intramuscular injection is brief—so short as to limit their use in effective therapies. For DMT, the onset of subjective effects on perception and consciousness (sometimes referred to as the “psychedelic state”) are rapid and overwhelming, with profound and intense visual peak experiences noted within 2 minutes of administration. The psychedelic state dissipates rapidly, with subjective effects returning to baseline (or close to) following roughly 20 to 30 minutes post administration [Strassman R J, Qualls C R, Uhlenhuth E H, Kellner R. Dose-response study of N,N-Dimethyltryptamine in humans II: Subjective effects and preliminary results of a new rating scale. Arch Gen Psychiatry. 1994; 51: 98-108]. On one hand, the short time course spent in the psychedelic state is seen as therapeutically limiting and makes sophisticated granular assessment of pharmacodynamic (PD) effects and safety profiles inherently difficult. On the other hand, short-acting tryptamine psychedelics are potentially more controllable and clinically scalable compared to longer-acting psychedelic compounds such as LSD and psilocybin, which often require 7 to 8 hours or more of supervised clinical observation of a patient before discharge.

To improve treatment profiles of DMT by avoiding excessive drug levels associated with bolus IV injections or inhalation administration, and to provide more scalable duration of effects (e.g., peak effects of about 30 to 120 minutes), continuous-controlled IV infusions have been studied [Gallimore A R, Strassman R J. A model for the application of target-controlled intravenous infusion for a prolonged immersive DMT psychedelic experience. Front Pharmacol. 2016; 7: 1-11]. DMT infusions administered over about 90 minutes allowed for expanding, in a time-controlled manner, the exposure while maximizing the effects of the drug. However, infusion protocols (e.g., where the patient is administered the drug over prolonged periods such as over an hour or longer) are not generally clinically practical, as they require significant clinical resources and personnel that represent major challenges in scaling up the treatment for larger populations of patients.

Consequently, there is a need for new formulations and delivery approaches which retain the pharmacokinetic and pharmacologic benefits of intravenous infusion administration (i.e., provide a target-controlled duration of peak effects lasting about 30 to 120 minutes), yet offer an alternative that is clinically practical, convenient for the patients, and economical for sponsors and service providers.

### SUMMARY

Accordingly, the present disclosure is based at least in part on the identification of novel injectable pharmaceutical formulations, kits, and treatment methods which allow for a time-restricted temporal controlled-release of a psychopharmaceutical agent. In the case of short-acting tryptamine psychedelics such as DMT, 5-MeO-DMT, and their analogs (e.g., deuterated analogs), this controlled-release would provide a duration of peak effects of about 30 to 120 minutes to maximize therapeutic benefits and reduce side effects. By doing so, the injectable pharmaceutical formulations extend the time the patient spends in the psychedelic state compared to bolus IV injection of the same tryptamine psychedelic, without overextending the release and the resulting duration of peak effects beyond about 120 minutes to avoid the prolonged supervised clinical observation requirements imposed by longer-acting psychopharmaceutical agents such as LSD and psilocybin.

These and other objects, which will become apparent during the following detailed description, have been achieved by the inventors' discovery of novel injectable pharmaceutical formulations containing a psychopharmaceutical agent in combination with specific release modifiers which can be administered as a bolus injection, including as a subcutaneous bolus injection, that display a tunable and linear release of the psychopharmaceutical agent. In the case of tryptamine psychedelics such as DMT, 5-MeO-DMT, and their analogs (e.g., deuterated analogs), a desirable controlled-release can be achieved to mimic the clinically advantageous 30 to 120 minute peak effect profile that has only been achieved by IV infusion of such psychopharmaceutical agents.

Thus, the present disclosure provides:

(1) An injectable pharmaceutical formulation, comprising:
a psychopharmaceutical agent;
a hyaluronate salt; and
an aqueous vehicle;
wherein the psychopharmaceutical agent is a pharmaceutically acceptable salt of a compound of Formula (I) or a stereoisomer, solvate, or prodrug thereof,

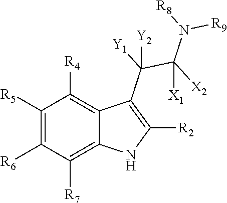

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, unsubstituted or substituted alkoxy, unsubstituted or substituted alkylthio, and unsubstituted or substituted acyloxy;
R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl; and
R₈ and R₉ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl,
or alternatively R₈ and R₉ together with the nitrogen atom attached thereto are optionally joined to form an unsubstituted or substituted heterocycloalkyl.

(2) The injectable pharmaceutical formulation of (1), wherein the compound has a structure of Formula (II), or a stereoisomer, solvate, or prodrug thereof

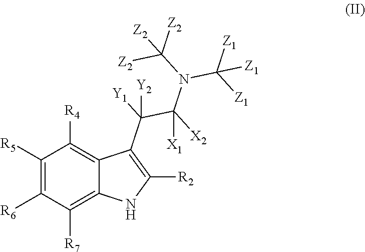

wherein:
X₁ and X₂ are independently hydrogen or deuterium,
Y₁ and Y₂ are independently hydrogen or deuterium,
each Z₁ is independently hydrogen or deuterium,
each Z₂ is independently hydrogen or deuterium, and
R₂, R₄, R₅, R₆, and R₇ are independently hydrogen or deuterium.

(3) The injectable pharmaceutical formulation of (1) or (2), wherein the psychopharmaceutical agent is a pharmaceutically acceptable salt of at least one compound selected from the group consisting of

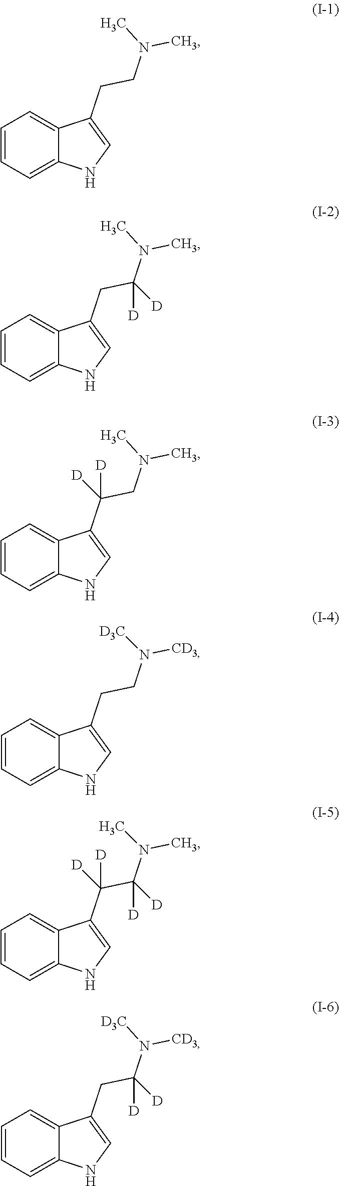 [SMILES: CN(C)CCc1c[nH]c2ccccc12 ; [2H]C([2H])(Cc1c[nH]c2ccccc12)N(C)C ; [2H]C([2H])(CN(C)C)c1c[nH]c2ccccc12 ; [2H]C([2H])([2H])N(CCc1c[nH]c2ccccc12)C([2H])([2H])[2H] ; [2H]C([2H])(c1c[nH]c2ccccc12)C([2H])([2H])N(C)C ; [2H]C([2H])([2H])N(C([2H])([2H])[2H])C([2H])([2H])Cc1c[nH]c2ccccc12]

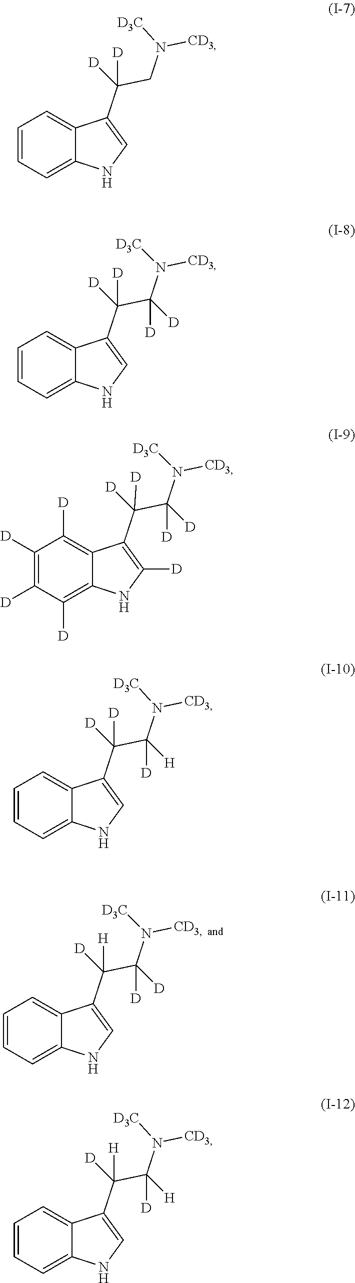

or a stereoisomer, solvate, or prodrug thereof.

(4) The injectable pharmaceutical formulation of any one of (1) to (3), wherein the psychopharmaceutical agent is a pharmaceutically acceptable salt of 2-(1H-indol-3-yl)-N,N-bis(methyl-d₃)ethan-1-amine-1,1,2,2-d₄ (I-8).

(5) The injectable pharmaceutical formulation of any one of (1) to (3), wherein the psychopharmaceutical agent is a pharmaceutically acceptable salt of 2-(1H-indol-3-yl)-N,N-bis(methyl-d₃)ethan-1-amine-1,1-d₂ (I-6).

(6) The injectable pharmaceutical formulation of any one of (1) to (3), wherein the psychopharmaceutical agent is a pharmaceutically acceptable salt of 2-(1H-indol-3-yl)-N,N-dimethylethan-1-amine-1,1-d₂ (I-2).

(7) The injectable pharmaceutical formulation of any one of (1) to (3), wherein the psychopharmaceutical agent is an active salt mixture comprising: (i) a pharmaceutically acceptable salt of 2-(1H-indol-3-yl)-N,N-bis(methyl-d₃)ethan-1-amine-1,1,2,2-d₄ (I-8); and (ii) a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11).

(8) The injectable pharmaceutical formulation of (7), wherein the active salt mixture comprises (i) from 60% to 99% by weight of the pharmaceutically acceptable salt of 2-(1H-indol-3-yl)-N,N-bis(methyl-d₃)ethan-1-amine-1,1,2,2-d₄ (I-8), based on a total weight of the active salt mixture; and (ii) from 1% to 40% by weight, in sum, of the pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), based on a total weight of the active salt mixture.

(9) The injectable pharmaceutical formulation of (1), wherein the compound has a structure of Formula (III), or a stereoisomer, solvate, or prodrug thereof

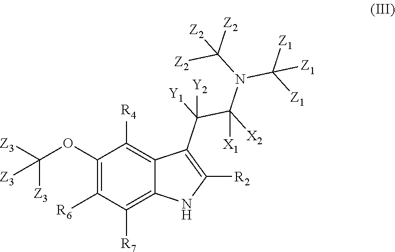

wherein
X₁ and X₂ are independently hydrogen or deuterium,
Y₁ and Y₂ are independently hydrogen or deuterium,
each Z₁ is independently hydrogen or deuterium,
each Z₂ is independently hydrogen or deuterium,
each Z₃ is independently hydrogen or deuterium, and
R₂, R₄, R₆, and R₇ are independently hydrogen or deuterium.

(10) The injectable pharmaceutical formulation of (1) or (9), wherein the psychopharmaceutical agent is a pharmaceutically acceptable salt of at least one compound selected from the group consisting of

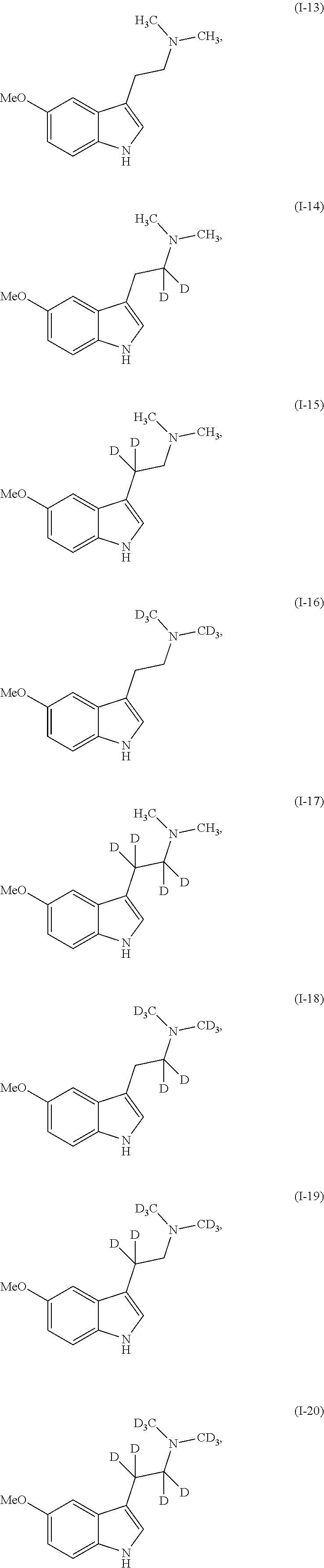

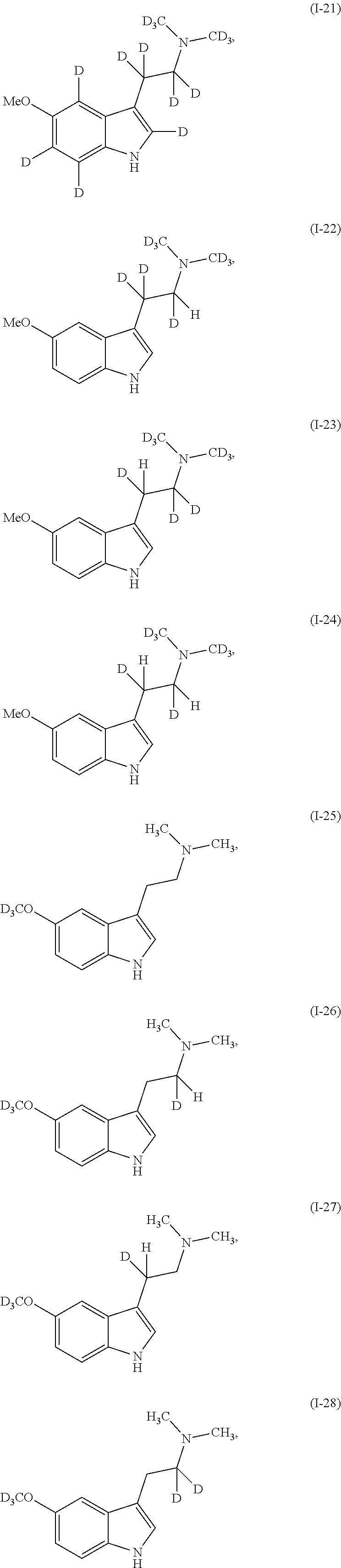

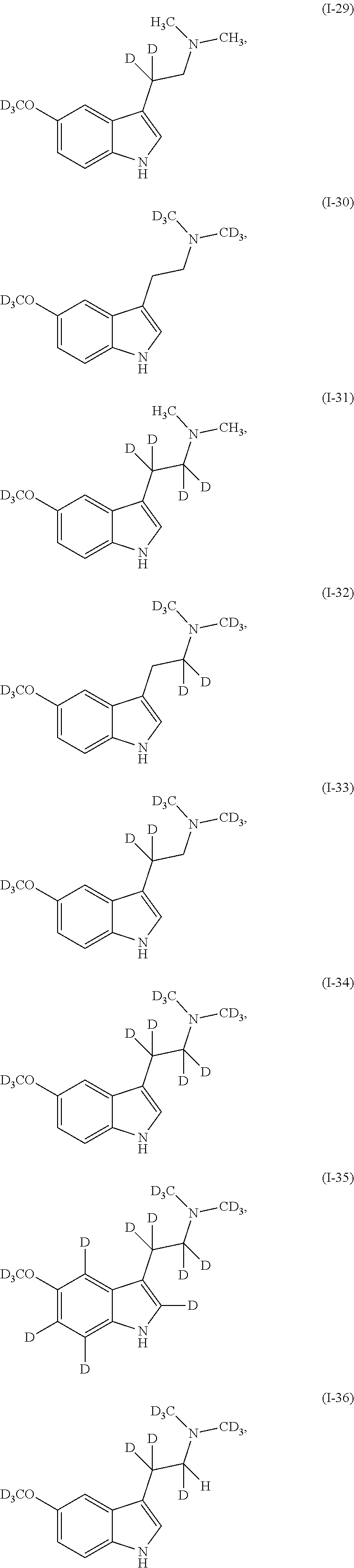

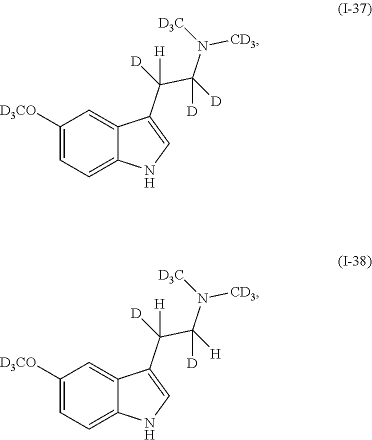

or a stereoisomer, solvate, or prodrug thereof.

(11) The injectable pharmaceutical formulation of any one of (1), (9), or (10) wherein the psychopharmaceutical agent is a pharmaceutically acceptable salt of 2-(5-methoxy-1H-indol-3-yl)-N,N-bis(methyl-d₃)ethan-1-amine-1,1,2,2-d₄ (I-20).

(12) The injectable pharmaceutical formulation of any one of (1) or (9) to (11), wherein the psychopharmaceutical agent is an active salt mixture comprising: (i) a pharmaceutically acceptable salt of 2-(5-methoxy-1H-indol-3-yl)-N,N-bis(methyl-d₃)ethan-1-amine-1,1,2,2-d₄ (I-20); and (ii) a pharmaceutically acceptable salt of one or more of 2-(5-methoxy-1H-indol-3-yl)-N,N-bis(methyl-d₃)ethan-1-amine-1,2,2-d₃ (I-22) and/or 2-(5-methoxy-1H-indol-3-yl)-N,N-bis(methyl-d₃)ethan-1-amine-1,1,2-d₃ (I-23).

(13) The injectable pharmaceutical formulation of any one of (1) to (12), wherein the pharmaceutically acceptable salt is a fumarate, a benzoate, a salicylate, a succinate, an oxalate, a glycolate, a hemi-oxalate, or a hemi-fumarate salt.

(14) The injectable pharmaceutical formulation of any one of (1) to (13), wherein the pharmaceutically acceptable salt is a fumarate, a benzoate, a salicylate, or a succinate salt.

(15) The injectable pharmaceutical formulation of any one of (1) to (14), wherein a concentration of the psychopharmaceutical agent by weight (in terms of free base equivalence) per total volume of the pharmaceutical formulation is about 1 mg/mL to about 100 mg/mL.

(16) The injectable pharmaceutical formulation of any one of (1) to (15), wherein a concentration of the psychopharmaceutical agent by weight (in terms of free base equivalence) per total volume of the pharmaceutical formulation is about 10 mg/mL to about 50 mg/mL.

(17) The injectable pharmaceutical formulation of any one of (1) to (16), wherein the hyaluronate salt is sodium hyaluronate.

(18) The injectable pharmaceutical formulation of any one of (1) to (17), wherein the hyaluronate salt has a weight average molecular weight of about 500 kDa to about 2,000 kDa.

(19) The injectable pharmaceutical formulation of any one of (1) to (18), wherein the hyaluronate salt has a weight average molecular weight of about 1,000 kDa to about 1,800 kDa.

(20) The injectable pharmaceutical formulation of any one of (1) to (19), wherein a concentration of the hyaluronate salt by weight per total volume of the pharmaceutical formulation is from about 0.1% to about 2% (w/v).

(21) The injectable pharmaceutical formulation of any one of (1) to (20), wherein a concentration of the hyaluronate salt by weight per total volume of the pharmaceutical formulation is from about 0.1% to about 1% (w/v).

(22) The injectable pharmaceutical formulation of any one of (1) to (21), wherein a concentration of the hyaluronate salt by weight per total volume of the pharmaceutical formulation is from about 0.1% to about 0.75% (w/v).

(23) The injectable pharmaceutical formulation of any one of (1) to (22), wherein a concentration of the hyaluronate salt by weight per total volume of the pharmaceutical formulation is from about 0.1% to about 0.5% (w/v).

(24) The injectable pharmaceutical formulation of any one of (1) to (23), wherein the aqueous vehicle comprises water and sodium chloride.

(25) The injectable pharmaceutical formulation of (24), wherein the pharmaceutical formulation comprises sodium chloride at a concentration, in terms of weight per total volume of the pharmaceutical formulation, of about 0.1% to about 0.8% (w/v).

(26) The injectable pharmaceutical formulation of any one of (1) to (25), which has a pH of about 3 to about 7.

(27) The injectable pharmaceutical formulation of any one of (1) to (26), which has an osmolality of about 150 mOsm/kg to about 600 mOsm/kg.

(28) The injectable pharmaceutical formulation of any one of (1) to (27), which has a viscosity of less than about 3,000 cP.

(29) The injectable pharmaceutical formulation of any one of (1) to (28), which is suitable for subcutaneous injection.

(30) The injectable pharmaceutical formulation of any one of (1) to (29), which is suitable for bolus subcutaneous injection.

(31) The injectable pharmaceutical formulation of any one of (1) to (30), wherein the injectable pharmaceutical formulation provides a duration of peak effects of about 30 minutes to about 120 minutes after being administered to a human subject via bolus subcutaneous injection.

(32) A kit suitable for preparing the injectable pharmaceutical formulation of any one of (1) to (31), the kit comprising:
(a1) a first solution comprising the psychopharmaceutical agent and the aqueous vehicle; and
(b1) a second solution comprising the hyaluronate salt and the aqueous vehicle.

(33) A method of treating a central nervous system (CNS) disorder and/or psychological disorder in a subject in need thereof, comprising administering to the subject a therapeutically effective amount the injectable pharmaceutical formulation of any one of (1) to (31).

(34) The method of (33), wherein the CNS disorder and/or psychological disorder is a substance use disorder.

(35) The method of (34), wherein the substance use disorder is alcohol use disorder.

(36) The method of (33), wherein the CNS disorder and/or psychological disorder is an anxiety disorder.

(37) The method of (36), wherein the anxiety disorder is generalized anxiety disorder (GAD).

(38) The method of (37), wherein the generalized anxiety disorder is comorbid with depression.

(39) The method of (36), wherein the anxiety disorder is social anxiety disorder.

(40) The method of (33), wherein the CNS disorder and/or psychological disorder is a depressive disorder.

(41) The method of (40), wherein the depressive disorder is major depressive disorder (MDD) or treatment-resistant depression (TRD).

(42) The method of (33), wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

(43) The method of any one of (33) to (42), wherein the injectable pharmaceutical formulation is administered via injection.

(44) The method of any one of (33) to (43), wherein the injectable pharmaceutical formulation is administered via subcutaneous injection.

(45) The method of any one of (33) to (44), wherein the injectable pharmaceutical formulation is administered via bolus subcutaneous injection.

(46) The method of (45), wherein the bolus subcutaneous injection provides a duration of peak effects of about 30 minutes to about 120 minutes after being administered.

(47) An injectable pharmaceutical formulation, comprising:
a psychopharmaceutical agent;
a carboxymethyl cellulose salt; and
an aqueous vehicle;
wherein the psychopharmaceutical agent is a pharmaceutically acceptable salt of a compound of Formula (I) or a stereoisomer, solvate, or prodrug thereof,

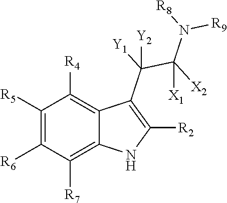

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, unsubstituted or substituted alkoxy, unsubstituted or substituted alkylthio, and unsubstituted or substituted acyloxy;
R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl; and
R₈ and R₉ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl,
or alternatively R₈ and R₉ together with the nitrogen atom attached thereto are optionally joined to form an unsubstituted or substituted heterocycloalkyl.

(48) The injectable pharmaceutical formulation of (47), wherein the carboxymethyl cellulose salt is sodium carboxymethyl cellulose.

(49) The injectable pharmaceutical formulation of (47) or (48), wherein the carboxymethyl cellulose salt has a weight average molecular weight of about 50 kDa to about 450 kDa.

(50) The injectable pharmaceutical formulation of any one of (47) to (49), wherein a concentration of the carboxymethyl cellulose salt by weight per total volume of the pharmaceutical formulation is from about 0.55% to about 1% (w/v).

(51) A kit suitable for preparing the injectable pharmaceutical formulation of any one of (47) to (50), the kit comprising:
(a1) a first solution comprising the psychopharmaceutical agent and the aqueous vehicle; and
(b1) a second solution comprising the carboxymethyl cellulose salt and the aqueous vehicle.

(52) A method of treating a central nervous system (CNS) disorder and/or psychological disorder in a subject in need thereof, comprising administering to the subject a therapeutically effective amount the injectable pharmaceutical formulation of any one of (47) to (50).

(53) The method of (52), wherein the CNS disorder and/or psychological disorder is a substance use disorder.

(54) The method of (53), wherein the substance use disorder is alcohol use disorder.

(55) The method of (52), wherein the CNS disorder and/or psychological disorder is an anxiety disorder.

(56) The method of (55), wherein the anxiety disorder is generalized anxiety disorder (GAD).

(57) The method of (56), wherein the generalized anxiety disorder is comorbid with depression.

(58) The method of (55), wherein the anxiety disorder is social anxiety disorder.

(59) The method of (52), wherein the CNS disorder and/or psychological disorder is a depressive disorder.

(60) The method of (59), wherein the depressive disorder is major depressive disorder (MDD) or treatment-resistant depression (TRD).

(61) The method of (52), wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

(62) The method of any one of (52) to (61), wherein the injectable pharmaceutical formulation is administered via injection.

(63) The method of any one of (52) to (62), wherein the injectable pharmaceutical formulation is administered via subcutaneous injection.

(64) The method of any one of (52) to (63), wherein the injectable pharmaceutical formulation is administered via bolus subcutaneous injection.

(65) The method of any one of (52) to (64), wherein the bolus subcutaneous injection provides a duration of peak effects of about 30 minutes to about 120 minutes after being administered.

(66) An injectable pharmaceutical formulation, comprising:
a psychopharmaceutical agent;
a hyaluronate salt; and
an aqueous vehicle;
wherein the psychopharmaceutical agent is a pharmaceutically acceptable salt of ketamine, or a stereoisomer, solvate, or prodrug thereof.

(67) The injectable pharmaceutical formulation of (66), wherein the pharmaceutically acceptable salt is an inorganic salt.

(68) The injectable pharmaceutical formulation of (66) or (67), wherein the pharmaceutically acceptable salt is a hydrochloride salt.

(69) The injectable pharmaceutical formulation of any one of (66) to (68), wherein a concentration of the psychopharmaceutical agent by weight (in terms of free base equivalence) per total volume of the pharmaceutical formulation is about 1 mg/mL to about 100 mg/mL.

(70) The injectable pharmaceutical formulation of any one of (66) to (69), wherein a concentration of the psychopharmaceutical agent by weight (in terms of free base equivalence) per total volume of the pharmaceutical formulation is about 10 mg/mL to about 50 mg/mL.

(71) The injectable pharmaceutical formulation of any one of (66) to (70), wherein the hyaluronate salt is sodium hyaluronate.

(72) The injectable pharmaceutical formulation of any one of (66) to (71), wherein the hyaluronate salt has a weight average molecular weight of about 500 kDa to about 2,000 kDa.

(73) The injectable pharmaceutical formulation of any one of (66) to (72), wherein the hyaluronate salt has a weight average molecular weight of about 1,000 kDa to about 1,800 kDa.

(74) The injectable pharmaceutical formulation of any one of (66) to (73), wherein a concentration of the hyaluronate salt by weight per total volume of the pharmaceutical formulation is from about 0.1% to about 2% (w/v).

(75) The injectable pharmaceutical formulation of any one of (66) to (74), wherein a concentration of the hyaluronate salt by weight per total volume of the pharmaceutical formulation is from about 0.1% to about 1% (w/v).

(76) The injectable pharmaceutical formulation of any one of (66) to (75), wherein a concentration of the hyaluronate salt by weight per total volume of the pharmaceutical formulation is from about 0.1% to about 0.75% (w/v).

(77) The injectable pharmaceutical formulation of any one of (66) to (76), wherein a concentration of the hyaluronate salt by weight per total volume of the pharmaceutical formulation is from about 0.1% to about 0.5% (w/v).

(78) The injectable pharmaceutical formulation of any one of (66) to (77), wherein the aqueous vehicle comprises water and sodium chloride.

(79) The injectable pharmaceutical formulation of (78), wherein the pharmaceutical formulation comprises sodium chloride at a concentration, in terms of weight per total volume of the pharmaceutical formulation, of about 0.1% to about 0.8% (w/v).

(80) The injectable pharmaceutical formulation of any one of (66) to (79), which has a pH of about 3 to about 7.

(81) The injectable pharmaceutical formulation of any one of (66) to (80), which has an osmolality of about 150 mOsm/kg to about 600 mOsm/kg.

(82) The injectable pharmaceutical formulation of any one of (66) to (81), which has a viscosity of less than about 3,000 cP.

(83) The injectable pharmaceutical formulation of any one of (66) to (82), which is suitable for subcutaneous injection.

(84) The injectable pharmaceutical formulation of any one of (66) to (83), which is suitable for bolus subcutaneous injection.

(85) The injectable pharmaceutical formulation of any one of (66) to (84), wherein the injectable pharmaceutical formulation provides a duration of peak effects of about 30 minutes to about 120 minutes after being administered to a human subject via bolus subcutaneous injection.

(86) A kit suitable for preparing the injectable pharmaceutical formulation of any one of (66) to (85), the kit comprising:
(a1) a first solution comprising the psychopharmaceutical agent and the aqueous vehicle; and
(b1) a second solution comprising the hyaluronate salt and the aqueous vehicle.

(87) A method of treating a central nervous system (CNS) disorder and/or psychological disorder in a subject in need thereof, comprising administering to the subject a therapeutically effective amount of the injectable pharmaceutical formulation of any one of (66) to (85).

(88) Use of the injectable pharmaceutical formulation of any one of (1) to (31) for treating a patient with a central nervous system (CNS) disorder and/or psychological disorder.

(89) The injectable pharmaceutical formulation of any one of (1) to (31) for use in therapy.

(90) Use of the injectable pharmaceutical formulation of any one of (47) to (50) for treating a patient with a central nervous system (CNS) disorder and/or psychological disorder.

(91) The injectable pharmaceutical formulation of any one of (47) to (50) for use in therapy.

(92) Use of the injectable pharmaceutical formulation of any one of (66) to (85) for treating a patient with a central nervous system (CNS) disorder and/or psychological disorder.

(93) The injectable pharmaceutical formulation of any one of (66) to (85) for use in therapy.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIG. 1 illustrates a general synthetic route for making Compounds of Formula (I), e.g., compounds I-2 and I-6;

FIG. 2 illustrates a general synthetic route for making Compounds of Formula (I), e.g., compounds I-1, I-4, I-5, and I-8;

FIG. 3 shows the percent drug release versus time profile of Formulations 1-6 compared to control when subjected to the Dialysis—Drug Release Test;

FIG. 4 shows the percent drug release versus time profile of Formulation 7 compared to control when subjected to the Dialysis—Drug Release Test;

FIG. 5 shows the percent drug release versus time profile of Formulation 8 compared to control when subjected to the Dialysis—Drug Release Test;

FIG. 6 shows the percent drug release versus time profile of Formulation 9 compared to control when subjected to the Dialysis—Drug Release Test;

FIGS. 7A and 7B show the percent drug release versus time profile of Formulations 10-12 compared to control when subjected to the Dialysis—Drug Release Test (FIG. 7A) and the corresponding first order release kinetic plot (FIG. 7B);

FIGS. 8A and 8B show the percent drug release versus time profile of Formulations 13-15 compared to control when subjected to the Dialysis—Drug Release Test (FIG. 8A) and the corresponding first order release kinetic plot (FIG. 8B);

FIGS. 9A and 9B show the percent drug release versus time profile of Formulations 16-18 compared to control when subjected to the Dialysis—Drug Release Test (FIG. 9A) and the corresponding first order release kinetic plot (FIG. 9B);

FIGS. 10A and 10B show the percent drug release versus time profile of Formulations 19-21 compared to control when subjected to the Dialysis—Drug Release Test (FIG. 10A) and the corresponding first order release kinetic plot (FIG. 10B);

FIG. 11 shows the percent drug release versus time profile of Formulations 22-27 compared to control when subjected to the Dialysis—Drug Release Test;

FIGS. 12A and 12B show individual DMT-d₁₀ plasma concentration-time curves in male Beagle dogs (animal IDs: 068M, 069M, 070M) after subcutaneous administration of DMT-d₁₀ at 0.1 mg/kg from control (FIG. 12A) and Formulation 28 (FIG. 12B);

FIGS. 13A and 13B show mean DMT-d₁₀ plasma concentration-time curves in male Beagle dogs after subcutaneous administration of DMT-d₁₀ at 0.1, 0.5, and 1 mg/kg from Formulations 28-30, respectively, in linear scale (FIG. 13A) and log scale (FIG. 13B);

FIG. 14 shows the dose proportionality of Cₘₐₓ and AUC_{inf} from the mean DMT-d₁₀ plasma concentration-time curves in male Beagle dogs after subcutaneous administration of DMT-d₁₀ at 0.1, 0.5, and 1 mg/kg from Formulations 28-30, respectively;

FIG. 15 shows the effect of sodium hyaluronate concentration on mean concentration-time profiles of DMT-d₁₀ after SC administration of DMT-d₁₀ at a dose of 1 mg/kg from Formulations 30-33; *Formulation 31 dosed at 0.5 mg/kg free base was dose adjusted to 1 mg/kg;

FIG. 16 shows the effect of sodium hyaluronate concentration on mean residence time (MRT_{inf}) of DMT-d₁₀ after SC administration of DMT-d₁₀ at a dose of 1 mg/kg from Formulations 30-33; *Formulation 31 dosed at 0.5 mg/kg free base was dose adjusted to 1 mg/kg;

FIG. 17 shows the effect of sodium hyaluronate concentration on plasma half-life (t_{1/2}) of DMT-d₁₀ after SC administration of DMT-d₁₀ at a dose of 1 mg/kg from Formulations 30-33; *Formulation 31 dosed at 0.5 mg/kg free base was dose adjusted to 1 mg/kg;

FIG. 18 shows the effect of sodium hyaluronate concentration on maximum plasma concentration (Cₘₐₓ) of DMT-d₁₀ after SC administration of DMT-d₁₀ at a dose of 1 mg/kg from Formulations 30-33; *Formulation 31 dosed at 0.5 mg/kg free base was dose adjusted to 1 mg/kg;

FIG. 19 shows the effect of sodium hyaluronate concentration on total exposure (AUC_{inf}) of DMT-d₁₀ after SC administration of DMT-d₁₀ at a dose of 1 mg/kg from Formulations 30-33; *Formulation 31 dosed at 0.5 mg/kg free base was dose adjusted to 1 mg/kg;

FIGS. 20A and 20B show mean DMT-d₁₀ plasma concentration-time curves in male Beagle dogs after subcutaneous administration of DMT-d₁₀ at 1 mg/kg free base from Formulation 34 (dose solution concentration of 20 mg/mL nominal delivered at dosing volume of 0.05 mL/kg) compared to control (dose solution concentration of 4 mg/mL nominal delivered at dosing volume of 0.25 mL/kg) in linear scale (FIG. 20A) and log scale (FIG. 20B);

FIGS. 21A and 21B show mean DMT and DMT-d₁₀ plasma concentration-time curves in male Beagle dogs after subcutaneous co-dose administration of DMT and DMT-d₁₀ at 0.5 mg/kg/analyte free base from Formulation 35 in linear scale (FIG. 21A) and log scale (FIG. 21B); and

FIG. 22 shows the percent drug release (ketamine) versus time profile of Formulations 36-38 compared to control when subjected to the Dialysis—Drug Release Test.

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

“Alkyl” refers to monovalent saturated aliphatic hydrocarbyl groups having from 1 to 10 carbon atoms and such as 1 to 6 carbon atoms, or 1 to 5, or 1 to 4, or 1 to 3, or 1 to 2 carbon atoms.

This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl (t-Bu)((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 10 substituents selected from the group consisting of deuterium, alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R′ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups having from 1 to 6, including, for example, 1 to 3 carbon atoms that are either straight-chained or branched, and which are optionally interrupted with one or more groups selected from —O—, —NR¹⁰—, —NR¹⁰C(O), —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms, for example 2 to 4 carbon atoms and having at least 1, for example from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms, for example, 2 to 3 carbon atoms and having at least 1 and for example, from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclohexyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from deuterium, alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and for example, from 1 to 2 double bonds.

The term “substituted cycloalkenyl” refers to cycloalkenyl groups having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzo[d][1,3]oxathiole, benzo[d][1,3]dioxole, and the like.

Unless otherwise constrained by the definition for the heterocyclic substituent, such heterocyclic groups can be optionally substituted with 1 to 5, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—. “Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, deuterium, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the disclosure and the other a typical counter ion such as chloride, or two ionized compounds disclosed herein can serve as counter ions for such divalent alkali earth ions, or a doubly ionized compound of the disclosure can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, deuterium, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S-M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in some embodiments, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein, unless specified otherwise. In such cases, the maximum number of such substitutions is three. For example, serial substitutions of substituted aryl groups specifically contemplated herein are limited to substituted aryl-(substituted aryl)-substituted aryl. However, substituent groups defined as e.g., polyethers may contain serial substitution greater than three, e.g., —O—(CH₂CH₂O)ₙ—H, where n can be 1, 2, 3, or greater.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

When it is stated that a substituent or group “comprise(s) deuterium” or is “comprising deuterium,” it is to be understood that the substituent or group may itself be deuterium, or the substituent or group may contain at least one deuterium substitution in its chemical structure. For example, when substituent “—R” is defined to comprise deuterium, it is to be understood that —R may be -D (-deuterium), or a group such as —CD₃ that is consistent with the other requirements set forth of —R.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrase “pharmaceutically acceptable salt” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like. When in the form of a solid, salts designated as “hemi-” salts indicate that the stoichiometry of subject compound to counterion is about 2:1, whereas solid salt forms without the “hemi-” descriptor possess a subject compound to counterion stoichiometry of about 1:1. For example, DMT hemi-fumarate indicates that the ratio of DMT to fumarate is 2:1, whereas DMT fumarate indicates that the ratio of DMT to fumarate is 1:1.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

A “crystalline” solid is a type of solid whose fundamental three-dimensional structure contains a highly regular pattern of atoms or molecules—with long range order—forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity—less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of a known standard or an internal standard. Other characterization techniques, such as differential scanning calorimetry (DSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

When referencing X-ray powder diffraction (XRPD) patterns of materials of the present disclosure, the phrase “characterized by an X-ray powder diffraction pattern containing at least three characteristic peaks at diffraction angles (2θ±0.2°) selected from . . . ” should be understood to include those materials characterized as having 3, 4, 5, 6, 7, 8, 9, 10, 11, 12, 13, 14, 15 or more (including all) of the recited characteristic XRPD diffraction peaks. Further, this phrase is intended to be open to the inclusion of other XRPD diffraction peaks not recited.

It will be appreciated that the compounds herein can exist in different salt, solvate, stereoisomer, crystalline/amorphous (including polymorphic) forms, and the present disclosure is intended to include all permutations thereof, such as a solvate of a pharmaceutically acceptable salt of a stereoisomer of the subject compound.

The language “tamper resistant” is art-recognized to describe aspects of a drug formulation that make it more difficult to use the formulation to abuse the drug moiety of the formulation through e.g., extraction for intravenous use, or crushing for freebase use; and therefore, reduce the risk for abuse of the drug.

The term “stable,” “stability,” and the like, as used herein includes chemical stability and solid state (physical) stability. The term “chemical stability” means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g., hydration, dehydration, solvatization, desolvatization, crystallization, recrystallization or solid-state phase transition).

As used herein, the term “composition” is equivalent to the term “formulation.”

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating one or more symptoms of the disease or medical condition in a patient. A treatment can provide a therapeutic benefit such as the eradication or amelioration of one or more of the physiological or psychological symptoms associated with the underlying condition, disease, or disorder such that an improvement is observed in the patient, notwithstanding the fact that the patient may still be affected by the condition. In some embodiments, treatment may refer to prophylaxis, i.e., preventing the disease or medical condition from occurring or otherwise delaying the onset of the disease or medical condition in a patient.

A “patient” or “subject,” used interchangeably herein, can be any mammal including, for example, a human. A patient or subject can have a condition to be treated or can be susceptible to a condition to be treated.

A “psychopharmaceutical agent” is a chemical substance with the ability to cross the blood-brain barrier and act on the nervous system, resulting in alterations in perception, mood, consciousness, cognition, and/or behavior. Categories of psychopharmaceutical agents include anxiolytics (e.g., benzodiazepines, barbiturates, etc.), empathogen-entactogens (e.g., MDMA, MDA, AMT, etc.), stimulants (e.g., amphetamines, modafinil, etc.), depressants (e.g., sedatives, hypnotics, and opioids), and hallucinogens such as psychedelics, dissociatives, and deliriants (e.g., psilocybin, LSD, DMT, mescaline, salvia divinorum, scopolamine, etc.).

As used herein, and unless otherwise specified, the terms “manage,” “managing” and “management” refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the term “managing” encompasses treating a subject who had suffered from the particular disease, disorder, or condition in an attempt to prevent or minimize the recurrence of the disease, disorder, or condition, or of one or more symptoms thereof.

“Therapeutically effective amount” refers to an amount of a compound(s) or its salt form sufficient to treat a specified disorder or disease or one or more of its symptoms and/or to prevent the occurrence of the disease or disorder (prophylactically effective amount). As used herein, and unless otherwise specified, a “prophylactically effective amount” of an active agent, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

As used herein, a “bolus” is where a discrete amount of active pharmaceutical ingredient (API) (e.g., a psychopharmaceutical agent) is administered (e.g., by injection) within 30 minutes or less such that the concentration of the API in the body quickly increases. Bolus injections are typically administered intravenously (directly into the vein), intramuscularly (within the muscle), intradermally (beneath the skin), or subcutaneously (within the fat or skin). A bolus injection thus differs from an “infusion,” whereby a discrete amount of API (e.g., a psychopharmaceutical agent) is administered by single injection or multiple injections over a prolonged period of greater than 30 minutes, such that the concentration of the API in the body follows a more stable kinetic profile, in some cases reaching a steady-state, with a prolonged exposure period.

The language “toxic spikes” is used herein to describe spikes in concentration of any compound described herein that would produce side-effects of sedation or psychotomimetic effects, e.g., hallucination, dizziness, and nausea; which can not only have immediate repercussions, but also influence treatment compliance. In particular, side effects may become more pronounced at blood concentration levels above about 300 ng/mL (e.g. above about 300, 400, 500, 600 or more ng/mL).

According to IUPAC, “osmolality” is the quotient of the negative natural logarithm of the rational activity of water and the molar mass of water. In simpler terms, osmolality is an expression of the number of osmotically active particles (the number of solute particles) in 1 kg of a solution, represented herein as the number of milliosmoles (mOsm) per 1 kg of solution. Thus, osmolality is a function only of the number of particles, and is not related to particle molecular weight, size, shape, or charge (see D. K. Faria et al., M. E. Mendes and N. M. Sumita, J. Bras. Pata!. Med. Lab., 53, 1, 38-45 (2017) for a review of the measurement of serum osmolality). For example, one mole of a nondissociating substance (e.g., glucose) dissolved in 1 kg of water has an osmolality of 1 Osm/kg (1000 mOsm/kg), whilst one mole of a substance that dissociates into two separate species in solution (e.g., sodium chloride) dissolved in 1 kg of water has an osmolality of 2 Osm/kg (2000 mOsm/kg).

Where solutions are defined herein to be “isotonic” with one another, the solutions have the same osmolality. For example, where a formulation is defined to be isotonic with human blood serum, the formulation has the same osmolality as human blood serum. Human blood serum typically has an osmolality of about 275 to about 300 mOsm/kg (L. Hooper et al., BMJ Open, 2015; 5(10):e008846).

“Syringeable” or “syringeability” refers to the force required to inject a given solution at a given rate via a chosen needle length and gauge, and relates to whether the formulation is administrable through a syringe. Flow through a hollow needle is characterized by the Hagen-Poiseuille equation (1):

$$F = \frac{128 Q μ L A}{π {D}^{4}} \qquad ( 1 )$$

where F=syringe stopper (plunger) force, Q=volumetric flow rate, μ=dynamic viscosity, L=needle length, D=needle bore diameter, and A=syringe area.

Unless stated otherwise, concentrations expressed in terms of weight per volume (w/v) are calculated from grams (g) per milliliter (mL). These concentrations may be expressed as a percentage (% w/v), for example, the concentration of 1 g of solute in 100 mL of a solution is 1% w/v.

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, schizophrenia, cognitive deficits in schizophrenia, attention deficit disorder, attention deficit hyperactivity disorder, cognitive deficit disorders, seizures, palsies, headache disorders, addictions, eating disorders, anger, bipolar and manic disorders, depression disorders, anxiety disorders, or any combinations thereof.

“Inflammatory conditions” or “inflammatory disease,” as used herein, refers broadly to chronic or acute inflammatory diseases. Inflammatory conditions and inflammatory diseases include, but are not limited to, rheumatic diseases (e.g., rheumatoid arthritis, osteoarthritis, psoriatic arthritis); spondyloarthropathies (e.g., ankylosing spondylitis, reactive arthritis, Reiter's syndrome); crystal arthropathies (e.g., gout, pseudogout, calcium pyrophosphate deposition disease); multiple sclerosis; Lyme disease; polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, glaucoma, cataracts, or any combinations thereof.

All diseases and disorders listed herein may be defined as described in the Diagnostic and Statistical Manual of Mental Disorders (DSM-5), published by the American Psychiatric Association, or in International Classification of Diseases (ICD), published by the World Health Organization.

As used herein “adjunctive therapy,” “adjuvant therapy,” and the like, refers to a therapy that is given in addition to a primary or initial therapy to improve or maximize effectiveness. For example, a subject diagnosed with a depressive disorder that is taking one or more antidepressant medications (e.g., an SSRI) as a primary or initial therapy, but has an inadequate response to or has otherwise failed to achieve a desirable outcome with the antidepressant medication may be administered, as “adjunctive therapy,” a pharmaceutical formulation of the present disclosure, to improve or maximize treatment effectiveness. In this example with a depressive disorder, the adjunctive therapy may improve or maximize treatment effectiveness by reducing depressive symptom(s) compared to the primary or initial therapy alone. The primary or initial therapy and the adjunctive therapy involving the pharmaceutical formulation of the present disclosure, can be, but need not be, prescribed and/or administered by the same person (e.g., clinician). For example, the primary or initial therapy (e.g., SSRI therapy) may be prescribed by a first clinician and self-administered by the patient, while the adjunctive therapy involving a pharmaceutical formulation of the present disclosure, may be prescribed and/or administered by a second clinician. Alternatively, the primary or initial therapy (e.g., SSRI therapy) may be prescribed by a first clinician and self-administered by the patient, while the adjunctive therapy involving a pharmaceutical formulation of the present disclosure, may be prescribed and/or administered by the same (first) clinician.

The term “inadequate response” as used herein refers to a lack of clinically meaningful improvement in symptoms, for example as measured by one or more of the rating scales described herein. The inadequate response to an adequate course of treatment with an antidepressant medication(s) may be determined retrospectively or prospectively. Prospective determination of inadequate response refers to a determination made by the prescribing clinician or therapist following administration of part of a course of treatment. Retrospective determination refers to a determination made by the prescribing clinician or therapist following administration of a full adequate course of treatment.

As used herein, “peak effects,” “peak psychedelic effects,” “peak experiences,” and the like, refer to subjective effects on perception and consciousness which are intense—peak effects normally correlate with a score of ≥70 mm on the VAS any drug effect rating scale. In the case of short-acting tryptamine psychedelics such as DMT, 5-MeO-DMT, and their analogs (e.g., deuterated analogs), peak effects in humans are associated with a drug plasma concentration of ≥40 ng/mL.

The phrase “effect size” refers to a statistical calculation that can be used to compare the efficacy of different agents by quantifying the size of the difference between treatments (“between-group”). It is a dimensionless measure of the difference in outcomes under two different treatment interventions. Effect sizes thus inform clinicians about the magnitude of treatment effects. Unless stated otherwise, as used herein, the effect size is calculated from the difference between the mean within-group efficacy endpoint change resulting from a treatment group (e.g., administration of a pharmaceutically acceptable salt of a compound of Formula (I) through (III)) and the mean within-group efficacy endpoint change resulting from placebo, using Cohen's d method with the following formula:

$$Cohen ’ s d = \frac{\begin{matrix}Mean endpoint change from treatment - \\ Mean endpoint change from placebo\end{matrix}}{Pooled standard deviation for entire sample}$$

Cohen's d is also known as the standardized mean difference. An effect size (Cohen's d score) of zero means that the treatment and placebo have no differences in effect. An effect size (Cohen's d score) greater than zero indicates the degree to which treatment is more efficacious than placebo. Conventionally, it is considered that an effect size (Cohen's d score) of 0.2 is small, 0.5 is medium, and 0.8 or higher is large.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value may vary up or down by 5%. For example, for a value of about 100, means 95 to 105 (or any value between 95 and 105).

### Pharmaceutical Formulation

Disclosed herein is an injectable pharmaceutical formulation comprising a psychopharmaceutical agent or drug, a release modifier such as a hyaluronate salt or a carboxymethyl cellulose salt, and an aqueous vehicle.

The pharmaceutical formulation is suitable for injection, thus its administration in therapy typically comprises parenteral injection of the formulation through the skin or other external boundary tissue, rather than through the alimentary canal, so that the active pharmaceutical ingredient(s) contained therein is administered, using gravity or force, directly into a blood vessel, organ, tissue, or lesion. To be suitable for injection, i.e., “injectable” or “injectability,” it is meant that the formulation is in accordance with Pharmacopeial requirements of injections, for example as set forth in The United States Pharmacopeial (USP) Convention, General Requirements/<1> Injections, 33. For example, the injectable pharmaceutical formulations are prepared by methods designed to ensure that they meet Pharmacopeial requirements for sterility, pyrogens, particulate matter, and other contaminants, and where appropriate, contain inhibitors of the growth of organisms (e.g., antimicrobial preservatives) and/or anti-oxidants. Examples of Pharmacopeial requirements include, but are not limited to, USP Pyrogen Test <151>, USP Bacterial Endotoxins Test <85>, USP Antimicrobial Effectiveness Testing <51>, USP Antimicrobial Agents-Content <341>, USP Sterilization and Sterility Assurance of Compendial Articles <1211>, USP Particulate Matter in Injections <788>, and USP Sterility Tests <71>. For instance, to be suitable for injection, inter alia, the endotoxin limit defined on the basis of dose is equal to K/M, where K is the threshold human pyrogenic dose of endotoxin per kg of body weight, and M is equal to the maximum recommended human dose of product per kg of body weight in a single hour period; Escherichia coli should be absent in 1 g of formulation; the total aerobic microbial count (TAMC) should be under 1,000 colony forming units (CFU)/g; and the total yeast and mold count (TYMC) should not exceed 100 CFU/g.

To be suitable for injection, i.e., “injectable” or “injectability,” it is further meant that the pharmaceutical formulation is characterized as having physiochemical properties, such as pH, osmolality, and viscosity, which enables administration through the skin or other external boundary tissue via needle, syringe, canula, catheter, or other suitable injection device without causing excessive tissue necrosis, pain, or inflammation (e.g., phlebitis) at the injection site. Injectable drug products generally have a pH of about 2 to 11 for IV and intramuscular injection, and a pH of about 3 to 9 for subcutaneous injection (Usach I, et al. Subcutaneous Injection of Drugs: Literature Review of Factors Influencing Pain Sensation at the Injection Site. Adv Ther. 2019 November; 36(11):2986-2996). pH values which are too high are associated with tissue necrosis, whereas pH values which are too low are associated with pain and inflammation at the injection site. Injectable drug products generally have an osmolality of 150 to 600 mOsm/kg, with osmolalities closest to that of human blood serum (275 to about 300 mOsm/kg) being preferred. Hypertonic injection solutions with an osmolality above 600 mOsm/kg have been reported to possibly cause crenation of red blood cells and significant pain, while hypotonic solutions with an osmolality below 150 mOsm/kg may cause hemolysis and pain at the injection site (Roethlisberger D. et al. If Euhydric and Isotonic Do Not Work, What Are Acceptable pH and Osmolality for Parenteral Drug Dosage Forms?, Journal of Pharmaceutical Sciences, 106(2), 2017, 446-456). Injectable drug products administered with common syringes and needle gauge generally have a viscosity of less than about 50 centipoise (cP), with higher viscosities sometimes requiring injection forces too high for common syringes and gauge needles to withstand. The drug may not even be administrable through a syringe (may not be “syringeable”). However, some injectable drug products with much higher viscosities (>100 to 10,000+ cP) can be administered by injection, for example, when administering non-Newtonian fluids or when using injection devices designed for high viscosity fluids such as auto-injectors for high viscosity fluids. The pH, osmolality, and viscosity of the pharmaceutical formulation of the present disclosure fall within the ranges reported to be suitable for injection, and suitable for subcutaneous injection in particular.

The pharmaceutical formulation is typically in the form of a solution, although other dosage forms are also contemplated such as suspensions, emulsions, micelles, liposomes, microspheres, and nanosystems which are suitable for injection. Solid forms which are suitable for solutions or suspensions in liquid prior to injection are also disclosed. In some embodiments, the pharmaceutical formulations are disclosed as ready-to-use sterile solutions. In some embodiments, the pharmaceutical formulations are disclosed as reconstituted solutions prepared from sterile dry soluble products, including lyophilized powders and hypodermic tablets, reconstituted with an aqueous vehicle prior to use. In some embodiments, the pharmaceutical formulations are disclosed as ready-to-use sterile suspensions. In some embodiments, the pharmaceutical formulations are disclosed as reconstituted solutions prepared from sterile dry insoluble products reconstituted with an aqueous vehicle prior to use. In some embodiments, the pharmaceutical formulations are disclosed as ready-to-use sterile emulsions.

In some embodiments, the injectable pharmaceutical formulation is suitable for intravenous administration (directly into the vein), i.e., is an intravenous pharmaceutical formulation. In some embodiments, the injectable pharmaceutical formulation is suitable for intramuscular administration (within the muscle), i.e., is an intramuscular pharmaceutical formulation. In some embodiment, the injectable pharmaceutical formulation is suitable for intradermal administration intradermally (beneath the skin), i.e., is an intradermal pharmaceutical formulation. In some embodiments, the injectable pharmaceutical formulation is suitable for subcutaneous administration (within the fat or the layer of skin directly below the dermis and epidermis), i.e., is a subcutaneous pharmaceutical formulation.

Subcutaneous administration is a minimally invasive mode of administration. Subcutaneous tissue has few blood vessels and so drugs injected into it are intended for slow, sustained rates of absorption, often with some amount of depot effect. Compared with other routes of administration, it is slower than intravenous and intramuscular injections but still faster than intradermal injections. The convenience and speed of subcutaneous delivery allows increased patient compliance and quicker access to medication when needed. Subcutaneous administration can be performed by injection or by implantation of a sustained or timed-release device beneath the surface of the skin. The site of the injection or device can be rotated when multiple injections or devices are needed. Subcutaneous formulations are usually much easier to handle for both the patient and practitioner. A particular advantage of the subcutaneous delivery route in the therapeutic methods of the present disclosure is that it allows the medical practitioner to perform the administration in a rather short intervention with the patient, compared to intravenous infusion protocols associated with DMT-based therapy. Moreover, the patient can be trained to perform self-administration. Such self-administration may be particularly useful during maintenance dosing where clinical observation and/or psychotherapy may play less prominent roles in the overall treatment. Usually, injection volumes of up to about 3 mL are tolerated via the subcutaneous route, especially those given in the patient's abdomen, with injection volumes of about 2 mL or less being well tolerated across various injection sites. Large subcutaneous injection volumes greater than about 3 mL are often associated with pain. For patients requiring multiple doses, several unit dose formulations can be injected at multiple sites of the body surface. Particular pharmacopeial requirements for subcutaneous injections include, but are not limited to, passing specifications of USP Particulate Matter in Injections <788>.

The pharmaceutical formulation may be suitable for bolus injection, in which a discrete amount of the psychopharmaceutical agent is administered by injection within 30 minutes or less, 25 minutes or less, 20 minutes or less, 15 minutes or less, 10 minutes or less, 5 minutes or less, 4 minutes or less, 3 minutes or less, 2 minutes or less, 1 minute or less, 30 seconds or less, 20 seconds or less, 10 seconds or less, or 5 seconds or less. The bolus injection may involve a single injection or multiple injections performed within the above-described time range. Thus, administering multiple bolus injections within the above-mentioned time range of 30 minutes or less (e.g., two injections lasting 30 seconds each, administered within 5 minutes of one another) would be considered a bolus administration herein. In some embodiments, the bolus injection involves a single injection within the above time range. The pharmaceutical formulation may be suitable for bolus subcutaneous injection, such as a single bolus subcutaneous injection, i.e., the pharmaceutical formulation is a bolus subcutaneous pharmaceutical formulation. The pharmaceutical formulation may be suitable for bolus intramuscular injection, such as a single bolus intramuscular injection, i.e., the pharmaceutical formulation is a bolus intramuscular pharmaceutical formulation. The pharmaceutical formulation may be suitable for bolus intradermal injection, such as a single bolus intradermal injection, i.e., the pharmaceutical formulation is a bolus intradermal pharmaceutical formulation. The pharmaceutical formulation may be suitable for bolus intravenous injection, such as a single bolus intravenous injection, i.e., the pharmaceutical formulation is a bolus intravenous pharmaceutical formulation.

The pharmaceutical formulation may be suitable for infusion injection, in which a discrete amount of the psychopharmaceutical agent is administered by injection over a prolonged period of greater than 30 minutes, greater than 40 minutes, greater than 50 minutes, greater than 60 minutes, greater than 70 minutes, greater than 80 minutes, greater than 90 minutes, greater than 100 minutes, greater than 110 minutes, greater than 120 minutes. The infusion injection may involve a single prolonged injection, or multiple injections (short or prolonged) within the above-described time range. Thus, administering multiple bolus injections over a prolonged period of greater than 30 minutes would be considered an infusion administration herein. In some embodiments, the infusion injection involves a single injection within the above time range. The pharmaceutical formulation may be suitable for infusion subcutaneous injection. The pharmaceutical formulation may be suitable for infusion intramuscular injection. The pharmaceutical formulation may be suitable for infusion intravenous injection.

### Psychopharmaceutical Agent

The injectable pharmaceutical formulation comprises a psychopharmaceutical agent. The psychopharmaceutical agent may be an anxiolytic (e.g., benzodiazepines, barbiturates, etc.), an empathogen-entactogen (e.g., MDMA, MDA, AMT, etc.), a stimulant (e.g., amphetamines, modafinil, etc.), a depressant (e.g., sedatives, hypnotics, and opioids), and/or a hallucinogen such as a psychedelic (e.g., a tryptamine psychedelic), a dissociative, or a deliriant (e.g., psilocybin, LSD, DMT, mescaline, salvia divinorum, scopolamine, etc.). The psychopharmaceutical agent may be a free base compound, or a pharmaceutically acceptable salt of the free base compound. Combinations of psychopharmaceutical agents may also be used.

In some embodiments, the psychopharmaceutical agent is a dissociative, a dissociative hallucinogen, an anesthetic, an arylcyclo-hexylamine, a 1,2-diarylethylamine, a j-keto-arylcyclohexylamine, and/or a compound that modulates the NMDA receptor. Examples of such psychopharmaceutical agents include, but are not limited to, ketamine, methoxetamine, deschloroketamine, N-ethyl deschloroketamine (eticyclidone), 3-methoxyphencyclidine, methoxieticyclidine, ephenidine, lanicemine, dextromethorphan, dextrorphan, methoxyketamine, norketamine (e.g., (R)-norketamine, (S)-norketamine, or mixtures thereof), hydroxynorketamine (e.g., 2R,6R-hydroxynorketamine, 2S,6S-hydroxynorketamine, or mixtures thereof), or a pharmaceutically acceptable salt, a stereoisomer, solvate, or prodrug thereof, or a combination thereof.

In some embodiments, the psychopharmaceutical agent is an opioid. Examples of opioids include, but are not limited to, racemorphan, levorphanol, racemethorphan, buprenorphine, morphine, loperamide, morphine, codeine, hydrocodone, oxymorphone, buprenorphine, fentanyl, methadone, tramadol, alpha-methyl acetyl fentanyl, alfentanil, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, 3-methylthiofentanyl, naloxone, and naltrexone, or a pharmaceutically acceptable salt, a stereoisomer, solvate, or prodrug thereof, or a combination thereof.

In some embodiments, the psychopharmaceutical agent is a cathinone, a 3,4-methylenedioxyamphetamine compound, an aminoalkyl-substituted benzofuran, a substituted amphetamine, an aminoindane, a stimulant, diphenhydramine, hydroxazine, phenylephrine, dopamine, adrenaline, lidocaine, oxymetazoline, clemastine, chlorpheniramine, or 6-chloro-2-aminotetralin. In some embodiments, the pharmaceutical compound is a cathinone, an aminoalkyl-substituted benzofuran, and an aminoindane, or a pharmaceutically acceptable salt, a stereoisomer, solvate, or prodrug thereof.

In some embodiments, the psychopharmaceutical agent is a lysergamide. Examples of lysergamides include, but are not limited to, methylisopropyllysergamide, ethylisopropyllysergamide, 6-allyl-6-nor-LSD, 6-ethyl-6-nor-lysergic acid diethylamide, 1-acetyl-LSD, 1-propionyl-6-ethyl-6-nor-lysergic acid diethylamide, 1-propionyl-lysergic acid diethylamide, 1-cyclopropionyl-d-lysergic acid diethylamide, N1-butyryl-lysergic acid diethylamide, and 6-propyl-6-nor-lysergic acid diethylamide, or a pharmaceutically acceptable salt, a stereoisomer, solvate, or prodrug thereof, or a combination thereof.

In some embodiments, the psychopharmaceutical agent is a phenethylamine. Examples of phenethylamines include, but are not limited to, mescaline, 2,5-dimethoxy-4-bromophenethylamine (2C-B), 2-(4-iodo-2,5-dimethoxyphenyl)ethan-1-amine (2C-I), 2-(4-chloro-2,5-dimethoxyphenyl)ethan-1-amine (2C-C), 2,5-dimethoxy-4-iodoamphetamine, 2-[2,5-dimethoxy-4-(propylsulfanyl)phenyl]ethan-1-amine, and 2-(4-iodo-2,5-dimethoxyphenyl)-N-[(2-methoxyphenyl)methyl]ethanamine, or a pharmaceutically acceptable salt, a stereoisomer, solvate, or prodrug thereof, or a combination thereof.

In some embodiments, the psychopharmaceutical agent is a tryptamine psychedelic. Examples of tryptamine psychedelics include, but are not limited to, N,N-dimethyltryptamine, N,N-diethyltryptamine, N,N-dipropyltryptamine, N-Methyl-N-propyltryptamine, N-methyl-N-isopropyltryptamine, N,N-diallyltryptamine, N-methyl-N-allyltryptamine, N-methyl-N-ethyltryptamine, N,N-diisopropyltryptamine, 4-hydroxy-N-methyl-N-ethyltryptamine, 5-methoxy-N,N-diisopropyltryptamine, 5-methoxy-N,N-dimethyltryptamine, O-acetylpsilocin, psilocin, as well as those tryptamine psychedelics described hereinafter, or a pharmaceutically acceptable salt, a stereoisomer, solvate, or prodrug thereof, or a combination thereof.

Examples of psychopharmaceutical agents which can be categorized as hallucinogens such as a psychedelic (e.g., a tryptamine psychedelic), a dissociative, or a deliriant include, but are not limited to, 9,10-didehydro-6-allyl-N,N-diethylergoline-8β-carboxamide, 9,10-didehydro-6,N,N-triethylergoline-8β-carboxamide, N,N-dimethyltryptamine, N,N-diethyltryptamine, 5-methoxy-N,N-dimethyltryptamine, N,N-dibutyltryptamine, N,N-diethyltryptamine, N,N-diisopropyltryptamine, N,N-dipropyltryptamine, N-methyl-N-propyltryptamine, N-methyl-N-isopropyltryptamine, N,N-diallyltryptamine, N-methyl-N-allyltryptamine, N-methyl-N-ethyltryptamine, 4-hydroxy-N-methyl-N-ethyltryptamine, 5-methoxy-N,N-diisopropyltryptamine, 5-Methoxy-α-methyltryptamine, 2,α-dimethyltryptamine, α,N-dimethyltryptamine, N,N-dipropyltryptamine, N-ethyl-N-isopropyltryptamine, α-ethyltryptamine, O-acetylpsilocin, psilocin, harmaline (7-methoxy-1-methyl-p-carboline), harmine (7-Methoxy-p-carboline), 4-hydroxy-diethyltryptamine and phosphate ester, 4-hydroxy-diisopropyltryptamine, 4-hydroxy-methyl-tryptamine, 4-hydroxy-tryptamine, 5-hydroxy-tryptamine, 4-hydroxy-dipropyltryptamine, 4-hydroxy-N-methyl-N-ethyl-tryptamine, 4-hydroxy-N-methyl-N-isopropyltryptamine, 4-hydroxy-N—N-tetramethylene-tryptamine, d-iso-LSD, I-LSD, I-iso-LSD, N,N-diisopropyl-4,5-methylenedioxy tryptamine, N,N-diisopropyl-5,6-methylenedioxy tryptamine, N,N-dimethyl-4,5-methylenedioxy tryptamine, N,N-dimethyl-5,6-methylenedioxy tryptamine, 2-methyl-DMT, 5-MeO-diethyltryptamine, 5-MeO-diisopropyltryptamine, 4-MeO—N-isopropyl-N-methyl-tryptamine, 5-MeO—N-isopropyl-N-methyl-tryptamine, 5-MeO-NMT, 5-MeO-2,N,N-trimetyltryptamine, N-isopropyl-N-methyl-tryptamine, alpha-methyltryptamine, alpha-methyl-4-OH-tryptamine, N-methyl-tryptamine, 5-MeO-α,N-dimethyl-tryptamine, 4-allyloxy-3,5-dimethoxyphenethylamine, 2,5-dimethoxy-4-methylthioamphetamine, 2,5-dimethoxy-4-ethylthioamphetamine, 2,5-dimethoxy-4-i-propylthioamphetamine, 2,5-dimethoxy-4-phenylthioamphetamine, 2,5-dimethoxy-4-n-propylthioamphetamine, 2,5-dimethoxy-α-ethyl-4-methylphenethylamine, 3,4-diethoxy-5-methoxy-phenethylamine, 4-n-butoxy-3,5-dimethoxyphenethylamine, 2,5-dimethoxy-4,N-dimethylamphetamine, 4-bromo-2,5-O-trimethoxyphenethylamine, 4-methyl-2,5,0-trimethoxyphenethylamine, β-methoxy-3,4-methylenedioxyphenethylamine, 3,4,5,0-tetramethoxyphenethylamine, 3,5-dimethoxy-4-bromoamphetamine, 2-bromo-4,5-methylenedioxyamphetamine, 4-bromo-2,5,dimethoxyphenethylamine, 4-benzyloxy-3,5-dimethoxyamphetamine, 2,5-dimethoxy-4-chlorophenethylamine, 2,5-dimethoxy-4-methylphenethylamine, 2,5-dimethoxy-4-ethylphenethylamine, 3,5-dimethoxy-4-ethoxyamphetamine, 2,5-dimethoxy-4-fluorophenethylamine, 2,5-dimethoxy-3,4-dimethylphenethylamine, 2,5-dimethoxy-3,4-(trimethylene)phenethylamine, 2,5-dimethoxy-3,4-(tetramethylene)phenethylamine, 3,6-dimethoxy-4-(2-aminoethyl)benzonorborane, 1,4-dimethoxynapthyl-2-ethylamine, 2,5-dimethoxyphenethylamine, 2,5-dimethoxy-4-iodoophenethylamine, 2,5-dimethoxy-4-nitrophenethylamine, 2,5-dimethoxy-4-i-propoxyphenethylamine, 2,5-dimethoxy-4-n-propoxyphenethylamine, 4-cyclopropyl-3,5-dimethoxyphenethylamine, 2,5-dimethoxy-4-methylseleneophenethylamine, 2,5-dimethoxy-4-methylthiophenethylamine, 2,5-dimethoxy-4-ethylthiophenethylamine, 2,5-dimethoxy-4-i-propylthiophenethylamine, 2,6-dimethoxy-4-i-propylthiophenethylamine, 2,5-dimethoxy-4-n-propylthiophenethylamine, 2,5-dimethoxy-4-cyclopropylmethylthiothiophenethylamine, 2,5-dimethoxy-4-t-butylthiophenethylamine, 2,5-dimethoxy-4-(2-methoxyrthylthio)phenethylamine, 2,5-dimethoxy-4-cyclopropylthiophenethylamine, 2,5-dimethoxy-4-s-butylthiophenethylamine, 2,5-dimethoxy-4-(2-fluorothio)phenethylamine, 2,5-dimethoxy-4-trideuteromethoxyphenethylamine, 2,4,5-trimethoxy-β,β-dideuterophenethylamine, 2,5-dimethoxy-4-methylphenethylamine, 2,4-dimethoxyamphetamine, 2,5-dimethoxyamphetamine, 2,4-dimethoxyamphetamine, 2,5-dimethoxy-3,4-methylenedioxyamphetamine, 2,5-dimethoxy-4-bromoamphetamine, 2,5-dimethoxy-4-chloroamphetamine, 2,5-dimethoxy-4-(2-fluoroethyl)-amphetamine, 2,5-dimethoxy-4-iodoamphetamine, 2,5-dimethoxy-4-methylamphetamine, 2,6-dimethoxy-4-methylamphetamine, 2,5-dimethoxy-4-n-propylamphetamine, 3,5-dimethoxy-4-ethoxyphenethylamine, 2,4,5-triethoxyamphetamine, 2,4-diethoxy-5-methoxyamphetamine, 2,5-diethoxy-4-methoxyamphetamine, 4,5-dimethoxy-2-ethoxyamphetamine, N-hydroxy-N-methyl-3,4-methylenedioxyamphetamine, 2,5-dimethoxy-3,4-(trimethylene)amphetamine, 3,6-dimethoxy-4-(2-aminopropyl)benzonorborane, 2,5-dimethoxy-3,4-dimethylamphetamine, 2,5-dimethoxy-4-ethylthio-N-hydroxyphenethylamine, 2,5-dimethoxy-N-hydroxy-4-n-propylthiophenethylamine, 2,5-dimethoxy-4-s-butylthio-N-hydroxyphenethylamine, 3,5-dimethoxy-4-i-propoxyphenethylamine, 5-ethoxy-2-methoxy-4-methylamphetamine, 2-amino-(3,4-methylenedioxyphenyl)butane, 3-methoxy-4,5-methylenedioxyphenethylamine, 3,4,5-trimethoxyphenethylamine, 3,5-dimethoxy-4-methalloxyphenethylamine, 3,4-methylenedioxyamphetamine, 3,4-methylenedioxy-N-ethylamphetamine, 3,4-methylenedioxy-N-methylamphetamine, 3,4-methylenedioxy-N-hydroxyamphtamine, 3,4-methylenedioxy-5-ethoxyphenethylamine, 2,5-dimethoxy-4-ethoxyapmphetamine, 3-methoxy-4-ethoxyphenethylamine, 2-methylamino-1-(3,4-methylenedioxyphenyl)butane, 3-methoxy-4,5-methylenedioxyamphetamine, 2-methoxy-4,5-methylenedioxyamphetamine, 2-methoxy-3,4-methylenedioxyamphetamine, 4-methoxy-2,3-methylenedioxyamphetamine, 3,5-dimethoxy-4-n-propoxyphenethylamine, 4-ethoxy-5-methoxy-3-methylthiophenethylamine, 3,5-dimethoxy-4-ethylthiophenethylamine, 3,4-dimethoxy-5-methylthiophenethylamine, 3,5-dimethoxy-5-methylthiophenethylamine, 3,4,5-trimethoxyamphetamine, 2,4,5-trimethoxyamphetamine, 2,3,5-trimethoxyamphetamine, 2,3,6-trimethoxyamphetamine, 2,4,6-trimethoxyamphetamine, 4,5-dimethoxy-3-ethylthiophenethylamine, 4-ethyl-2-methoxy-5-methylthioamphetamine, 5-methoxy-4-methyl-2-methylthioamphetamine, 2-methoxy-4-methyl-5-methylthioamphetamine, 2-methoxy-4-methyl-5-methylsulfinylamphetamine, 3,5-dimethoxy-4-n-propylthiophenethylamine, salvinorin A, ibotenic acid, muscimol, dextromethorphan, ketamine, esketamine (the S(+)-enantiomer of ketamine), norketamine (e.g., (R)-norketamine, (S)-norketamine, or mixtures thereof), hydroxynorketamine (e.g., 2R,6R-hydroxynorketamine, 2S,6S-hydroxynorketamine, or mixtures thereof), phencyclidine, dizocilpine (MK-801), scopolamine, hyoscyamine, aporphine, lysergic acid amide, cathine, cathinone, and voacangine, or a pharmaceutically acceptable salt, a stereoisomer, solvate, or prodrug thereof, or a combination thereof.

### Tryptamine Psychedelic

In some embodiments, the psychopharmaceutical agent is a tryptamine psychedelic. Tryptamine psychedelics generally share a basic core structure of an indole (a fused a fused benzene and pyrrole ring), and a 2-aminoethyl group at the second carbon (third aromatic atom, with the first one being the heterocyclic nitrogen), as represented below. Many tryptamine psychedelics are 5-HT_{2A} receptor agonists, i.e., they increase the activity of a 5-HT_{2A} receptor, which is a subtype of the 5-HT₂ receptor that belongs to the serotonin receptor family, and includes both partial and full agonists.

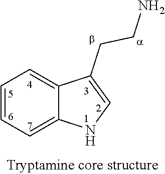 [SMILES: NCCc1c[nH]c2ccccc12]

In some embodiments, the tryptamine psychedelic is optionally substituted on the tryptamine ring. In some embodiments, the tryptamine psychedelic is an N,N-dialkyltryptamine. In some embodiments, the tryptamine psychedelic is N,N-dimethyltryptamine, N,N-diethyltryptamine, N,N-dipropyltryptamine, N-methyl-N-propyltryptamine, N-methyl-N-isopropyltryptamine, N,N-diallyltryptamine, N-methyl-N-allyltryptamine, N-methyl-N-ethyltryptamine, N,N-diisopropyltryptamine, wherein the tryptamine is optionally substituted, or a combination thereof. In some embodiments, the tryptamine psychedelic is substituted with one or more deuterium atoms. In some embodiments, the tryptamine psychedelic is optionally substituted at the 4- or 5-position of the tryptamine ring with a substituent selected from hydroxy, acetoxy, or methoxy. In some embodiments, the tryptamine is 4-hydroxy-N-methyl-N-ethyltryptamine, psilocin, 5-methoxy-N,N-diisopropyltryptamine, 5-methoxy-N,N-dimethyltryptamine, or O-acetylpsilocin (4-acetoxy-N,N-dimethyltryptamine), or a combination thereof.

The tryptamine psychedelic may be a pharmaceutically acceptable salt of a compound of the present disclosure, e.g., a compound of Formula (I) through (III), defined hereinafter. The tryptamine psychedelic may be a pharmaceutically acceptable salt of a single compound of the present disclosure or a pharmaceutically acceptable salt of a mixture of compounds of the present disclosure. The injectable pharmaceutical formulation comprises (as the tryptamine psychedelic) a pharmaceutically acceptable salt of a compound of the present disclosure, e.g., a compound of Formula (I) through (III), when it comprise ions (protonated forms) of the compounds of the present disclosure and ions that counter the charge of the compounds of the present disclosure (counterions) in solution. Accordingly, the pharmaceutically acceptable salt of a compound of the present disclosure (e.g., e.g., a compound of Formula (I) through (III)) may be pre-formed, for example as a fumarate salt of a compound of the present disclosure, and subsequently combined with the release modifier and the aqueous vehicle, to form the injectable pharmaceutical formulation. In other words, the injectable pharmaceutical formulation may be prepared from a pre-formed, typically solid form and in some cases crystalline solid form, of the pharmaceutically acceptable salt of a compound of the present disclosure (e.g., a compound of Formula (I) through (III)). Alternatively, the pharmaceutically acceptable salt of a compound of the present disclosure within the pharmaceutical formulation may be formed in-situ, for example, by contacting the compound of the present disclosure (e.g., a compound of Formula (I) through (III)) as a free base with an aqueous vehicle comprising available H⁺ (aq) ions capable of ionizing/protonating the compounds of the present disclosure. While a distribution of free base and protonated species (salt form) of the compound may exist in solution depending on the pH of the pharmaceutical formulation, the pharmaceutical formulation generally contains a molar proportion of protonated compound (salt form) of least 10%, at least 20%, at least 30%, at least 40%, at least 50%, at least 60%, at least 70%, at least 80%, at least 90%, at least 95%, at least 99%. In most cases, the pharmaceutical formulation contains a molar proportion of protonated compound (salt form) of least 80%, at least 90%, at least 95%, at least 99%, at least 99.5%, at least 99.9% for most favorable aqueous solubility.

In some embodiments, the tryptamine psychedelic is a pharmaceutically acceptable salt of a compound of Formula (I), or a stereoisomer, solvate, or prodrug thereof,

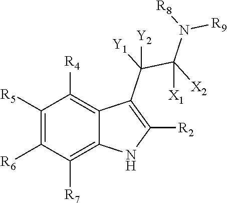

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, unsubstituted or substituted alkoxy, unsubstituted or substituted alkylthio, and unsubstituted or substituted acyloxy;
R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl; and
R₈ and R₉ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl,
or alternatively R₈ and R₉ together with the nitrogen atom attached thereto are optionally joined to form an unsubstituted or substituted heterocycloalkyl.

X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is a substituted or unsubstituted C₁-C₆ alkyl. In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, one or more of X₁ and X₂ is a substituted or unsubstituted C₃-C₁₀ cycloalkyl. In some embodiments, one or more of X₁ and X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, one or more of X₁ and X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, X₁ and/or X₂ is an unsubstituted or substituted alkenyl, e.g., an unsubstituted or substituted allyl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₂ is deuterium. In some embodiments, R₂ is hydrogen. In some embodiments, R₂ is a halogen, e.g., fluoro, chloro, bromo, and iodo. In some embodiments, R₂ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₂ is a substituted C₁-C₆ alkyl. When R₂ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₂ is an unsubstituted or substituted alkenyl, e.g., an unsubstituted or substituted allyl. In some embodiments, R₂ is an unsubstituted or substituted alkynyl. In some embodiments, R₂ is a substituted or unsubstituted C₃-C₁₀ cycloalkyl. In some embodiments, R₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₂ is an unsubstituted or substituted heterocycloalkyl. In some embodiments, R₂ is an unsubstituted or substituted aryl. In some embodiments, R₂ is an unsubstituted or substituted heteroaryl.

R₄ and R₅ may be the same, or different. In some embodiments, R₄ is deuterium. In some embodiments, R₄ is hydrogen. In some embodiments, R₄ is hydroxyl. In some embodiments, R₄ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₄ is a substituted C₁-C₆ alkyl. When R₄ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₄ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₄ is a substituted alkoxy. When R₄ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc. In some embodiments, R₄ is an unsubstituted alkylthio, examples of which include, but are not limited to, methylthio, ethylthio, n-propylthio, isopropylthio, n-butylthio, isobutylthio, sec-butylthio, t-butylthio, n-pentylthio, neopentylthio, and hexylthio. In some embodiments, R₄ is a substituted alkylthio. The alkylthio group may contain one, or more than one, substituent. In some embodiments, R₄ is an alkylthio group substituted with one or more deuterium. The alkylthio group may contain one, or more than one, deuterium substituent. For example, when the alkylthio group is a C₁ alkylthio group (i.e., a methylthio group), the deuterium substituted C₁ alktlthio group may be —SCDH₂, —SCD₂H, and —SCD₃. In some embodiments, R₄ is a haloalkylthio (an alkylthio substituted with one or more halogen atoms), examples of which include, but are not limited to, —SCH₂F, —SCHF₂, —SCF₃, —SCH₂CH₂F, —SCH₂CHF₂, —SCH₂CF₃, —SCH₂CH₂CH₂F, —SCH₂CH₂CHF₂, —SCH₂CH₂CF₃, —SCH₂CH₂CH₂CH₂F, —SCH₂CH₂CH₂CHF₂, and —SCH₂CH₂CH₂CF₃, with particular mention being made to —SCH₂F, —SCHF₂, —SCF₃. In some embodiments, R₄ is an unsubstituted or substituted acyloxy, examples of which include, but are not limited to, acetoxy (—OCOCH₃), propionoxy (—OCOCH₂CH₃), and butyroxy (—OCOCH₂CH₂CH₃).

In some embodiments, R₅ is deuterium. In some embodiments, R₅ is hydrogen. In some embodiments, R₅ is hydroxyl. In some embodiments, R₅ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₅ is a substituted C₁-C₆ alkyl. When R₅ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₅ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is a substituted alkoxy. When R₅ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc. In some embodiments, R₅ is an unsubstituted alkylthio, examples of which include, but are not limited to, methylthio, ethylthio, n-propylthio, isopropylthio, n-butylthio, isobutylthio, sec-butylthio, t-butylthio, n-pentylthio, neopentylthio, and hexylthio. In some embodiments, R₅ is a substituted alkylthio. The alkylthio group may contain one, or more than one, substituent. In some embodiments, R₅ is an alkylthio group substituted with one or more deuterium. The alkylthio group may contain one, or more than one, deuterium substituent. For example, when the alkylthio group is a C₁ alkylthio group (i.e., a methylthio group), the deuterium substituted C₁ alktlthio group may be —SCDH₂, —SCD₂H, and —SCD₃. In some embodiments, R₅ is a haloalkylthio (an alkylthio substituted with one or more halogen atoms), examples of which include, but are not limited to, —SCH₂F, —SCHF₂, —SCF₃, —SCH₂CH₂F, —SCH₂CHF₂, —SCH₂CF₃, —SCH₂CH₂CH₂F, —SCH₂CH₂CHF₂, —SCH₂CH₂CF₃, —SCH₂CH₂CH₂CH₂F, —SCH₂CH₂CH₂CHF₂, and —SCH₂CH₂CH₂CF₃, with particular mention being made to —SCH₂F, —SCHF₂, —SCF₃. In some embodiments, R₅ is an unsubstituted or substituted acyloxy, examples of which include, but are not limited to, acetoxy (—OCOCH₃), propionoxy (—OCOCH₂CH₃), and butyroxy (—OCOCH₂CH₂CH₃).

R₆ and R₇ may be the same, or different. In some embodiments, R₆ and R₇ are the same. In some embodiments, R₆ and R₇ are different. In some embodiments, R₆ is hydrogen. In some embodiments, R₆ is deuterium. In some embodiments, R₆ is a halogen, e.g., fluoro, chloro, bromo, and iodo. In some embodiments, R₆ is an unsubstituted or substituted alkyl (e.g., an unsubstituted or substituted a C₁-C₆ alkyl). In some embodiments, R₆ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₆ is a substituted C₁-C₆ alkyl. When R₆ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₆ is an unsubstituted or substituted alkenyl, e.g., an unsubstituted or substituted allyl. In some embodiments, R₆ is an unsubstituted or substituted alkynyl. In some embodiments, R₆ is an unsubstituted or substituted C₃-C₁₀ cycloalkyl. In some embodiments, R₆ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₆ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₆ is an unsubstituted or substituted heterocycloalkyl. In some embodiments, R₆ is an unsubstituted or substituted aryl. In some embodiments, R₆ is an unsubstituted or substituted heteroaryl.

In some embodiments, R₇ is hydrogen. In some embodiments, R₇ is deuterium. In some embodiments, R₇ is a halogen, e.g., fluoro, chloro, bromo, and iodo. In some embodiments, R₇ is an unsubstituted or substituted alkyl (e.g., an unsubstituted or substituted a C₁-C₆ alkyl). In some embodiments, R₇ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₇ is a substituted C₁-C₆ alkyl. When R₇ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₇ is an unsubstituted or substituted alkenyl, e.g., an unsubstituted or substituted allyl. In some embodiments, R₇ is an unsubstituted or substituted alkynyl. In some embodiments, R₇ is an unsubstituted or substituted C₃-C₁₀ cycloalkyl. In some embodiments, R₇ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₇ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₇ is an unsubstituted or substituted heterocycloalkyl. In some embodiments, R₇ is an unsubstituted or substituted aryl. In some embodiments, R₇ is an unsubstituted or substituted heteroaryl.

R₈ and R₉ may be the same, or different. In some embodiments, R₈ and R₉ are the same. In some embodiments, R₈ and R₉ are hydrogen. In some embodiments, R₈ and R₉ are deuterium. In some embodiments, R₈ and R₉ are unsubstituted or substituted alkyl, such as an unsubstituted or substituted C₁-C₆ alkyl. In some embodiments, R₈ and R₉ are different. In some embodiments, R₈ is hydrogen, and R₉ is an unsubstituted or substituted C₁-C₆ alkyl.

In some embodiments, R₈ and/or R₉ is an unsubstituted C₁-C₆ alkyl, examples of which include, but are not limited to, methyl, ethyl, n-propyl, and isopropyl, preferably methyl. In some embodiments, R₈ and/or R₉ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₈ and/or R₉ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃. In some embodiments, R₈ and/or R₉ is a haloalkyl, examples of which include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃. In some embodiments, R₈ and/or R₉ is an unsubstituted or substituted alkenyl, e.g., an unsubstituted or substituted allyl.

In some embodiments, R₈ and/or R₉ is an unsubstituted or substituted alkynyl. In some embodiments, R₈ and/or R₉ is a substituted or unsubstituted C₃-C₁₀ cycloalkyl. In some embodiments, R₈ and/or R₉ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₈ and/or R₉ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₈ and/or R₉ is an unsubstituted or substituted heterocycloalkyl. In some embodiments, R₈ and/or R₉ is an unsubstituted or substituted aryl. In some embodiments, R₈ and/or R₉ is an unsubstituted or substituted heteroaryl.

In some embodiments, R₈ and R₉ together with the nitrogen atom attached thereto are joined to form an unsubstituted or substituted heterocycloalkyl. In some embodiments, R₈ and R₉ together with the nitrogen atom attached thereto are joined to form an unsubstituted heterocycloalkyl. The unsubstituted heterocycloalkyl group may be, e.g., a 3-membered ring, a 4-membered ring, 5-membered ring, a 6-membered ring, a 7-membered ring, an 8-membered ring, etc., which may be optionally fused to other ring(s). The unsubstituted heterocycloalkyl group contains a minimum of one nitrogen ring atom (the nitrogen atom intervening R₈ and R₉), and may optionally contain at least one additional hetero-ring atom, which may be one or more of nitrogen, sulfur, or oxygen, for a total of 1, 2, 3, or 4 hetero-ring atoms (at least one of which is a nitrogen ring atom). Examples of unsubstituted heterocycloalkyl groups formed from joining R₈ and R₉ together with the nitrogen atom attached thereto include, but are not limited to,

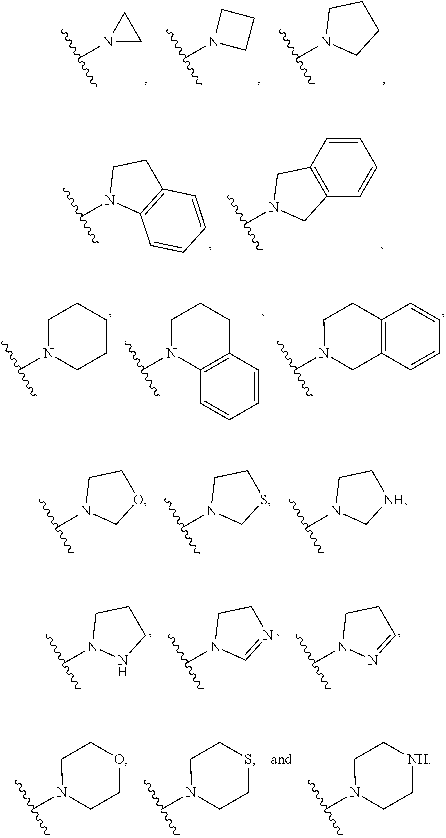

In some embodiments, R₈ and R₉ together with the nitrogen atom attached thereto are joined to form a substituted heterocycloalkyl. The substituted heterocycloalkyl group may be, e.g., a 3-membered ring, a 4-membered ring, a 5-membered ring, a 6-membered ring, a 7-membered ring, an 8-membered ring, etc., which may be optionally fused to other ring(s). The substituted heterocycloalkyl group contains a minimum of one nitrogen ring atom (the nitrogen atom intervening R₈ and R₉), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the substituted heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, 1,2,3,4-tetrahydroisoquinoline, thiazolidine, morpholine, or thiomorpholine, which is substituted with at least one substituent. The substituent(s) may be any recited herein, including, but not limited to, deuterium, halogen (e.g., fluorine), polar substituents such as hydroxyl, oxo, unsubstituted alkoxy, substituted alkoxy (e.g., polyether groups), unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The substituted heterocycloalkyl formed from joining R₈ and R₉ together with the nitrogen atom attached thereto contains a heterocycloalkyl group substituted with one, two, three, four, or more substituents. The substituent may be located on a carbon ring atom or on a hetero-ring atom.

Examples of substituted heterocycloalkyl groups formed from joining R₈ and R₉ together with the nitrogen atom attached thereto include, but are not limited to,

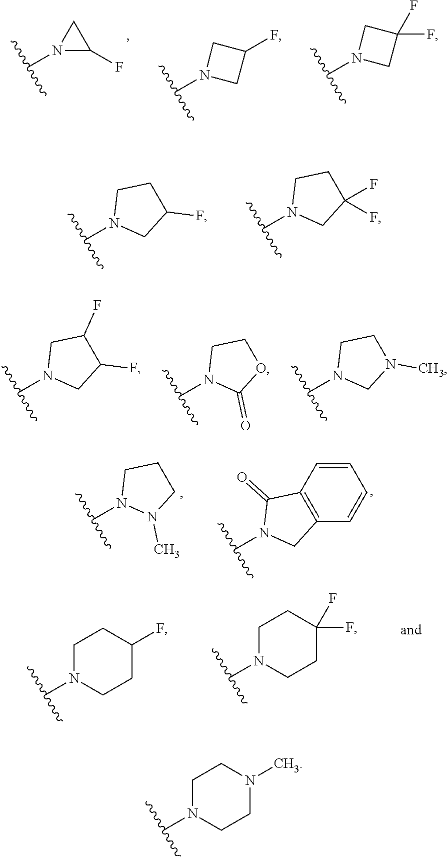

In some embodiments, the tryptamine psychedelic is a pharmaceutically acceptable salt of a compound of Formula (I), or a stereoisomer, solvate, or prodrug thereof, wherein any one or more of X₁, X₂, Y₁, Y₂, R₂, R₄, R₅, R₆, R₇, R₈, and R₉ optionally comprises deuterium. In some embodiments, at least one of X₁, X₂, Y₁, Y₂, R₂, R₄, R₅, R₆, R₇, R₈, and R₉ comprises deuterium.

In some embodiments, at least one of X₁, X₂, Y₁, Y₂, R₅, R₈, and R₉ comprises deuterium. In some embodiments, at least one of X₁, X₂, Y₁, Y₂, R₈, and R₉ comprises deuterium. In some embodiments, X₁, X₂, R₈, and R₉ comprise deuterium. In some embodiments, X₁, X₂, Y₁, Y₂, R₈, and R₉ comprise deuterium. In some embodiments, X₁, X₂, and R₅ comprise deuterium. In some embodiments, X₁, X₂, Y₁, Y₂, R₅, R₈, and R₉ comprise deuterium.

In some embodiments, the tryptamine psychedelic is a pharmaceutically acceptable salt of a compound of Formula (II), or a stereoisomer, solvate, or prodrug thereof

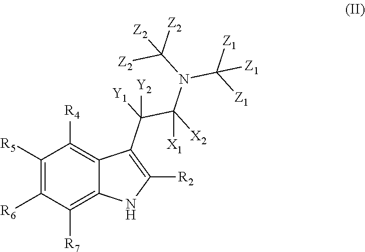

wherein:
X₁ and X₂ are independently hydrogen or deuterium,
Y₁ and Y₂ are independently hydrogen or deuterium,
each Z₁ is independently hydrogen or deuterium,
each Z₂ is independently hydrogen or deuterium, and
R₂, R₄, R₅, R₆, and R₇ are independently hydrogen or deuterium.

X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is deuterium and X₂ is hydrogen.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, Y₁ is deuterium and Y₂ is hydrogen.

In some embodiments, X₁, X₂, Y₁, and Y₂ are hydrogen. In some embodiments, X₁, X₂, Y₁, and Y₂ are deuterium.

In some embodiments, each Z₁ is hydrogen. In some embodiments, each Z₁ is deuterium. In some embodiments, one Z₁ is hydrogen, while the other two Z₁'s are deuterium. In some embodiments, one Z₁ is deuterium, while the other two Z₁'s are hydrogen. In some embodiments, each Z₂ is hydrogen. In some embodiments, each Z₂ is deuterium. In some embodiments, one Z₂ is hydrogen, while the other two Z₂'s are deuterium. In some embodiments, one Z₂ is deuterium, while the other two Z₂'s are hydrogen. In some embodiments, each Z₁ and Z₂ is hydrogen. In some embodiments, each Z₁ and Z₂ is deuterium.

In some embodiments, R₂ is deuterium. In some embodiments, R₂ is hydrogen. In some embodiments, R₄ is deuterium. In some embodiments, R₄ is hydrogen. In some embodiments, R₅ is deuterium. In some embodiments, R₅ is hydrogen. In some embodiments, R₆ is deuterium. In some embodiments, R₆ is hydrogen. In some embodiments, R₇ is deuterium. In some embodiments, R₇ is hydrogen. R₂, R₄, R₅, R₆, and R₇ may be the same, for example, R₂, R₄, R₅, R₆, and R₇ may each be hydrogen, or alternatively, R₂, R₄, R₅, R₆, and R₇ may each be deuterium. In some embodiments, at least one of R₂, R₄, R₅, R₆, and R₇ is deuterium, or at least two of R₂, R₄, R₅, R₆, and R₇ are deuterium, or at least three of R₂, R₄, R₅, R₆, and R₇ are deuterium, or at least four of R₂, R₄, R₅, R₆, and R₇ are deuterium.

In some embodiments, at least one of X₁, X₂, Y₁, Y₂, Z₁, Z₂, R₂, R₄, R₅, R₆, and R₇ is deuterium. In some embodiments, X₁, X₂, Z₁ and Z₂ are deuterium. In some embodiments, X₁, X₂, Y₁, and Y₂ are deuterium. In some embodiments, X₁, X₂, Y₁, Y₂, Z₁, and Z₂ are deuterium.

In some embodiments, the tryptamine psychedelic is a pharmaceutically acceptable salt of a compound of Formula (III), or a stereoisomer, solvate, or prodrug thereof

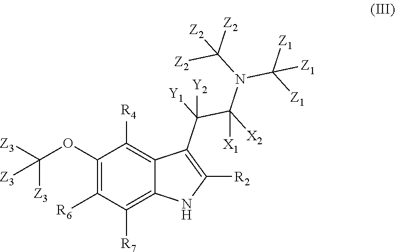

wherein:
X₁ and X₂ are independently hydrogen or deuterium,
Y₁ and Y₂ are independently hydrogen or deuterium,
each Z₁ is independently hydrogen or deuterium,
each Z₂ is independently hydrogen or deuterium,
each Z₃ is independently hydrogen or deuterium, and
R₂, R₄, R₆, and R₇ are independently hydrogen or deuterium.

X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is deuterium and X₂ is hydrogen.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, Y₁ is deuterium and Y₂ is hydrogen.

In some embodiments, X₁, X₂, Y₁, and Y₂ are hydrogen. In some embodiments, X₁, X₂, Y₁, and Y₂ are deuterium.

In some embodiments, each Z₁ is hydrogen. In some embodiments, each Z₁ is deuterium. In some embodiments, one Z₁ is hydrogen, while the other two Z₁'s are deuterium. In some embodiments, one Z₁ is deuterium, while the other two Z₁'s are hydrogen. In some embodiments, each Z₂ is hydrogen. In some embodiments, each Z₂ is deuterium. In some embodiments, one Z₂ is hydrogen, while the other two Z₂'s are deuterium. In some embodiments, one Z₂ is deuterium, while the other two Z₂'s are hydrogen. In some embodiments, each Z₁ and Z₂ is hydrogen. In some embodiments, each Z₁ and Z₂ is deuterium.

In some embodiments, each Z₃ is hydrogen. In some embodiments, each Z₃ is deuterium. In some embodiments, one Z₃ is hydrogen, while the other two Z₃'s are deuterium. In some embodiments, one Z₃ is deuterium, while the other two Z₃'s are hydrogen. In some embodiments, each Z₁, Z₂, and Z₃ is hydrogen. In some embodiments, each Z₁ and Z₂ is hydrogen and each Z₃ is deuterium. In some embodiments, each Z₁, Z₂, and Z₃ is deuterium. In some embodiments, each Z₁ and Z₂ is deuterium, and each Z₃ is hydrogen.

In some embodiments, R₂ is deuterium. In some embodiments, R₂ is hydrogen. In some embodiments, R₄ is deuterium. In some embodiments, R₄ is hydrogen. In some embodiments, R₆ is deuterium. In some embodiments, R₆ is hydrogen. In some embodiments, R₇ is deuterium. In some embodiments, R₇ is hydrogen. R₂, R₄, R₆, and R₇ may be the same, for example, R₂, R₄, R₆, and R₇ may each be hydrogen, or alternatively, R₂, R₄, R₆, and R₇ may each be deuterium. In some embodiments, at least one of R₂, R₄, R₆, and R₇ is deuterium, or at least two of R₂, R₄, R₆, and R₇ are deuterium, or at least three of R₂, R₄, R₆, and R₇ are deuterium.

In some embodiments, at least one of X₁, X₂, Y₁, Y₂, Z₁, Z₂, Z₃, R₂, R₄, R₆, and R₇ is deuterium. In some embodiments, X₁, X₂, Z₁ and Z₂ are deuterium and each Z₃ is hydrogen. In some embodiments, X₁, X₂, Y₁, and Y₂ are deuterium and each Z₃ is hydrogen. In some embodiments, X₁, X₂, Y₁, Y₂, Z₁, and Z₂ are deuterium and each Z₃ is hydrogen. In some embodiments, X₁, X₂, and Z₃ are deuterium. In some embodiments, X₁, X₂, Z₁, Z₂, and Z₃ are deuterium. In some embodiments, X₁, X₂, Y₁, Y₂, and Z₃ are deuterium. In some embodiments, X₁, X₂, Y₁, Y₂, Z₁, Z₂, and Z₃ are deuterium.

In some embodiments, the tryptamine psychedelic is a pharmaceutically acceptable salt of a compound of Formula (I) through (III), which include, but are not limited to, the following exemplary compounds

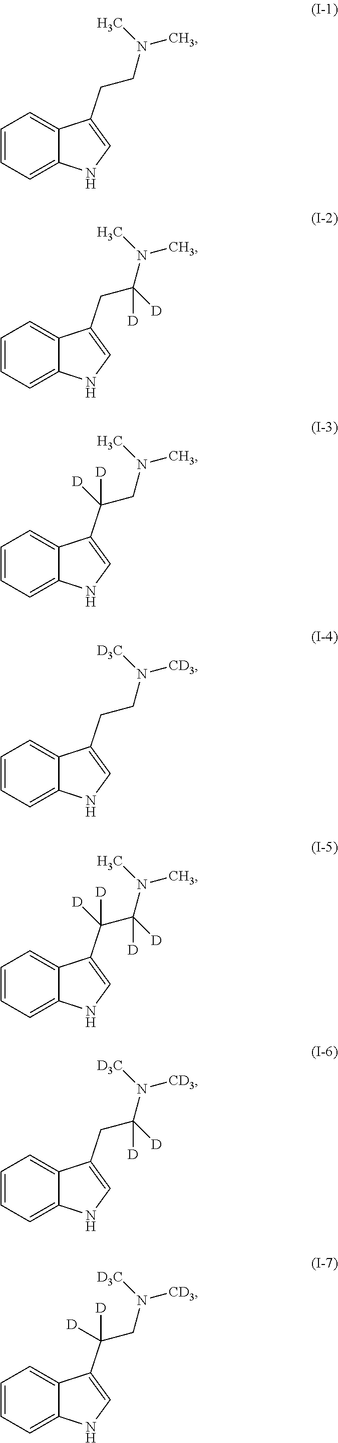

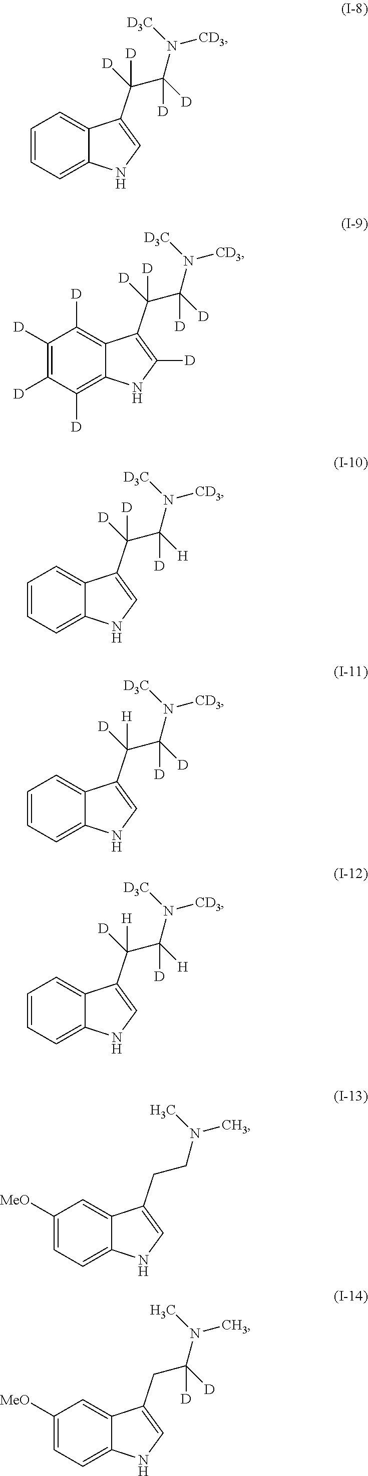

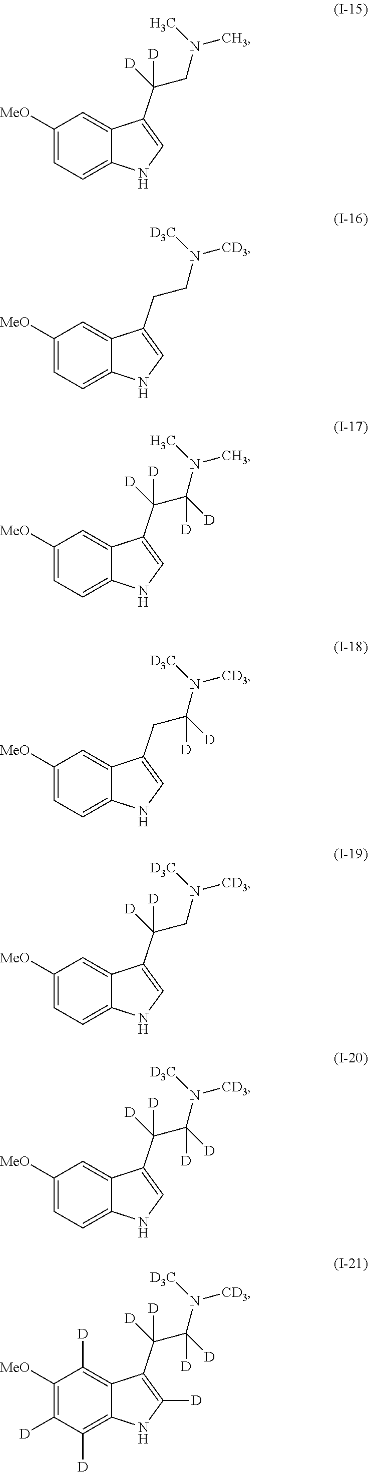

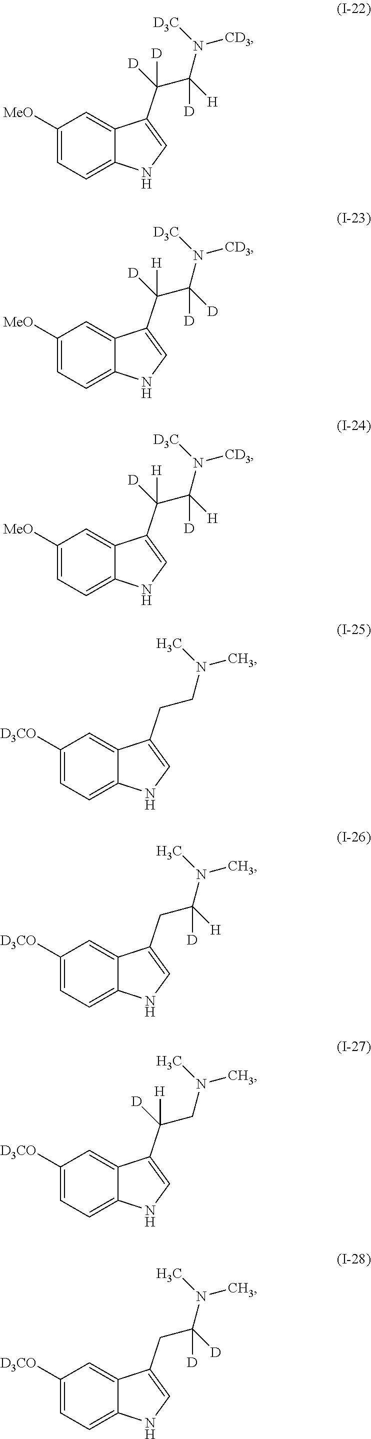

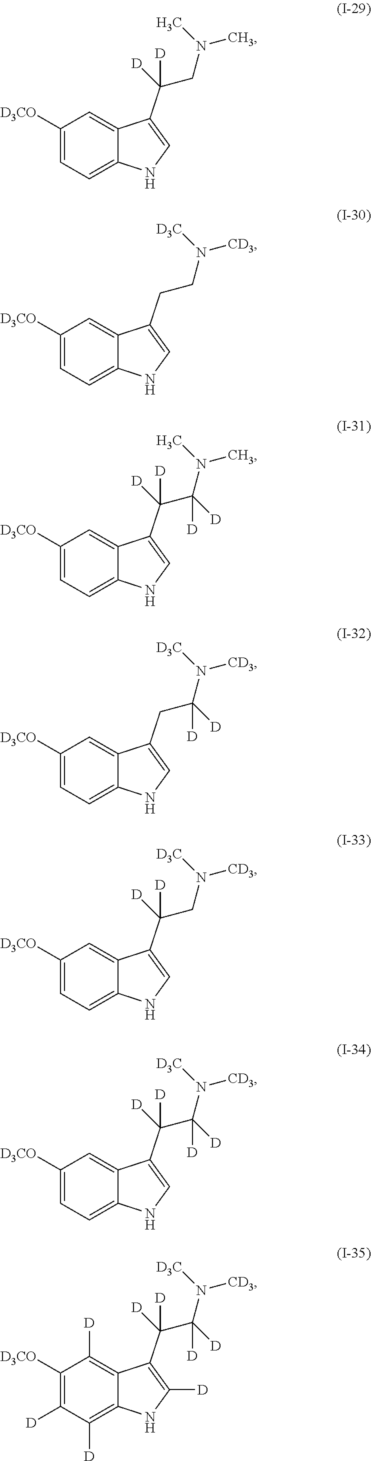

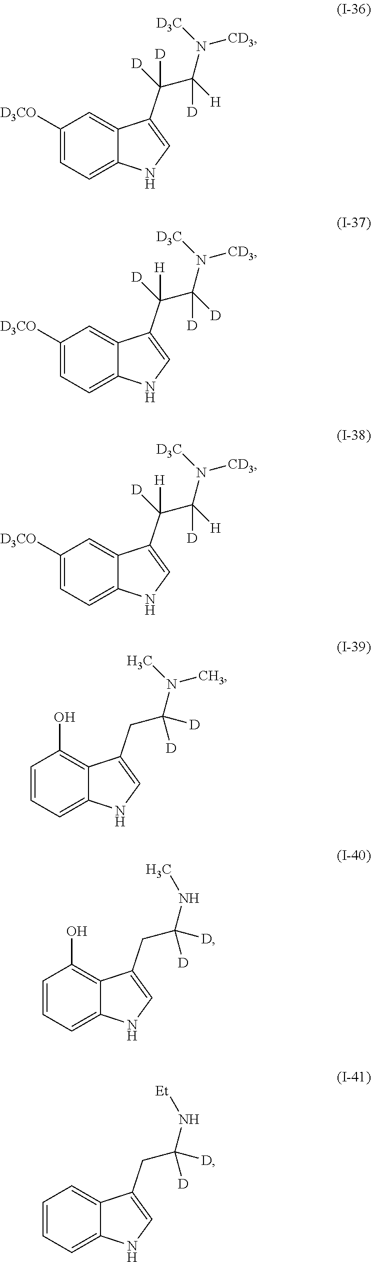

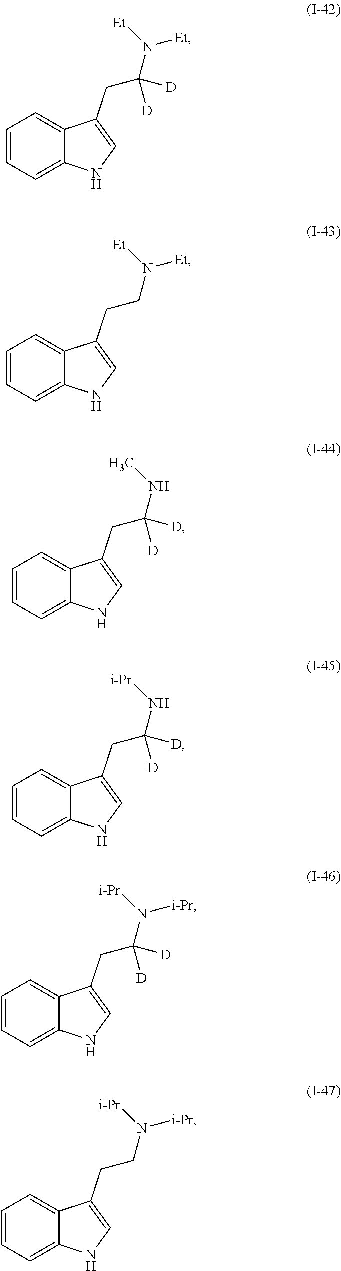

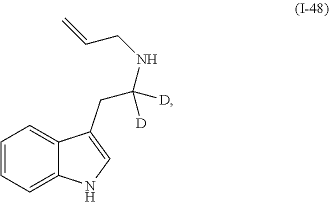

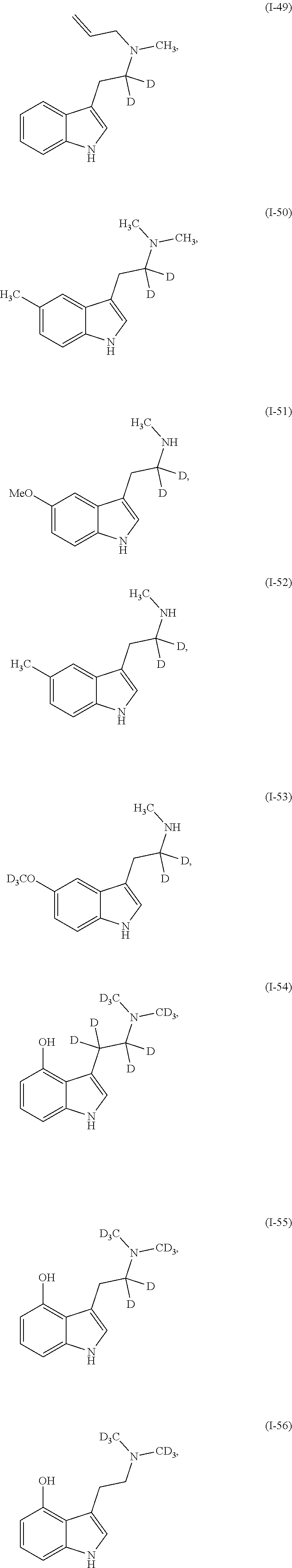

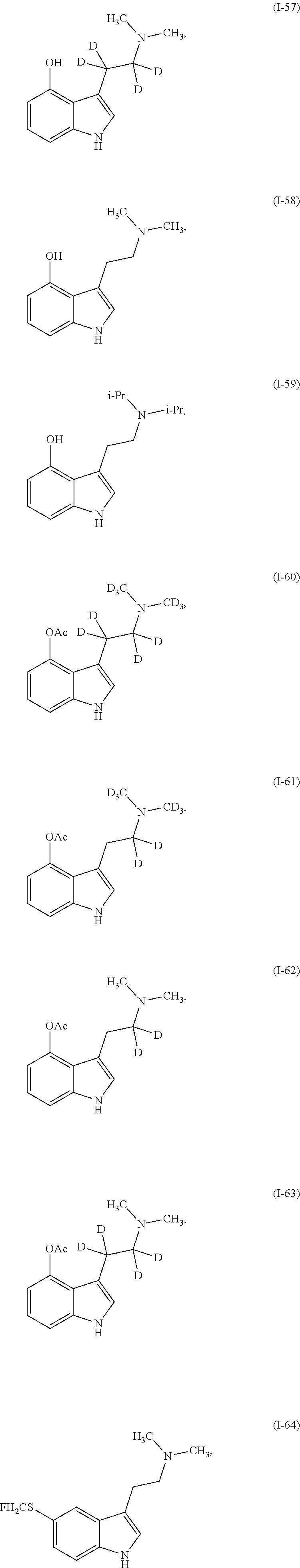

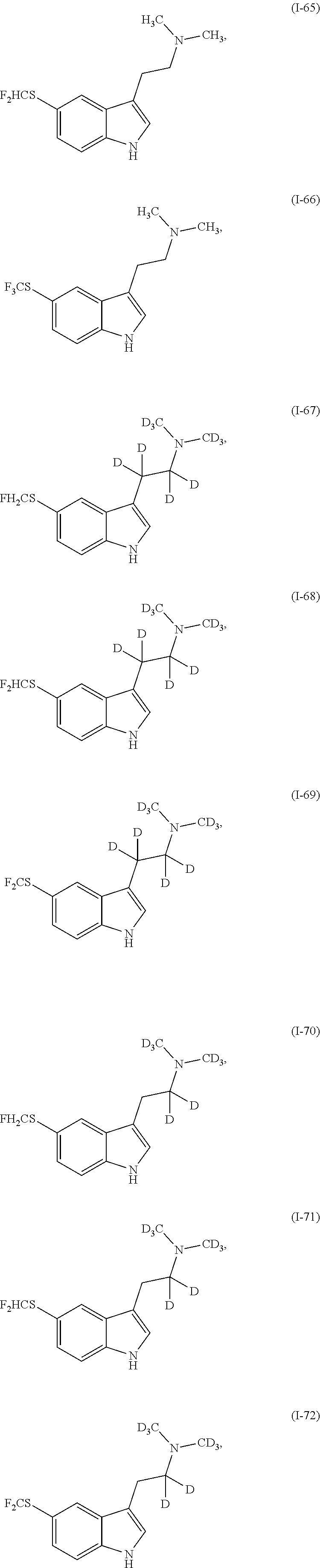

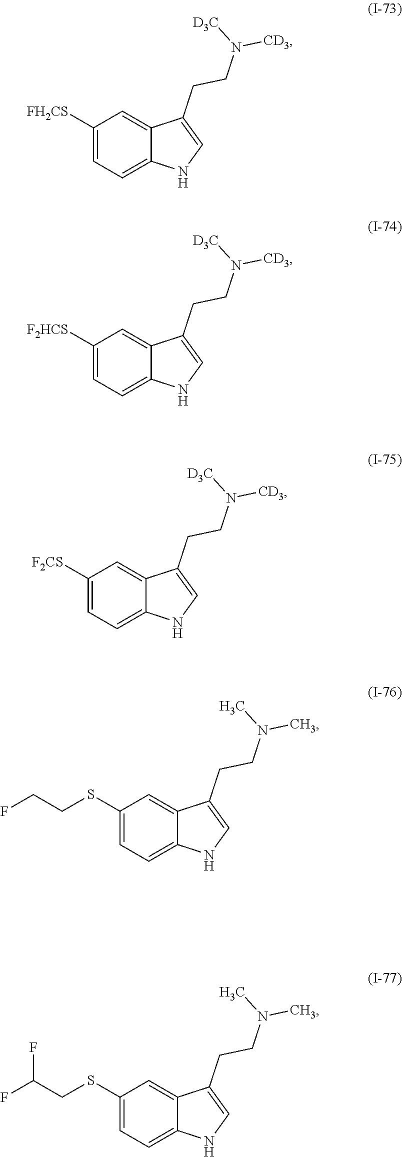

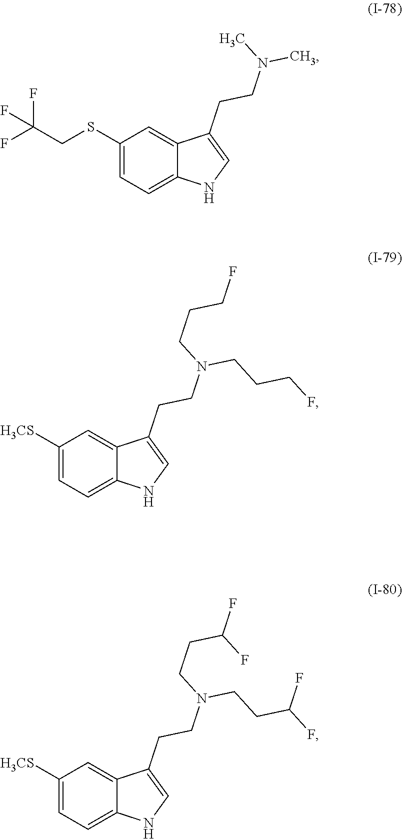

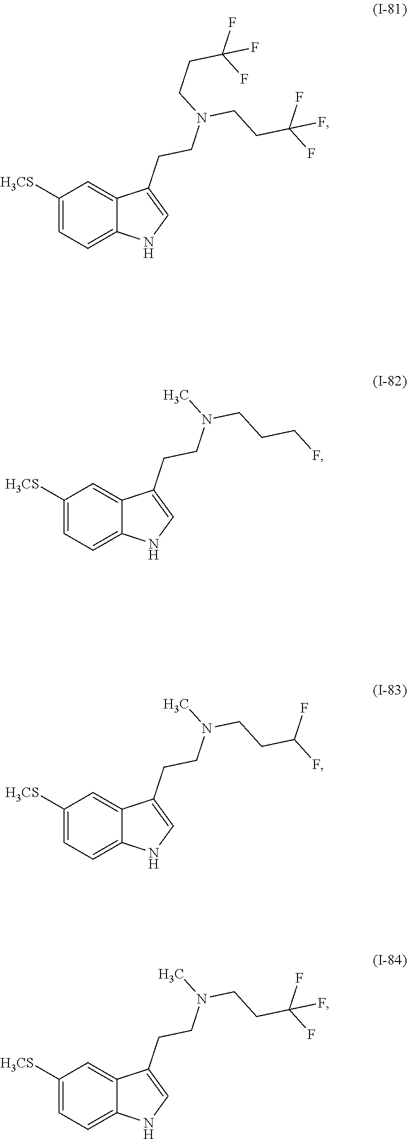

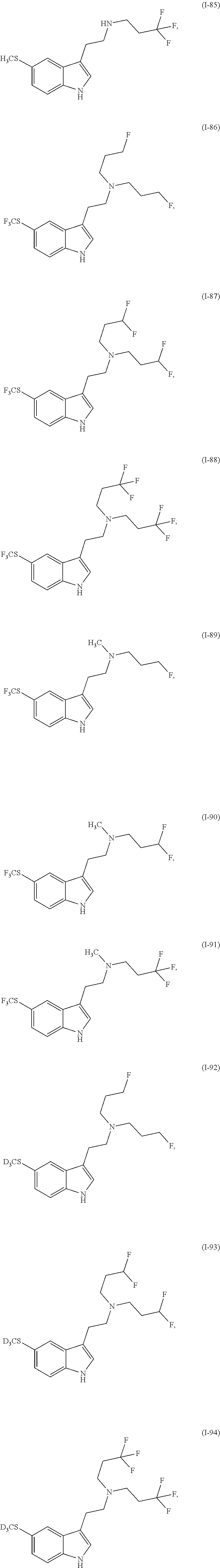

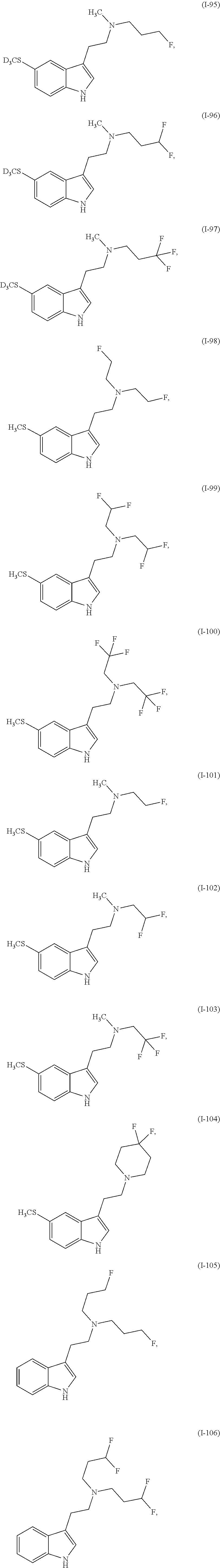

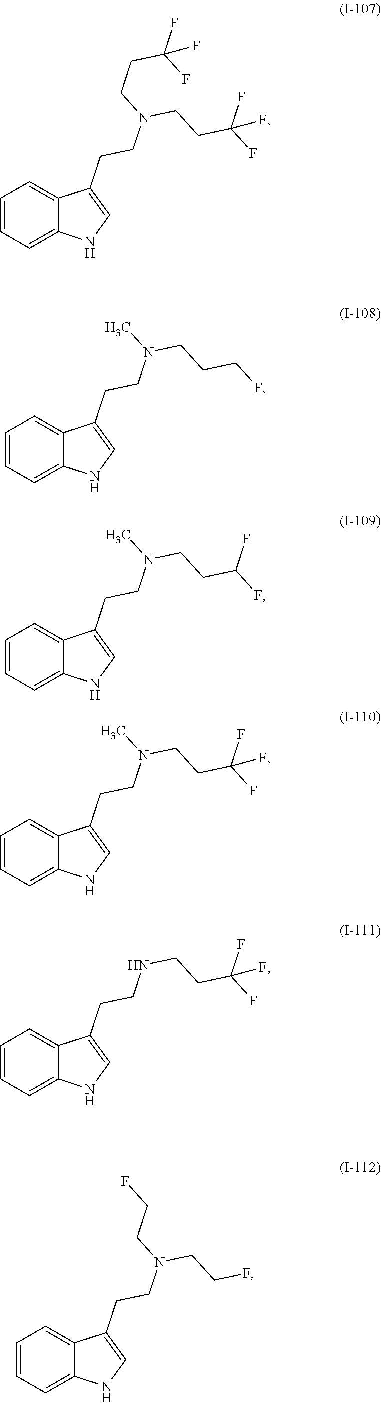 [SMILES: FC(F)(F)CCN(CCc1c[nH]c2ccccc12)CCC(F)(F)F ; CN(CCCF)CCc1c[nH]c2ccccc12 ; CN(CCc1c[nH]c2ccccc12)CCC(F)F ; CN(CCc1c[nH]c2ccccc12)CCC(F)(F)F ; FC(F)(F)CCNCCc1c[nH]c2ccccc12 ; FCCN(CCF)CCc1c[nH]c2ccccc12]

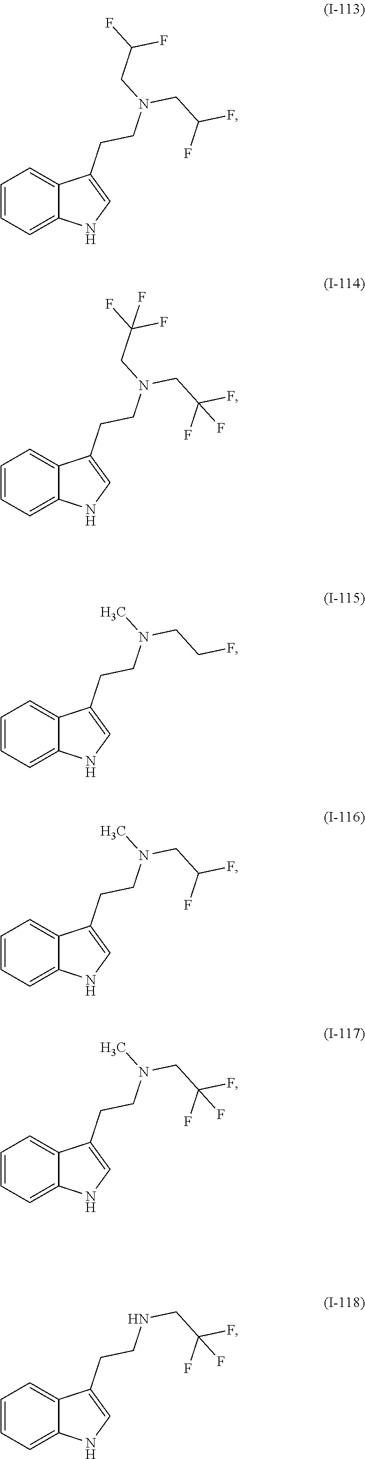 [SMILES: FC(F)CN(CCc1c[nH]c2ccccc12)CC(F)F ; FC(F)(F)CN(CCc1c[nH]c2ccccc12)CC(F)(F)F ; CN(CCF)CCc1c[nH]c2ccccc12 ; CN(CCc1c[nH]c2ccccc12)CC(F)F ; CN(CCc1c[nH]c2ccccc12)CC(F)(F)F ; FC(F)(F)CNCCc1c[nH]c2ccccc12]

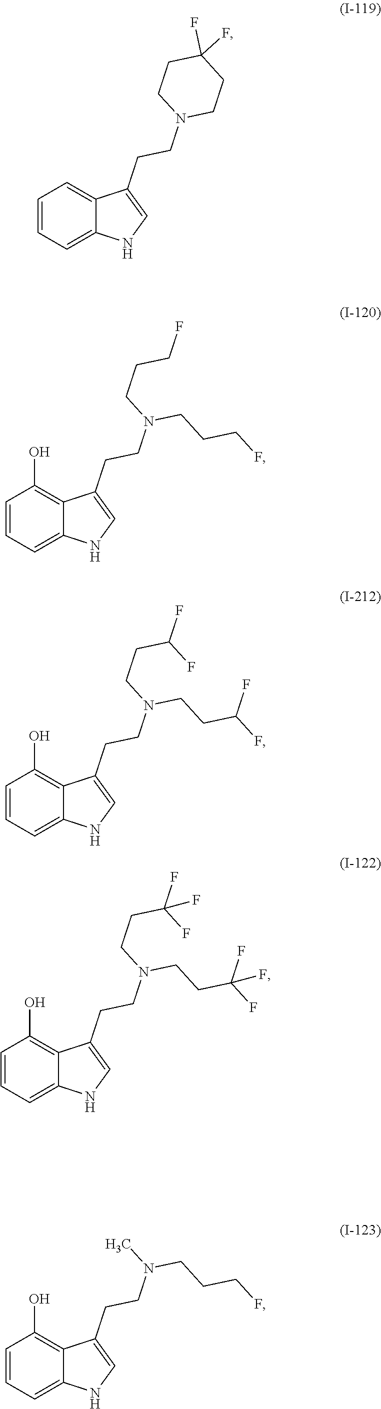 [SMILES: FC1(F)CCN(CCc2c[nH]c3ccccc23)CC1 ; Oc1cccc2[nH]cc(CCN(CCCF)CCCF)c12 ; Oc1cccc2[nH]cc(CCN(CCC(F)F)CCC(F)F)c12 ; Oc1cccc2[nH]cc(CCN(CCC(F)(F)F)CCC(F)(F)F)c12 ; CN(CCCF)CCc1c[nH]c2cccc(O)c12]

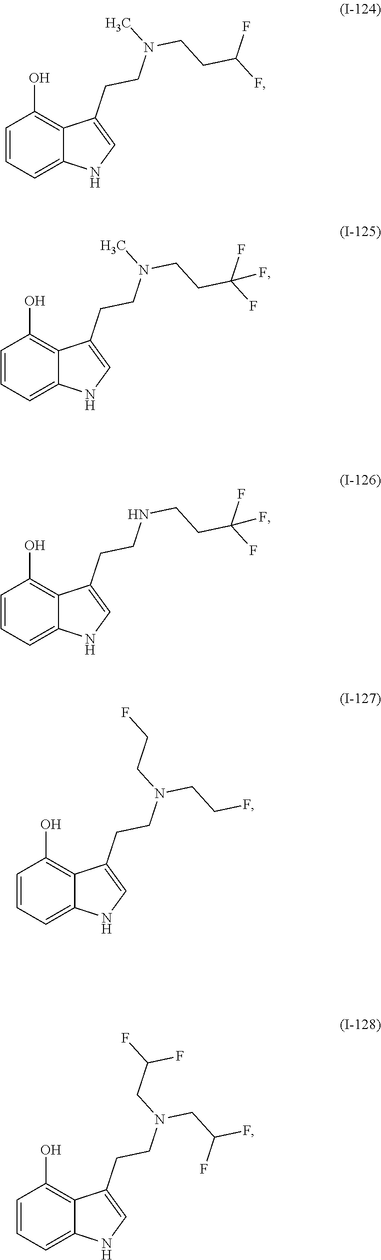 [SMILES: CN(CCc1c[nH]c2cccc(O)c12)CCC(F)F ; CN(CCc1c[nH]c2cccc(O)c12)CCC(F)(F)F ; Oc1cccc2[nH]cc(CCNCCC(F)(F)F)c12 ; Oc1cccc2[nH]cc(CCN(CCF)CCF)c12 ; Oc1cccc2[nH]cc(CCN(CC(F)F)CC(F)F)c12]

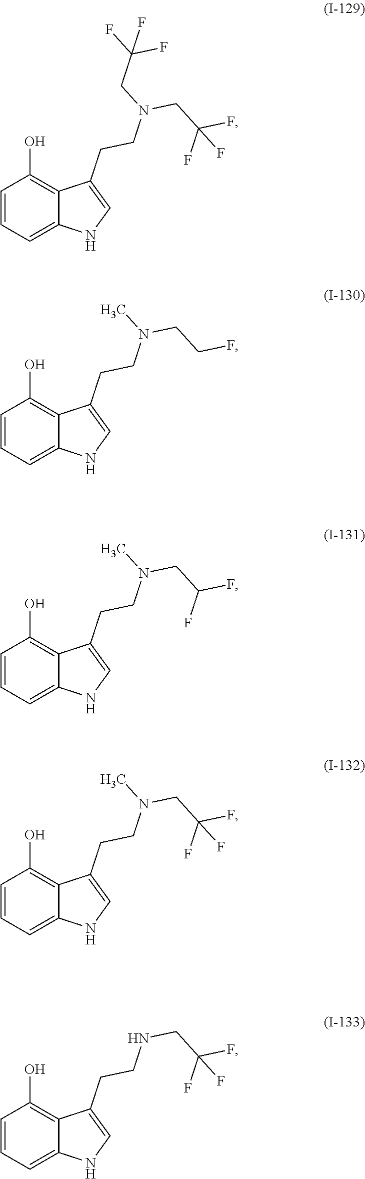 [SMILES: Oc1cccc2[nH]cc(CCN(CC(F)(F)F)CC(F)(F)F)c12 ; CN(CCF)CCc1c[nH]c2cccc(O)c12 ; CN(CCc1c[nH]c2cccc(O)c12)CC(F)F ; CN(CCc1c[nH]c2cccc(O)c12)CC(F)(F)F ; Oc1cccc2[nH]cc(CCNCC(F)(F)F)c12]

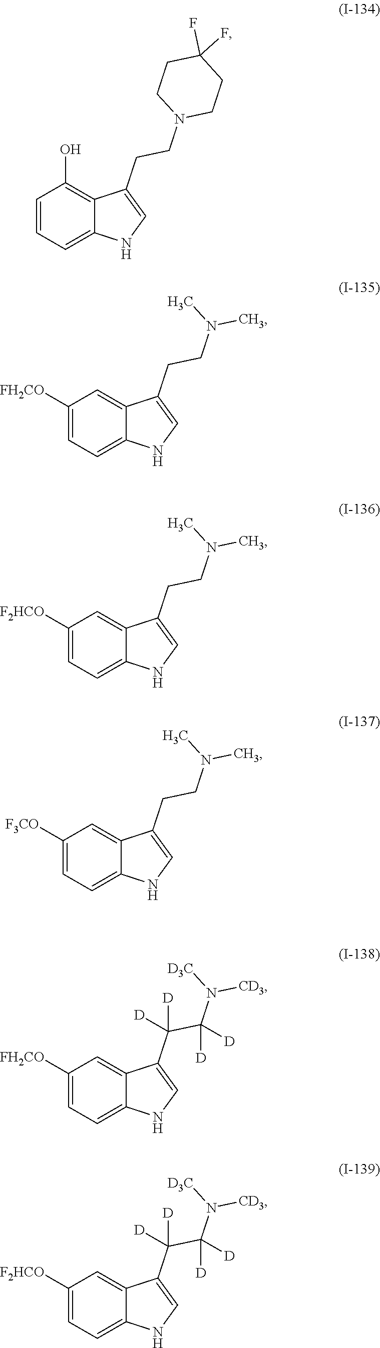

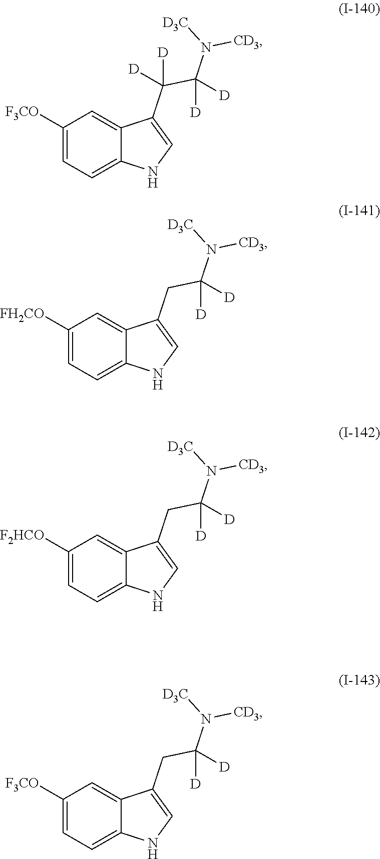

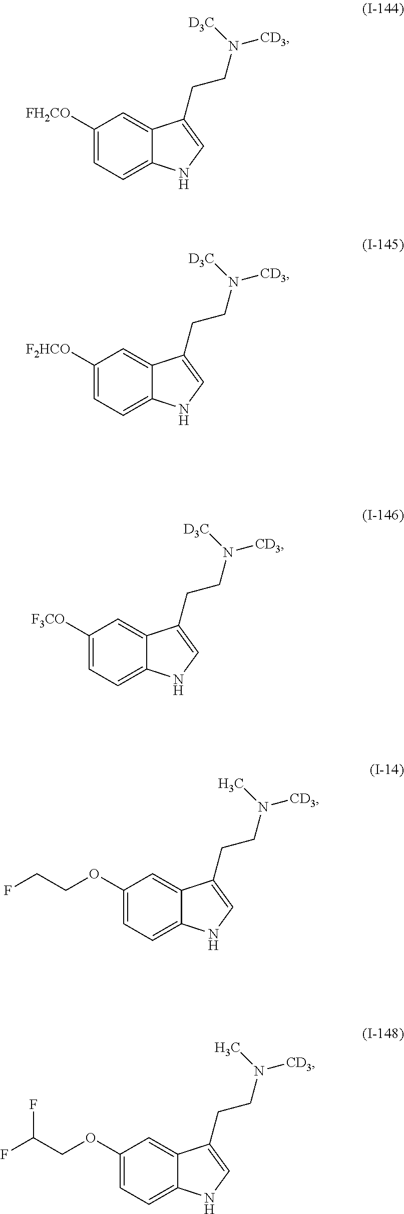

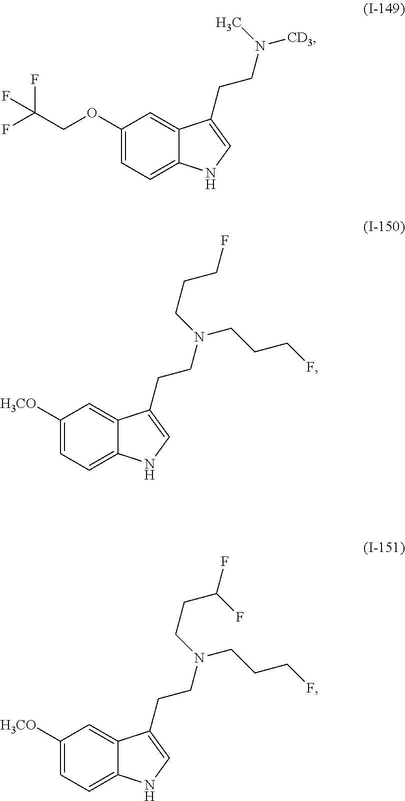 [SMILES: [2H]C([2H])([2H])N(C)CCc1c[nH]c2ccc(OCC(F)(F)F)cc12 ; COc1ccc2[nH]cc(CCN(CCCF)CCCF)c2c1 ; COc1ccc2[nH]cc(CCN(CCCF)CCC(F)F)c2c1]

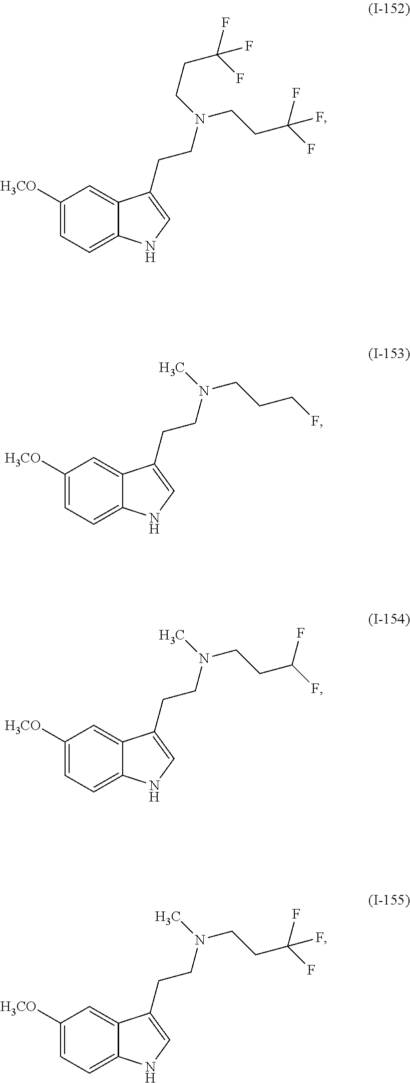 [SMILES: COc1ccc2[nH]cc(CCN(CCC(F)(F)F)CCC(F)(F)F)c2c1 ; COc1ccc2[nH]cc(CCN(C)CCCF)c2c1 ; COc1ccc2[nH]cc(CCN(C)CCC(F)F)c2c1 ; COc1ccc2[nH]cc(CCN(C)CCC(F)(F)F)c2c1]

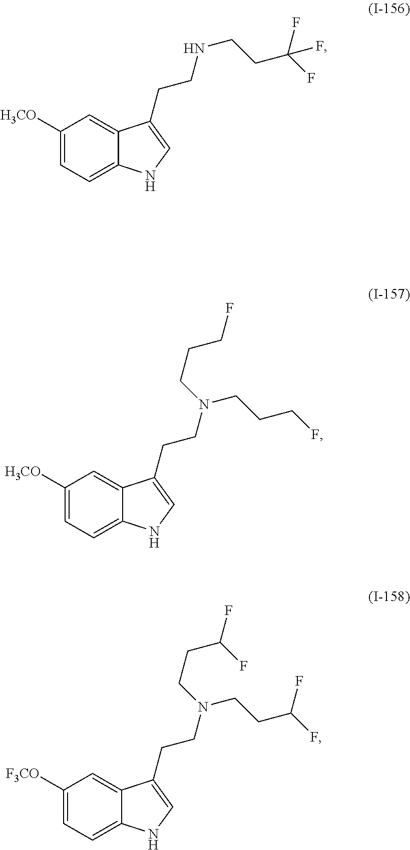 [SMILES: COc1ccc2[nH]cc(CCNCCC(F)(F)F)c2c1 ; COc1ccc2[nH]cc(CCN(CCCF)CCCF)c2c1 ; FC(F)CCN(CCc1c[nH]c2ccc(OC(F)(F)F)cc12)CCC(F)F]

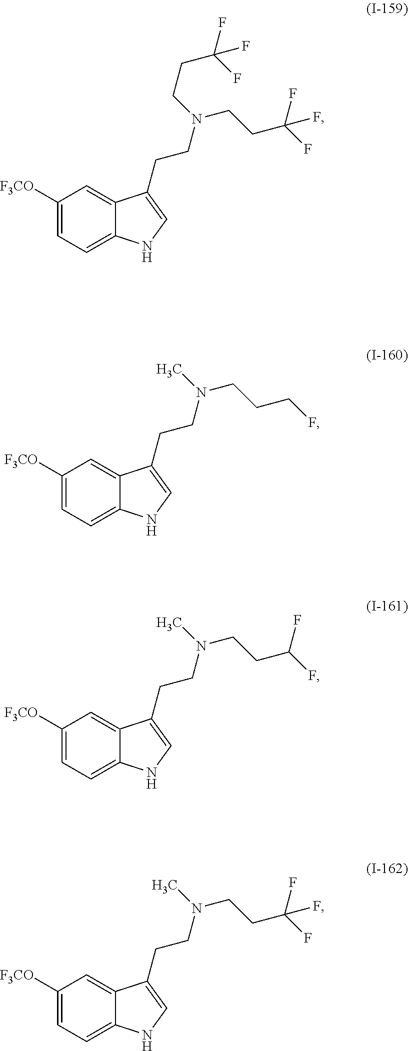 [SMILES: FC(F)(F)CCN(CCc1c[nH]c2ccc(OC(F)(F)F)cc12)CCC(F)(F)F ; CN(CCCF)CCc1c[nH]c2ccc(OC(F)(F)F)cc12 ; CN(CCc1c[nH]c2ccc(OC(F)(F)F)cc12)CCC(F)F ; CN(CCc1c[nH]c2ccc(OC(F)(F)F)cc12)CCC(F)(F)F]

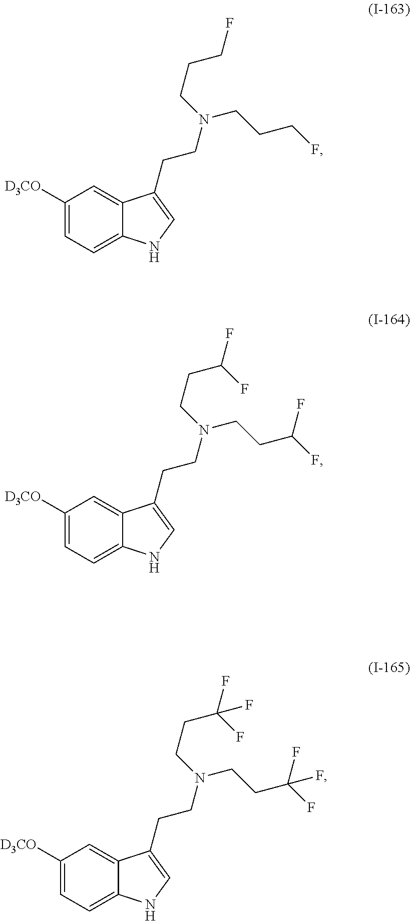

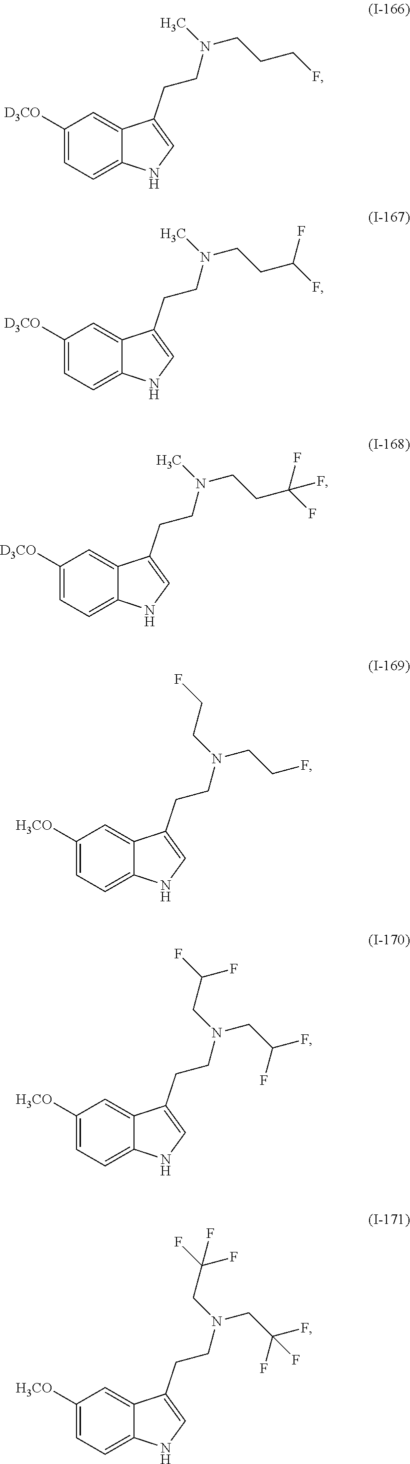

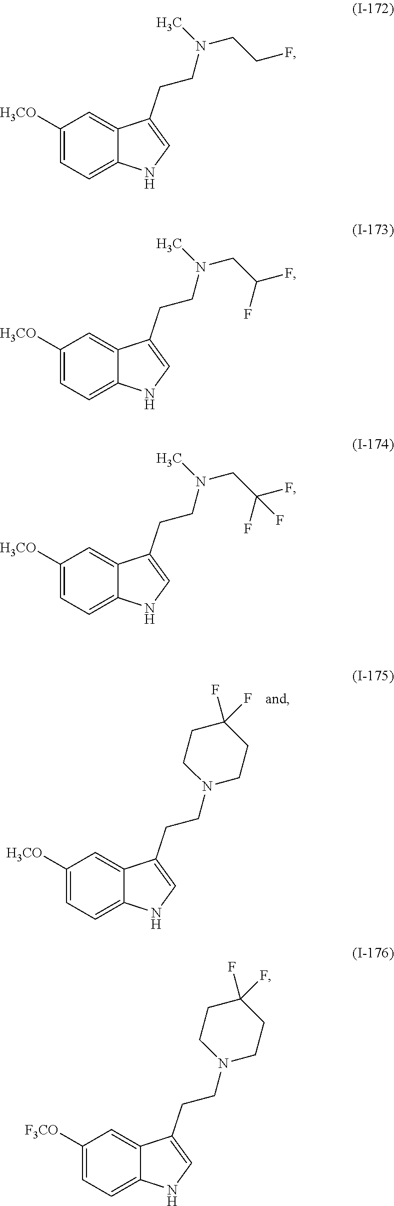

or a stereoisomer, solvate, or prodrug thereof, or a combination thereof.

In some embodiments, the compound, e.g., a compound of Formula (I) or (II), is a deuterated analog of DMT, examples of which include, but are not limited to,

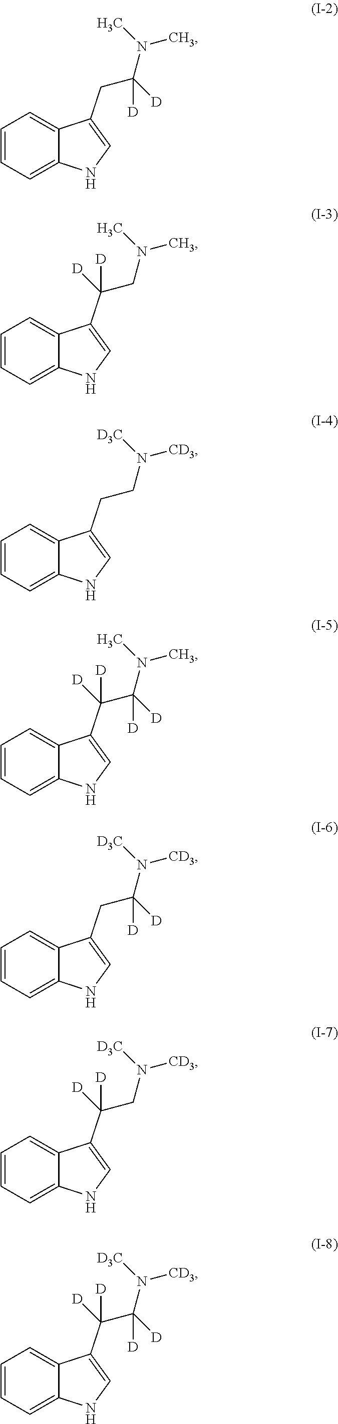

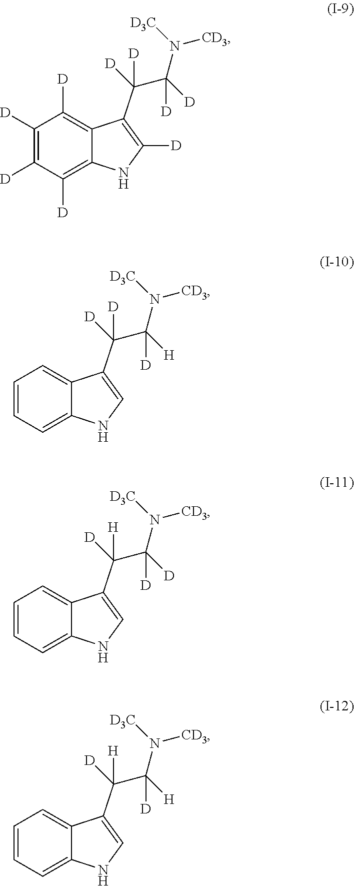 [SMILES: [2H]c1[nH]c2c([2H])c([2H])c([2H])c([2H])c2c1C([2H])([2H])C([2H])([2H])N(C([2H])([2H])[2H])C([2H])([2H])[2H] ; [2H]C(N(C([2H])([2H])[2H])C([2H])([2H])[2H])C([2H])([2H])c1c[nH]c2ccccc12 ; [2H]C(c1c[nH]c2ccccc12)C([2H])([2H])N(C([2H])([2H])[2H])C([2H])([2H])[2H] ; [2H]C(c1c[nH]c2ccccc12)C([2H])N(C([2H])([2H])[2H])C([2H])([2H])[2H]]

or a combination thereof.

In some embodiment, the deuterated analog of DMT is one or more of 2-(1H-indol-3-yl)-N,N-dimethylethan-1-amine-1,1-d₂ (I-2); 2-(1H-indol-3-yl)-N,N-bis(methyl-d₃)ethan-1-amine-1,1,2,2-d₄ (I-8); 2-(1H-indol-3-yl)-N,N-bis(methyl-d₃)ethan-1-amine-1,2,2-d₃ (I-10); 2-(1H-indol-3-yl)-N,N-bis(methyl-d₃)ethan-1-amine-1,1,2-d₃ (I-11); 2-(1H-indol-3-yl)-N,N-bis(methyl-d₃)ethan-1-amine-1,1-d₂ (I-6); 2-(1H-indol-3-yl)-N,N-bis(methyl-d₃)ethan-1-amine-2,2-d₂ (I-7); and 2-(1H-indol-3-yl)-N,N-bis(methyl-d₃)ethan-1-amine-1,2-d₂ (I-12).

In some embodiments, the compound, e.g., a compound of Formula (I) or (III), is a deuterated analog of 5-MeO-DMT, examples of which include, but are not limited to,

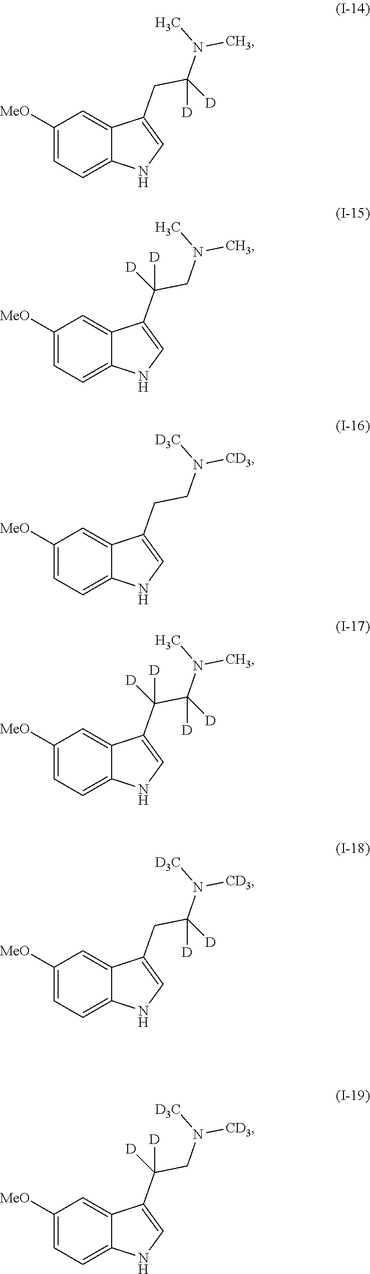 [SMILES: [2H]C([2H])(Cc1c[nH]c2ccc(OC)cc12)N(C)C ; [2H]C([2H])(CN(C)C)c1c[nH]c2ccc(OC)cc12 ; [2H]C([2H])([2H])N(CCc1c[nH]c2ccc(OC)cc12)C([2H])([2H])[2H] ; [2H]C([2H])(c1c[nH]c2ccc(OC)cc12)C([2H])([2H])N(C)C ; [2H]C([2H])([2H])N(C([2H])([2H])[2H])C([2H])([2H])Cc1c[nH]c2ccc(OC)cc12 ; [2H]C([2H])(CN(C([2H])([2H])[2H])C([2H])([2H])[2H])c1c[nH]c2ccc(OC)cc12]

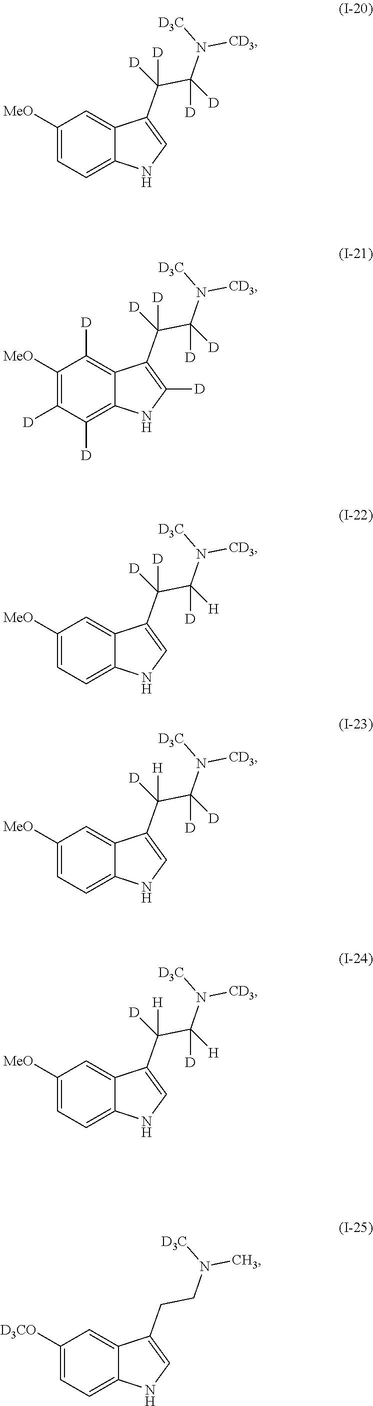

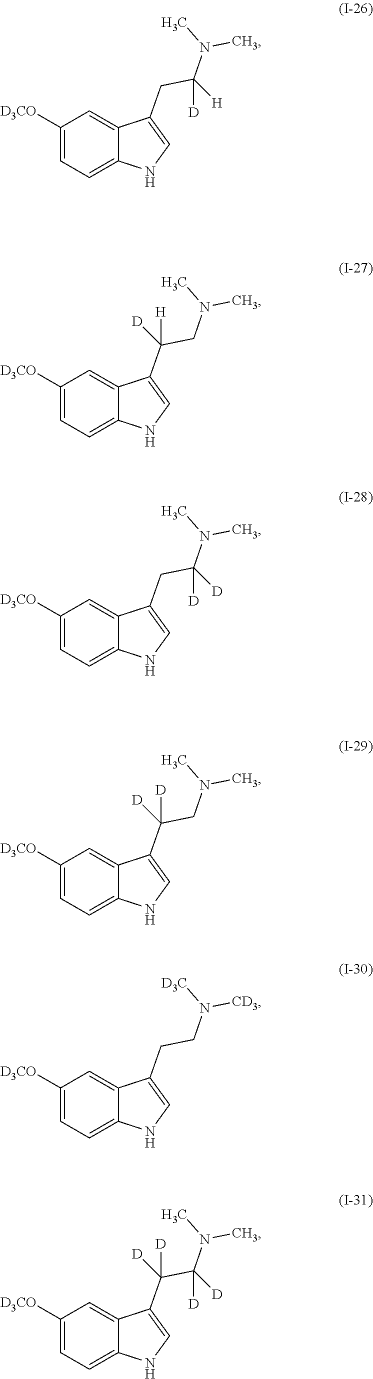

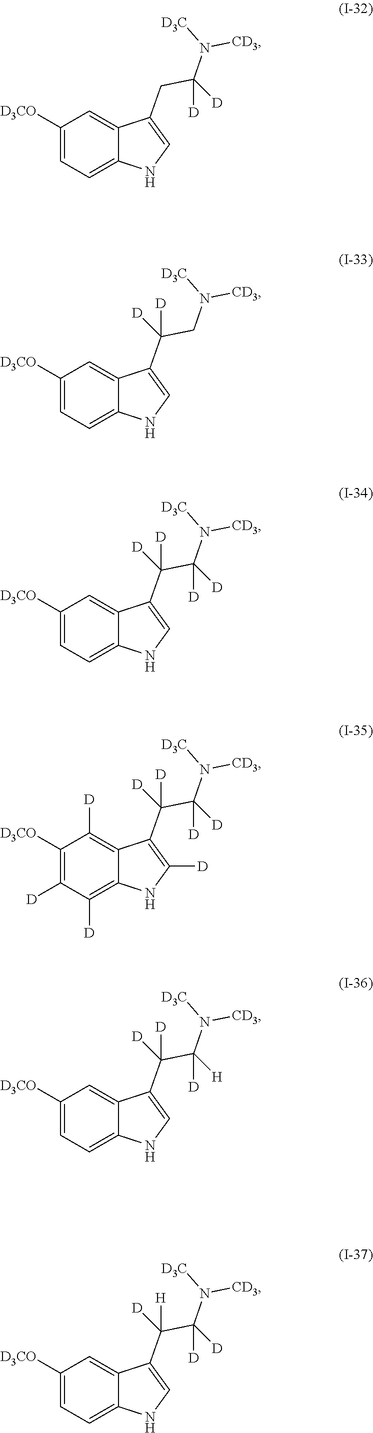

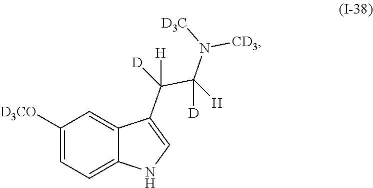

or a combination thereof.

In some embodiment, the deuterated analog of 5-MeO-DMT is one or more of 2-(5-methoxy-1H-indol-3-yl)-N,N-bis(methyl-d₃)ethan-1-amine-1,1,2,2-d₄ (I-20); 2-(5-methoxy-1H-indol-3-yl)-N,N-bis(methyl-d₃)ethan-1-amine-1,2,2-d₃ (I-22); 2-(5-methoxy-1H-indol-3-yl)-N,N-bis(methyl-d₃)ethan-1-amine-1,1,2-d₃ (I-23); 2-(5-methoxy-1H-indol-3-yl)-N,N-bis(methyl-d₃)ethan-1-amine-1,1-d₂ (I-18); 2-(5-methoxy-1H-indol-3-yl)-N,N-bis(methyl-d₃)ethan-1-amine-2,2-d₂ (I-19); 2-(5-methoxy-1H-indol-3-yl)-N,N-bis(methyl-d₃)ethan-1-amine-1,2-d₂ (I-24); 2-(5-(methoxy-d₃)-1H-indol-3-yl)-N,N-bis(methyl-d₃)ethan-1-amine-1,1,2,2-d₄ (I-34); 2-(5-(methoxy-d₃)-1H-indol-3-yl)-N,N-bis(methyl-d₃)ethan-1-amine-1,2,2-d₃ (I-36); 2-(5-(methoxy-d₃)-1H-indol-3-yl)-N,N-bis(methyl-d₃)ethan-1-amine-1,1,2-d₃ (I-37); 2-(5-(methoxy-d₃)-1H-indol-3-yl)-N,N-bis(methyl-d₃)ethan-1-amine-1,1-d₂ (I-32); 2-(5-(methoxy-d₃)-1H-indol-3-yl)-N,N-bis(methyl-d₃)ethan-1-amine-2,2-d₂ (I-33); 2-(5-(methoxy-d₃)-1H-indol-3-yl)-N,N-bis(methyl-d₃)ethan-1-amine-1,2-d₂ (I-38); 2-(5-(methoxy-d₃)-1H-indol-3-yl)-N,N-dimethylethan-1-amine-1,1-d₂ (I-28); 2-(5-(methoxy-d₃)-1H-indol-3-yl)-N,N-dimethylethan-1-amine-2,2-d₂ (I-29); 2-(5-(methoxy-d₃)-1H-indol-3-yl)-N,N-dimethylethan-1-amine-1-d (I-26); 2-(5-(methoxy-d₃)-1H-indol-3-yl)-N,N-dimethylethan-1-amine-2-d (I-27); and 2-(5-(methoxy-d₃)-1H-indol-3-yl)-N,N-dimethylethan-1-amine (I-25).

Any position in the compounds defined herein as having deuterium have a minimum deuterium incorporation that is greater than that found naturally occurring in hydrogen (about 0.016 atom %). In some embodiments, any position in the compound defined as having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

The compounds described herein, e.g., compounds of Formula (I) through (III), may contain a stereogenic center. In such cases, the compounds may exist as different stereoisomeric forms, even though Formula (I) through (III) are drawn without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

In some embodiments, the compounds described herein, e.g., compounds of Formula (I) through (III), are non-stereogenic. In some embodiments, the compounds described herein, e.g., compounds of Formula (I) through (III), are racemic. In some embodiments, the compounds described herein, e.g., compounds of Formula (I) through (III), are enantiomerically enriched (one enantiomer is present in a higher percentage), including enantiomerically pure. In some embodiments, the compounds described herein, e.g., compounds of Formula (I) through (III), are provided as a single diastereomer. In some embodiments, the compounds described herein, e.g., compounds of Formula (I) through (III), are provided as a mixture of diastereomers. When provided as a mixture of diastereomers, the mixtures may include equal mixtures, or mixtures which are enriched with a particular diastereomer (one diastereomer is present in a higher percentage than another).

As described herein, a racemic compound, e.g., a compound of Formula (I) through (III), may contain about 50% of the R- and S-stereoisomers based on a molar ratio (about 48 to about 52 mol %, or about a 1:1 ratio)) of one of the isomers. In some embodiments, a pharmaceutical formulation, medicament, or method of treatment may involve combining separately produced compounds of the R- and S-stereoisomers in an approximately equal molar ratio (e.g., about 48 to 52%). In some embodiments, a medicament or pharmaceutical formulation may contain a mixture of separate compounds of the R- and S-stereoisomers in different ratios. In some embodiments, the pharmaceutical formulation contains an excess (greater than 50%) of the R-enantiomer. Suitable molar ratios of R/S may be from about 1.5:1, 2:1, 3:1, 4:1, 5:1, 10:1, or higher. In some embodiments, a pharmaceutical formulation may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In other embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included. Pharmaceutical formulations may contain a mixture of the racemate and a separate compound of Formula (I) through (III), in salt form.

In some embodiments, the compound of Formula (I) through (III) is an agonist of a serotonin 5-HT₂ receptor. In some embodiments, the compound of Formula (I) through (III) is an agonist of a serotonin 5-HT_{2A} receptor. In some embodiments, the compound of Formula (I) through (III) is an agonist of a serotonin 5-HT_{1A} receptor. In some embodiments, the compound of Formula (I) through (III) is an agonist of a serotonin 5-HT_{2C} receptor.

In some embodiments, the tryptamine psychedelic used in the preparation of the pharmaceutical formulation is chemically pure, for example has a purity of greater than 90%, 92%, 94%, 96%, 97%, 98%, or 99% by UPLC or HPLC. In some embodiments, the tryptamine psychedelic has no single impurity of greater than 1%, greater than 0.5%, greater than 0.4%, greater than 0.3%, or greater than 0.2%, measured by UPLC or HPLC. In some embodiments, the tryptamine psychedelic has a chemical purity of greater than 97 area %, greater than 98 area %, or greater than 99 area % by UPLC or HPLC. In some embodiments, the tryptamine psychedelic has no single impurity greater than 1 area %, greater than 0.5 area %, greater than 0.4 area %, greater than 0.3 area %, or greater than 0.2 area % as measured by UPLC or HPLC.

Acids which may be used to form the pharmaceutically acceptable (acid addition) salts of the compounds disclosed herein, e.g., compounds of Formula (I) through (III), include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.). Combinations of acids may also be used to form a mixture of acid addition salts.

When the injectable pharmaceutical formulation is prepared from a pre-formed pharmaceutically acceptable salt of a compound disclosed herein, certain salt forms are preferred among the list above because they possess physical and pharmaceutical characteristics/properties which make them well-suited for pharmaceutical preparation and administration. For example, preferred salt forms of the compounds disclosed herein (e.g., compounds of Formula (I) through (III)) are those that possess one or more of the following characteristics: are easy to prepare in high yield with a propensity towards salt formation; are stable and have well-defined physical properties such as crystallinity, lack of polymorphism, and high melting/enthalpy of fusion; have slight or no hygroscopicity; are free flowing, do not cohere/adhere to surfaces, and possess a regular morphology; have acceptable aqueous solubility for the intended route of administration; and/or are physiologically acceptable, e.g., do not cause irritation when administered to mammals.

### Crystallinity

When pre-formed as a solid, the pharmaceutically acceptable salt of the compound of the present disclosure (e.g., a compound of Formula (I) through (III)) may be crystalline or amorphous, preferably crystalline, as determined e.g., by X-ray powder diffraction (XRPD). In some embodiments, the pharmaceutically acceptable salt of the compound of the present disclosure is amorphous, e.g., as determined by XRPD and/or DSC. The pharmaceutically acceptable salt of the compound of the present disclosure can be in a stable amorphous form. In some embodiments, a highly pure amorphous form of a pharmaceutically acceptable salt of a compound of the present disclosure is provided, wherein at least 92%, at least 94%, at least 96%, at least 98%, at least 99%, or at least 99.5% by weight of the pharmaceutically acceptable salt of the compound of the present disclosure is in amorphous form, e.g., as determined by X-ray powder diffraction and/or DSC. In some embodiments, the pharmaceutically acceptable salt of the compound of the present disclosure is crystalline. Crystalline forms are advantageous in terms of e.g., stability and providing well-defined physical properties, which is desirable for pharmaceutical preparation and administration. The pharmaceutically acceptable salt of the compound of the present disclosure can be in a stable crystalline form. In some embodiments, the pharmaceutically acceptable salt of the compound of the present disclosure has a percent crystallinity of at least 50%, at least 60%, at least 70%, at least 80%, at least 90%, at least 95%, at least 99%, or at least 99.5%, and up to 100%, as determined by XRPD and/or DSC analysis. In some embodiments, a highly pure crystalline form of a pharmaceutically acceptable salt of a compound of the present disclosure is provided, wherein at least 90%, at least 95%, at least 99%, or at least 99.5% by weight of the pharmaceutically acceptable salt of the compound of present disclosure is in crystalline form, e.g., as determined by X-ray powder diffraction and/or DSC. Preference is given to those pre-formed salt forms with high crystallinity, as determined e.g., by discrete and sharp Bragg diffractions in the X-ray diffractograms.

XRPD analyses can be carried out, e.g., on a Bruker D5000 X-ray powder diffractometer using CuKα radiation (wavelength=1.54060 Å). The instrument may be equipped with a fine focus X-ray tube. The tube voltage and amperage can be set to 40 kV and 30 mA, respectively. The divergence and scattering slit widths can be set at 2 mm and the detector slit width can be set at 0.2 mm. Diffracted radiation can be detected by a NaI scintillation detector. A theta-two theta continuous scan from 2.0 to 400 (4 seconds per step; 0.01° step size) can be used.

In terms of pharmaceutical production processes, advantageous salt forms of the compounds of the present disclosure are those that readily afford a crystalline solid on crystallization in acceptable yield without proceeding via an oil, and with favorable volume factors, making them suitable for mass production.

Salts forms of the compound of the present disclosure (e.g., a compound of Formula (I) through (III)) can in some cases exist in different polymorphs (i.e., forms having a different crystal structure), however, preferred salt forms of the present disclosure are those which can be crystallized into a single crystalline form or single polymorph, as determined by XRPD and/or differential scanning calorimetry (DSC). It is also generally desirable for the salt forms to be free flowing, not cohere/adhere to surfaces, and possess a regular morphology.

### Chemical/Solid-State Stability

In some embodiments, the pharmaceutically acceptable salt of the compound of the present disclosure has a melt onset of from about 100° C., from about 110° C., from about 120° C., from about 130° C., from about 140° C., from about 150° C., from about 160° C., from about 170° C., from about 180° C., from about 190° C., and up to about 250° C., up to about 225° C., up to about 210° C., up to about 200° C., as determined by DSC.

In some embodiments, the pharmaceutically acceptable salt of the compound of the present disclosure has an enthalpy of fusion of from about 90 J·g⁻¹, from about 100 J·g⁻¹, from about 110 J·g⁻¹, from about 120 J·g⁻¹, from about 130 J·g⁻¹, from about 140 J·g⁻¹, from about 150 J·g⁻¹, from about 160 J·g⁻¹, and up to about 190 J·g⁻¹, up to about 180 J·g⁻¹, up to about 170 J·g⁻¹, as determined by DSC.

Pre-formed pharmaceutically acceptable salts of the compound of the present disclosure suitable for pharmaceutical manufacture may also be characterized as non-hygroscopic or slightly hygroscopic, preferably non-hygroscopic. The hygroscopicity may be measured herein by performing a moisture adsorption-desorption isotherm using a dynamic vapor sorption (DVS) analyzer with a starting exposure of 30% relative humidity (RH), increasing humidity up to 95% RH, decreasing humidity to 0%, and finally increasing the humidity back to the starting 30% RH, and classified according to the following:
non-hygroscopic: <0.2%; slightly hygroscopic: ≥0.2% and <2%; hygroscopic: ≥2% and <15%; very hygroscopic: ≥15%; deliquescent: sufficient water is absorbed to form a liquid; all values measured as weight increase (w/w due to acquisition of water) at >95% RH and 25° C.
   In some embodiments, the pharmaceutically acceptable salt of the compound of the present disclosure has a weight increase at >95% RH of less than 1% w/w, less than 0.8% w/w, less than 0.6% w/w, less than 0.5% w/w, less than 0.4% w/w, less than 0.3% w/w, less than 0.2% w/w, less than 0.1% w/w, less than 0.08% w/w, less than 0.06% w/w, less than 0.05% w/w, less than 0.02% w/w, as determined by DVS.

Pre-formed pharmaceutically acceptable salts of the compounds of the present disclosure can be maintained/stored in open or closed environments, such as in open or closed flasks/vials, under ambient or stress conditions e.g., 25° C./60% RH, 25° C./90+% RH, 40° C./75% RH, etc. without appreciable degradation (e.g., without appreciably diminished chemical purity) or physical changes (e.g., changed forms, deliquesced, etc.). For example, dry powder samples of salt forms disclosed herein may have a purity change or form change of less than 10%, less than 5%, less than 1%, when stored under ambient conditions or stress conditions (e.g., increased temperature, e.g., 40° C., and/or humidity).

### Physiologically Acceptability

Suitable salt forms of the compounds of the present disclosure are physiologically acceptable and do not cause excessive irritation or tissue damage at the injection site. Accordingly, preferred pharmaceutical salts of the compounds of the present disclosure, e.g., compounds of Formula (I) through (III), are those formed with an organic acid, preferably an organic acid with a mild acidity, for example an organic acid with a pKₐ in water of no less than 1.0, no less than 1.5, no less than 2.0, no less than 2.5, no less than 3.0, no less than 3.5, no less than 4.0, no less than 4.5, for example, from 3.0 to 6.5.

### Solubility

The aqueous solubility of the pharmaceutical salts of the compounds of the present disclosure can be determined by equilibrating excess solid with 1 mL of water for 24 hours at 22° C. A 200 uL aliquot can be centrifuged at 15,000 revolutions per minute (rpm) for 15 minutes. The supernatant can be analyzed by UPLC or HPLC and the solubility can be expressed as its free base equivalent (mg FB/mL). For example, pharmaceutically acceptable salt forms of the compounds of the present disclosure can be prepared and the solubility and solution pH can be measured.

In some embodiments, the pharmaceutically acceptable salt of the compound of the present disclosure, e.g., a compound of Formula (I) through (III), has a water solubility at 22° C. of from about 5 mg/mL to about 400 mg/mL. In some embodiments, the pharmaceutically acceptable salt of the compound of the present disclosure has a water solubility of from about 1 mg/mL, from about 2 mg/mL, from about 3 mg/mL, from about 5 mg/mL, from about 10 mg/mL, from about 20 mg/mL, from about 30 mg/mL, from about 40 mg/mL, from about 50 mg/mL, from about 60 mg/mL, from about 70 mg/mL, from about 80 mg/mL, from about 90 mg/mL, from about 100 mg/mL, from about 110 mg/mL, from about 120 mg/mL, from about 130 mg/mL, from about 140 mg/mL, from about 150 mg/mL, and up to about 400 mg/mL, up to about 380 mg/mL, up to about 360 mg/mL, up to about 340 mg/mL, up to about 320 mg/mL, up to about 300 mg/mL, up to about 280 mg/mL, up to about 260 mg/mL, up to about 250 mg/mL, or any range therebetween. In some embodiments, the pharmaceutically acceptable salt of the compound of the present disclosure has a water solubility from about 200 mg/mL to about 400 mg/mL. In some embodiments, the pharmaceutically acceptable salt of the compound of the present disclosure has a water solubility from about 150 mg/mL to about 250 mg/mL. In some embodiments, the pharmaceutically acceptable salt of the compound of the present disclosure has a water solubility of greater than about 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL.

In some embodiments, the pharmaceutically acceptable salt of the compound of the present disclosure, e.g., the compound of Formula (I) through (III), is a fumarate, a benzoate, a salicylate, a succinate, an oxalate, a glycolate, a hemi-oxalate, or a hemi-fumarate salt. In terms of providing desirable physical and pharmaceutical characteristics, such as those described above, preferred pharmaceutically acceptable salts are fumarate salts, hemi-fumarate salts, benzoate salts, salicylates, and succinate salts of the compounds disclosed herein, e.g., the compounds of Formula (I) through (III), with fumarate, benzoate, and salicylate salts being particularly preferred. In some embodiments, the pharmaceutically acceptable salt of the compound of the present disclosure, e.g., the compound of Formula (I) through (III), is a benzenesulfonate salt.

In some embodiments, the pharmaceutically acceptable salt is a fumarate, a benzoate, a salicylate, a succinate, an oxalate, a glycolate, a hemi-oxalate, or a hemi-fumarate salt of N,N-dimethyltryptamine (DMT). In some embodiments, the pharmaceutically acceptable salt is a fumarate, a benzoate, a salicylate, a succinate, an oxalate, a glycolate, a hemi-oxalate, or a hemi-fumarate salt of 5-hydroxy-N,N-dimethyltryptamine (5-OH-DMT). In some embodiments, the pharmaceutically acceptable salt is a fumarate, a benzoate, a salicylate, a succinate, an oxalate, a glycolate, a hemi-oxalate, or a hemi-fumarate salt of 5-methoxy-N,N-dimethyltryptamine (5-MeO-DMT). In some embodiments, the pharmaceutically acceptable salt is a fumarate, a benzoate, a salicylate, a succinate, an oxalate, a glycolate, a hemi-oxalate, or a hemi-fumarate salt of 2-(1H-indol-3-yl)-N,N-bis(methyl-d₃)ethan-1-amine-1,1,2,2-d₄ (DMT-d₁₀). In some embodiments, the pharmaceutically acceptable salt is a fumarate, a benzoate, a salicylate, a succinate, an oxalate, a glycolate, a hemi-oxalate, or a hemi-fumarate salt of 2-(1H-indol-3-yl)-N,N-bis(methyl-d₃)ethan-1-amine-1,1-d₂ (a DMT-d₈). In some embodiments, the pharmaceutically acceptable salt is a fumarate, a benzoate, a salicylate, a succinate, an oxalate, a glycolate, a hemi-oxalate, or a hemi-fumarate salt of a pharmaceutically acceptable salt of 2-(1H-indol-3-yl)-N,N-dimethylethan-1-amine-1,1-d₂ (a DMT-d₂). In some embodiments, the pharmaceutically acceptable salt is a fumarate, a benzoate, a salicylate, a succinate, an oxalate, a glycolate, a hemi-oxalate, or a hemi-fumarate salt of 2-(5-methoxy-1H-indol-3-yl)-N,N-bis(methyl-d₃)ethan-1-amine-1,1,2,2-d₄ (5-MeO-DMT-d₁₀). In some embodiments, the pharmaceutically acceptable salt is a fumarate, a benzoate, a salicylate, a succinate, an oxalate, a glycolate, a hemi-oxalate, or a hemi-fumarate salt of 2-(5-(methoxy-d₃)-1H-indol-3-yl)-N,N-dimethylethan-1-amine-1,1-d₂ (5-MeO-DMT-d₅). In some embodiments, the pharmaceutically acceptable salt is a fumarate, a benzoate, a salicylate, a succinate, an oxalate, a glycolate, a hemi-oxalate, or a hemi-fumarate salt of 2-(5-(methoxy-d₃)-1H-indol-3-yl)-N,N-bis(methyl-d₃)ethan-1-amine-1,1,2,2-d₄ (5-MeO-DMT-d₁₃).

In some embodiments, the pharmaceutically acceptable salt of DMT or a deuterated analog of DMT (e.g., DMT-d₁₀) is a crystalline solid as disclosed in PCT/EP2023/050702, which is incorporated herein by reference in its entirety.

Non-limiting examples of pharmaceutically acceptable salts of compounds of Formula (I) and (II) are provided in Table 1.

TABLE 1
Exemplary pharmaceutically acceptable
salts of compounds of Formula (I)/(II)

| Salt form identifier | Salt type of compound |
|---|---|
| I-1a | Fumarate of I-1 |
| I-1b | Benzoate of I-1 |
| I-1c | Salicylate of I-1 |
| I-1d | Succinate of I-1 |
| I-2a | Fumarate of I-2 |
| I-2b | Benzoate of I-2 |
| I-2c | Salicylate of I-2 |
| I-2d | Succinate of I-2 |
| I-3a | Fumarate of I-3 |
| I-3b | Benzoate of I-3 |
| I-3c | Salicylate of I-3 |
| I-3d | Succinate of I-3 |
| I-4a | Fumarate of I-4 |
| I-4b | Benzoate of I-4 |
| I-4c | Salicylate of I-4 |
| I-4d | Succinate of I-4 |
| I-5a | Fumarate of I-5 |
| I-5b | Benzoate of I-5 |
| I-5c | Salicylate of I-5 |
| I-5d | Succinate of I-5 |
| I-6a | Fumarate of I-6 |
| I-6b | Benzoate of I-6 |
| I-6c | Salicylate of I-6 |
| I-6d | Succinate of I-6 |
| I-7a | Fumarate of I-7 |
| I-7b | Benzoate of I-7 |
| I-7c | Salicylate of I-7 |
| I-7d | Succinate of I-7 |
| I-8a | Fumarate of I-8 |
| I-8b | Benzoate of I-8 |
| I-8c | Salicylate of I-8 |
| I-8d | Succinate of I-8 |
| I-9a | Fumarate of I-9 |
| I-9b | Benzoate of I-9 |
| I-9c | Salicylate of I-9 |
| I-9d | Succinate of I-9 |
| I-10a | Fumarate of I-10 |
| I-10b | Benzoate of I-10 |
| I-10c | Salicylate of I-10 |
| I-10d | Succinate of I-10 |
| I-11a | Fumarate of I-11 |
| I-11b | Benzoate of I-11 |
| I-11c | Salicylate of I-11 |
| I-11d | Succinate of I-11 |
| I-12a | Fumarate of I-12 |
| I-12b | Benzoate of I-12 |
| I-12c | Salicylate of I-12 |
| I-12d | Succinate of I-12 |

In some embodiments, the pharmaceutically acceptable salt of a compound of the present disclosure is a fumarate salt of 2-(1H-indol-3-yl)-N,N-dimethylethan-1-amine (I-1a) (i.e., a fumarate salt of compound I-1, depicted below). In some embodiments, when salt I-1a is in a crystalline solid form it is characterized by an X-ray powder diffraction pattern containing at least three characteristic peaks at diffraction angles (2θ±0.20) selected from 7.80, 10.30, 10.90, 13.60, 15.80, 16.10, 17.00, 18.40, 19.70, 19.90, 20.60, 21.30, 21.70, 22.50, 23.90, 24.10, 25.10, 26.20, 33.60, and 34.90, as determined by XRPD using a CuKα radiation source.

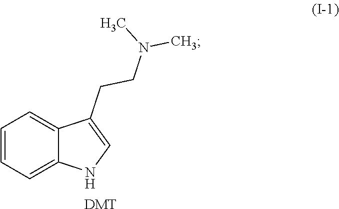

In some embodiments, the pharmaceutically acceptable salt of a compound of the present disclosure is a benzoate salt of 2-(1H-indol-3-yl)-N,N-dimethylethan-1-amine (I-1b) (i.e., a benzoate salt of compound I-1 depicted above). In some embodiments, when salt I-1b is in a crystalline solid form it is characterized by an X-ray powder diffraction pattern containing at least three characteristic peaks at diffraction angles (2θ±0.2°) selected from 9.6°, 11.1°, 12.6°, 13.5°, 15.8°, 16.1°, 17.1°, 17.9°, 19.8°, 20.1°, 20.8°, 21.2°, 22.7°, 23.8°, 24.6°, 26.9°, 29.2°, 32.3° 35.1°, and 36.10, as determined by XRPD using a CuKα radiation source.

In some embodiments, the pharmaceutically acceptable salt of a compound of the present disclosure is a salicylate salt of 2-(1H-indol-3-yl)-N,N-dimethylethan-1-amine (I-1c) (i.e., a salicylate salt of compound I-1 depicted above). In some embodiments, when salt I-1c is in a crystalline solid form it is characterized by an X-ray powder diffraction pattern containing at least three characteristic peaks at diffraction angles (2θ±0.2°) selected from 9.6°, 10.5°, 14.9°, 17.10, 18.1°, 19.1°, 20.1°, 20.7°, 21.0°, 21.3°, 24.6°, 25.6°, 28.5°, 28.8°, 29.4°, 30.3°, 31.3°, 32.1°, 33.5° and 34.4°, as determined by XRPD using a CuKα radiation source.

In some embodiments, the pharmaceutically acceptable salt of a compound of the present disclosure is a succinate salt of 2-(1H-indol-3-yl)-N,N-dimethylethan-1-amine (I-1d) (i.e., a succinate salt of compound I-1 depicted above). In some embodiments, when salt I-1d is in a crystalline solid form it is characterized by an X-ray powder diffraction pattern containing at least three characteristic peaks at diffraction angles (2θ±0.2°) selected from 9.8°, 11.7°, 14.3°, 14.7°, 17.0°, 17.4°, 19.6°, 20.6°, 22.3°, 22.6°, 22.9°, 23.1°, 23.4°, 24.9°, 25.2°, 26.3°, 26.8°, 27.3°, 27.7°, 28.8°, 29.10, 30.9°, 31.5°, 33.8°, 34.5°, 36.5°, and 39.2°, as determined by XRPD using a CuKα radiation source.

In some embodiments, the pharmaceutically acceptable salt of a compound of the present disclosure is an oxalate salt of 2-(1H-indol-3-yl)-N,N-dimethylethan-1-amine (I-1e) (i.e., an oxalate salt of compound I-1 depicted above). In some embodiments, when salt I-1e is in a crystalline solid form it is characterized by an X-ray powder diffraction pattern containing at least three characteristic peaks at diffraction angles (2θ±0.2°) selected from 11.3°, 12.3°, 15.6°, 17.7°, 19.5°, 20.0°, 20.8°, 21.4°, 22.3°, 22.7°, 24.8°, 25.7°, 26.7°, 27.9°, 28.7°, 29.5°, 31.4°, 33.0°, 35.4° 36.5°, and 38.6°, as determined by XRPD using a CuKα radiation source.

In some embodiments, the pharmaceutically acceptable salt of a compound of the present disclosure is a glycolate salt of 2-(1H-indol-3-yl)-N,N-dimethylethan-1-amine (I-1f) (i.e., a glycolate salt of compound I-1 depicted above). In some embodiments, when salt I-1f is in a crystalline solid form it is characterized by an X-ray powder diffraction pattern containing at least three characteristic peaks at diffraction angles (2θ±0.2°) selected from 8.2°, 12.2°, 12.9°, 15.8°, 16.3°, 17.8°, 19.2°, 20.1°, 21.7°, 23.6°, 24.4°, 24.6°, 24.9°, 26.0°, 26.6°, 27.8°, 29.6°, 30.2°, 32.0°, 32.3°, 33.0°, 33.9°, and 34.6°, as determined by XRPD using a CuKα radiation source.

In some embodiments, the pharmaceutically acceptable salt of a compound of the present disclosure is a hemi-oxalate salt of 2-(1H-indol-3-yl)-N,N-dimethylethan-1-amine (I-1g) (i.e., a hemi-oxalate salt of compound I-1 depicted above). In some embodiments, when salt I-1g is in a crystalline solid form it is characterized by an X-ray powder diffraction pattern containing at least three characteristic peaks at diffraction angles (2θ±0.2°) selected from 8.7°, 11.5°, 13.6°, 14.2°, 15.2°, 17.4°, 17.6°, 18.0°, 19.3°, 19.6°, 20.1°, 20.6°, 21.9°, 22.1°, 22.9°, 23.2°, 23.5°, 24.5°, 25.0°, 25.5°, 26.1°, 26.4°, 27.1°, 28.4°, 28.7°, 29.8°, 30.4°, 30.7°, 31.4°, 31.8°, 33.4° and 33.9° as determined by XRPD using a CuKα radiation source.

In some embodiments, the pharmaceutically acceptable salt of a compound of the present disclosure is a hemi-fumarate salt of 2-(1H-indol-3-yl)-N,N-dimethylethan-1-amine (I-1h) (i.e., a hemi-fumarate salt of compound I-1 depicted above). In some embodiments, when salt I-1h is in a crystalline solid form it is characterized by an X-ray powder diffraction pattern containing at least three characteristic peaks at diffraction angles (2θ±0.2°) selected from 8.10, 11.3°, 12.2°, 13.3°, 14.2°, 16.2°, 17.6°, 18.3°, 18.6°, 19.5°, 19.8°, 20.0°, 20.2°, 20.9°, 21.4°, 21.9°, 22.3°, 22.7°, 22.9°, 23.8°, 24.5°, 25.0°, 25.2°, 26.1°, 26.4°, 26.9°, 28.4°, 28.8°, 29.5°, 29.8°, 30.9°, and 32.7°, as determined by XRPD using a CuKα radiation source.

In some embodiments, the pharmaceutically acceptable salt of a compound of the present disclosure is a fumarate salt of 2-(1H-indol-3-yl)-N,N-bis(methyl-d₃)ethan-1-amine-1,1,2,2-d₄ (I-8a) (i.e., a fumarate salt of compound I-8 depicted below). In some embodiments, when salt I-8a is in a crystalline solid form it is characterized by an X-ray powder diffraction pattern containing at least three characteristic peaks at diffraction angles (2θ±0.2°) selected from 7.8°, 10.3°, 10.9°, 12.5°, 13.6°, 14.6°, 15.2°, 15.5°, 15.8°, 16.1°, 16.6°, 17.0°, 18.4°, 19.0°, 19.7°, 19.9°, 20.6°, 21.3°, 21.8°, 22.5°, 23.3°, 23.8°, 24.1°, 25.1°, 26.2°, 26.8°, 27.3°, 27.9°, 28.3°, 28.9°, 29.3°, 29.6°, 29.9°, 30.6°, 31.0°, 31.3°, 32.4°, 32.9°, 33.3°, 33.6°, 34.3°, 34.9°, 35.7°, 36.1°, 37.4°, 38.0°, and 38.5°, as determined by XRPD using a CuKα radiation source. In some embodiments, when salt I-8a is in a crystalline solid form it is characterized by an X-ray powder diffraction pattern containing at least three characteristic peaks at diffraction angles (2θ±0.2°) selected from 7.8°, 10.3°, 10.9°, 13.6°, 15.8°, 16.1°, 17.0°, 18.4°, 19.7°, 19.9°, 20.6°, 21.3°, 21.8°, 22.5°, 23.8°, 24.1°, 25.1°, 26.2°, 33.6°, and 34.9°, as determined by XRPD using a CuKα radiation source. In some embodiments, when salt I-8a is in a crystalline solid form it is characterized by an X-ray powder diffraction pattern containing at least three characteristic peaks at diffraction angles (2θ±0.2°) selected from 10.9°, 13.6°, 15.8°, 16.1°, 17.0°, 18.4°, 19.7°, 19.9°, 20.6°, 23.8°, 24.1°, and 25.1°, as determined by XRPD using a CuKα radiation source.

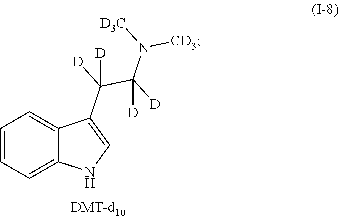 [SMILES: [2H]C([2H])([2H])N(C([2H])([2H])[2H])C([2H])([2H])C([2H])([2H])c1c[nH]c2ccccc12]

In some embodiments, the pharmaceutically acceptable salt of a compound of the present disclosure is a benzoate salt of 2-(1H-indol-3-yl)-N,N-bis(methyl-d₃)ethan-1-amine-1,1,2,2-d₄ (I-8b) (i.e., a benzoate salt of compound I-8 depicted above). In some embodiments, when salt I-8b is in a crystalline solid form it is characterized by an X-ray powder diffraction pattern containing at least three characteristic peaks at diffraction angles (2θ±0.2°) selected from 9.6°, 11.10, 12.7°, 13.5°, 15.8°, 16.1°, 17.2°, 17.9°, 19.8°, 20.1°, 20.8°, 21.2°, 22.8°, 23.8°, 24.3°, 24.6°, 25.1°, 25.3°, 25.5°, 26.9°, 28.3°, 28.9°, 29.3°, 31.4°, 31.6°, 32.0°, 32.3°, 32.8°, 35.1°, and 36.1°, as determined by XRPD using a CuKα radiation source. In some embodiments, when salt I-8b is in a crystalline solid form it is characterized by an X-ray powder diffraction pattern containing at least three characteristic peaks at diffraction angles (2θ±0.2°) selected from 9.6°, 11.1°, 12.7°, 13.5°, 15.8°, 16.1°, 17.2°, 17.9°, 19.8°, 20.1°, 20.8°, 21.2°, 22.8°, 23.8°, 24.6°, 26.9°, 29.3°, 32.3° 35.1°, and 36.1°, as determined by XRPD using a CuKα radiation source. In some embodiments, when salt I-8b is in a crystalline solid form it is characterized by an X-ray powder diffraction pattern containing at least three characteristic peaks at diffraction angles (2θ±0.2°) selected from 12.7°, 13.5°, 15.8°, 16.1°, 17.2°, 17.9°, 19.8°, 20.1°, 20.8°, 23.8°, 24.6°, 26.9°, 29.3°, and 35.1° as determined by XRPD using a CuKα radiation source.

In some embodiments, the pharmaceutically acceptable salt of a compound of the present disclosure is a salicylate salt of 2-(1H-indol-3-yl)-N,N-bis(methyl-d₃)ethan-1-amine-1,1,2,2-d₄ (I-8c) (i.e., a salicylate salt of compound I-8 depicted above). In some embodiments, when salt I-8c is in a crystalline solid form it is characterized by an X-ray powder diffraction pattern containing at least three characteristic peaks at diffraction angles (2θ±0.2°) selected from 9.6°, 10.5°, 11.4°, 12.3°, 13.4°, 14.2°, 14.9°, 15.6°, 16.1°, 17.1°, 18.1°, 18.7°, 19.1°, 20.1°, 20.8°, 21.1°, 21.3°, 22.2°, 22.6°, 23.7°, 24.6°, 25.2°, 25.6°, 26.1°, 26.4°, 27.4°, 27.5°, 27.8°, 28.5°, 28.8°, 29.4°, 29.7°, 30.3°, 31.0°, 31.3°, 32.1°, 32.7°, 33.1°, 33.5°, 34.4°, and 35.0°, as determined by XRPD using a CuKα radiation source. In some embodiments, when salt I-8c is in a crystalline solid form it is characterized by an X-ray powder diffraction pattern containing at least three characteristic peaks at diffraction angles (2θ±0.2°) selected from 9.6°, 10.5°, 14.9°, 17.1°, 18.1°, 19.1°, 20.1°, 20.8°, 21.1°, 21.3°, 24.6°, 25.6°, 28.5°, 28.8°, 29.4°, 30.3°, 31.3°, 32.1°, 33.5° and 34.4° as determined by XRPD using a CuKα radiation source. In some embodiments, when salt I-8c is in a crystalline solid form it is characterized by an X-ray powder diffraction pattern containing at least three characteristic peaks at diffraction angles (2θ±0.2°) selected from 9.6°, 14.9°, 17.1°, 18.1°, 19.1°, 20.1°, 20.8°, 21.3°, 24.6°, 25.6°, 28.5°, and 32.1°, as determined by XRPD using a CuKα radiation source.

Non-limiting examples of pharmaceutically acceptable salts of compounds of Formula (I) and (III) are provided in Table 2.

TABLE 2
Exemplary pharmaceutically acceptable
salts of compounds of Formula (I)/(III)

| Salt form identifier | Salt type of compound |
|---|---|
| I-13a | Fumarate of I-13 |
| I-13b | Benzoate of I-13 |
| I-13c | Salicylate of I-13 |
| I-13d | Succinate of I-13 |
| I-14a | Fumarate of I-14 |
| I-14b | Benzoate of I-14 |
| I-14c | Salicylate of I-14 |
| I-14d | Succinate of I-14 |
| I-15a | Fumarate of I-15 |
| I-15b | Benzoate of I-15 |
| I-15c | Salicylate of I-15 |
| I-15d | Succinate of I-15 |
| I-16a | Fumarate of I-16 |
| I-16b | Benzoate of I-16 |
| I-16c | Salicylate of I-16 |
| I-16d | Succinate of I-16 |
| I-17a | Fumarate of I-17 |
| I-17b | Benzoate of I-17 |
| I-17c | Salicylate of I-17 |
| I-17d | Succinate of I-17 |
| I-18a | Fumarate of I-18 |
| I-18b | Benzoate of I-18 |
| I-18c | Salicylate of I-18 |
| I-18d | Succinate of I-18 |
| I-19a | Fumarate of I-19 |
| I-19b | Benzoate of I-19 |
| I-19c | Salicylate of I-19 |
| I-19d | Succinate of I-19 |
| I-20a | Fumarate of I-20 |
| I-20b | Benzoate of I-20 |
| I-20c | Salicylate of I-20 |
| I-20d | Succinate of I-20 |
| I-21a | Fumarate of I-21 |
| I-21b | Benzoate of I-21 |
| I-21c | Salicylate of I-21 |
| I-21d | Succinate of I-21 |
| I-22a | Fumarate of I-22 |
| I-22b | Benzoate of I-22 |
| I-22c | Salicylate of I-22 |
| I-22d | Succinate of I-22 |
| I-23a | Fumarate of I-23 |
| I-23b | Benzoate of I-23 |
| I-23c | Salicylate of I-23 |
| I-23d | Succinate of I-23 |
| I-24a | Fumarate of I-24 |
| I-24b | Benzoate of I-24 |
| I-24c | Salicylate of I-24 |
| I-24d | Succinate of I-24 |
| I-25a | Fumarate of I-25 |
| I-25b | Benzoate of I-25 |
| I-25c | Salicylate of I-25 |
| I-25d | Succinate of I-25 |
| I-26a | Fumarate of I-26 |
| I-26b | Benzoate of I-26 |
| I-26c | Salicylate of I-26 |
| I-26d | Succinate of I-26 |
| I-27a | Fumarate of I-27 |
| I-27b | Benzoate of I-27 |
| I-27c | Salicylate of I-27 |
| I-27d | Succinate of I-27 |
| I-28a | Fumarate of I-28 |
| I-28b | Benzoate of I-28 |
| I-28c | Salicylate of I-28 |
| I-28d | Succinate of I-28 |
| I-29a | Fumarate of I-29 |
| I-29b | Benzoate of I-29 |
| I-29c | Salicylate of I-29 |
| I-29d | Succinate of I-29 |
| I-30a | Fumarate of I-30 |
| I-30b | Benzoate of I-30 |
| I-30c | Salicylate of I-30 |
| I-30d | Succinate of I-30 |
| I-31a | Fumarate of I-31 |
| I-31b | Benzoate of I-31 |
| I-31c | Salicylate of I-31 |
| I-31d | Succinate of I-31 |
| I-32a | Fumarate of I-32 |
| I-32b | Benzoate of I-32 |
| I-32c | Salicylate of I-32 |
| I-32d | Succinate of I-32 |
| I-33a | Fumarate of I-33 |
| I-33b | Benzoate of I-33 |
| I-33c | Salicylate of I-33 |
| I-33d | Succinate of I-33 |
| I-34a | Fumarate of I-34 |
| I-34b | Benzoate of I-34 |
| I-34c | Salicylate of I-34 |
| I-34d | Succinate of I-34 |
| I-35a | Fumarate of I-35 |
| I-35b | Benzoate of I-35 |
| I-35c | Salicylate of I-35 |
| I-35d | Succinate of I-35 |
| I-36a | Fumarate of I-36 |
| I-36b | Benzoate of I-36 |
| I-36c | Salicylate of I-36 |
| I-36d | Succinate of I-36 |
| I-37a | Fumarate of I-37 |
| I-37b | Benzoate of I-37 |
| I-37c | Salicylate of I-37 |
| I-37d | Succinate of I-37 |
| I-38a | Fumarate of I-38 |
| I-38b | Benzoate of I-38 |
| I-38c | Salicylate of I-38 |
| I-38d | Succinate of I-38 |

In some embodiments, the pharmaceutically acceptable salt is hydrochloride salt of N,N-dimethyltryptamine (DMT). In some embodiments, the pharmaceutically acceptable salt is a hydrochloride salt of 5-hydroxy-N,N-dimethyltryptamine (5-OH-DMT). In some embodiments, the pharmaceutically acceptable salt is a hydrochloride salt of 5-methoxy-N,N-dimethyltryptamine (5-MeO-DMT). In some embodiments, the pharmaceutically acceptable salt is a hydrochloride salt of 2-(1H-indol-3-yl)-N,N-bis(methyl-d₃)ethan-1-amine-1,1,2,2-d₄ (DMT-d₁₀). In some embodiments, the pharmaceutically acceptable salt is a hydrochloride salt of 2-(1H-indol-3-yl)-N,N-bis(methyl-d₃)ethan-1-amine-1,1-d₂ (a DMT-d₈). In some embodiments, the pharmaceutically acceptable salt is a hydrochloride salt of 2-(1H-indol-3-yl)-N,N-dimethylethan-1-amine-1,1-d₂ (a DMT-d₂). In some embodiments, the pharmaceutically acceptable salt is a hydrochloride salt of 2-(5-methoxy-1H-indol-3-yl)-N,N-bis(methyl-d₃)ethan-1-amine-1,1,2,2-d₄ (5-MeO-DMT-d₁₀). In some embodiments, the pharmaceutically acceptable salt is a hydrochloride salt of 2-(5-(methoxy-d₃)-1H-indol-3-yl)-N,N-dimethylethan-1-amine-1,1-d₂ (5-MeO-DMT-d₅). In some embodiments, the pharmaceutically acceptable salt is a hydrochloride salt of 2-(5-(methoxy-d₃)-1H-indol-3-yl)-N,N-bis(methyl-d₃)ethan-1-amine-1,1,2,2-d₄ (5-MeO-DMT-d₁₃).

Various methods and procedures may be used to prepare pre-formed pharmaceutically acceptable salts of the compounds of the present disclosure, such methods and procedures being generally known to those of ordinary skill in the art. In some embodiments, the pre-formed pharmaceutically acceptable salt of a compound of the present disclosure is prepared by:
(a) suspending or dissolving a free base of the compound of the present disclosure (e.g., a compound of Formula (I) through (III) in a solvent or mixture of solvents;
(b) contacting an acid with the compound of the present disclosure to provide a mixture;
(c) optionally heating the mixture;
(d) optionally cooling the mixture; and
(e) isolating the salt.

Various solvents may be used, including one or more protic solvents, one or more aprotic solvents, or mixtures thereof. In some embodiments, the solvent(s) is a protic solvent(s). In some embodiments, the solvent is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, and octane, and combinations thereof. In some embodiments, the solvent is ethanol.

Suitable acids for use during the contacting step may include those described heretofore. The acid may be an inorganic acid (e.g., hydrochloric acid) or an organic acid, with organic acids being preferred. In some embodiments, the acid is an organic acid selected from the group consisting of fumaric acid, benzoic acid, salicylic acid, succinic acid, oxalic acid, and glycolic acid. In some embodiments, the acid is an organic acid selected from the group consisting of fumaric acid, benzoic acid, salicylic acid, and succinic acid, with fumaric acid, benzoic acid, and salicylic acid being preferred. In some embodiments, a stoichiometric (or superstoichiometric) quantity of the acid is contacted with the compound of the present disclosure. In some embodiments, a sub-stoichiometric (e.g., 0.5 molar equivalents) quantity of the acid is contacted with the compound of the present disclosure. The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

### Active Salt Mixture

The pharmaceutical formulation may comprise, as the psychopharmaceutical agent, a pharmaceutically acceptable salt of a single compound of the present disclosure (e.g., a single of compound of Formula (I) through (III)) or a pharmaceutically acceptable salt of a mixture of compounds of the present disclosure (e.g., a mixture of compounds of Formula (I) through (III)). In one example, the pharmaceutical formulation may contain an isotopologue mixture of compounds of the present disclosure in salt form as the psychopharmaceutical agent. In some embodiments, a subject compound of Formula (I) through (III) in salt form may be present in the pharmaceutical formulation at a purity of at least 20% by weight, at least 30% by weight, at least 40% by weight, at least 50% by weight, at least 60% by weight, at least 70% by weight, at least 80% by weight, at least 90% by weight, at least 95% by weight, at least 99% by weight, based on a total weight of the isotopologue mixture of compounds of Formula (I) through (III) in salt form present in the pharmaceutical formulation. For example, a pharmaceutical formulation formulated with a DMT-d₁₀ salt (salt form of 2-(1H-indol-3-yl)-N,N-bis(methyl-d₃)ethan-1-amine-1,1,2,2-d₄), as the subject compound in salt form, may additionally contain isotopologues of the subject compound in salt form, e.g., DMT-d₉ salt, a DMT-d₈ salt, etc. In some embodiments, the pharmaceutical formulation is substantially free of other isotopologues of the subject compound in salt form, e.g., the pharmaceutical formulation has less than 20, 15, 10, 9, 8, 7, 6, 5, 4, 3, 2 or 1 or 0.5 mole percent of other isotopologues of the subject compound in salt form.

When the injectable pharmaceutical formulation comprises, as the psychopharmaceutical agent, a pharmaceutically acceptable salt of a mixture of compounds of the present disclosure (e.g., a mixture of compounds of Formula (I) through (III)), that mixture may be referred to herein an “active salt mixture”. In some embodiments, the active salt mixture is a fumarate salt mixture, wherein the salt forms recited are fumarate salts. In some embodiments, the active salt mixture is a benzoate salt mixture, wherein the salt forms recited are benzoate salts. In some embodiments, the active salt mixture is a salicylate salt mixture, wherein the salt forms recited are salicylate salts. In some embodiments, the active salt mixture is a succinate salt mixture, wherein the salt forms recited are succinate salts.

In some embodiments, the pharmaceutical formulation comprises an active salt mixture comprising: (i) a pharmaceutically acceptable salt of DMT-d₁₀, i.e., a pharmaceutically acceptable salt of 2-(1H-indol-3-yl)-N,N-bis(methyl-d₃)ethan-1-amine-1,1,2,2-d₄ (I-8); (ii) a pharmaceutically acceptable salt of DMT-d₉, i.e., a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and optionally (iii) a pharmaceutically acceptable salt of DMT-d₈, i.e., a pharmaceutically acceptable salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a pharmaceutically acceptable salt of DMT-d₁₀, i.e., a pharmaceutically acceptable salt of 2-(1H-indol-3-yl)-N,N-bis(methyl-d₃)ethan-1-amine-1,1,2,2-d₄ (I-8). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a pharmaceutically acceptable salt of DMT-d₉, i.e., a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a pharmaceutically acceptable salt of DMT-d₈, i.e., a pharmaceutically acceptable salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture consists of or consists essentially of (i) a pharmaceutically acceptable salt of DMT-d₁₀, i.e., a pharmaceutically acceptable salt of 2-(1H-indol-3-yl)-N,N-bis(methyl-d₃)ethan-1-amine-1,1,2,2-d₄ (I-8); and (ii) a pharmaceutically acceptable salt of DMT-d₉, i.e., a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11).

In some embodiments, the pharmaceutical formulation comprises an active salt mixture comprising: (i) from 90% to 99% by weight of a pharmaceutically acceptable salt of DMT-d₁₀, i.e., a pharmaceutically acceptable salt of 2-(1H-indol-3-yl)-N,N-bis(methyl-d₃)ethan-1-amine-1,1,2,2-d₄ (I-8), or any range therebetween, based on a total weight of the active salt mixture; and (ii) from 1% to 10% by weight, in sum, of a pharmaceutically acceptable salt of DMT-d₉, i.e., a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), or any range therebetween, based on a total weight of the active salt mixture. In some embodiments, the active salt mixture (and thus the pharmaceutical formulation) contains no detectable amount of, or is otherwise substantially free of: (1) a pharmaceutically acceptable salt of DMT-d₈, i.e., a pharmaceutically acceptable salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12); (2) a pharmaceutically acceptable salt of DMT-d₇; (3) a pharmaceutically acceptable salt of DMT-d₆ (a pharmaceutically acceptable salt of 2-(1H-indol-3-yl)-N,N-bis(methyl-d₃)ethan-1-amine (I-4)); (4) a pharmaceutically acceptable salt of DMT-d₅; (5) a pharmaceutically acceptable salt of DMT-d₄ (a salt of 2-(1H-indol-3-yl)-N,N-dimethylethan-1-amine-1,1,2,2-d₄ (I-5)); (6) a pharmaceutically acceptable salt of DMT-d₃; (7) a pharmaceutically acceptable salt of DMT-d₂ (a salt of one or more of 2-(1H-indol-3-yl)-N,N-dimethylethan-1-amine-1,1-d₂ (I-2) and/or 2-(1H-indol-3-yl)-N,N-dimethylethan-1-amine-2,2-d₂ (I-3)); (8) a pharmaceutically acceptable salt of DMT-d₁; and (9) a pharmaceutically acceptable salt of DMT (a salt of 2-(1H-indol-3-yl)-N,N-dimethylethan-1-amine (I-1)). For example, in some embodiments, a weight, in sum, of pharmaceutically acceptable salts of isotopologues of DMT not listed in (i) or (ii), such as those listed in (1) through (9), is less than 1% by weight, less than 0.75% by weight, less than 0.5% by weight, less than 0.4% by weight, less than 0.3% by weight, less than 0.25% by weight, less than 0.2% by weight, less than 0.1% by weight, or 0% by weight, based on a total weight of the active salt mixture.

In some embodiments, the pharmaceutical formulation comprises an active salt mixture comprising: (i) a fumarate salt of DMT-d₁₀, i.e., a fumarate salt of 2-(1H-indol-3-yl)-N,N-bis(methyl-d₃)ethan-1-amine-1,1,2,2-d₄ (I-8); (ii) a fumarate salt of DMT-d₉, i.e., a fumarate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and optionally (iii) a fumarate salt of DMT-d₈, i.e., a fumarate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a fumarate salt of DMT-d₁₀, i.e., a fumarate salt of 2-(1H-indol-3-yl)-N,N-bis(methyl-d₃)ethan-1-amine-1,1,2,2-d₄ (I-8). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a fumarate salt of DMT-d₉, i.e., a fumarate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a fumarate salt of DMT-d₈, i.e., a fumarate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture consists of or consists essentially of (i) a fumarate salt of DMT-d₁₀, i.e., a fumarate salt of 2-(1H-indol-3-yl)-N,N-bis(methyl-d₃)ethan-1-amine-1,1,2,2-d₄ (I-8); and (ii) a fumarate salt of DMT-d₉, i.e., a fumarate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11).

In some embodiments, the pharmaceutical formulation comprises an active salt mixture comprising: (i) a benzoate salt of DMT-d₁₀, i.e., a benzoate salt of 2-(1H-indol-3-yl)-N,N-bis(methyl-d₃)ethan-1-amine-1,1,2,2-d₄ (I-8); (ii) a benzoate salt of DMT-d₉, i.e., a benzoate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and optionally (iii) a benzoate salt of DMT-d₈, i.e., a benzoate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a benzoate salt of DMT-d₁₀, i.e., a benzoate salt of 2-(1H-indol-3-yl)-N,N-bis(methyl-d₃)ethan-1-amine-1,1,2,2-d₄ (I-8). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a benzoate salt of DMT-d₉, i.e., a benzoate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a benzoate salt of DMT-d₈, i.e., a benzoate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture consists of or consists essentially of (i) a benzoate salt of DMT-d₁₀, i.e., a benzoate salt of 2-(1H-indol-3-yl)-N,N-bis(methyl-d₃)ethan-1-amine-1,1,2,2-d₄ (I-8); and (ii) a benzoate salt of DMT-d₉, i.e., a benzoate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11).

In some embodiments, the pharmaceutical formulation comprises an active salt mixture comprising: (i) a salicylate salt of DMT-d₁₀, i.e., a salicylate salt of 2-(1H-indol-3-yl)-N,N-bis(methyl-d₃)ethan-1-amine-1,1,2,2-d₄ (I-8); (ii) a salicylate salt of DMT-d₉, i.e., a salicylate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and optionally (iii) a salicylate salt of DMT-d₈, i.e., a salicylate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a salicylate salt of DMT-d₁₀, i.e., a salicylate salt of 2-(1H-indol-3-yl)-N,N-bis(methyl-d₃)ethan-1-amine-1,1,2,2-d₄ (I-8). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a salicylate salt of DMT-d₉, i.e., a salicylate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a salicylate salt of DMT-d₈, i.e., a salicylate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture consists of or consists essentially of (i) a salicylate salt of DMT-d₁₀, i.e., a salicylate salt of 2-(1H-indol-3-yl)-N,N-bis(methyl-d₃)ethan-1-amine-1,1,2,2-d₄ (I-8); and (ii) a salicylate salt of DMT-d₉, i.e., a salicylate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11).

In some embodiments, the pharmaceutical formulation comprises an active salt mixture comprising: (i) a succinate salt of DMT-d₁₀, i.e., a succinate salt of 2-(1H-indol-3-yl)-N,N-bis(methyl-d₃)ethan-1-amine-1,1,2,2-d₄ (I-8); (ii) a succinate salt of DMT-d₉, i.e., a succinate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and optionally (iii) a succinate salt of DMT-d₈, i.e., a succinate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a succinate salt of DMT-d₁₀, i.e., a succinate salt of 2-(1H-indol-3-yl)-N,N-bis(methyl-d₃)ethan-1-amine-1,1,2,2-d₄ (I-8). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a succinate salt of DMT-d₉, i.e., a succinate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a succinate salt of DMT-d₈, i.e., a succinate salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12). In some embodiments, the active salt mixture consists of or consists essentially of (i) a succinate salt of DMT-d₁₀, i.e., a succinate salt of 2-(1H-indol-3-yl)-N,N-bis(methyl-d₃)ethan-1-amine-1,1,2,2-d₄ (I-8); and (ii) a succinate salt of DMT-d₉, i.e., a succinate salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11).

In some embodiments, the pharmaceutical formulation comprises an active salt mixture comprising: (i) a pharmaceutically acceptable salt of 2-(1H-indol-3-yl)-N,N-bis(methyl-d₃)ethan-1-amine-1,1-d₂ (I-6); (ii) a pharmaceutically acceptable salt of DMT-d₇, i.e., a pharmaceutically acceptable salt of 2-(1H-indol-3-yl)-N,N-bis(methyl-d₃)ethan-1-amine-1-d; and optionally (iii) a pharmaceutically acceptable salt of DMT-d₆, i.e., a pharmaceutically acceptable salt of 2-(1H-indol-3-yl)-N,N-bis(methyl-d₃)ethan-1-amine (I-4). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a pharmaceutically acceptable salt of 2-(1H-indol-3-yl)-N,N-bis(methyl-d₃)ethan-1-amine-1,1-d₂ (I-6). In some embodiments, the active salt mixture comprises from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a pharmaceutically acceptable salt of DMT-d₇, i.e., a pharmaceutically acceptable salt of 2-(1H-indol-3-yl)-N,N-bis(methyl-d₃)ethan-1-amine-1-d. In some embodiments, the active salt mixture comprises from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a pharmaceutically acceptable salt of DMT-d₆, i.e., a pharmaceutically acceptable salt of 2-(1H-indol-3-yl)-N,N-bis(methyl-d₃)ethan-1-amine (I-4). In some embodiments, the active salt mixture consists of or consists essentially of (i) a pharmaceutically acceptable salt of 2-(1H-indol-3-yl)-N,N-bis(methyl-d₃)ethan-1-amine-1,1-d₂ (I-6); and (ii) a pharmaceutically acceptable salt of DMT-d₇, i.e., a pharmaceutically acceptable salt of 2-(1H-indol-3-yl)-N,N-bis(methyl-d₃)ethan-1-amine-1-d.

In some embodiments, the pharmaceutical formulation comprises an active salt mixture comprising: (i) a pharmaceutically acceptable salt of 2-(1H-indol-3-yl)-N,N-dimethylethan-1-amine-1,1-d₂ (I-2); (ii) a pharmaceutically acceptable salt of DMT-d₁, i.e., a pharmaceutically acceptable salt of 2-(1H-indol-3-yl)-N,N-dimethylethan-1-amine-1-d; and optionally (iii) a pharmaceutically acceptable salt of DMT, i.e., a pharmaceutically acceptable salt of 2-(1H-indol-3-yl)-N,N-dimethylethan-1-amine. In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a pharmaceutically acceptable salt of 2-(1H-indol-3-yl)-N,N-dimethylethan-1-amine-1,1-d₂ (I-2). In some embodiments, the active salt mixture comprises from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a pharmaceutically acceptable salt of DMT-d₁, i.e., a pharmaceutically acceptable salt of 2-(1H-indol-3-yl)-N,N-dimethylethan-1-amine-1-d. In some embodiments, the active salt mixture comprises from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a pharmaceutically acceptable salt of DMT, i.e., a pharmaceutically acceptable salt of 2-(1H-indol-3-yl)-N,N-dimethylethan-1-amine. In some embodiments, the active salt mixture consists of or consists essentially of (i) a pharmaceutically acceptable salt of 2-(1H-indol-3-yl)-N,N-dimethylethan-1-amine-1,1-d₂ (I-2); and (ii) a pharmaceutically acceptable salt of DMT-d₁, i.e., a pharmaceutically acceptable salt of 2-(1H-indol-3-yl)-N,N-dimethylethan-1-amine-1-d.

In some embodiments, the pharmaceutical formulation comprises an active salt mixture comprising: (i) a pharmaceutically acceptable salt of 5-MeO-DMT-d₁₀, i.e., a pharmaceutically acceptable salt of 2-(5-methoxy-1H-indol-3-yl)-N,N-bis(methyl-d₃)ethan-1-amine-1,1,2,2-d₄ (I-20); (ii) a pharmaceutically acceptable salt of 5-MeO-DMT-d₉, i.e., a pharmaceutically acceptable salt of one or more of 2-(5-methoxy-1H-indol-3-yl)-N,N-bis(methyl-d₃)ethan-1-amine-1,2,2-d₃ (I-22) and/or 2-(5-methoxy-1H-indol-3-yl)-N,N-bis(methyl-d₃)ethan-1-amine-1,1,2-d₃ (I-23); and optionally (iii) a pharmaceutically acceptable salt of 5-MeO-DMT-d₈, i.e., a pharmaceutically acceptable salt of one or more of 2-(5-methoxy-1H-indol-3-yl)-N,N-bis(methyl-d₃)ethan-1-amine-1,1-d₂ (I-18), 2-(5-methoxy-1H-indol-3-yl)-N,N-bis(methyl-d₃)ethan-1-amine-2,2-d₂ (I-19), and 2-(5-methoxy-1H-indol-3-yl)-N,N-bis(methyl-d₃)ethan-1-amine-1,2-d₂ (I-24). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a pharmaceutically acceptable salt of 5-MeO-DMT-d₁₀, i.e., a pharmaceutically acceptable salt of 2-(5-methoxy-1H-indol-3-yl)-N,N-bis(methyl-d₃)ethan-1-amine-1,1,2,2-d₄ (I-20). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a pharmaceutically acceptable salt of 5-MeO-DMT-d₉, i.e., a pharmaceutically acceptable salt of one or more of 2-(5-methoxy-1H-indol-3-yl)-N,N-bis(methyl-d₃)ethan-1-amine-1,2,2-d₃ (I-22) and/or 2-(5-methoxy-1H-indol-3-yl)-N,N-bis(methyl-d₃)ethan-1-amine-1,1,2-d₃ (I-23). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a pharmaceutically acceptable salt of 5-MeO-DMT-d₈, i.e., a pharmaceutically acceptable salt of one or more of 2-(5-methoxy-1H-indol-3-yl)-N,N-bis(methyl-d₃)ethan-1-amine-1,1-d₂ (I-18), 2-(5-methoxy-1H-indol-3-yl)-N,N-bis(methyl-d₃)ethan-1-amine-2,2-d₂ (I-19), and 2-(5-methoxy-1H-indol-3-yl)-N,N-bis(methyl-d₃)ethan-1-amine-1,2-d₂ (I-24). In some embodiments, the active salt mixture (and thus the pharmaceutical formulation) contains no detectable amount of, or is otherwise substantially free of (iii) a pharmaceutically acceptable salt of 5-MeO-DMT-d₈, i.e., a pharmaceutically acceptable salt of one or more of 2-(5-methoxy-1H-indol-3-yl)-N,N-bis(methyl-d₃)ethan-1-amine-1,1-d₂ (I-18), 2-(5-methoxy-1H-indol-3-yl)-N,N-bis(methyl-d₃)ethan-1-amine-2,2-d₂ (I-19), and 2-(5-methoxy-1H-indol-3-yl)-N,N-bis(methyl-d₃)ethan-1-amine-1,2-d₂ (I-24). In some embodiments, the active salt mixture consists of or consists essentially of (i) a pharmaceutically acceptable salt of 5-MeO-DMT-d₁₀, i.e., a pharmaceutically acceptable salt of 2-(5-methoxy-1H-indol-3-yl)-N,N-bis(methyl-d₃)ethan-1-amine-1,1,2,2-d₄ (I-20); and (ii) a pharmaceutically acceptable salt of 5-MeO-DMT-d₉, i.e., a pharmaceutically acceptable salt of one or more of 2-(5-methoxy-1H-indol-3-yl)-N,N-bis(methyl-d₃)ethan-1-amine-1,2,2-d₃ (I-22) and/or 2-(5-methoxy-1H-indol-3-yl)-N,N-bis(methyl-d₃)ethan-1-amine-1,1,2-d₃ (I-23).

In some embodiments, the pharmaceutical formulation comprises an active salt mixture comprising: (i) a pharmaceutically acceptable salt of 5-MeO-DMT-d₁₃, i.e., a pharmaceutically acceptable salt of 2-(5-(methoxy-d₃)-1H-indol-3-yl)-N,N-bis(methyl-d₃)ethan-1-amine-1,1,2,2-d₄ (I-34); (ii) a pharmaceutically acceptable salt of 5-MeO-DMT-d₁₂, i.e., a pharmaceutically acceptable salt of one or more of 2-(5-(methoxy-d₃)-1H-indol-3-yl)-N,N-bis(methyl-d₃)ethan-1-amine-1,2,2-d₃ (I-36) and/or 2-(5-(methoxy-d₃)-1H-indol-3-yl)-N,N-bis(methyl-d₃)ethan-1-amine-1,1,2-d₃ (I-37); and optionally (iii) a pharmaceutically acceptable salt of 5-MeO-DMT-d₁₁, i.e., a pharmaceutically acceptable salt of one or more of 2-(5-(methoxy-d₃)-1H-indol-3-yl)-N,N-bis(methyl-d₃)ethan-1-amine-1,1-d₂ (I-32), 2-(5-(methoxy-d₃)-1H-indol-3-yl)-N,N-bis(methyl-d₃)ethan-1-amine-2,2-d₂ (I-33), and 2-(5-(methoxy-d₃)-1H-indol-3-yl)-N,N-bis(methyl-d₃)ethan-1-amine-1,2-d₂ (I-38). In some embodiments, the active salt mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a pharmaceutically acceptable salt of 5-MeO-DMT-d₁₃, i.e., a pharmaceutically acceptable salt of 2-(5-(methoxy-d₃)-1H-indol-3-yl)-N,N-bis(methyl-d₃)ethan-1-amine-1,1,2,2-d₄ (I-34). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a pharmaceutically acceptable salt of 5-MeO-DMT-d₁₂, i.e., a pharmaceutically acceptable salt of one or more of 2-(5-(methoxy-d₃)-1H-indol-3-yl)-N,N-bis(methyl-d₃)ethan-1-amine-1,2,2-d₃ (I-36) and/or 2-(5-(methoxy-d₃)-1H-indol-3-yl)-N,N-bis(methyl-d₃)ethan-1-amine-1,1,2-d₃ (I-37). In some embodiments, the active salt mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a pharmaceutically acceptable salt of 5-MeO-DMT-d₁₁, i.e., a pharmaceutically acceptable salt of one or more of 2-(5-(methoxy-d₃)-1H-indol-3-yl)-N,N-bis(methyl-d₃)ethan-1-amine-1,1-d₂ (I-32), 2-(5-(methoxy-d₃)-1H-indol-3-yl)-N,N-bis(methyl-d₃)ethan-1-amine-2,2-d₂ (I-33), and 2-(5-(methoxy-d₃)-1H-indol-3-yl)-N,N-bis(methyl-d₃)ethan-1-amine-1,2-d₂ (I-38). In some embodiments, the active salt mixture (and thus the pharmaceutical formulation) contains no detectable amount of, or is otherwise substantially free of (iii) a pharmaceutically acceptable salt of 5-MeO-DMT-d₁₁, i.e., a pharmaceutically acceptable salt of one or more of 2-(5-(methoxy-d₃)-1H-indol-3-yl)-N,N-bis(methyl-d₃)ethan-1-amine-1,1-d₂ (I-32), 2-(5-(methoxy-d₃)-1H-indol-3-yl)-N,N-bis(methyl-d₃)ethan-1-amine-2,2-d₂ (I-33), and 2-(5-(methoxy-d₃)-1H-indol-3-yl)-N,N-bis(methyl-d₃)ethan-1-amine-1,2-d₂ (I-38). In some embodiments, the active salt mixture consists of or consists essentially of (i) a pharmaceutically acceptable salt of 5-MeO-DMT-d₁₃, i.e., a pharmaceutically acceptable salt of 2-(5-(methoxy-d₃)-1H-indol-3-yl)-N,N-bis(methyl-d₃)ethan-1-amine-1,1,2,2-d₄ (I-34); and (ii) a pharmaceutically acceptable salt of 5-MeO-DMT-d₁₂, i.e., a pharmaceutically acceptable salt of one or more of 2-(5-(methoxy-d₃)-1H-indol-3-yl)-N,N-bis(methyl-d₃)ethan-1-amine-1,2,2-d₃ (I-36) and/or 2-(5-(methoxy-d₃)-1H-indol-3-yl)-N,N-bis(methyl-d₃)ethan-1-amine-1,1,2-d₃ (I-37).

In some embodiments, the pharmaceutical formulation comprises an active salt mixture comprising: (i) a pharmaceutically acceptable salt of 5-MeO-DMT-d₅, i.e., a pharmaceutically acceptable salt of one or more of 2-(5-(methoxy-d₃)-1H-indol-3-yl)-N,N-dimethylethan-1-amine-1,1-d₂ (I-28) and/or 2-(5-(methoxy-d₃)-1H-indol-3-yl)-N,N-dimethylethan-1-amine-2,2-d₂ (I-29); (ii) a pharmaceutically acceptable salt of 5-MeO-DMT-d₄, i.e., a pharmaceutically acceptable salt of one or more of 2-(5-(methoxy-d₃)-1H-indol-3-yl)-N,N-dimethylethan-1-amine-1-d (I-26) and/or 2-(5-(methoxy-d₃)-1H-indol-3-yl)-N,N-dimethylethan-1-amine-2-d (I-27); and optionally (iii) a pharmaceutically acceptable salt of 5-MeO-DMT-d₃, i.e., a pharmaceutically acceptable salt of 2-(5-(methoxy-d₃)-1H-indol-3-yl)-N,N-dimethylethan-1-amine (I-25). In some embodiments, the active salt mixture comprises, in sum, from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 90% to 99% by weight, or any range therebetween, based on a total weight of the active salt mixture, of (i) a pharmaceutically acceptable salt of 5-MeO-DMT-d₅, i.e., a pharmaceutically acceptable salt of one or more of 2-(5-(methoxy-d₃)-1H-indol-3-yl)-N,N-dimethylethan-1-amine-1,1-d₂ (I-28) and/or 2-(5-(methoxy-d₃)-1H-indol-3-yl)-N,N-dimethylethan-1-amine-2,2-d₂ (I-29). In some embodiments, the active salt mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 1% to 10% by weight, or any range therebetween, based on a total weight of the active salt mixture, of (ii) a pharmaceutically acceptable salt of 5-MeO-DMT-d₄, i.e., a pharmaceutically acceptable salt of one or more of 2-(5-(methoxy-d₃)-1H-indol-3-yl)-N,N-dimethylethan-1-amine-1-d (I-26) and/or 2-(5-(methoxy-d₃)-1H-indol-3-yl)-N,N-dimethylethan-1-amine-2-d (I-27). In some embodiments, the active salt mixture comprises from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the active salt mixture, of (iii) a pharmaceutically acceptable salt of 5-MeO-DMT-d₃, i.e., a pharmaceutically acceptable salt of 2-(5-(methoxy-d₃)-1H-indol-3-yl)-N,N-dimethylethan-1-amine (I-25). In some embodiments, the active salt mixture (and thus the pharmaceutical formulation) contains no detectable amount of, or is otherwise substantially free of (iii) a pharmaceutically acceptable salt of 5-MeO-DMT-d₃, i.e., a pharmaceutically acceptable salt of 2-(5-(methoxy-d₃)-1H-indol-3-yl)-N,N-dimethylethan-1-amine (I-25). In some embodiments, the active salt mixture consists of or consists essentially of (i) a pharmaceutically acceptable salt of 5-MeO-DMT-d₅, i.e., a pharmaceutically acceptable salt of one or more of 2-(5-(methoxy-d₃)-1H-indol-3-yl)-N,N-dimethylethan-1-amine-1,1-d₂ (I-28) and/or 2-(5-(methoxy-d₃)-1H-indol-3-yl)-N,N-dimethylethan-1-amine-2,2-d₂ (I-29); and (ii) a pharmaceutically acceptable salt of 5-MeO-DMT-d₄, i.e., a pharmaceutically acceptable salt of one or more of 2-(5-(methoxy-d₃)-1H-indol-3-yl)-N,N-dimethylethan-1-amine-1-d (I-26) and/or 2-(5-(methoxy-d₃)-1H-indol-3-yl)-N,N-dimethylethan-1-amine-2-d (I-27).

### Psychopharmaceutical Agent Content

For use in the treatment of a disease or disorder disclosed herein, namely a neuropsychiatric disease or disorder or an inflammatory disease or disorder, such as a central nervous system (CNS) disorder and/or psychological disorder, including those associated with a 5-HT₂ receptor, the pharmaceutical formulation comprises a therapeutically effective amount of the psychopharmaceutical agent. In terms of unit dose, the pharmaceutical formulation comprising the psychopharmaceutical agent (e.g., a tryptamine psychedelic such as a pharmaceutically acceptable salt of the compound of Formula (I) through (III)) typically contains a free base dose (free base equivalence when a salt form is used) of about 1 mg, about 2 mg, about 3 mg, about 5 mg, about 10 mg, about 12 mg, about 14 mg, about 16 mg, about 18 mg, about 20 mg, about 25 mg, about 30 mg, about 40 mg, about 50 mg, about 75 mg, about 100 mg, about 150 mg, about 200 mg, about 300 mg, or any range therebetween. For example, a pharmaceutical formulation prepared with 40.4 mg of DMT fumarate (molar mass of 304.34 g/mol) would have a free base equivalence of DMT (molar mass of 188.27 g/mol) of about 25 mg as the unit dose. The pharmaceutical formulation can, if desired, also contain other compatible therapeutic agents.

The pharmaceutical formulations may have a free base concentration of psychopharmaceutical agent, e.g., a free base concentration of a compound of Formula (I) through (III) (free base equivalence when a salt form is used), by weight per total volume of pharmaceutical formulation of about 1 mg/mL, about 2 mg/mL, about 4 mg/mL, about 6 mg/mL, about 8 mg/mL, about 10 mg/mL, about 12 mg/mL, about 15 mg/mL, about 18 mg/mL, about 20 mg/mL, about 22 mg/mL, about 25 mg/mL, about 28 mg/mL, about 30 mg/mL, about 32 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, or any range therebetween, such as from about 5 mg/mL to about 70 mg/mL, from about 10 mg/mL to about 50 mg/mL, from about 15 mg/mL to about 40 mg/mL, from about 20 mg/mL to about 35 mg/mL. For example, a pharmaceutical formulation prepared from 40.4 mg of DMT fumarate (molar mass of 304.34 g/mol) in 1 mL of total volume of pharmaceutical formulation would have a free base concentration of psychopharmaceutical agent (in this example DMT free base; molar mass of 188.27 g/mol), of about 25 mg/mL. Of these concentrations, free base concentrations of psychopharmaceutical agent below about 70 mg/mL provide advantageous controlled-release profiles across the broadest range of release modifier molecular weight and release modifier concentrations. However, difficulties with the pharmaceutical formulation may be encountered at free base concentrations of about 70 mg/mL or higher. Firstly, to achieve the targeted controlled-release effects at these higher free base concentrations, it may be necessary to correspondingly increase the concentration of release modifier. Pushing the limits of the release modifier concentration may be problematic as the higher viscosities thus obtained may complicate or preclude the use of sterile filtration (which in the case of pharmaceutical formulations containing hyaluronate salts is effectively the only suitable sterilization technique available) and may cause increased pain at the injection site. Secondly, a deterioration of pharmaceutical formulation stability may be experienced at the higher free base concentrations of about 70 mg/mL or more, with precipitation being a main concern owing to the pharmacopeial requirements for particulate matter in injectables such as subcutaneous dosage forms (USP Particulate Matter in Injections <788>). Attempts to lower the free base concentration by dilution (increasing injection volume) may not be possible in subcutaneous dosage forms, for example, since higher injection volumes, especially those above 3 mL, are associated with pain at the injection site. Consequently, for subcutaneous pharmaceutical formulations, the free base concentration of psychopharmaceutical agent (e.g., tryptamine psychedelic such as a compound of Formula (I) through (III)) is typically kept below about 70 mg/mL, below about 65 mg/mL, below about 60 mg/mL, below about 55 mg/mL, with 10 to 50 mg/mL being preferred.

In terms of pharmaceutical formulations containing a tryptamine psychedelic, it is for these reasons that tryptamine psychedelics with longer half-lives, such as deuterated tryptamine psychedelics may provide significant advantages over their non-deuterated counterparts, especially in subcutaneous dosage forms. Specifically deuterated tryptamine psychedelics possess advantageous metabolic degradation profiles which can lead to higher plasma concentrations and enhanced brain penetration, so that in some embodiments the therapeutic doses may be reduced. For example, initial human clinical studies indicate that a therapeutically relevant psychedelic dose of DMT (non-deuterium enriched) may be in the range of about 70 mg or higher (free base), whereas for deuterated analogs of DMT such as 2-(1H-indol-3-yl)-N,N-bis(methyl-d₃)ethan-1-amine-1,1,2,2-d₄ (DMT-d₁₀) less drug (e.g., 10 to 50 mg, 15 to 50 mg, 20 to 40 mg, 30 to 50 mg, free base) may be needed to maintain desired blood concentrations owing to its longer half-life in vivo. The lower dosing requirements of deuterated analogs of DMT such as DMT-d₁₀ allow for lower concentrations of release modifier to be used to achieve desirable controlled-release profiles, and for lower injection volumes.

Release Modifier The pharmaceutical formulation comprises a release modifier. The release modifier is the component primarily responsible for providing a controlled, tunable, and linear release of the psychopharmaceutical agent (e.g., a tryptamine psychedelic such as a pharmaceutically acceptable salt of a compound of Formula (I) through (III)) upon injection of the pharmaceutical formulation, such as upon subcutaneous injection. Without being limited by theory, it is believed that the release modifier acts by thickening and building viscosity of the pharmaceutical formulation, while potentially also providing electrostatic attraction with the psychopharmaceutical agent, such that upon injection the psychopharmaceutical agent can be slowly released from the injection site (e.g., in the case of subcutaneous injection, within the fat or the layer of skin directly below the dermis and epidermis) and absorbed more slowly, generating a depot-like release effect. The release modifier may be a polymeric material, such as a hyaluronate salt or a carboxymethyl cellulose salt, which may, or may not, be crosslinked. The rate of release of the psychopharmaceutical agent can be controlled through cross-linking or the lack thereof, or the extent of cross-linking of the release modifier. Release modifiers which are not cross-linked will typically provide a shorter release profile than those which are crosslinked, with crosslinking capable of extending the release significantly, such as over the course of a day or multiple days. For delivery of tryptamine psychedelics (e.g., a pharmaceutically acceptable salt of a compound of Formula (I) through (III)), whereby a controlled-release is sought which provides a duration of peak effects of about 30 to 120 minutes, the release modifier is generally not crosslinked so as not to overextend the release period. For clarity, the release modifier is considered a separate component from the psychopharmaceutical agent. The release modifier is also considered a separate component from the pharmaceutically acceptable additive(s) described hereinafter (such as a buffering agent, a tonicity agent, a pH adjusting agent, etc.) when such pharmaceutically acceptable additives are included in the pharmaceutical formulation of the present disclosure, even though the release modifier may perform a similar function to a particular additive(s). For example, the release modifier is considered different from a tonicity agent even though the release modifier contributes to the overall osmolality of the pharmaceutical formulation. In another example, the release modifier is considered different from a buffering agent or a pH adjusting agent, even though the release modifier may influence the pH of the pharmaceutical formulation.

### Hyaluronate Salt

In some embodiments, the release modifier is a hyaluronate salt (anionic salt form of hyaluronic acid), which is non-sulfated glycosaminoglycan and long-chain polymer of disaccharide units of glucuronate-N-acetylglucosamine. Hyaluronate salts are biocompatible and are distributed widely throughout human connective, epithelial, and neural tissues. The hyaluronate salt may include, but is not limited to, a sodium salt of hyaluronate (sodium hyaluronate), a potassium salt of hyaluronate (potassium hyaluronate), a calcium salt of hyaluronate (calcium hyaluronate), a zinc salt of hyaluronate (zinc hyaluronate), and a magnesium salt of hyaluronate (magnesium hyaluronate), or a combination thereof. In some embodiments, the release modifier is sodium hyaluronate. While not limited thereto, the hyaluronate salt may be produced by a microbial fermentation and purification process and is preferably pharmacopoeia compliant. In terms of purification, hyaluronate salts are known to be heat sensitive, and so cannot be typically sterilized by thermal sterilization methods such as steam sterilization, dry-heat sterilization/depyrogenation, etc., which can cause polymer degradation. Instead, hyaluronate salts are sterilized by sterile filtration such as sterile filtration through a 0.25 m filter size or less. After sterile filtration, hyaluronate salts may be optionally sterilized with a secondary sterilization process such as ethylene oxide (ETO) gas sterilization or gamma sterilization under less harsh conditions.

In some embodiments, the hyaluronate salt is a native hyaluronate salt, meaning it is not substituted, modified with pendant groups, conjugated, crosslinked, or otherwise covalently modified. Rather, the native hyaluronate salt possesses unmodified disaccharide units of glucuronate-N-acetylglucosamine. Examples may include, but are not limited to, sodium hyaluronate products available from Lifecore Biomedical, Inc.

In some embodiments, the hyaluronate salt is a non-native hyaluronate salt, i.e., those that are substituted, modified with pendant groups, conjugated, crosslinked, deacetylated, or otherwise covalently modified. The non-native hyaluronate salts may be, inter alia, acetylated, deacetylated, alkylated, esterified, amidated, hydrazidated, epoxy grafted, silylated, sulfated, and/or crosslinked hyaluronate salts. These modifications such as crosslinking may enable the formation of hydrogels of hyaluronate salts. The degree of modification or substitution in non-native hyaluronate salts is typically about 0.1%, 0.5%, 1%, 2%, 3%, 4%, 5%, 6%, 7%, 8%, 9%, 10%, or any range therebetween. Examples of non-native hyaluronate salts include, but are not limited to, sodium hyaluronate salts modified with pendant tyramine groups (an amidated hyaluronate salt) whereby tyramine is introduced onto glucuronate units using amide bond chemistry (EDC chemistry); and Corgel® BioHydrogel products available from Lifecore Biomedical, Inc., whereby the tyramine substituted sodium hyaluronate (TS-NaHy) from above is subsequently cross-linked by forming stable dihydroxyphenyl covalent bonds through an enzyme driven reaction involving e.g., horseradish peroxidase. While crosslinked or hydrogels of hyaluronate salts may be employed in some cases, for the delivery of tryptamine psychedelics (e.g., a pharmaceutically acceptable salt of a compound of Formula (I) through (III)) in which a controlled-release is sought that provides a duration of peak effects of about 30 to 120 minutes, the hyaluronate salt is generally not crosslinked or in hydrogel form so as not to overextend the release period and the resulting duration of peak effects beyond about 120 minutes.

The weight average molecular weight (Mw) of the hyaluronate salt may be about 500 kDa, about 550 kDa, about 600 kDa, about 650 kDa, about 700 kDa, about 750 kDa, about 800 kDa, about 850 kDa, about 900 kDa, about 950 kDa, about 1,000 kDa, about 1,200 kDa, about 1,300 kDa, about 1,400 kDa, about 1,500 kDa, about 1,600 kDa, about 1,700 kDa, about 1,800 kDa, about 1,900 kDa, about 2,000 kDa, or any range therebetween such as from about 500 kDa to about 2,000 kDa, from about 600 kDa to about 1,500 kDa, from about 750 kDa to about 1,000 kDa, from about 1,000 kDa to about 2,000 kDa, from about 1,000 kDa to about 1,900 kDa, from about 1,000 kDa to about 1,800 kDa, from about 1,200 kDa to about 1,800 kDa, from about 1,500 kDa to about 1,700 kDa, from about 1,600 kDa to about 1,800 kDa. A weight average molecular weight of the hyaluronate salt which is above the aforementioned upper limit may result in pharmaceutical formulations which are too viscous, thereby complicating the sterile filtration process and resulting in painful injections, especially in the case of subcutaneous injections. Further, hyaluronate salts with a weight average molecular weight above the aforementioned upper limit may provide a release profile which is too slow for achieving the desired peak effects time course of about 30 to 120 minutes in the case of some tryptamine psychedelics (e.g., a pharmaceutically acceptable salt of a compound of Formula (I) through (III)). On the other hand, hyaluronate salts with a weight average molecular weight below the aforementioned lower limit may not provide any meaningful controlled-release effects, and the pharmaceutical formulation may instead behave similarly to those which are formulated without any release modifier. In some embodiments, the hyaluronate salt has a molecular weight range in between about 500 kDa, about 600 kDa, about 700 kDa, about 800 kDa, about 900 kDa, about 1,000 kDa, about 1,100 kDa, about 1,200 kDa, about 1,300 kDa, about 1,400 kDa, about 1,500 kDa, about 1,600 kDa, about 1,700 kDa, about 1,800 kDa, about 1,900 kDa, and about 2,000 kDa, or any intermediate range between any of these values. In some embodiments, the hyaluronate salt has a molecular weight range of about 500 kDa to about 2,000 kDa. In some embodiments, the hyaluronate salt has a molecular weight range of about 500 kDa to about 750 kDa. In some embodiments, the hyaluronate salt has a molecular weight range of about 750 kDa to about 1,000 kDa. In some embodiments, the hyaluronate salt has a molecular weight range of about 750 kDa to about 1,500 kDa. In some embodiments, the hyaluronate salt has a molecular weight range of about 1,000 kDa to about 1,800 kDa. In some embodiments, the hyaluronate salt has a molecular weight range of about 900 kDa to about 1,400 kDa. The weight average molecular weight of the hyaluronate salt would then fall somewhere within the molecular weight range.

Examples of suitable hyaluronate salts include, but are not limited to, sodium hyaluronate products HA700K (molecular weight range of 500-<750 kDa), HA1M (molecular weight range of 750-1,000 kDa), and HA15M (molecular weight range of >1,000-1,800 kDa), available from Lifecore Biomedical, Inc., and Hyatrue® HA-EP1.8 (molecular weight range of 900-1,400 kDa) available from Bloomage Freda Biopharm Co. Ltd.

A concentration of hyaluronate salt by weight per total volume of the pharmaceutical formulation expressed as a percentage (% w/v) may be about 0.02%, about 0.03%, about 0.04%, about 0.05%, about 0.06%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.1% to about 2%, about 0.1% to about 1.5%, about 0.1% to about 1%, about 0.1% to about 0.75%, about 0.1% to about 0.5%, about 0.15% to about 1%, about 0.2% to about 0.75%, or about 0.25% to about 0.5%. As the concentration of hyaluronate salt is increased, so to is the viscosity of the pharmaceutical formulation. Exceeding viscosity specifications is problematic for sterile filtration (and hyaluronate salts are sensitive to thermal sterilization techniques) and may cause increased pain at the injection site. Moreover, in the case of tryptamine psychedelics (e.g., a pharmaceutically acceptable salt of a compound of Formula (I) through (III)) in which a release is sought that provides a peak effects time course of about 30 to 120 minutes, too high a concentration of hyaluronate salt may overextend the release profile and the resulting duration of peak effects beyond about 120 minutes, which is not clinically favorable as it would necessitate prolonged supervised clinical observation. For these reasons, in such embodiments, the concentration of hyaluronate salt preferably does not exceed about 1%, about 0.95%, about 0.9%, about 0.85%, about 0.8%, about 0.75%, about 0.7%, about 0.65%, about 0.6%, about 0.55%, about 0.5% w/v. In preferred embodiments, the concentration of hyaluronate salt is from about 0.1% to about 0.5% w/v. Attempts to lower the concentration of hyaluronate salt by dilution (increasing injection volume) may not be possible in subcutaneous dosage forms, for example, since higher injection volumes, especially those above 3 mL, are associated with pain at the injection site. Conversely, hyaluronate salt concentrations which are below the aforementioned lower limit may not provide any meaningful controlled-release effects, and the pharmaceutical formulation may instead behave similarly to those which are formulated without any release modifier.

In some embodiments, a ratio of free base concentration of psychopharmaceutical agent, (e.g., a free base concentration of a compound of Formula (I) through (III)), in terms of weight per total volume of pharmaceutical formulation (mg/mL), to the concentration of hyaluronate salt by weight per total volume of the pharmaceutical formulation expressed as a percentage (% w/v) is about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 105:1, about 110:1, about 115:1, about 120:1, about 125:1, about 130:1, or any range therebetween, such as from about 10:1 to about 130:1, about 15:1 to about 120:1, about 20:1 to about 110:1, or about 25:1 to about 100:1. In the case of tryptamine psychedelics (e.g., a pharmaceutically acceptable salt of a compound of Formula (I) through (III)) in which a release profile is sought that provides a peak effects duration of about 30 to 120 minutes, ratios below the aforementioned lower limit may result in pharmaceutical formulations with a release profile which is overextended (too slow) in terms of clinical practicality. Ratios above the aforementioned upper limit tend to provide a release that is not meaningfully different from formulations lacking release modifier (too fast). Even so, there are instances where higher ratios of free base concentration of psychopharmaceutical agent (mg/mL) to the concentration of hyaluronate salt (% w/v) can be used, for example up to about 500:1, about 400:1, about 300:1, about 200:1, or about 140:1, such as when a faster release profile is desirable or where other parameters in the formulation can counteract any fast release effects attributable to the use of such higher ratios.

In some embodiments, a ratio of the weight average molecular weight (Mw) of the hyaluronate salt (in kDa) to a free base concentration of psychopharmaceutical agent, (e.g., a free base concentration of a compound of Formula (I) through (III)), in terms of weight per total volume of pharmaceutical formulation (mg/mL), is about 17:1, about 18:1, about 19:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, or any range therebetween, such as from about 17:1 to about 100:1, about 20:1 to about 100:1, about 22:1 to about 80:1, about 24:1 to about 60:1, or about 25:1 to about 55:1, or about 26:1 to about 100:1, or about 28:1 to about 100:1, or about 30:1 to about 75:1. Ratios below the aforementioned lower limit may result in pharmaceutical formulations with a release profile similar to those which are formulated without any release modifier (too fast), whereas ratios above the aforementioned upper limit may overextend the release (too slow) and the resulting duration of peak effects, which is not favorable in the case of certain tryptamine psychedelics (e.g., a pharmaceutically acceptable salt of a compound of Formula (I) through (III)). In some embodiments, such as when the psychopharmaceutical agent is a pharmaceutically acceptable salt of a compound of Formula (III), a ratio of the weight average molecular weight of the hyaluronate salt (in kDa) to a free base concentration of psychopharmaceutical agent, in terms of weight per total volume of pharmaceutical formulation (mg/mL), is preferably about 30:1, about 31:1, about 32:1, about 33:1, about 34:1, about 35:1, about 40:1, about 45:1, about 50:1, about 55:1, about 60:1, about 65:1, about 70:1, about 75:1, about 80:1, about 85:1, about 90:1, about 95:1, about 100:1, or any range therebetween, such as from about 35:1 to about 100:1, about 40:1 to about 100:1, about 42:1 to about 80:1, about 44:1 to about 60:1, or about 45:1 to about 55:1.

### Carboxymethyl Cellulose Salt

In some embodiments, the release modifier is a carboxymethyl cellulose salt, which is a cellulose derivative with carboxymethyl groups (—CH₂COO⁻) bound to some of the hydroxyl groups of the glucopyranose monomers that make up the cellulose backbone. The carboxymethyl cellulose salt may include, but is not limited to, a sodium salt of carboxymethyl cellulose (sodium carboxymethyl cellulose). While not limited thereto, the carboxymethyl cellulose salt may be produced by reacting alkali cellulose with sodium monochloroacetate, and this reaction may be performed under rigidly controlled conditions to control the degree of substitution (DS), which is the average number of hydroxyl groups of the glucopyranose monomers that are carboxymethylated, with the theoretical limit being a DS of 3.0. The resultant polymer is purified and dried for pharmacopoeia compliance.

In some embodiments, the carboxymethyl cellulose salt is not substituted, modified with pendant groups, conjugated, crosslinked, or otherwise covalently modified. Rather, the carboxymethyl cellulose salt possesses a cellulose backbone formed from glucopyranose monomers which are substituted only with carboxymethyl groups. For example, in some embodiments, the carboxymethyl cellulose salt is not crosslinked with glycolic acid to form a croscarmellose salt such as sodium croscarmellose.

The carboxymethyl cellulose salt may have a degree of substitution (DS) of 0.7, 0.75, 0.8, 0.85, 0.9, 0.95, 1.0, 1.05, 1.1, 1.15, 1.2, or any range therebetween, such as from 0.45 to less than 0.9, from 0.9 to less than 1.2, or from 1.2 to 1.5. In some embodiments, the carboxymethyl cellulose salt has a DS of 0.85 to 1.15 or 0.9 to 1.0.

The weight average molecular weight (Mw) of the carboxymethyl cellulose salt is typically below about 500 kDa, for example, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, about 300 kDa, about 350 kDa, about 400 kDa, about 450 kDa, or any range therebetween, such as from about 90 kDa to about 300 kDa, from about 100 kDa to about 300 kDa, from about 100 kDa to about 275 kDa, from about 150 kDa to about 250 kDa. A weight average molecular weight of the carboxymethyl cellulose salt which is above the aforementioned upper limit generally results in pharmaceutical formulations that exceed viscosity specifications for being injectable and syringeable, and are not generally approved by the Food & Drug Administration (FDA) for use in injectables. Accordingly, suitable carboxymethyl cellulose salts are typically those with a Brookfield viscosity, measured as 2% aqueous solutions using spindle number 3 at 30 rpm, of from about 400 cP, about 600 cP, about 800 cP, about 1,000 cP, about 1,200 cP, about 1,500 cP, about 1,750 cP, about 2,000 cP, about 2,250 cP, about 2,500 cP, about 2,750 cP, about 3,000 cP, about 3,100 cP, or any range therebetween, such as from about 400 cP to about 3,100 cP or about 1,500 cP to about 3,100 cP.

Examples of the carboxymethyl cellulose salt may include, but are not limited to, Aqualon™ and Blanose™ sodium carboxymethyl cellulose products available from Ashland, such as Aqualon™/Blanose™ grades 9M8F PH, 9M8XF, 9M31F PH, 9M31XF PH, and 7MF PH. In some embodiments, the carboxymethyl cellulose salt is 7MF PH from Ashland (sodium carboxymethyl cellulose; Mw=250 kDa; DS=0.7; Brookfield viscosity, measured as 2% aqueous solution using spindle number 3 at 30 rpm, of from 400 cP to 800 cP).

A concentration of carboxymethyl cellulose salt by weight per total volume of the pharmaceutical formulation expressed as a percentage (% w/v) may be about 0.55%, about 0.6%, about 0.65%, about 0.7%, about 0.75%, about 0.8%, about 0.85%, about 0.9%, about 0.95%, about 1%, or any range therebetween, such as from about 0.6% to about 1%, about 0.7% to about 1%, about 0.75% to about 1%, about 0.75% to about 0.9%, about 0.75% to about 0.8%. Concentrations of carboxymethyl cellulose salt which exceed the aforementioned upper limit provide pharmaceutical formulations which are too viscous for sterile filtration, injectability, and/or syringeability, for example may cause increased pain at the injection site. On the contrary, carboxymethyl cellulose salt concentrations which are below the aforementioned lower limit may not provide any meaningful controlled-release effects, and the pharmaceutical formulation may instead behave similarly to those which are formulated without any release modifier.

In some embodiments, a ratio of free base concentration of psychopharmaceutical agent, (e.g., a free base concentration of a compound of Formula (I) through (III)), in terms of weight per total volume of pharmaceutical formulation (mg/mL), to the concentration of carboxymethyl cellulose salt by weight per total volume of the pharmaceutical formulation expressed as a percentage (% w/v) is about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, or any range therebetween, such as from about 10:1 to about 100:1, about 13:1 to about 90:1, about 15:1 to about 75:1, or about 20:1 to about 50:1.

In some embodiments, a ratio of the weight average molecular weight of the carboxymethyl cellulose salt (in kDa) to a free base concentration of psychopharmaceutical agent, (e.g., a free base concentration of a compound of Formula (I) through (III)), in terms of weight per total volume of pharmaceutical formulation (mg/mL), is about 1:1, about 2:1, about 3:1, about 4:1, about 5:1, about 6:1, about 8:1, about 10:1, about 12:1, about 14:1, about 16:1, about 18:1, about 20:1, about 22:1, about 24:1, about 26:1, about 28:1, about 30:1, about 32:1, about 34:1, about 36:1, about 38:1, about 40:1, about 42:1, about 44:1, about 46:1, about 48:1, about 50:1, or any range therebetween, such as from about 1:1 to about 50:1, about 5:1 to about 40:1, about 10:1 to about 30:1, about 15:1 to about 20:1, or about 1:1 to about 10:1.

### Aqueous Vehicle

The pharmaceutical formulation comprises an aqueous vehicle. The term “vehicle” herein refers to a diluent, adjuvant, excipient, carrier, and/or any other auxiliary or supporting ingredient with which a psychopharmaceutical agent and release agent of present disclosure is formulated for administration to a mammal. The aqueous vehicle, and thus the pharmaceutical formulation, comprises water, such as water for injection (WFI). Suitable aqueous vehicles include, but are not limited to, water, saline, physiological or isotonic saline, phosphate buffered saline (PBS), sodium chloride injection, Ringers injection, isotonic dextrose injection, sterile water injection, dextrose and lactated Ringers injection.

In addition to water, the aqueous vehicle, and thus the pharmaceutical formulation, may optionally contain one or more pharmaceutically acceptable additives, as desired/needed. “Pharmaceutically acceptable additives” may be diluents, adjuvants, excipients, carriers, or any other auxiliary or supporting ingredient approved by a regulatory agency of the Federal or a state government or listed in the U.S. Pharmacopeia or other generally recognized pharmacopeia for use in mammals, such as humans. Examples of pharmaceutically acceptable additives include, but are not limited to, water-miscible vehicles, non-aqueous vehicles, antimicrobial agents or preservatives against the growth of microorganisms, stabilizing agents, tonicity agents, buffering agents, antioxidants, local anesthetics, complexing agents, sequestering or chelating agents, pH adjusting agents, absorption enhancers, including combinations thereof. It should be understood that many pharmaceutically acceptable additives may serve several functions, even within the same pharmaceutical formulation, for example, a buffering agent may also act as a tonicity agent and vice versa.

Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide, or a combination thereof.

Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil, or a combination thereof.

Antimicrobial agents or preservatives include, but are not limited to, phenols (e.g., phenol), cresols, mercurials, benzyl alcohol, chlorobutanol, methyl and propyl p-hydroxybenzates, thimerosal, benzalkonium chloride, benzethonium chloride, methyl-, ethyl-, and propyl-parabens, benzoic acid, sodium benzoate, and sorbic acid, or a combination thereof.

Stabilizing agents include, but are not limited to, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, glycerol, methionine, monothioglycerol, ascorbic acid, citric acid, polysorbate, arginine, and sorbitol, or a combination thereof. For example, fatty acids may act as lipid carriers. The fatty acid may have from 4 to 30 carbon atoms, 6 to 28 carbon atoms, 8 to 24 carbon atoms, 10 to 20 carbon atoms, or 12 to 18 carbon atoms. The fatty acid may be a fatty monoacid or a fatty diacid. Exemplary fatty acids may include, but are not limited to, adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, arachidic acid, behenic acid, lignoceric acid, palmitolic acid, linolenic acid, arachidonic acid, eicosapentaenoic acid, docosahexaenoic acid, and combinations thereof. In some embodiments, the pharmaceutical formulation is formulated without a fatty acid to prevent hydrogel formation and an overextended release (for an example of a hydrogel that generates a release over days, see Kang N W, et al. Subcutaneously Injectable Hyaluronic Acid Hydrogel for Sustained Release of Donepezil with Reduced Initial Burst Release: Effect of Hybridization of Microstructured Lipid Carriers and Albumin. Pharmaceutics. 2021 Jun. 11; 13(6):864).

A tonicity agent is a chemical that, on inclusion within a pharmaceutical formulation, modulates the osmolality of the pharmaceutical formulation. Sometimes, the concentration of psychopharmaceutical agent and the release modifier in the pharmaceutical formulation provides a desired osmolality, and so no tonicity agent is needed/included. Alternatively, the concentration of psychopharmaceutical agent and the release modifier in the pharmaceutical formulation does not provide the osmolality specifications for injection, and so one or more tonicity agents may be included to reach a desired osmolality. When the pharmaceutical formulation comprises a tonicity agent, the concentration of the tonicity agent will be adjusted considering the osmolality contributions from the concentrations of psychopharmaceutical agent and the release modifier to provide a pharmaceutical formulation with a desirable osmolality range (e.g., 150 to 600 mOsm/kg). Tonicity agents include, but are not limited to, sodium chloride; potassium chloride; calcium chloride; magnesium chloride; dextrose; glucose; mannitol; lactose; sorbitol; sucrose; alanine; ethanol; benzyl alcohol; creatinine; glycine; glycerol; histidine; polyethylene glycol; propylene glycol; sodium bicarbonate; sodium hydroxide; hydrochloric acid; phosphoric acid; a phosphate salt such as sodium phosphate or potassium phosphate; acetic acid; an acetate salt such as sodium acetate, potassium acetate, or ammonium acetate; citric acid; a citrate salt such as sodium citrate or potassium citrate; arginine; ascorbic acid; an ascorbate salt such as potassium ascorbate or sodium ascorbate; edetic acid; an edetate salt such as sodium edetate or calcium edetate; lactic acid; a lactate salt such as potassium lactate or sodium lactate; tartaric acid; a tartrate salt such as sodium tartrate or potassium tartrate, as well as combinations thereof. In some embodiments, the tonicity agent is at least one selected from the group consisting of sodium chloride, potassium chloride, calcium chloride, sodium bicarbonate, magnesium chloride, dextrose, glucose, mannitol, lactose, sorbitol, sucrose, and sodium lactate. Typically, the tonicity agent is sodium chloride. In some embodiments, the pharmaceutical formulation comprises sodium chloride at a concentration, in terms of weight per total volume of the pharmaceutical formulation expressed as a percentage (% w/v), of about 0.1%, about 0.2%, about 0.3%, about 0.4%, about 0.5%, about 0.6%, about 0.7%, about 0.8%, or any range therebetween such as from about 0.1% to about 0.6%, about 0.2% to about 0.55%, about 0.3% to about 0.5% w/v.

A buffering agent is a chemical that on inclusion into the pharmaceutical formulation comprises a weak acid and its conjugate base in equilibrium, which resist changes in pH on addition of acid or base to the pharmaceutical formulation. In other words, addition of acid or base to the pharmaceutical formulation shifts the position of equilibrium in favor of the weak acid or conjugate base, respectively, and as a consequence, the concentration of free protons in the pharmaceutical formulation (and thus the pH) is relatively unchanged or changed by less than the amount expected for the quantity of acid or base added (until the buffer capacity is reached). For clarity, the buffering agent is considered a separate component from the psychopharmaceutical agent (e.g., a pharmaceutically acceptable salt of a compound of the present disclosure such as a pharmaceutically acceptable salt of a compound of Formula (I) through (III)). In this sense, the buffering agent is not merely a counterion to the protonated form of the compound of the present disclosure. Rather the buffering agent, when included, provides a buffering effect to resist changes in pH above that which may be provided by the psychopharmaceutical agent. The buffering agent is also considered a separate component from the release modifier, and again, provides a buffering effect to resist changes in pH above that which may be provided by the release modifier. Buffering agents include, but are not limited to, a phosphate buffer (a phosphate salt and phosphoric acid; pKa=2.14, 7.20, and 12.37), an acetate buffer (an acetate salt and acetic acid; pKa=4.76), a citrate buffer (a citrate salt and citric acid; pKa=3.13, 4.76, and 6.40), an ascorbate buffer (an ascorbate salt and ascorbic acid; pKa=4.10 and 11.6), a benzoate buffer (a benzoate salt and benzoic acid; pKa=4.20), an oxalate buffer (an oxalate salt and oxalic acid; pKa=1.25 and 4.14), a formate buffer (a formate salt and formic acid; pKa=3.75), or a combination thereof. Generally, a suitable buffer is optionally selected that comprises an acid with a pKa value (or an acid having at least one pKa value in the case of polyprotic acids) that lies within ±1 of the desired pH of the pharmaceutical formulation. In some embodiments, the pharmaceutical formulation is not formulated with a buffering agent.

Antioxidants include, but are not limited to, bisulfite and sodium metabisulfite, ascorbic acid, citric acid, tartaric acid, thiol derivatives, or combinations thereof. In some embodiments, the pharmaceutical formulation has an oxygen content of less than 2 ppm, such as between 0.1 ppm and 2 ppm.

Local anesthetics include, but are not limited to, procaine hydrochloride.

Complexing agents include, but are not limited to, cyclodextrins, including ca-cyclodextrin, β-cyclodextrin, hydroxypropyl-3-cyclodextrin, sulfobutylether-β-cyclodextrin, and sulfobutylether 7-O-cyclodextrin (CAPTISOL®, CyDex, Lenexa, Kans.), or combinations thereof.

Sequestering or chelating agents include, but are not limited to EDTA.

pH adjusting agents include, but are not limited to, sodium hydroxide, potassium hydroxide, sodium carbonate, ammonium hydroxide, calcium hydroxide, magnesium hydroxide, hydrochloric acid, citric acid, and lactic acid, or combinations thereof.

Absorption enhancers include, but are not limited to, a hyaluronidase enzyme. In pharmaceutical formulations containing a hyaluronate salt, the hyaluronidase enzyme may have the added effect of breaking down the hyaluronate salt to speed drug release in cases where increased release rates are desired. This additive may be added to the pharmaceutical formulation immediately prior to injection or may be injected separately as part of a multi-component injection, such as using a dual chamber syringe or a multi-syringe (e.g., two syringe) set up. In some embodiments, the pharmaceutical formulation does not contain a hyaluronidase enzyme, nor is a hyaluronidase enzyme employed during or post injection of the pharmaceutical formulation.

In some embodiments, the pharmaceutical formulation comprises the psychopharmaceutical agent (e.g., a tryptamine psychedelic such as a pharmaceutically acceptable salt of a compound of Formula (I) through (III)); the release modifier; and an aqueous vehicle made up of saline, optionally a buffering agent, optionally a pH adjusting agent (e.g., sodium hydroxide), and optionally a tonicity agent other than sodium chloride. In some embodiments, the pharmaceutical formulation comprises the psychopharmaceutical agent (e.g., a tryptamine psychedelic such as a pharmaceutically acceptable salt of a compound of Formula (I) through (III)); the release modifier; and an aqueous vehicle made up of water for injection, optionally a buffering agent, optionally a pH adjusting agent (e.g., sodium hydroxide), and optionally a tonicity agent. In some embodiments, the pharmaceutical formulation comprises the psychopharmaceutical agent (e.g., a tryptamine psychedelic such as a pharmaceutically acceptable salt of a compound of Formula (I) through (III)); the release modifier; and an aqueous vehicle made up of water for injection, optionally a buffering agent, optionally a pH adjusting agent (e.g., sodium hydroxide), wherein the aqueous vehicle does not contain a tonicity agent such as sodium chloride. In some embodiments, the pharmaceutical formulation comprises the psychopharmaceutical agent (e.g., a tryptamine psychedelic such as a pharmaceutically acceptable salt of a compound of Formula (I) through (III)); the release modifier; and an aqueous vehicle made up of water for injection or saline, and optionally a pH adjusting agent (e.g., sodium hydroxide), wherein the pharmaceutical formulation is formulated without a buffering agent. In some embodiments, the pharmaceutical formulation consists of, or consists essentially of, the psychopharmaceutical agent (e.g., a tryptamine psychedelic such as a pharmaceutically acceptable salt of a compound of Formula (I) through (III)); the release modifier; and water and optional tonicity agent (e.g., sodium chloride) as the aqueous vehicle. By consists essentially of, it is meant that the presence of additional components within the pharmaceutical formulation is permitted, provided the amounts of such additional components do not materially affect the essential characteristics of the pharmaceutical formulation-namely that the pharmaceutical formulation is suitable for injection and provides time-restricted temporal controlled-release of a psychopharmaceutical agent. In some embodiments of administering tryptamine psychedelics (e.g., a pharmaceutically acceptable salt of a compound of Formula (I) through (III)), this time-restricted controlled-release would provide a duration of peak effects of about 30 to 120 minutes.

### Physiochemical Properties

The pharmaceutical formulation may have a pH of about 2, about 2.25, about 2.5, about 2.75, about 3, about 3.25, about 3.5, about 3.75, about 4, about 4.25, about 4.5, about 4.75, about 5, about 5.25, about 5.5, about 5.75, about 6, about 6.25, about 6.5, about 6.75, about 7, about 7.25, about 7.5, about 7.75, about 8, about 8.25, about 8.5, about 8.75, about 9, about 9.25, about 9.5, about 9.75, about 10, about 10.25, about 10.5, about 10.75, about 11, or any range therebetween. Generally, pH values which are too high are associated with tissue necrosis, whereas pH values which are too low are associated with pain and inflammation at the injection site. In some embodiments, the pharmaceutical formulation is suitable for intravenous injection (is an intravenous pharmaceutical formulation) or intramuscular injection (is an intramuscular pharmaceutical formulation), and has a pH ranging from about 2 to about 11, about 3 to about 9, about 4 to about 7, about 4.5 to about 6. In some embodiments, the pharmaceutical formulation is suitable for subcutaneous injection (is a subcutaneous pharmaceutical formulation), and has a pH ranging from about 3 to about 9, about 3 to about 7, about 4 to about 9, about 4 to about 7.5, about 4 to about 7, about 4.5 to about 7.5, about 4.5 to about 7, about 4.5 to about 6.5, about 4.5 to about 6.

The pharmaceutical formulation may have an osmolality of about 150 mOsm/kg, about 155 mOsm/kg, about 160 mOsm/kg, about 165 mOsm/kg, about 170 mOsm/kg, about 175 mOsm/kg, about 180 mOsm/kg, about 185 mOsm/kg, about 190 mOsm/kg, about 195 mOsm/kg, about 200 mOsm/kg, about 225 mOsm/kg, about 250 mOsm/kg, about 275 mOsm/kg, about 300 mOsm/kg, about 325 mOsm/kg, about 350 mOsm/kg, about 375 mOsm/kg, about 400 mOsm/kg, about 425 mOsm/kg, about 450 mOsm/kg, about 475 mOsm/kg, about 500 mOsm/kg, about 525 mOsm/kg, about 550 mOsm/kg, about 575 mOsm/kg, about 600 mOsm/kg, or any range therebetween, such as from about 150 to about 600 mOsm/kg, about 200 to about 500 mOsm/kg, about 250 to about 550 mOsm/kg, about 275 to about 500 mOsm/kg, about 300 to about 450 mOsm/kg, about 150 mOsm/kg to about 200 mOsm/kg, about 150 mOsm/kg to about 175 mOsm/kg. Osmolalities which are outside of these values are reported to cause pain. In some embodiments, the pharmaceutical formulation is isotonic with human blood serum, i.e., has an osmolality of about 275 to about 300 mOsm/kg.

The pharmaceutical formulation may have viscosity of less than about 10,000 cP, less than about 9,000 cP, less than about 8,000 cP, less than about 7,000 cP, less than about 6,000 cP, less than about 5,000 cP, less than about 4,000 cP, less than about 3,000 cP, less than about 2,000 cP, less than about 1,000 cP, less than about 500 cP, less than about 100 cP, less than about 50 cP, less than about 45 cP, less than about 40 cP, less than about 35 cP, less than about 30 cP, less than about 25 cP, less than about 20 cP, for example, about 1 cP, about 2 cP, about 3 cP, about 4 cP, about 5 cP, about 8 cP, about 10 cP, about 12 cP, about 15 cP, about 18 cP, about 20 cP, about 22 cP, about 25 cP, about 28 cP, about 30 cP, about 32 cP, about 35 cP, about 38 cP, about 40 cP, about 42 cP, about 45 cP, about 50 cP, about 60 cP, about 70 cP, about 80 cP, about 90 cP, about 100 cP, about 150 cP, about 200 cP, about 250 cP, about 300 cP, about 350 cP, about 400 cP, about 500 cP, about 600 cP, about 800 cP, about 1,000 cP, about 1,200 cP, about 1,500 cP, about 1,750 cP, about 2,000 cP, about 2,250 cP, about 2,500 cP, about 2,750 cP, about 3,000 cP, about 3,100 cP, about 3,500 cP, about 4,000 cP, about 4,500 cP, about 5,000 cP, about 6,000 cP, about 7,000 cP, about 8,000 cP, about 9,000 cP, about 10,000 cP, or any range therebetween. Such viscosity values allow the pharmaceutical formulation to be syringeable and injectable, for example without causing excessive pain at the injection site. In some embodiments, such as when the release modifier employed is a hyaluronate salt, suitable viscosity values also enable the use of sterile filtration as the sterilization technique. In some embodiments, the pharmaceutical formulation is suitable for subcutaneous injection (is a subcutaneous pharmaceutical formulation), and preferably has a viscosity of less than about 3,000 cP, less than about 2,500 cP, less than about 2,000 cP, less than about 1,500 cP, less than about 1,000 cP, less than about 500 cP, less than about 100 cP, less than about 50 cP, less than about 25 cP, less than about 20 cP, for example, about 1 cP, about 2 cP, about 3 cP, about 4 cP, about 5 cP, about 8 cP, about 10 cP, about 12 cP, about 15 cP, about 18 cP, about 20 cP, about 22 cP, or any range therebetween.

In some embodiments, the pharmaceutical formulation has a shelf-life as an aqueous solution of at least 1 day, 2 days, 3 days, 4 days 5 days, 6 days, 1 week, 2 weeks, 3 weeks, 4 weeks, 5 weeks, 6 weeks, or longer, without significant product degradation or physical changes such as precipitation. In some embodiments, the pharmaceutical formulation can be maintained/stored as an aqueous solution in open or closed environments, such as in open or closed flasks/vials under sub-ambient, ambient, or stress conditions (elevated temperatures) without appreciable degradation or physical changes such as precipitation. Pharmaceutical formulations with a prolonged shelf-life of at least several days or at least several weeks are advantageous because they may be prepared well in advance of administration if desired, and optionally stored, without materially affecting efficacy or injectability. In some embodiments, pharmaceutical formulations formed from a pharmaceutically acceptable salt of a compound of the present disclosure, e.g., a compound of Formula (I) through (III), as the psychopharmaceutical agent, are characterized by increased stability compared to formulations prepared using the same compound as free base but are otherwise substantially the same. For example, the pharmaceutical formulation of the present disclosure formed from a pharmaceutically acceptable salt of a compound of the present disclosure, e.g., a compound of Formula (I) through (III), may be at least 5%, at least 10%, at least 15%, at least 20%, at least 25%, at least 30%, at least 35%, at least 40%, at least 45%, at least 50%, at least 55%, at least 60%, at least 65%, at least 70% more stable upon storage for 24 hours, 48 hours, 72 hours, 1 week, 2 weeks, 3 weeks, 4 weeks, or longer, in terms of degradation or physical changes such as precipitation, compared to formulations prepared from a free base counterpart but are otherwise substantially the same.

### Release Kinetics

The pharmaceutical formulation enables time-restricted temporal controlled-release of the psychopharmaceutical agent via bolus injection to humans, and via bolus subcutaneous injection in particular. In the case of delivering a tryptamine psychedelic (e.g., a pharmaceutically acceptable salt of a compound of Formula (I) through (III)), the pharmaceutical formulations enable controlled-release of the tryptamine psychedelic via bolus injection to humans, and via bolus subcutaneous injection in particular, that mimics the clinically advantageous peak effects time course of about 30 to 120 minutes achievable by IV infusion of such psychopharmaceutical agents over about 90 minutes. In some embodiments, the duration of peak effects following bolus injection of the pharmaceutical formulation, preferably bolus subcutaneous injection of the pharmaceutical formulation, is about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, or any range therebetween, such as from about 30 to about 120 minutes, from about 30 to about 45 minutes, from about 40 to about 100 minutes, from about 45 to about 90 minutes, from about 50 to about 75 minutes, from about 60 to about 70 minutes.

In some embodiments, the controlled-release of a psychedelic (e.g., tryptamine psychedelic) places the patient into a psychedelic state (a correlate of positive clinical outcomes) for about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 130 minutes, about 140 minutes, about 150 minutes, about 160 minutes, about 170 minutes, about 180 minutes, or any range therebetween, such as a psychedelic state time course of from about 35 minutes to about 100 minutes, from about 40 minutes to about 80 minutes, from about 35 to about 45 minutes, from about 50 minutes to about 60 minutes, from about 40 minutes to about 50 minutes, from about 90 minutes to about 120 minutes.

Non-limiting examples of PD clinical scales, diary assessments, and assessments by a clinician or caregiver which can be used to assess the duration of action, duration of psychedelic state, duration of peak effects, mental state, mood, drug effect, etc. of the patient following injection include, but are not limited to, Mystical Experience Questionnaire (MEQ30), the 5-Dimensional Altered States of Consciousness Rating Scale (5D-ASC), the Hallucinogen Rating Scale (HRS), the visual analogue scale (VAS) ratings of ‘Any drug effect’, a 5-item Persisting Effects Questionnaire (PEQ) assessing the meaningfulness, spirituality and psychological insights and challenges of the experience and overall drug effect, the NeuroCart test battery (e.g., saccadic eye movements, smooth pursuit eye movements, adaptive tracking, body sway, pupil size ratio, Visual analogs Scales Bond and Lader (VAS B&L) and Bowdle (VAS Bowdle)), pharmaco electroencephalogram (EEG), State-Trait Anxiety Inventory (STAI), The Dutch Temperament and Character Inventory (TCI), The Dutch Personality Questionnaire-2-Revised (NPV-2-R), The Profile of Mood States (POMS), the Real-Time Intensity scale, and MINI. The time course that the patient experiences peak effects may also be assessed by the time the patient has a drug concentration in the blood of ≥40 ng/mL, for example, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, or any range therebetween. The time course that the patient spends in the psychedelic state may also be assessed by the time the patient has a therapeutically relevant concentration of the drug in the blood, e.g., the time that the patient has a drug concentration between about 20 ng/mL and about 150 ng/mL, for example, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, or any range therebetween.

The Mystical Experiences Questionnaire was first developed during an online survey on psilocybin-containing mushrooms and validated using data from experimental studies with controlled doses of psilocybin. The revised version contains 30 items (MEQ30) regarding subjective drug effects and is completed retrospectively. Effects are scored in total and on four subdomains (mystical, positive mood, space/time, ineffability), based on a percentage of maximum possible score. A Mystical Experience Questionnaire (MEQ30) score of greater than 60%, the criteria for a complete mystical experience, has been shown to be a mediator of symptom improvement in previous psychedelic clinical trials. In some embodiments, the methods herein provide the patient with a Mystical Experience Questionnaire (MEQ30) score of at least 60%, at least 65%, at least 70%, at least 75%, at least 80%, at least 85%, at least 90%, at least 95%. In some embodiments, at least 50%, at least 55%, at least 60%, at least 65%, at least 70%, at least 75%, at least 80%, at least 90% of subjects treated with the methods described herein have a Mystical Experience Questionnaire (MEQ30) score of at least 60%, at least 65%, at least 70%, at least 75%, at least 80%, at least 85%, at least 90%, at least 95%.

The 5-Dimensional Altered States of Consciousness Rating Scale (5D-ASC) measures altered states of consciousness and contains 94 items (visual analog scales). The 5D-ASC scale measures alterations in mood, perception, experience of self in relation to environment, and thought disorder. The instrument consists of five subscales/dimensions and 11 lower-order scales. The 5D-ASC dimension “Oceanic Boundlessness” (27 items) measures derealization and depersonalization associated with positive emotional states, ranging from heightened mood to euphoric exaltation. The corresponding lower-order scales include “experience of unity,” “spiritual experience,” “blissful state,” and “insightfulness.” The dimension “Anxious Ego Dissolution” (21 items) summarizes ego disintegration and loss of self-control phenomena associated with anxiety. The corresponding lower-order scales include “disembodiment,” “impaired control of cognition,” and “anxiety.” The dimension “Visionary Restructuralization” (18 items) consists of the lower-order scales “complex imagery,” “elementary imagery,” “audio-visual synesthesia,” and “changed meaning of percepts.” Two additional dimensions describe “Auditory Alterations” (15 items) and “Reduction of Vigilance” (12 items). Scoring is based on a percentage of maximum possible score. The scale is well-validated and widely used to characterize the subjective effects of various psychedelic drugs. In some embodiments, the 5D-ASC is used to measure changes in the subjects' subjective psychedelic experience. In some embodiments, the methods herein provide the patient with a 5D-ASC score (e.g., on the oceanic boundlessness subscale) of at least 60%, at least 65%, at least 70%, at least 75%, at least 80%, at least 85%, at least 90%, at least 95%. In some embodiments, at least 50%, at least 55%, at least 60%, at least 65%, at least 70%, at least 75%, at least 80%, at least 90% of subjects treated with the methods described herein have a 5D-ASC score (e.g., on the oceanic boundlessness subscale) of at least 60%, at least 65%, at least 70%, at least 75%, at least 80%, at least 85%, at least 90%, at least 95%.

The Hallucinogen Rating Scale (HRS) is a questionnaire with up to 100 items, and the questionnaire is designed to assess the subjective effects of hallucinogenic substances. Participants rate responses to the majority of questions on a 5-point intensity scale: 0=not at all; 1=slightly; 2=moderately; 3=quite a bit; and 4=extremely. Some questions have a slightly modified scale, and one question asks to rate the amount of time between when the drug was administered and feeling an effect from: no effect, 0-5 minutes, 5-15 minutes, 15-30 minutes, 30-60 minutes, or more than one hour. Questions are grouped into factors or domains including: 1) somaesthesia, 2) affect, 3) perception, 4) cognition, 5) volition, and 6) intensity. In some embodiments, the methods herein provide the patient with a Hallucinogen Rating Scale (HRS) score (e.g., on the intensity subscale) of at least 1.8, at least 2.0, at least 2.2, at least 2.4, at least 2.6, at least 2.8, at least 3.0, at least 3.2, at least 3.4, at least 3.6, at least 3.8, or 4.0. In some embodiments, at least 50%, at least 55%, at least 60%, at least 65%, at least 70%, at least 75%, at least 80%, at least 90% of subjects treated with the methods described herein have a score on the Intensity subscale of the HRS of greater than >2.79, for example, a score of at least 2.8, at least 3.0, at least 3.2, at least 3.4, at least 3.6, at least 3.8, or 4.0.

The visual analogue scale (VAS) is a psychometric response scale that can be used in questionnaires. It is a measurement instrument for subjective characteristics or attitudes that cannot be directly measured. When responding to a VAS item, respondents specify their level of agreement to a statement by indicating a position along a continuous line between two end points, usually a horizontal 100-mm line marked from “not at all,” “definitely not,” or the like, on the left to “extremely,” “definitely so,” or the like, on the right. The Bond and Lader VAS involves the subject indicating (with vertical marks) on sixteen horizontal 100-mm visual analogue scales how he/she feels. From these measurements, three main factors are calculated as described by Bond and Lader: alertness (from nine scores), contentedness (often called mood; from five scores), and calmness (from two scores). The Bowdle VAS and Drug Rating VAS provides the subject with items of the drug rating questionnaire: feel drug, like drug, and dislike drug. The VAS any drug effect involves the subject indicating (with vertical marks) on horizontal 100-mm visual analogue scales with the any drug effects item (“I can feel any drug effect”). In some embodiments, the subject has a maximum VAS score (e.g., Bond and Lader VAS, Bowdle VAS and Drug Rating VAS, and/or VAS any drug effect) of ≥60 mm or ≥70 mm after treatment. In some embodiments the subject has a VAS score (e.g., Bond and Lader VAS, Bowdle VAS and Drug Rating VAS, and/or VAS any drug effect) of greater than or equal to 60 mm, 65 mm, 70 mm, 75 mm, 80 mm, 85 mm, 90 mm, or 95 mm, after treatment. In some embodiments, at least 50%, at least 55%, at least 60%, at least 65%, at least 70%, at least 75%, at least 80%, at least 85%, at least 90% of subjects treated with the methods described herein report a maximum VAS score (e.g., Bond and Lader VAS, Bowdle VAS and Drug Rating VAS, and/or VAS any drug effect) of greater than or equal to 60 mm, 65 mm, 70 mm, 75 mm, 80 mm, 85 mm, 90 mm, or 95 mm following treatment. In some embodiments, following treatment, the subject experiences a VAS any drug effect score of ≥70 mm for a duration of about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, or any range therebetween, such as from about 30 to about 120 minutes, from about 30 to about 45 minutes, from about 40 to about 100 minutes, from about 45 to about 90 minutes, from about 50 to about 75 minutes, from about 60 to about 70 minutes.

The 5-item Persisting Effects Questionnaire (PEQ) is a 5-item questionnaire that assesses the meaningfulness, spiritual significance, psychological insightfulness, and how psychologically challenging a participant experience was during the medicine session. Scores are assessed on a scale from 0 (not at all) to 5 (extremely). Higher scores (under consideration of reverse-scored items) indicate stronger persisting treatment effects. In some embodiments, the patient reports their experience as the most or among the top five most meaningful experiences of their life according to the Persisting Effects Questionnaire (PEQ). In some embodiments, the patient reports their experience as the most or among the top five most psychologically insightful experiences of their life, according to the Persisting Effects Questionnaire (PEQ).

The Profile of Mood States (POMS) measures six identifiable mood- or affective states: Tension-Anxiety, Depression-Rejection, Anger-Hostility, Vigor-Activity, and Fatigue-Inertia. In the original POMS, these states are addressed through 65 five-point adjective rating scales, and a sixth dimension (confusion-bewilderment) is added that does not appear in the abbreviated POMS wherein 32 questions are used to assess different periods (usually past two hours, sometimes past week). The scales use a five-point score system, ranging from 0 (not at all) to 4 (extremely). The scores of the different states are calculated using a scoring algorithm.

In the Real-Time Intensity scale, subjects are asked to verbally rate the psychological intensity of their experience on a scale from 0-4 (0=not at all; 1=slightly; 2=moderately; 3 very much; 4=extremely) on three levels: visual intensity, bodily intensity, and emotional/metacognitive intensity. Their answers are recorded during the drug administration (during the experience). In some embodiments, the methods herein provide the patient with a Real-Time Intensity scale score (e.g., on the emotional/metacognitive subscale) of at least 1.8, at least 2.0, at least 2.2, at least 2.4, at least 2.6, at least 2.8, at least 3.0, at least 3.2, at least 3.4, at least 3.6, at least 3.8, or 4.0. In some embodiments, at least 50%, at least 55%, at least 60%, at least 65%, at least 70%, at least 75%, at least 80%, at least 90% of subjects treated with the methods described herein have a score on the Real-Time Intensity scale of greater than >2.79, for example, a score of at least 2.8, at least 3.0, at least 3.2, at least 3.4, at least 3.6, at least 3.8, or 4.0.

In some embodiments, the pharmaceutical formulation provides a controlled-release of the psychopharmaceutical agent such as a tryptamine psychedelic (e.g., a pharmaceutically acceptable salt of a compound of Formula (I) through (III)) following bolus injection, including bolus subcutaneous injection, which achieves an onset of effects within about 30 minutes, about 25 minutes, about 20 minutes, about 15 minutes, about 10 minutes post administration, such as from about 1 minute to about 30 minutes, about 5 minutes to about 25 minutes, about 10 minutes to about 20 minutes, about 15 minutes to about 30 minutes, about 5 minutes to about 10 minutes. In some embodiments, the pharmaceutical formulation provides a controlled-release of the psychopharmaceutical agent such as a tryptamine psychedelic (e.g., a pharmaceutically acceptable salt of a compound of Formula (I) through (III)) following bolus injection, including bolus subcutaneous injection, which achieves an offset of effects of greater than about 35 minutes and up to about 180 minutes post administration, such as about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 160 minutes, about 170 minutes, about 180 minutes, post administration, or any range therebetween.

In some embodiments, the pharmaceutical formulation provides a controlled-release of the psychopharmaceutical agent such as a tryptamine psychedelic (e.g., a pharmaceutically acceptable salt of a compound of Formula (I) through (III)) following bolus injection, including bolus subcutaneous injection, with a drug concentration in the blood of ≥40 ng/mL, corresponding to peak psychedelic effects. For example, following bolus injection, including bolus subcutaneous injection, the pharmaceutical formulation may provide a drug concentration in the blood of about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, or any range therebetween. For maximum therapeutic benefits and clinical scalability, these drug concentrations (and thus peak effects) are preferably provided for a duration of about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, or any range therebetween.

In some embodiments, the pharmaceutical formulation provides a controlled-release of the psychopharmaceutical agent such as a tryptamine psychedelic (e.g., a pharmaceutically acceptable salt of a compound of Formula (I) through (III)) following bolus injection, including bolus subcutaneous injection, which places the subject into a psychedelic state with a therapeutically relevant concentration of the drug in the blood of about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, or any range therebetween, such as from about 20 to about 150 ng/mL, or from about 30 to about 100 ng/mL, or from about 60 to about 100 ng/mL or from about 40 to about 80 ng/mL, or from about 45 to about 60 ng/mL, or from about 60 to about 80 ng/mL, or from about 50 to about 80 ng/mL. The psychedelic state and therapeutically relevant concentration of the drug may last for about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 130 minutes, about 140 minutes, about 150 minutes, about 160 minutes, about 170 minutes, about 180 minutes, or any range therebetween.

Further, desirable pharmaceutical formulations provide a controlled-release of the tryptamine psychedelic (e.g., a pharmaceutically acceptable salt of a compound of Formula (I) through (III)) following bolus injection, including bolus subcutaneous injection, to achieve the aforementioned duration of peak effects and/or psychedelic state time course, but do so without a burst release of the tryptamine psychedelic exceeding maximum plasma levels (Cₘₐₓ) of about 200 ng/mL, about 195 ng/mL, about 190 ng/mL, about 185 ng/mL, about 180 ng/mL, about 175 ng/mL, about 170 ng/mL, about 165 ng/mL, about 160 ng/mL, or about 155 ng/mL. Consequently, pharmaceutical formulations of the present disclosure, and in particular subcutaneous pharmaceutical formulations of the present disclosure, provide a smoother, more controlled delivery of the psychopharmaceutical agent contained therein compared to bolus IV injections of the same agent formulated without release modifier, which are known to cause high levels of drug spiking immediately following IV injection. In preferred embodiments, the pharmaceutical formulations provide a controlled-release of the tryptamine psychedelic (e.g., a pharmaceutically acceptable salt of a compound of Formula (I) through (III)) following bolus injection, including bolus subcutaneous injection, to achieve the aforementioned duration of peak effects and/or psychedelic state time course, along with a maximum concentration of drug in the blood (Cₘₐₓ) of about 60 ng/mL, about 65 ng/mL, about 70 ng/mL, about 75 ng/mL, 80 ng/mL, or any range therebetween, such as a Cₘₐₓ of about 60 ng/mL to about 80 ng/mL.

In some embodiments, the pharmaceutical formulation provides a controlled-release of the psychopharmaceutical agent such as a tryptamine psychedelic (e.g., a pharmaceutically acceptable salt of a compound of Formula (I) through (III)) following bolus injection, including bolus subcutaneous injection, which achieves a concentration of the drug in the blood corresponding to peak effects of about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, or any range therebetween, such as from about 60 ng/mL to about 80 ng/mL, followed by a steady-state exposure of from about 20 ng/mL to about 60 ng/mL, or any range therebetween, such as from about 20 ng/mL to about 40 ng/mL, about 25 ng/mL to about 35 ng/mL, about 20 ng/mL to about 30 ng/mL, or about 40 ng/mL to about 50 ng/mL, the steady-state exposure lasting for at least 30 minutes, e.g., about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, or any range therebetween. In some embodiments, the pharmaceutical formulation provides a controlled-release of the psychopharmaceutical agent such as a tryptamine psychedelic (e.g., a pharmaceutically acceptable salt of a compound of Formula (I) through (III)) following bolus injection, including bolus subcutaneous injection, which achieves a concentration of the drug in the blood corresponding to peak effects of about 60 ng/mL, about 65 ng/mL, about 70 ng/mL, about 75 ng/mL, about 80 ng/mL, or any range therebetween, followed by a steady-state exposure of from about 40 ng/mL to about 60 ng/mL, or any range therebetween, such as from about 40 ng/mL to about 50 ng/mL, the steady-state exposure lasting for at least 30 minutes, e.g., about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, or any range therebetween.

In some embodiments, the pharmaceutical formulation provides a controlled-release of the psychopharmaceutical agent such as tryptamine psychedelic (e.g., a pharmaceutically acceptable salt of a compound of Formula (I) through (III)) following bolus injection, including bolus subcutaneous injection, which achieves a plasma half-life (t_{1/2}) of about 30 minutes, about 35 minutes, about 40 minutes, about 45 minutes, about 50 minutes, about 55 minutes, about 60 minutes, or any range therebetween, such as from about 30 minutes to about 60 minutes, or from about 35 minutes to about 50 minutes, or from about 40 minutes to about 45 minutes.

In some embodiments, the pharmaceutical formulation provides a controlled-release of the psychopharmaceutical agent such as tryptamine psychedelic (e.g., a pharmaceutically acceptable salt of a compound of Formula (I) through (III)) following bolus injection, including bolus subcutaneous injection, which achieves a time to maximum drug concentration (Tₘₐₓ) of about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any range therebetween, such as from about 20 minutes to about 60 minutes, or from about 25 minutes to about 50 minutes, or from about 30 minutes to about 45 minutes, or from about 10 minutes to about 30 minutes.

The pharmaceutical formulation may display first order release kinetics of the psychopharmaceutical agent, as determined by the Dialysis—Drug Release Test (see Example section for experimental protocol). Accordingly, the release kinetics may fit best to the first order mathematical equation (2):

$$\frac{Q}{{Q}_{0}} = 1 - {e}^{( - kt )} \qquad ( 2 )$$

where Q is the concentration of the drug, Q₀ is the initial concentration of the drug, k is the first order rate constant, and t is the time.

The pharmaceutical formulation may extend the release of the psychopharmaceutical agent compared to the release of the same psychopharmaceutical agent from a formulation without release modifier but is otherwise substantially the same. In the case of short-acting tryptamine psychedelics (e.g., a pharmaceutically acceptable salt of a compound of Formula (I) through (III)) for example, a longer release period may result in a more clinically advantageous peak effects time course and clinical efficacy. In some embodiments, the time required for release of 25% of a total psychopharmaceutical agent content (free base equivalence) in the pharmaceutical formulation (t_{25%}) is at least about 35% longer, at least about 40% longer, at least about 45% longer, at least about 50% longer, at least about 55% longer, at least about 60% longer, at least about 65% longer, at least about 70% longer, at least about 75% longer, at least about 80% longer, at least about 85% longer, at least about 90% longer, at least about 95% longer, at least about 100% longer than the t_{25%} of a formulation prepared without release modifier but is otherwise substantially the same, as determined by the Dialysis—Drug Release Test (see Example section for experimental protocol). In some embodiments, the time required for release of 50% of a total psychopharmaceutical agent content (free base equivalence) in the pharmaceutical formulation (t_{50%}) is at least about 35% longer, at least about 40% longer, at least about 45% longer, at least about 50% longer, at least about 55% longer, at least about 60% longer, at least about 65% longer, at least about 70% longer, at least about 75% longer, at least about 80% longer, at least about 85% longer, at least about 90% longer, at least about 95% longer, at least about 100% longer than the t₅₀% of a formulation prepared without release modifier but is otherwise substantially the same, as determined by the Dialysis Drug Release Test. In some embodiments, the pharmaceutical formulation provides a time-restricted controlled-release of the psychopharmaceutical agent such as a tryptamine psychedelic (e.g., a pharmaceutically acceptable salt of a compound of Formula (I) through (III)) so as not to overextend the release and the resulting duration of peak effects beyond about 120 minutes to avoid the prolonged supervised clinical observation requirements imposed by longer-acting psychopharmaceutical agents such as LSD and psilocybin. In some embodiments, the t_{25%} is no more than about 250% longer, no more than about 225% longer, no more than about 200% longer, no more than about 190% longer, no more than about 180% longer, no more than about 170% longer, no more than about 160% longer, no more than about 150% longer, no more than about 140% longer, no more than about 130% longer than the t_{25%} of a formulation prepared without release modifier but is otherwise substantially the same, as determined by the Dialysis—Drug Release Test. In some embodiments, the t_{50%} is no more than about 250% longer, no more than about 225% longer, no more than about 200% longer, no more than about 190% longer, no more than about 180% longer, no more than about 170% longer, no more than about 160% longer, no more than about 150% longer, no more than about 140% longer, no more than about 130% longer than the t_{50%} of a formulation prepared without release modifier but is otherwise substantially the same, as determined by the Dialysis—Drug Release Test. In some embodiments, the time required for release of 50% of a total psychopharmaceutical agent content in the pharmaceutical formulation (t_{50%}) is no more than about 120 minutes, no more than about 110 minutes, no more than about 100 minutes, no more than about 90 minutes, no more than about 80 minutes, no more than about 70 minutes, no more than about 60 minutes, no more than about 50 minutes, no more than about 40 minutes, no more than about 30 minutes, as determined by the Dialysis—Drug Release Test.

### Kits

Also disclosed herein is a kit suitable for preparing the injectable pharmaceutical formulation of the present disclosure. The kit comprises a psychopharmaceutical agent, a release modifier such as a hyaluronate salt or a carboxymethyl cellulose salt, and an aqueous vehicle. Any of the embodiments disclosed herein for the pharmaceutical formulation and the ingredients contained therein apply to the kit of the present disclosure.

In some embodiments, the kit comprises (a1) a first solution comprising a psychopharmaceutical agent and an aqueous vehicle, and (b1) a second solution comprising a release modifier such as a hyaluronate salt or a carboxymethyl cellulose salt and an aqueous vehicle. The kit components (a1) and (b1) are intended to be combined, contacted, or otherwise brought together to generate the injectable pharmaceutical formulation of the present disclosure.

In some embodiments, (a1) the first solution comprises (as psychopharmaceutical agent) a tryptamine psychedelic, such as a pharmaceutically acceptable salt of a compound of Formula (I) through (III). The (a1) first solution may be prepared from a pre-formed, typically solid form and in some cases crystalline solid form, of the pharmaceutically acceptable salt of a compound of the present disclosure (e.g., a compound of Formula (I) through (III)) by contacting the pre-formed pharmaceutically acceptable salt of a compound of the present disclosure with aqueous vehicle. Alternatively, (a1) the first solution may be prepared by contacting the compound of the present disclosure (e.g., a compound of Formula (I) through (III)) as a free base with an aqueous vehicle comprising available H⁺ (aq) ions capable of ionizing/protonating the free base to form the pharmaceutically acceptable salt of a compound of the present disclosure within (a1) the first solution in-situ. In some embodiments, (a1) the first solution comprises a free base concentration of psychopharmaceutical agent, e.g., a free base concentration of a compound of Formula (I) through (III) (free base equivalence when a salt form is used), by weight per total volume of first solution of about 2 mg/mL, about 4 mg/mL, about 6 mg/mL, about 8 mg/mL, about 10 mg/mL, about 12 mg/mL, about 15 mg/mL, about 18 mg/mL, about 20 mg/mL, about 22 mg/mL, about 25 mg/mL, about 28 mg/mL, about 30 mg/mL, about 32 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, or any range therebetween, such as from about 10 mg/mL to about 140 mg/mL, from about 20 mg/mL to about 100 mg/mL, from about 30 mg/mL to about 80 mg/mL, from about 40 mg/mL to about 70 mg/mL.

In some embodiments, (b1) the second solution comprises as the release modifier a hyaluronate salt such as sodium hyaluronate, and the aqueous vehicle. A concentration of the hyaluronate salt by weight per total volume of (b1) the second solution expressed as a percentage (% w/v) may be about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, or any range therebetween, such as from about 0.1% to about 4%, about 0.1% to about 3%, about 0.1% to about 2%, about 0.1% to about 1.5%, about 0.1% to about 1%, about 0.15% to about 1%, about 0.2% to about 1%, or about 0.25% to about 0.75%.

In some embodiments, (b1) the second solution comprises as the release modifier a carboxymethyl cellulose salt such as sodium carboxymethyl cellulose, and the aqueous vehicle. A concentration of carboxymethyl cellulose salt by weight per total volume of (b1) the second solution expressed as a percentage (% w/v) may be about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.6% to about 2%, about 0.7% to about 1.8%, about 0.8% to about 1.7%, about 0.9% to about 1.6%, about 1% to about 1.5%.

A suitable volume of each of kit components (a1) and (b1) may be combined, contacted, or otherwise brought together to generate the pharmaceutical formulation with suitable physiochemical and controlled-release characteristics, as well as suitable unit doses and psychopharmaceutical agent concentrations. Kits may be provided with different concentrations of psychopharmaceutical agent in component (a1), different concentrations of release modifier in component (b1), and/or different volume ratios of kit components (a1) and (b1) may be combined, contacted, or otherwise brought together to generate different pharmaceutical formulations with differentiated release characteristics, unit doses, and/or psychopharmaceutical agent concentrations. For example, the same kit may be used to generate a range of differentiated pharmaceutical formulations in terms of, inter alia, unit dose, psychopharmaceutical agent concentration, release modifier, and release characteristics, by using different volume ratios of kit components (a1):(b1). A suitable volume ratio of (a1):(b1) used to generate the pharmaceutical formulation may be from about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, or any range therebetween, such as from about 2:1 to about 1:2, about 1.5:1 to about 1:1.5, about 1:1. The combining, contacting, or otherwise bringing together of kit components (a1) and (b1) to generate the pharmaceutical formulation may be performed well in advance of administration with the pharmaceutical formulation being optionally stored, or immediately prior to use. The kit may include instructions for what volume ratios of (a1):(b1) can be used to prepare suitable injectable pharmaceutical formulations, for example, based on physiochemical properties, indication to be treated, dosing schedule and requirements, etc.

In some embodiments, the kit comprises (a2) a psychopharmaceutical agent in solid form and (b2) a solution comprising a release modifier such as a hyaluronate salt or a carboxymethyl cellulose salt, and an aqueous vehicle. The kit components (a2) and (b2) are intended to be combined, contacted, or otherwise brought together to generate the injectable pharmaceutical formulation of the present disclosure.

In some embodiments, (a2) the psychopharmaceutical agent is a tryptamine psychedelic in solid form, such as a pharmaceutically acceptable salt of a compound of Formula (I) through (III), in solid form. Here, any pre-formed, solid form and in some cases crystalline solid form, of the pharmaceutically acceptable salt of a compound of Formula (I) through (III) of the present disclosure can be used in kit component (a2).

In some embodiments, (b2) the solution comprises as the release modifier a hyaluronate salt such as sodium hyaluronate, and the aqueous vehicle. A concentration of the hyaluronate salt by weight per total volume of (b2) the solution expressed as a percentage (% w/v) may be about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.1% to about 2%, about 0.1% to about 1.5%, about 0.1% to about 1%, about 0.1% to about 0.75%, about 0.1% to about 0.5%, about 0.15% to about 1%, about 0.2% to about 0.75%, or about 0.25% to about 0.5%.

In some embodiments, (b2) the solution comprises as the release modifier a carboxymethyl cellulose salt such as sodium carboxymethyl cellulose, and the aqueous vehicle. A concentration of carboxymethyl cellulose salt by weight per total volume of (b2) the solution expressed as a percentage (% w/v) may be about 0.55%, about 0.6%, about 0.65%, about 0.7%, about 0.75%, about 0.8%, about 0.85%, about 0.9%, about 0.95%, about 1%, or any range therebetween, such as from about 0.6% to about 1%, about 0.7% to about 1%, about 0.75% to about 1%, about 0.75% to about 0.9%, about 0.75% to about 0.8%.

A suitable volume of kit component (b2) may be combined, contacted, or otherwise brought together with kit component (a2) to generate the pharmaceutical formulation with suitable physiochemical and controlled-release characteristics, as well as suitable psychopharmaceutical agent concentrations. Kits may be provided with different concentrations of release modifier in component (b2) and/or different volumes of kit component (b2) may be combined, contacted, or otherwise brought together with kit component (a2) to generate different pharmaceutical formulations with differentiated release characteristics and psychopharmaceutical agent concentrations. Kits may be provided with different amounts of psychopharmaceutical agent in solid form in component (a2) to generate different pharmaceutical formulations with differentiated unit doses and psychopharmaceutical agent concentrations. The combining, contacting, or otherwise bringing together the kit component (b2) with the solid dosage form of the psychopharmaceutical agent in kit component (a2) can be accurately described as an act of reconstituting the solid dosage form of the psychopharmaceutical agent (e.g., a tryptamine psychedelic such as a pharmaceutically acceptable salt of a compound of Formula (I) through (III), in solid form and in some case in crystalline solid form). Reconstitution may be performed well in advance of administration with the pharmaceutical formulation being optionally stored, or immediately prior to use. The kit may include instructions for what volume of kit component (b2) can be combined, contacted, or otherwise brought together with kit component (a2) to prepare suitable injectable pharmaceutical formulations, for example, based on physiochemical properties, indication to be treated, dosing schedule and requirements, etc.

As described previously, in addition to water, the aqueous vehicle used in any of the aforementioned kit components (e.g., kit component (a1), kit component (b1), kit component (b2)) may optionally contain one or more pharmaceutically acceptable additives, as desired/needed, such as water-miscible vehicles, non-aqueous vehicles, antimicrobial agents or preservatives against the growth of microorganisms, stabilizing agents, tonicity agents, buffering agents, antioxidants, local anesthetics, complexing agents, sequestering or chelating agents, pH adjusting agents, absorption enhancers, including combinations thereof. Suitable aqueous vehicles include, but are not limited to, water, saline, physiological or isotonic saline, phosphate buffered saline (PBS), sodium chloride injection, Ringers injection, isotonic dextrose injection, sterile water injection, dextrose and lactated Ringers injection. In some embodiments, the aqueous vehicle is made up of saline, optionally a buffering agent, optionally a pH adjusting agent (e.g., sodium hydroxide), and optionally a tonicity agent other than sodium chloride. In some embodiments, the aqueous vehicle is made up of water for injection, optionally a buffering agent, optionally a pH adjusting agent (e.g., sodium hydroxide), and optionally a tonicity agent. In some embodiments, the aqueous vehicle is made up of water or saline, and optionally a pH adjusting agent (e.g., sodium hydroxide), wherein the aqueous vehicle is formulated without a buffering agent. In some embodiments, the aqueous vehicle is formulated without a tonicity agent such as sodium chloride. In some embodiments, the aqueous vehicle used in one or more of kit components (a1), (a2), and/or (b2), independent of one another, is water, such as water for injection (WFI). In some embodiments, the aqueous vehicle used in one or more of kit components (a1), (a2), and/or (b2), independent of one another, comprises sodium chloride at a concentration, in terms of weight per volume expressed as a percentage (% w/v), of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.1% to about 1.5%, about 0.2% to about 1%, about 0.3% to about 0.5% w/v.

In some embodiments, a pH adjustment may be performed with a pH adjusting agent on one or more of kit component (a1), kit component (b1), and/or kit component (b2). In some embodiments, after combining, contacting, or otherwise bringing together the respective kit components (kit components (a1) with (b1) or kit components (a2) with (b2)), a pH adjustment may be performed with a pH adjusting agent to generate the final pharmaceutical formulation. Therefore, the kit may optionally comprise (c) a pH adjusting agent. In some embodiments, the pH adjusting agent is at least one selected from the group consisting of sodium hydroxide, potassium hydroxide, sodium carbonate, ammonium hydroxide, calcium hydroxide, magnesium hydroxide, hydrochloric acid, citric acid, and lactic acid. In some embodiments, the pH adjustment involves increasing the pH, and so kit component (c) comprises a suitable base as pH adjusting agent, for example one or more of sodium hydroxide, potassium hydroxide, sodium carbonate, ammonium hydroxide, calcium hydroxide and magnesium hydroxide. In some embodiments, the pH is adjusted with sodium hydroxide or potassium hydroxide, for example to generate the final pharmaceutical formulation. In some embodiments, the pharmaceutical formulation does not contain a buffering agent.

### Preparative Methods

Also disclosed herein is a method for preparing the pharmaceutical formulation of the present disclosure, the pharmaceutical formulation being suitable for injection. The method comprises contacting a psychopharmaceutical agent, a release modifier such as a hyaluronate salt or a carboxymethyl cellulose salt, and an aqueous vehicle. The contacting of the method may be conducted in a variety of ways, including through the use of conventional techniques known to those skilled in the art of pharmaceutical science (see, Remington: The Science and Practice of Pharmacy). The method for preparing the pharmaceutical formulation may be performed well in advance of administration with the pharmaceutical formulation being optionally stored, or immediately prior to use.

In some embodiments, the psychopharmaceutical agent is a tryptamine psychedelic, such as a pharmaceutically acceptable salt of a compound of Formula (I) through (III). In some embodiments, the method involves reconstituting a pre-formed, typically solid form and in some cases crystalline solid form of the pharmaceutically acceptable salt of a compound of Formula (I) through (III) in an aqueous vehicle. In some embodiments, the method involves contacting a solution comprising a pharmaceutically acceptable salt of a compound of Formula (I) through (III) in an aqueous vehicle with a release modifier in solid form or as a solution in an aqueous vehicle. Alternatively, the method may involve contacting the compound of the present disclosure (e.g., a compound of Formula (I) through (III)) as a free base with an aqueous vehicle comprising available H⁺ (aq) ions capable of ionizing/protonating the compounds of the present disclosure, thereby forming the pharmaceutically acceptable salt of a compound of Formula (I) through (III) in-situ.

The method may comprise contacting a psychopharmaceutical agent, a release modifier such as a hyaluronate salt or a carboxymethyl cellulose salt, and an aqueous vehicle from components of a kit intended to be combined, contacted, or otherwise brought together. Any of the embodiments disclosed herein for the kit may be optionally used in the preparative methods herein.

In some embodiments, the method comprises contacting (a1) a first solution comprising a psychopharmaceutical agent and an aqueous vehicle, with (b1) a second solution comprising a release modifier such as a hyaluronate salt or a carboxymethyl cellulose salt and an aqueous vehicle.

In some embodiments, (a1) the first solution comprises (as psychopharmaceutical agent) a tryptamine psychedelic, such as a pharmaceutically acceptable salt of a compound of Formula (I) through (III). The (a1) first solution may be prepared from a pre-formed, typically solid form and in some cases crystalline solid form, of the pharmaceutically acceptable salt of a compound of the present disclosure (e.g., a compound of Formula (I) through (III)) by contacting the pre-formed pharmaceutically acceptable salt of a compound of the present disclosure with aqueous vehicle. Alternatively, (a1) the first solution may be prepared by contacting the compound of the present disclosure (e.g., a compound of Formula (I) through (III)) as a free base with an aqueous vehicle comprising available H⁺ (aq) ions capable of ionizing/protonating the free base to form the pharmaceutically acceptable salt of a compound of the present disclosure within (a1) the first solution in-situ. In some embodiments, (a1) the first solution comprises a free base concentration of psychopharmaceutical agent, e.g., a free base concentration of a compound of Formula (I) through (III) (free base equivalence when a salt form is used), by weight per total volume of first solution of about 2 mg/mL, about 4 mg/mL, about 6 mg/mL, about 8 mg/mL, about 10 mg/mL, about 12 mg/mL, about 15 mg/mL, about 18 mg/mL, about 20 mg/mL, about 22 mg/mL, about 25 mg/mL, about 28 mg/mL, about 30 mg/mL, about 32 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, or any range therebetween, such as from about 10 mg/mL to about 140 mg/mL, from about 20 mg/mL to about 100 mg/mL, from about 30 mg/mL to about 80 mg/mL, from about 40 mg/mL to about 70 mg/mL.

In some embodiments, (b1) the second solution comprises as the release modifier a hyaluronate salt such as sodium hyaluronate, and the aqueous vehicle. A concentration of the hyaluronate salt by weight per total volume of (b1) the second solution expressed as a percentage (% w/v) may be about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, or any range therebetween, such as from about 0.1% to about 4%, about 0.1% to about 3%, about 0.1% to about 2%, about 0.1% to about 1.5%, about 0.1% to about 1%, about 0.15% to about 1%, about 0.2% to about 1%, or about 0.25% to about 0.75%.

In some embodiments, (b1) the second solution comprises as the release modifier a carboxymethyl cellulose salt such as sodium carboxymethyl cellulose, and the aqueous vehicle. A concentration of carboxymethyl cellulose salt by weight per total volume of (b1) the second solution expressed as a percentage (% w/v) may be about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.6% to about 2%, about 0.7% to about 1.8%, about 0.8% to about 1.7%, about 0.9% to about 1.6%, about 1% to about 1.5%.

A suitable volume of each of the first solution (a1) and the second solution (b1) may be contacted to generate the pharmaceutical formulation with suitable physiochemical and controlled-release characteristics, as well as suitable unit doses and psychopharmaceutical agent concentrations. Different concentrations of psychopharmaceutical agent in (a1) the first solution, different concentrations of release modifier in (b1) the second solution, and/or different volume ratios of (a1) the first solution to (b1) the second solution may be contacted to generate different pharmaceutical formulations with differentiated release characteristics, unit doses, and/or psychopharmaceutical agent concentrations. For example, when the preparative method involves the use of a kit, the same kit may be used to generate a range of differentiated pharmaceutical formulations in terms of, inter alia, unit dose, psychopharmaceutical agent concentration, release modifier, and release characteristics, by combining different volume ratios of (a1) the first solution to (b1) the second solution. A suitable volume ratio of (a1) the first solution to (b1) the second solution used to generate the pharmaceutical formulation may be from about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, or any range therebetween, such as from about 2:1 to about 1:2, about 1.5:1 to about 1:1.5, about 1:1. The contacting of (a1) the first solution to (b1) the second solution to generate the pharmaceutical formulation may be performed well in advance of administration with the pharmaceutical formulation being optionally stored, or immediately prior to use. The method may involve following instructions for what volume ratios of (a1):(b1) can be used to prepare suitable injectable pharmaceutical formulations, for example, based on physiochemical properties, indication to be treated, dosing schedule and requirements, etc.

In some embodiments, the method involves contacting (a2) a psychopharmaceutical agent in solid form with (b2) a solution comprising a release modifier such as a hyaluronate salt or a carboxymethyl cellulose salt, and an aqueous vehicle.

In some embodiments, (a2) the psychopharmaceutical agent is a tryptamine psychedelic in solid form, such as a pharmaceutically acceptable salt of a compound of Formula (I) through (III), in solid form. Here, any pre-formed, solid form and in some cases crystalline solid form, of the pharmaceutically acceptable salt of a compound of Formula (I) through (III) of the present disclosure can be used as (a2).

In some embodiments, (b2) the solution comprises as the release modifier a hyaluronate salt such as sodium hyaluronate, and the aqueous vehicle. A concentration of the hyaluronate salt by weight per total volume of (b2) the solution expressed as a percentage (% w/v) may be about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.1% to about 2%, about 0.1% to about 1.5%, about 0.1% to about 1%, about 0.1% to about 0.75%, about 0.1% to about 0.5%, about 0.15% to about 1%, about 0.2% to about 0.75%, or about 0.25% to about 0.5%.

In some embodiments, (b2) the solution comprises as the release modifier a carboxymethyl cellulose salt such as sodium carboxymethyl cellulose, and the aqueous vehicle. A concentration of carboxymethyl cellulose salt by weight per total volume of (b2) the solution expressed as a percentage (% w/v) may be about 0.55%, about 0.6%, about 0.65%, about 0.7%, about 0.75%, about 0.8%, about 0.85%, about 0.9%, about 0.95%, about 1%, or any range therebetween, such as from about 0.6% to about 1%, about 0.7% to about 1%, about 0.75% to about 1%, about 0.75% to about 0.9%, about 0.75% to about 0.8%.

A suitable volume of (b2) the solution may be contacted with (a2) the psychopharmaceutical agent in solid form to generate the pharmaceutical formulation with suitable physiochemical and controlled-release characteristics, as well as suitable psychopharmaceutical agent concentrations. Different concentrations of release modifier in (b2) the solution and/or different volumes of (b2) the solution may be contacted with (a2) the psychopharmaceutical agent in solid form to generate different pharmaceutical formulations with differentiated release characteristics and psychopharmaceutical agent concentrations. For example, when the preparative method involves the use of a kit, the same kit may be used to generate a range of differentiated pharmaceutical formulations in terms of, inter alia, psychopharmaceutical agent concentration, etc., by contacting different volumes of (b2) the solution with (a2) the psychopharmaceutical agent in solid form. Further, different amounts of (a2) the psychopharmaceutical agent in solid form may be used to generate different pharmaceutical formulations with differentiated unit doses and psychopharmaceutical agent concentrations. The contacting of (b2) the solution with (a2) the solid dosage form of the psychopharmaceutical agent can be accurately described as an act of reconstituting the solid dosage form of the psychopharmaceutical agent (e.g., a tryptamine psychedelic such as a pharmaceutically acceptable salt of a compound of Formula (I) through (III), in solid form and in some case in crystalline solid form). Reconstitution may be performed well in advance of administration with the pharmaceutical formulation being optionally stored, or immediately prior to use. The method may involve following instructions for what volume of (b2) the solution can be contacted with (a2) the psychopharmaceutical agent in solid form to prepare suitable injectable pharmaceutical formulations, for example, based on physiochemical properties, indication to be treated, dosing schedule and requirements, etc.

As described previously, in addition to water, the aqueous vehicle used in any of (a1) the first solution, (b1) the second solution, and/or (b2) the solution may optionally contain one or more pharmaceutically acceptable additives, as desired/needed, such as water-miscible vehicles, non-aqueous vehicles, antimicrobial agents or preservatives against the growth of microorganisms, stabilizing agents, tonicity agents, buffering agents, antioxidants, local anesthetics, complexing agents, sequestering or chelating agents, pH adjusting agents, absorption enhancers, including combinations thereof. Suitable aqueous vehicles include, but are not limited to, water, saline, physiological or isotonic saline, phosphate buffered saline (PBS), sodium chloride injection, Ringers injection, isotonic dextrose injection, sterile water injection, dextrose and lactated Ringers injection. In some embodiments, the aqueous vehicle is made up of saline, optionally a buffering agent, optionally a pH adjusting agent (e.g., sodium hydroxide), and optionally a tonicity agent other than sodium chloride. In some embodiments, the aqueous vehicle is made up of water, optionally a buffering agent, optionally a pH adjusting agent (e.g., sodium hydroxide), and optionally a tonicity agent. In some embodiments, the aqueous vehicle is made up of water or saline, and optionally a pH adjusting agent (e.g., sodium hydroxide), wherein the aqueous vehicle is formulated without a buffering agent. In some embodiments, the aqueous vehicle is formulated without a tonicity agent such as sodium chloride. In some embodiments, the aqueous vehicle used in one or more of (a1) the first solution, (b1) the second solution, and/or (b2) the solution, independent of one another, is water, such as water for injection (WFI). In some embodiments, the aqueous vehicle used in one or more of (a1) the first solution, (b1) the second solution, and/or (b2) the solution, independent of one another, comprises sodium chloride at a concentration, in terms of weight per volume expressed as a percentage (% w/v), of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, or any range therebetween, such as from about 0.1% to about 1.5%, about 0.2% to about 1%, about 0.3% to about 0.5% w/v.

In some embodiments, a pH adjustment may be performed with a pH adjusting agent on one or more of (a1) the first solution, (b1) the second solution, and/or (b2) the solution. In some embodiments, after contacting (a1) the first solution with (b1) the second solution or (a2) the psychopharmaceutical agent in solid form with (b2) the solution, the method may comprise adjusting the pH with a pH adjusting agent to generate the final pharmaceutical formulation. Here, when the preparative method involves the use of a kit, the kit may optionally comprise (c) a pH adjusting agent. In some embodiments, the pH adjusting agent is at least one selected from the group consisting of sodium hydroxide, potassium hydroxide, sodium carbonate, ammonium hydroxide, calcium hydroxide, magnesium hydroxide, hydrochloric acid, citric acid, and lactic acid. In some embodiments, the adjusting of the pH involves increasing the pH, and so (c) comprises a suitable base as pH adjusting agent, for example one or more of sodium hydroxide, potassium hydroxide, sodium carbonate, ammonium hydroxide, calcium hydroxide and magnesium hydroxide. In some embodiments, the pH is adjusted with sodium hydroxide or potassium hydroxide, for example to generate the final pharmaceutical formulation. In some embodiments, the pharmaceutical formulation does not contain a buffering agent.

To be injectable, the pharmaceutical formulations of the present disclosure must be sterile, as known and practiced in the art. The method of the present disclosure may optionally comprise sterilizing the pharmaceutical formulation, for example as a final preparative step. The method may optionally comprise sterilizing any one or more (including all) components used to prepare the pharmaceutical formulation, such as (a1) the first solution, (b1) the second solution, (a2) the psychopharmaceutical agent in solid form, and/or (b2) the solution. The method may optionally comprise sterilizing any one or more (including all) components used to prepare the pharmaceutical formulation, such as (a1) the first solution, (b1) the second solution, (a2) the psychopharmaceutical agent in solid form, and/or (b2) the solution, and optionally sterilizing the pharmaceutical formulation, for example as a final preparative step. When sterilization is performed, sterilization may be carried out by steam sterilization, dry-heat sterilization/depyrogenation, gas sterilization, sterilization by ionizing radiation, sterilization by filtration, or unidirectional aseptic processing, for example according to the requirements set forth in USP Sterilization and Sterility Assurance of Compendial Articles <1211> and/or USP Sterility Tests <71>. In some embodiments, such as when the release modifier employed is a hyaluronate salt, sterile filtration is used. While not limited thereto, sterile filtration is usually carried out with assemblies having membranes of nominal pore size rating of 0.2 μm or less or 0.1 μm or less, according to the requirements set forth in USP Sterilization and Sterility Assurance of Compendial Articles <1211>. Sterile filtration may be carried out by passing the pharmaceutical formulation (or various components making up the pharmaceutical formulation, separately) through a membrane, a syringe filter, a vacuum device, a capsule assembly, a high pressure cartridge or filter assembly, and the like, each equipped with a suitable membrane, typically a membrane which is capable of passing a bacterial challenge to retain a minimum of 10⁷ CFU/cm² of B. dimunita. Examples of membranes include, but are not limited to, those made from polyethersulfone (PES), polyvinylidene fluoride (PVDF), cellulose, cellulose acetate, mixed cellulose esters (MCE), nylon, and hydrophilic polytetrafluoroethylene (PTFE).

Alternatively, the preparative method for producing the pharmaceutical formulation may not involve any sterilization processing, and instead the method involves assembly of already sterilized components. For example, when the preparative method involves the use of a kit, the various kit components to be assembled (e.g., (a1) the first solution with (b1) the second solution, or (a2) the psychopharmaceutical agent in solid form with (b2) the solution) may be provided in sterilized form, where appropriate. Here, the end user such as a treating clinician, patient, or caregiver, would simply assemble the already sterilized kit components thereby forming the pharmaceutical formulation ready-to-use, with no additional sterilization processing needed.

The pharmaceutical formulations disclosed herein may be formulated for single or multiple dosage administration. The pharmaceutical formulation may be optionally stored and/or packaged in any suitable container, examples of which include, but are not limited to, an ampule, a vial, a syringe such as a pre-filled syringe, a cartridge, a reservoir or cartridge for an injection device such as an on-body drug delivery device or an auto-injector, etc. In some embodiments, to help prevent degradation or physical changes to the pharmaceutical formulation, the pharmaceutical formulation is stored and/or packaged in a container adapted to prevent penetration of ultraviolet light, such as amber glass vial. In some embodiments, the container within which the pharmaceutical formulation is stored and/or packaged is not so adapted (and may be, for example, made of clear glass) with protection against ultraviolet light, if desired, provided by secondary packaging (for example packaging within which the receptacle containing the pharmaceutical formulation may be placed). Often, the container is airtight and the pharmaceutical formulation is stored under an inert atmosphere, such as under nitrogen or argon, typically nitrogen. The formulation may be stored at room temperature, e.g., at about 20 to about 30° C., or at cooler temperatures, for example at about 2 to about 8° C. Alternatively, the pharmaceutical formulation may be stored in a freezer. Further, tamper resistant dosage forms/packaging of any of the disclosed pharmaceutical formulations are contemplated.

### Therapeutic Applications and Methods

Also disclosed herein is a method of treating a disease or disorder in a subject in need thereof, comprising administering to the subject a pharmaceutical formulation of the present disclosure. The disease or disorder may be a neuropsychiatric disease or disorder or an inflammatory disease or disorder, such as a central nervous system (CNS) disorder and/or psychological disorder, including those associated with a 5-HT₂ receptor.

The pharmaceutical formulation is suitable for injection, thus its administration in therapy typically comprises parenteral administration via injection to affect a beneficial therapeutic response. Injection may involve administration through a needle (hypodermic needle), microneedles including hollow microneedles, or using a needle-free injection system whereby a fine, high velocity jet is generated by driving liquid through an orifice at high pressure to pierce the skin and underlying tissue. In some embodiments, the method comprises administering the pharmaceutical formulation intravenously to the subject (directly into the vein). In some embodiments, the method comprises administering the pharmaceutical formulation intramuscularly to the subject (within the muscle). In some embodiments, the method comprises administering the pharmaceutical formulation intradermally to the subject (beneath the skin). In some embodiments, the method comprises administering the pharmaceutical formulation subcutaneously to the subject (within the fat or the layer of skin directly below the dermis and epidermis). Subcutaneous administration is a minimally invasive mode of administration. Subcutaneous tissue has few blood vessels and so drugs injected into it are intended for slow, sustained rates of absorption, often with some amount of depot effect. Compared with other routes of administration, it is slower than intravenous and intramuscular injections but still faster than intradermal injections. The convenience and speed of subcutaneous delivery allows increased patient compliance and quicker access to medication when needed. Subcutaneous administration can be performed by injection or by implantation of a sustained or timed-release device beneath the surface of the skin. The site of the injection or device can be rotated when multiple injections or devices are needed. When a subject requires multiple doses, the method may involve subcutaneously injecting several unit dose pharmaceutical formulations at multiple sites of the body surface. A particular advantage of the subcutaneous delivery route in the therapeutic methods of the present disclosure is that it allows the medical practitioner to perform the administration in a rather short intervention with the patient, compared to intravenous infusion protocols associated with DMT-based therapy. Moreover, the patient can be trained to perform self-administration. Such self-administration may be particularly useful during maintenance dosing where clinical observation and/or psychotherapy may play less prominent roles in the overall treatment.

In methods comprising administering the pharmaceutical formulation subcutaneously to the subject, the injection volume of the pharmaceutical formulation administered per injection is usually about 3 mL or less, such as about 0.1 mL, about 0.2 mL, about 0.4 mL, about 0.6 mL, about 0.8 mL, about 1 mL, about 1.2 mL, about 1.4 mL, about 1.6 mL, about 1.8 mL, about 2 mL, about 2.2 mL, about 2.4 mL, about 2.6 mL, about 2.8 mL, about 3 mL, or any range therebetween such as from about 0.5 to about 3 mL, from about 1.5 to about 2.5 mL, from about 0.5 to about 1.5 mL, or from about 2 to about 2.5 mL. Usually, single injection volumes of up to about 3 mL are tolerated via the subcutaneous route without undue pain, especially those given in the patient's abdomen, with injection volumes of about 2.5 mL or less, about 2 mL or less, or about 1.5 mL or less being well tolerated across various injection sites. In preferred embodiments, the injection volume of the pharmaceutical formulation administered is about 0.5 mL to about 1.5 mL. However, it may be possible to administer larger volumes by divided doses across multiple injections and injection sites. For example, in situations where an injection volume needed/desired exceeds 3 mL (e.g., 4 mL), administration may be carried out by dividing the total volume to be administered in multiple injections, e.g., two injections of 2 mL each, administered at different sites (e.g., two different injection sites) on the patient's body. Thus, the methods may involve administration of 1 injection at a single injection site, or 2, 3, 4, or more injections across multiple injection sites, whereby the multiple injections add up to the total dose and volume administered.

In some embodiments, the method comprises administering the pharmaceutical formulation as a bolus injection, in which a discrete amount of psychopharmaceutical agent (e.g., tryptamine psychedelic such as a pharmaceutically acceptable salt of a compound of Formula (I) through (III)) is administered by injection within 30 minutes or less, 25 minutes or less, 20 minutes or less, 15 minutes or less, 10 minutes or less, 5 minutes or less, 4 minutes or less, 3 minutes or less, 2 minutes or less, 1 minute or less, 30 seconds or less, 20 seconds or less, 10 seconds or less, or 5 seconds or less, or any range therebetween. The bolus injection may involve a single injection or multiple injections performed within the above-described time range. Thus, administering multiple bolus injections within the above-mentioned time range of 30 minutes or less (e.g., two injections lasting 30 seconds each, administered within 5 minutes of one another) would be considered a bolus administration herein. In some embodiments, the bolus injection involves a single injection within the above time range. In some embodiments, the method comprises administering the pharmaceutical formulation as a bolus subcutaneous injection, such as a single bolus subcutaneous injection. In some embodiments, the method comprises administering the pharmaceutical formulation as a bolus intramuscular injection, such as a single bolus intramuscular injection. In some embodiments, the method comprises administering the pharmaceutical formulation as a bolus intradermal injection, such as a single bolus intradermal injection. In some embodiments, the method comprises administering the pharmaceutical formulation as a bolus intravenous injection, such as a single bolus intravenous injection.

In some embodiments, the method comprises administering the pharmaceutical formulation as an infusion injection, in which a discrete amount of psychopharmaceutical agent (e.g., a tryptamine psychedelic such as a pharmaceutically acceptable salt of a compound of Formula (I) through (III)) is administered by injection over a prolonged period of greater than 30 minutes, greater than 40 minutes, greater than 50 minutes, greater than 60 minutes, greater than 70 minutes, greater than 80 minutes, greater than 90 minutes, greater than 100 minutes, greater than 110 minutes, greater than 120 minutes, etc. The infusion injection may involve a single prolonged injection, or multiple injections (short or prolonged) within the above-described time range. Thus, administering multiple bolus injections over a prolonged period of greater than 30 minutes would be considered an infusion administration herein. In some embodiments, the infusion injection involves a single injection within the above time range. In some embodiments, the method comprises administering the pharmaceutical formulation as an infusion subcutaneous injection. In some embodiments, the method comprises administering the pharmaceutical formulation as infusion intramuscular injection. In some embodiments, the method comprises administering the pharmaceutical formulation as infusion intravenous injection.

Any injection device that enables administration of the pharmaceutical formulation through the skin or other external boundary tissue of the subject may be used in the disclosed methods. Examples of injection devices include, but are not limited to, needle and syringe, pre-filled syringe, canula, catheter, pen, auto-injector (e.g., ACTpen®, ArQ®—Bios from Oval Medical Technologies), semi-auto injector (such as those from UnionMedico®) on-body drug delivery devices (e.g., patches, pumps, those with automatic needle insertion/retraction, belt worn devices, such as those available from Gerresheimer AG, etc.), implants, embedded needle tip devices, a needle-free injection system (e.g., a jet injector), automatic injection devices, or other suitable injection device. The injection device may be resuable or disposable (e.g., disposable hypodermic needle and syringe, disposable pen, disposable pump, disposable on-body drug delivery device, etc.), or may contain a reusable or disposable needle guide. The injection device may be actuated manually or automatically. Some automatic injection devices may be configured with automatic needle insertion/retraction, auto-pumps, or one or more microneedles, which in some embodiments may be coated with a pharmaceutical formulation disclosed herein. Alternatively, hollow microneedles may be used to provide a fluid channel for delivery of the disclosed pharmaceutical formulations below the outer layer of the skin to improve delivery. In needle-free injection systems (e.g., a jet injector) a fine, high velocity jet is generated by driving liquid through an orifice at high pressure to pierce the skin and underlying tissue. These needle-free injection systems may include those adapted with spring systems, lasers, or energy propelled systems such as Lorentz force, gas propelled/air forced, or shove wave driven devices. Examples of needle-free injection devices include, but are not limited to, Bioject® jet injectors such as Biojector® 2000, Viajet 3, DosePro™, ZetaJet™, Stratis® IM/SC, and Jupiter Jet™. Multi-component injections, such as using a dual chamber syringe or a multi-syringe (e.g., two syringe) set up, may also be performed. The injection device can be optionally manufactured with smart technology enabling remote activation and/or control of delivery. The remote activation can be performed via computer or mobile app. To ensure security, the remote activation device can be password encoded. This technology enables a healthcare provider to perform telehealth sessions with a patient, during which the healthcare provider can remotely activate and administer the pharmaceutical formulation via the desired delivery device while supervising the patient on the televisit.

Subcutaneous injections may be performed by cleaning the area to be injected followed by an injection, usually at about a 45-degree angle to the skin when using a syringe and needle, or at about a 90-degree angle (perpendicular) if using an injector pen or other suitable injector device. Of course, as is known in the art, the appropriate injection angle is based on the length of needle or injection device used, and the depth of the subcutaneous fat in the skin of the subject receiving the treatment. If administered at an angle, the skin and underlying tissue may be pinched upwards prior to injection (the “pinch-up” technique).

Subcutaneous injection may be administered in the subject's arm such as the outer area of the upper arm, thigh such as the front of the thigh (e.g., between 4 inches from the top of the thigh and 4 inches above the knee), abdomen such as the anterior abdomen usually avoiding the 2-inch circle around the navel, the upper back, the upper area of the buttock, just behind the hip bone, and the like. Any of which may be performed using the “pinch-up” technique. The choice of injection site is based on the pharmaceutical formulation being administered, for example taking into account the volume of injection needed, as well as preference. Injections administered frequently or repeatedly should be administered in a different location each time, either within the same general site or a different site, but at least one inch away from recent injections as known and practiced in the art. In most cases, subcutaneous injections can be easily performed by people with minor skill and training required. The injection sites for self-injection procedures are the same as for injection by a healthcare professional, and the skill can be taught to patients using pictures, videos, or models of the subcutaneous tissue for practice, and in some cases facilitated by use of semi- or auto-injector devices or on-body drug delivery devices for facile self-administration.

Administration may follow a continuous administration schedule, or an intermittent administration schedule. The administration schedule may be varied depending on the psychopharmaceutical agent employed, the condition being treated, etc. For example, administration may be performed once a day (QD), or in divided dosages throughout the day, such as 2-times a day (BID), 3-times a day (TID), 4-times a day (QID), or more. In some embodiments administration may be performed nightly (QHS). In some embodiments, the pharmaceutical formulation may be administered as needed (PRN). Administration may also be performed on a weekly or monthly basis, e.g., once a week, twice a week, three times a week, four times a week, every other week, every two weeks, every three weeks, every four weeks, every five weeks, every six weeks, every seven weeks, every eight weeks, every nine weeks, every ten weeks, every eleven weeks, every twelve weeks, etc., or less, or any range therebetween. In some embodiments, the pharmaceutical formulation may be administered monthly, for example, once monthly dosing or dosing every two months. The administration schedule may also designate a defined number of treatments per treatment course, for example, administration may be performed 1 time, 2 times, 3 times, 4 times, 5 times, 6 times, 7 times, or 8 times per treatment course. In some embodiments, one dose is administered to the subject in a treatment course. In some embodiments, multiple doses are administered to the subject in a treatment course. In some embodiments, two doses are administered to the subject one week apart, two weeks apart, three weeks apart, or four weeks apart, in a treatment course. For example, the treatment herein may involve two doses of the pharmaceutical formulation administered three weeks apart (e.g., day 1 and day 22). In some embodiments, three doses are administered to the subject one week apart, two weeks apart, three weeks apart, or four weeks apart, in a treatment course. Other administration schedules may also be deemed appropriate using sound medical judgement.

The dosing can be continuous (7 days of administration in a week) or intermittent, for example, depending on the pharmacokinetics and a particular subject's clearance/accumulation of the psychopharmaceutical agent. If intermittently, the schedule may be, for example, 4 days of administration and 3 days off (rest days) in a week or any other intermittent dosing schedule deemed appropriate using sound medical judgement. For example, intermittent dosing may involve administration of a single dose within a treatment course. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

The dosage and frequency (single or multiple doses) of administration can vary depending upon a variety of factors, including, but not limited to, the psychopharmaceutical agent to be administered; the disease/condition being treated; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

In order to treat the disease or disorder, the pharmaceutical formulation comprises a therapeutically effective amount of the psychopharmaceutical agent (e.g., a tryptamine psychedelic such as a pharmaceutically acceptable salt of a compound of Formula (I) through (III)), i.e., an amount that is sufficient to reduce or halt the rate of progression of the disease or disorder, to ameliorate or cure the disease or disorder and thus produce the desired therapeutic or inhibitory effect, or to alleviate one or more symptoms of the disease or disorder. Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards or downwards.

Dosages may be varied depending upon the requirements of the subject and the psychopharmaceutical agent being employed. The dose administered to a subject, in the context of the pharmaceutical formulations presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the psychopharmaceutical agents), and yet is entirely effective to treat the clinical symptoms demonstrated by the particular patient. This planning should involve the careful choice of psychopharmaceutical agent by considering factors such as compound potency, relative bioavailability, patient body weight, presence and severity of adverse side effects, preferred mode of administration, and the toxicity profile of the selected agent.

In some embodiments, in terms of weight-based dosing (per kilogram body weight of the subject), a therapeutically effective amount of the psychopharmaceutical agent (free base equivalence) provided by the pharmaceutical formulation is about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.8 mg/kg, about 3.0 mg/kg, about 3.2 mg/kg, about 3.4 mg/kg, about 3.6 mg/kg, about 3.8 mg/kg, about 4.0 mg/kg, about 4.2 mg/kg, about 4.4 mg/kg, about 4.6 mg/kg, about 4.8 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, or any range therebetween.

In some embodiments, the pharmaceutical formulation comprises a tryptamine psychedelic (e.g., a pharmaceutically acceptable salt of a compound of Formula (I) through (III)) as the psychopharmaceutical agent, and a therapeutically effective amount may be a psychedelic dose. Here, a psychedelic dose (therapeutically effective amount) in terms of weight-based dosing (free base equivalence) may in some embodiments be about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, or any range therebetween, such as from about 0.1 mg/kg to about 3 mg/kg, from about 0.3 mg/kg to about 2.0 mg/kg, from about 0.6 mg/kg to about 1.5 mg/kg, from about 0.8 mg/kg to about 1.2 mg/kg. Higher dosing may also be used in some embodiments, as described above. In terms of fixed doses, a psychedelic dose (therapeutically effective amount) of a tryptamine psychedelic (e.g., a pharmaceutically acceptable salt of a compound of Formula (I) through (III)) (in terms of free base equivalence) may in some embodiments be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, or any range therebetween, such as from about 15 mg to about 150 mg, from about 20 mg to about 100 mg, from about 25 mg to about 75 mg. In some embodiments, psychedelic doses are administered once, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose. These treatments are appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder), and combinations thereof such as an anxiety disorder with depression.

Furthermore, administration of pharmaceutical formulations comprising a tryptamine psychedelic (e.g., a pharmaceutically acceptable salt of a compound of Formula (I) through (III)) as the psychopharmaceutical agent in sub-psychedelic (yet still potentially serotonergic concentrations) doses may be performed in some embodiments to achieve durable therapeutic benefits, with decreased toxicity, and may thus be suitable for microdosing. Sub-psychedelic dosing may in some embodiments provide the tryptamine psychedelic (e.g., a pharmaceutically acceptable salt of a compound of Formula (I) through (III)) (in terms of free base equivalence) in an amount of about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.075 mg/kg, about 0.08 mg/kg, about 0.09 mg/kg, or about less than 0.1 mg/kg, or any range therebetween. Typically, sub-psychedelic doses are administered up to every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday. Sub-psychedelic doses can be used, e.g., for the chronic treatment or maintenance of a variety of diseases or disorders disclosed herein, examples of which include, but are not limited to, depression (e.g., MDD), inflammation, pain, and neuroinflammation.

The pharmaceutical formulations of present disclosure may be administered via injection for a maintenance regimen. As used herein, a “maintenance regimen” generally refers to the administration of a psychopharmaceutical agent following achievement of a target dose, e.g., following completion of an up-titration regimen, and/or following a positive clinical response, e.g., improvement of the patient's condition, either to the same agent or to a different agent. In some embodiments, the patient is administered a first psychopharmaceutical agent for a therapeutic regimen and a second psychopharmaceutical agent for a maintenance regimen, wherein the first and second psychopharmaceutical agents are different. For example, the patient may be administered a therapeutic regimen of a first psychopharmaceutical agent which is not a tryptamine psychedelic (e.g., the first psychopharmaceutical agent is LSD, MDMA, etc.), followed by a tryptamine psychedelic (as the second psychopharmaceutical agent) in a maintenance regimen. In another example, a different tryptamine psychedelic is used for the therapeutic regimen (first psychopharmaceutical agent) than is used for the maintenance regimen (second psychopharmaceutical agent). In some embodiments, the patient is administered the psychopharmaceutical agent for both a therapeutic regimen and a maintenance regimen. In any case, the maintenance dose may be used to ‘maintain’ the therapeutic response and/or to prevent occurrences of relapse. When the same psychopharmaceutical agent is used for both the original therapeutic regimen and for the maintenance regimen, the maintenance dose may be at or below the therapeutic dose. In some embodiments, the maintenance dose is a psychedelic dose. In some embodiments, the maintenance dose is a sub-psychedelic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

The methods provided herein may be used to treat a disease or disorder associated with a serotonin 5-HT₂ receptor. In some embodiments, the disease or disorder is a neuropsychiatric disease or disorder or an inflammatory disease or disorder. In some embodiments, the disease or disorder is a central nervous system (CNS) disorder and/or psychological disorder, including, but not limited to, post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders (including, but not limited to, bipolar I disorder, bipolar II disorder, cyclothymic disorder), obsessive-compulsive disorder (OCD), generalized anxiety disorder (GAD), acute psychedelic crisis, social anxiety disorder, substance use disorders (including, but not limited to, alcohol use disorder, opioid use disorder, amphetamine use disorder, nicotine use disorder, smoking, and cocaine use disorder), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, eating disorders (including, but not limited to, anorexia nervosa, bulimia nervosa, binge-eating disorder, etc.), and paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido), and obesity, including combinations thereof such as generalized anxiety disorder with a comorbid depression.

In some embodiments, the methods provided herein are used to treat a subject with a depressive disorder. As used herein, the terms “depressive disorder” or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions. In some embodiments, the depressive disorder is major depressive disorder (MDD), atypical depression, bipolar disorder, catatonic depression, depressive disorder due to a medical condition, postpartum depression, premenstrual dysphoric disorder, seasonal affective disorder, or treatment-resistant depression (TRD).

In some embodiments, the disease or disorder is major depressive disorder (MDD). As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all—or almost all—activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e., depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g., vitamin B12 deficiency), autoimmune conditions (e.g., lupus and rheumatoid arthritis), viral or other infections (hepatitis, mononucleosis, herpes), back pain, and cancer (e.g., pancreatic cancer).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Mental Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with treatment-resistant depression (TRD). The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, 5 treatment attempts, or more. In some embodiments, the subject with treatment-resistant depression has been diagnosed with major depressive disorder and has failed to respond to 3 or more treatment attempts. In some embodiments, the subject with treatment resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, the methods provided herein reduce at least one sign or symptom of MDD. In some embodiments, the methods provided herein reduce at least one sign or symptom of MDD by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is measured in a subject before, during, and/or after treatment with the methods described herein. In some embodiments, the sign or symptom of depression is measured according to a diary assessment, an assessment by clinician or caregiver, a clinical rating scale, an imaging test, or a blood of CSF test. In some embodiments, the sign or symptom of depression in a subject is measured using a neuropsychological assessment or clinical rating scale. In some embodiments, the neuropsychological assessment or clinical rating scale is the Hamilton Depression Rating Scale, the Clinical Global Impression (CGI) Scale, the Montgomery-Asberg Depression Rating Scale (MADRS), the Beck Depression Inventory (BDI), the Zung Self-Rating Depression Scale, the Raskin Depression Rating Scale, the Inventory of Depressive Symptomatology (IDS), the Quick Inventory of Depressive Symptomatology (QIDS), Young Mania Rating Scale (YMRS), the Columbia-Suicide Severity Rating Scale (C-SSRS), the Suicidal Ideation Attributes Scale (SIDAS), and/or Mini International Neuropsychiatric Interview (MINI).

In some embodiments, the sign or symptom of depression in a subject is measured using the Hamilton Depression Rating (HAM-D) scale. The HAM-D scale is a 17-item scale that measures depression severity before, during, or after treatment. The scoring is based on 17 items and generally takes 15-20 minutes to complete the interview and score the results. Eight items are scored on a 5-point scale, ranging from 0=not present to 4=severe. Nine items are scored on a 3-point scale, ranging from 0=not present to 2=severe. A score of 10-13 indicates mild depression, a score of 14-17 indicates mild to moderate depression, and a score over 17 indicates moderate to severe depression. In some embodiments, after treatment with the methods described herein, the subject's HAM-D score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Clinical Global Impression (CGI) scale. The CGI scale is a 3-item scale that measures illness severity, global improvement or change, and therapeutic response. The CGI is rated on a 7-point scale, with the severity of illness measured using a range of responses from 1 (normal) through 7 (amongst the most severely ill subjects). In some embodiments, after treatment with the methods described herein, the subject's CGI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Montgomery-Asberg Depression Rating Scale (MADRS). The MADRS scale is a 10-item scale that measures the core symptoms of depression. Nine of the items are based upon patient report, and 1 item is on the rater's observation during the rating interview. MADRS items are rated on a 0-6 continuum with 0=no abnormality and 6=severe abnormality. These individual item scores are added together to form a total score, which can range between 0 and 60 points. A score of 7 to 19 indicates mild depression, 20 to 34 indicates moderate depression, and over 34 indicates severe depression. In some embodiments, the subject to be treated has, prior to beginning treatment with the methods herein, received a mild diagnosis of depression according to the MADRS. In some embodiments, the subject to be treated has scored at least 7, at least 8, at least 9, at least 10, at least 11, at least 12, at least 13, at least 14, and up to 19, up to 18, up to 17, up to 16, up to 15 on the MADRS prior to treatment with the methods herein. In some embodiments, the subject to be treated has, prior to beginning treatment with the methods herein, received a moderate to severe diagnosis of depression according to the MADRS. In some embodiments, the subject to be treated has, prior to beginning treatment with the methods herein, received a moderate to severe diagnosis of MDD as defined by the DSM, 5th edition (DSM-5). In some embodiments, the subject to be treated has scored at least 20, at least 21, at least 22, at least 23, at least 24, at least 25, at least 26, at least 27, at least 28, at least 29, at least 30, and up to 60, up to 55, up to 50, up to 45, up to 40, up to 35 on the MADRS prior to treatment with the methods herein, for example a MADRS score of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or any range therebetween.

In evaluating treatment efficacy, a clinically meaningful “within-group” change from baseline (“CFB”) on the MADRS has been reported to range between a 6- to 9-point reduction in total score (i.e., a MADRS CFB of −6 to −9). As depression is more severe at baseline, the change needed to be considered meaningful moves towards the higher end of the range. In some embodiments, after treatment with the methods of the present disclosure the patient has a lower MADRS score compared with the subject's MADRS score prior to treatment (baseline), i.e., the methods result in a negative MADRS Change From Baseline (“CFB”) score. In some embodiments, the methods described herein result in a MADRS CFB score, e.g., at two-weeks post-dose, at three-weeks post-dose, etc., of −10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a MADRS CFB score, e.g., at two-weeks post-dose, at three-weeks post-dose, etc., of −10 to −20 points, −11 to −19 points, −12 to −18 points, or −13 to −17 points.

When groups are compared to each other (“between-group”), e.g., treatment versus placebo, a 2-point difference in MDRS CFB between groups has been found to be clinically meaningful. In some embodiments, the methods described herein result in a MADRS CFB score reduction over placebo (also referred to as a placebo subtracted difference in MADRS CFB), e.g., at two-weeks post-dose, at three-weeks post-dose, etc., of −8 points, −9 points, 10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, −32 points, or more, or any range therebetween. For example, in some embodiments, the methods described herein result in a MADRS CFB score reduction over placebo, e.g., at two-weeks post-dose, at three-weeks post-dose, etc., of −10 to −20 points, −12 to −18 points, −13 to 16 points, or −14 to −15 points. In some embodiments, the MADRS CFB score reduction over placebo described above may be accompanied by a p-value of 0.05 or less, 0.01 or less, 0.005 or less, 0.001 or less, 0.0005 or less, or 0.0001 or less. The between-group difference in MADRS CFB score described above can be converted into an effect size (Cohen's d score) in order to inform about the magnitude of treatment effects. In some embodiments, the methods described herein provide an effect size of about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, or any range therebetween, such as from 1 to 3, 1.5 to 2.5, 1.8 to 2.4, or 2 to 2.2. In some embodiments, the methods described herein provide an effect size of 1.7 to 2.5, or 1.8 to 2.3, or 2 to 2.2. Such effect sizes are considered to be surprisingly large, especially in MDD treatment, where effect sizes typically range from 0.2 to 0.6.

In some embodiments, after treatment with the methods described herein, the subject's MADRS score decreases from prior to treatment (baseline) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or any range therebetween, e.g., at two-weeks post-dose, three-weeks post-dose, etc. In some embodiments, after treatment with the methods described herein, the subject's MADRS score decreases from prior to treatment (baseline) by at least about 50%, at least about 55%, at least about 60%, at least about 65%, at least about 70%, at least about 75%, at least about 80%, at least about 85%, at least about 90%, at least about 95%, or about 100%, or any range therebetween, e.g., at three-weeks post-dose. In some embodiments, at least 50%, at least 55%, at least 60%, at least 65%, at least 70%, at least 75%, at least 80%, at least 85%, at least 90% of subjects treated with the methods described herein have a MADRS score decrease from baseline by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, e.g., at two-weeks post-dose, three-weeks post-dose, etc.

In some embodiments, the methods described herein provide a response rate (i.e., number of responders versus total number of subjects treated within a group) of at least 25%, at least 30%, at least 35%, at least 40%, at least 45%, at least 50%, at least 55%, at least 60% measured two-weeks post-dose, three-weeks post dose, etc., with responders being defined as subjects who had a reduction of MADRS score from baseline of at least 50%.

In some embodiments, the methods described herein provide a remission rate (i.e., number of remitters versus total number of subjects treated within a group) of at least 10%, at least 12%, at least 14%, at least 16%, at least 18%, at least 20%, at least 22%, at least 24%, at least 26%, at least 28%, at least 30% measured two-weeks post-dose, three-weeks post dose, etc., with remitters being defined as subjects who had a total MADRS score of 10 or less, 9 or less, 8 or less, 7 or less, 6 or less, 5 or less, 4 or less, 3 or less, 2 or less, 1 or less.

In some embodiments, the subjects have a sustained response to treatment with the methods of the present disclosure, whereby the reduction from baseline in the MADRS at two-weeks post-dose, three-weeks post dose, etc. (e.g., a MADRS score decreases from baseline of at least about 50%) is maintained at least four-weeks post-dose, at least five-weeks post-dose, at least six-weeks post-dose, at least seven-weeks post-dose, at least eight-weeks post-dose, at least nine-weeks post-dose, at least ten-weeks post-dose, at least eleven-weeks post-dose, at least twelve-weeks post-dose, at least 4 months post-dose, at least 5 months post-dose, at least 6 months post-dose.

In some embodiments, the sign or symptom of depression in a subject is measured using the Beck Depression Inventory (BDI). The BDI is a 21-item, self-report rating inventory that measures characteristic attitudes and symptoms of depression. The items are rated on 0-3 continuum with 0=no abnormality and 3=severe abnormality. A score of 17-20 indicates borderline clinical depression, a score of 21-30 indicates moderate depression, a score of 31-40 indicates severe depression, and over 40 indicates extreme depression. In some embodiments, after treatment with the methods of the present disclosure the patient has a lower BDI score compared with the subject's BDI score prior to treatment (baseline), i.e., the methods result in a negative BDI Change From Baseline (“CFB”) score. In some embodiments, the methods described herein result in a BDI CFB score, e.g., at two-weeks post-dose, at three-weeks post-dose, etc., of −10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a BDI CFB score, e.g., at two-weeks post-dose, at three-weeks post-dose, etc., of −10 to −20 points, −11 to −19 points, −12 to −18 points, or −13 to −17 points. In some embodiments, the methods described herein result in a BDI CFB score reduction over placebo, e.g., at two-weeks post-dose, at three-weeks post-dose, etc., of −8 points, −9 points, 10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a BDI CFB score reduction over placebo, e.g., at two-weeks post-dose, at three-weeks post-dose, etc., of −10 to −20 points, −12 to −18 points, −13 to 16 points, or −14 to −15 points. In some embodiments, the BDI CFB score reduction over placebo described above may be accompanied by ap-value of 0.05 or less, 0.01 or less, 0.005 or less, 0.001 or less, 0.0005 or less, or 0.0001 or less. In some embodiments, after treatment with the methods described herein, the subject's BDI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Zung Self-Rating Depression Scale. The Zung Self-Rating Depression Scale is a 20-item self-report questionnaire that measures the psychological and somatic symptoms associated with depression. The questionnaire takes about 10 minutes to complete, and items are framed in terms of positive and negative statements. Each item is scored on a Likert scale ranging from 1 to 4. A total score is derived by summing the individual item scores, and ranges from 20 to 80. Most people with depression score between 50 and 69, while a score of 70 and above indicates severe depression. In some embodiments, after treatment with the methods described herein, the subject's Zung Self-Rating Depression score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Raskin Depression Rating Scale. The Raskin Depression Rating Scale measures baseline levels of depression and change in depression severity overtime. Each item is scored on a scale ranging from 1 to 5 with 1=not at all and 5=very much. A total score is derived by summing the individual item scores, and scores of 9 or greater represents moderate depression. In some embodiments, after treatment with the methods described herein, the subject's Raskin Depression Rating Scale score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Inventory of Depressive Symptomatology (IDS). The IDS is a 30-item inventory that measures depressive signs and symptoms. Each item is scored on a scale of 0 to 3 with 0=absence of pathology and 3=severe pathology. A total score is derived by summing the individual item scores; scores between 26-38 indicates mild depression, scores between 39-48 indicate moderate depression, and scores 49 or greater indicate severe depression. In some embodiments, after treatment with the methods described herein, the subject's IDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Quick Inventory of Depressive Symptomatology (QIDS). The QIDS is a 16-item inventory that measures depressive signs and symptoms. Each item is scored on a scale of 0 to 3 with 0=absence of pathology and 3=severe pathology. A total score is derived by summing the individual item scores; scores between 11-15 indicate moderate depression, scores between 16-20 indicate severe depression, and scores 21 or greater indicate very severe depression. In some embodiments, after treatment with the methods described herein, the subject's QIDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Young Mania Rating Scale (YMRS). The YMRS is an 11-item inventory that measures manic signs and symptoms. There are 4 items that are graded on a 0 to 8 scale, ranging from 0=absent to 8=severe. The remaining 7 items are graded on a 0 to 4 scale, ranging from 0=absent to 4=severe. A total score is derived by summing the individual item scores; scores between 9-15 indicate mild mania, scores between 16-25 indicate moderate mania, and scores 26 or greater indicate severe mania. In some embodiments, after treatment with the methods described herein, the subject's YMRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Columbia-Suicide Severity Rating Scale (C-SSRS). The C-SSRS measures the severity of suicidal ideation and behavior. The scale contains 10 binary questions (no=0 points and yes=1 point) and each question addresses a different component of the subject's suicidal ideation severity and behavior. A subject is considered to have suicidal ideation and/or behavior if they answer “yes” to any of the 10 questions. In some embodiments, after treatment with the methods described herein, the subject's C-SSRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Suicidal Ideation Attributes Scale (SIDAS). The SIDAS measures the presence and severity of suicidal thoughts. The scale contains 5 questions measured on a 10-point scale with 0=never and 10=always. Total scores are calculated as the sum of the 5 items and range from 0 to 50. Scores of 21 or greater indicate high risk of suicidal behavior. In some embodiments, after treatment with the methods described herein, the subject's SIDAS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

The Mini International Neuropsychiatric Interview (MINI) (for example, version 7.0.2) is a diagnostic interview instrument for psychiatric disorders in the Diagnostic and Statistical Manual of Mental Disorders (DSM-5) and the International Classification of Diseases-10. In some embodiments, treating according to the methods of the disclosure results in an improvement in a psychiatric disorder (e.g., depression, etc.) compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to a Mini International Neuropsychiatric Interview assessment. In some embodiments, the subject has a lower MINI 7.0.2 score compared with the subject's MINI 7.0.2 score prior to treatment.

In some embodiments, the sign or symptom in subjects with depression is measured using a Spielberger's Trait and Anxiety Inventory, a Generalized Anxiety Disorder 7-Item Scale, a Warwick-Edinburgh Mental Wellbeing Scale, a Flourishing Scale, a Snaith Hamilton Anhedonia Pleasure Scale, a Life Orientation Test, a Meaning in Life Questionnaire, a Brief Resilience Scale, a Dysfunctional Attitudes Scale, a 44-item Big Five Inventory, a Peters 21-item Delusional Inventory, an Examination of Anomalous Self-Experience, a Ruminative Responses Scale, a White Bear Suppression Inventory, a Barrett Impulsivity Scale, a Brief Experiential Avoidance Questionnaire, a Modified Tellegen Absorption Questionnaire, a Scale to Assess Therapeutic Relationship, Credibility/Expectancy Questionnaire, a Connectedness to Nature Scale, a Political Perspective Questionnaire, a Social Connectedness Scale, a Bech-Rafaelsen Mania Rating Scale, a Revised Santa Clara brief compassion scale, a Gratitude Questionnaire, a Short Suggestibility Scale, a Rosenberg Self-Esteem Scale, a Universality Subscale of the Spiritual Transcendence Scale, an Oxford Questionnaire on the Emotional Side-effects of Antidepressants, a Lauks Emotional Intensity Scale, Sexual Dysfunction Questionnaire, a Brief Index of Sexual Functioning for Women, a Sexual Perceptions Questionnaire, a Barnes Akathisia Rating Scale, a Work Productivity and Activity Impairment Questionnaire, a Work and Social Adjustment Scale, a Connectedness Questionnaire, a Standard Assessment of Personality, a Positive and Negative Syndrome Scale, a Mastery Insight Scale, a Self-Reflection and Insight Scale, a Psychological Insight Scale, a Metaphysical Beliefs Questionnaire, a Spiritual Bypassing Scale, an Adverse Childhood Experience Questionnaire, a Therapeutic Music Experience Questionnaire, a Setting Questionnaire, an Absorption in Music Scale, a Psychedelic Predictor Scale, a Surrender Scale, a EuroQOL-5 Dimension-3 Level Scale, or any combinations thereof. In some embodiments, after treatment with the methods described herein, the sign or symptom of depression measured using any of the assessments listed above decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using an imaging test before, during, or after treatment with the methods described herein. In some embodiments, the imaging test is a CT scan. In some embodiments, the imaging test is a functional MRI scan. In some embodiments, the functional MRI scan measures the blood oxygen level-dependent (BOLD) response as an indicator of brain activity and/or functional connectivity. In some embodiments, the BOLD response is measured in the subject at resting state, in response to emotional faces, or in response to music. In some embodiments, after treatment with the methods described herein, the BOLD response in a region of the brain increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In other embodiments, after treatment with the methods described herein, the BOLD response in a region of the brain decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment. In some embodiments, after treatment with the methods described herein, the BOLD response in the amygdala decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression is measured using a marker for depression in the blood or cerebral spinal fluid. In some embodiments, the marker for depression is measured in the subject before, during, or after treatment with the methods or compositions described herein. In some embodiments, the marker for depression is red blood cell folate, serum folate, vitamin B12, plasma homocysteine, serum methylfolate, and/or testing for one or more of brain-derived neurotrophic factor (BDNF) Val66Met, bone morphogenetic protein rs41271330, and/or 5-HTTLPR polymorphisms. In some embodiments, the marker for depression decreases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment. In other embodiments, the marker for depression increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the disease or disorder is an anxiety disorder. As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring. Examples of anxiety disorders include, but are not limited to, generalized anxiety disorder (GAD), social anxiety disorder, panic disorder, panic attack, a phobia-related disorder (e.g., phobias related to flying, heights, specific animals such as spiders/dogs/snakes, receiving injections, blood, etc., agoraphobia), separation anxiety disorder, selective mutism, anxiety due to a medical condition, post-traumatic stress disorder (PTSD), obsessive-compulsive disorder (OCD), substance-induced anxiety disorder, etc.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, the disease or disorder is generalized anxiety disorder (GAD). Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks. In some embodiments, the methods herein are provided to a subject with generalized anxiety disorder also having symptoms of depression. In some embodiments, after treating the symptom(s) is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is social anxiety disorder. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, body dysmorphic disorder, hoarding disorder, dermatillomania, trichotillomania, excoriation, substance-induced obsessive compulsive and related disorder, or an obsessive-compulsive disorder due to another medical condition, etc., or a combination thereof. In some embodiments, the disease or disorder is obsessive-compulsive disorder (OCD).

In some embodiments, at least one sign or symptom of an anxiety disorder is improved following treatment disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), The Dutch Temperament and Character Inventory (TCI), Dutch Personality Questionnaire-2-Revised (NPV-2-R), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-I-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Disorder 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y-BOCS-SR), The Obsessive-Compulsive Inventory-Revised (OCI-R), and the Dimensional Obsessive-Compulsive Scale (DOCS), or a combination thereof. In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the Spielberger State-Trait Anxiety Inventory (STAI) is utilized to evaluate the safety and efficacy of the methods of the present disclosure for the treatment of anxiety disorders (e.g., GAD). The STAI is a widely used instrument that contains separate self-report scales for measuring “state” and “trait” anxiety. “State” anxiety is transient anxiety due to a stressful stimulus, whereas “trait” anxiety is the predisposition of a subject to react with anxiety to stressful situations. The STAI is self-reported and contains 40 items scored by a 4 point Likert scale with semantic guides taking approximately 10 minutes. The STAI contains 20 questions related to state anxiety and 20 questions related to trait anxiety. Each section is scored between 20 and 80, with higher scores correlating with greater anxiety. Low scores indicate a mild form of anxiety and high scores indicate a severe form of anxiety. Both scales have anxiety absent and anxiety present questions. Anxiety absent questions represent the absence of anxiety in a statement like, “I feel secure.” Anxiety present questions represent the presence of anxiety in a statement like “I feel worried.” In some embodiments, after treatment with the methods of the present disclosure the patient has a lower STAI score compared with the subject's STAI score prior to treatment (baseline), i.e., the methods result in a negative STAI Change From Baseline (“CFB”) score. In some embodiments, the methods described herein result in a STAI CFB score, e.g., at two-weeks post-dose, at three-weeks post-dose, etc., of −10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a STAI CFB score, e.g., at two-weeks post-dose, at three-weeks post-dose, etc., of −10 to −20 points, −11 to −19 points, −12 to −18 points, or −13 to −17 points. In some embodiments, the methods described herein result in a STAI CFB score reduction over placebo, e.g., at two-weeks post-dose, at three-weeks post-dose, etc., of −8 points, −9 points, 10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a STAI CFB score reduction over placebo, e.g., at two-weeks post-dose, at three-weeks post-dose, etc., of −10 to −20 points, −12 to −18 points, −13 to 16 points, or −14 to −15 points. In some embodiments, the STAI CFB score reduction over placebo described above may be accompanied by ap-value of 0.05 or less, 0.01 or less, 0.005 or less, 0.001 or less, 0.0005 or less, or 0.0001 or less. In some embodiments, after treatment with the methods described herein, the subject's STAI score decreases from prior to treatment (baseline) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, at least 50%, at least 55%, at least 60%, at least 65%, at least 70%, at least 75%, at least 80%, at least 85%, at least 90% of subjects treated with the methods described herein have a STAI score decrease from baseline by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Dutch Temperament and Character Inventory (TCI) is utilized to evaluate the safety and efficacy of the methods of the present disclosure for the treatment of anxiety disorders (e.g., GAD). The TCI involves 240 bi-directional true-false alternatives, allowing a detailed assessment of personality traits, with a good internal consistency. Temperament is characterized on four dimensions: novelty seeking, harm avoidance, reward dependence, and persistence; character on three dimensions: self-directedness, cooperativeness, and self-transcendence. The four temperament dimensions are assumed to be underlined by specific neurotransmission systems.

In some embodiments, the Dutch Personality Questionnaire-2-Revised (NPV-2-R) is utilized to evaluate the safety and efficacy of the methods of the present disclosure for the treatment of anxiety disorders (e.g., GAD). The NPV-2-R is the most frequently used (clinical) personality questionnaire in The Netherlands. The NPV-2-R has satisfactory reliability, validity and internal consistencies, and includes 140 items. All items are answered on a bi-directional three-point scale (true-?-false), equally divided into seven scales of personality aspects: neuroticism (inadequatie), social anxiety (sociale inadequatie), rigidity (rigiditeit), hostility (verongelijktheid), egoism (zelfgenoegzaamheid), dominance (dominantie), and self-esteem (zelfwaardering). The NPV-2-R also has subscales; neuroticism has the subscales: depression and anxiety (depressive en angst), social anxiety has the subscales shyness and social avoidance (verlegenheid en sociale vermijding), rigidity has subscales for neatness and inflexibility (ordelijkheid en inflexibiliteit) and dominance has subscales for leadership and independence (leidinggeven en autonomie).

The Mini International Neuropsychiatric Interview (MINI) (for example, version 7.0.2) is a diagnostic interview instrument for psychiatric disorders in the Diagnostic and Statistical Manual of Mental Disorders (DSM-5) and the International Classification of Diseases-10. In some embodiments, treating according to the methods of the disclosure results in an improvement in a psychiatric disorder (e.g., anxiety, etc.) compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to a Mini International Neuropsychiatric Interview assessment. In some embodiments, the subject has a lower MINI 7.0.2 score compared with the subject's MINI 7.0.2 score prior to treatment.

In some embodiments, the disease or disorder is attention deficit disorder (ADD). ADD is most commonly diagnosed in children under the age of 16 who have 6 or more symptoms of inattention (5 or more for older teenagers) for at least 6 consecutive months, but no signs of hyperactivity/impulsivity. The symptoms of inattention include, but are not limited to, trouble paying attention, avoids long mental tasks such as homework, trouble staying on task, disorganized or forgetful, doesn't appear to listen when spoken to, doesn't pay close attention to details. Loses things often, makes careless mistakes, and struggles to follow through with instructions. In some embodiments, the disease or disorder is attention deficit hyperactivity disorder (ADHD). ADHD is marked by an ongoing pattern of inattention and/or hyperactivity-impulsivity. Hyperactivity-impulsivity symptoms may often include, but are not limited to, fidgeting or squirming while seated, leaving their seats in situations where staying seated is expected, running, dashing, or climbing around at inappropriate times, being unable to engage in hobbies quietly, being constantly in motion, talking excessively, answering questions before they are fully asked, having difficulty waiting for one's turn, and interrupting or intruding on others during conversations or activities.

In some embodiments, the disease or disorder is a headache disorder. As used herein, the term “headache disorder” refers to a disorder characterized by recurrent headaches. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination. Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve—the fifth cranial nerve whose primary function is to provide sensory and motor innervation to the face. Attacks are also associated with prominent unilateral cranial autonomic symptoms and subjects often experience agitation and restlessness during attacks. In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain. In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine.” Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves. In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, the frequency of headaches and/or related symptoms decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, at least one sign or symptom of headache disorder is improved following administration of a compound disclosed herein. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQoL-5D, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-5 Dimensions (EQ-5D), the Short-form 36 (SF-36), or a combination thereof. In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art. In some embodiments, the sign or symptom of the headache disorder is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, the disease or disorder is a substance use disorder. Substance addictions which can be treated using the methods herein include addictions to addictive substances/agents such as recreational drugs and addictive medications. Examples of addictive substances/agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine. In some embodiments, the disease or disorder is alcohol use disorder (AUD). In some embodiments, the disease or disorder is nicotine use (e.g., smoking) disorder, and the therapy is used for e.g., smoking cessation. In some embodiments, a diary assessment, an assessment by a clinician or caregiver, or a clinical scale is used for measuring the substance use disorder and/or the response to treatment herein. Non-limiting examples of clinical scales, diary assessments, and assessments by a clinician or caregiver include: The Fagerstöm Test for Nicotine Dependence and the Questionnaire on Smoking Urges.

In some embodiments, the disclosure provides for the management of sexual dysfunction, which may include, but is not limited to, sexual desire disorders, for example, decreased libido; sexual arousal disorders, for example, those causing lack of desire, lack of arousal, pain during intercourse, and orgasm disorders such as anorgasmia; and erectile dysfunction; particularly sexual dysfunction disorders stemming from psychological factors.

In some embodiments, the disease or disorder is an eating disorder. As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring. In some embodiments, at least one sign or symptom of an eating disorder is improved following administration of a compound disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Asberg Depression Rating Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C-SSRS), the Life Changes Inventory (LCI), and combinations thereof. In some embodiments, treating according to the methods of the disclosure results in an improvement in an eating disorder compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is multiple sclerosis (MS). MS is a chronic, inflammatory disease of unknown etiology that involves an immune-mediated attack on the central nervous system. Myelin and the oligodendrocytes that form myelin appear to be the primary targets of the inflammatory attack, although the axons themselves are also damaged. MS disease activity can be monitored by cranial scans, including magnetic resonance imaging (MRI) of the brain, accumulation of disability, as well as rate and severity of relapses. The diagnosis of clinically definite MS as determined by the Poser criteria requires at least two neurological events suggesting demyelination in the CNS separated in time and in location. Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination. In some embodiments, the multiple sclerosis is relapsing multiple sclerosis. In some embodiments, the relapsing multiple sclerosis is relapsing-remitting multiple sclerosis. In some embodiments, the methods herein reduce a symptom of multiple sclerosis in the subject. In some embodiments, the symptom is a MRI-monitored multiple sclerosis disease activity, relapse rate, accumulation of physical disability, frequency of relapses, decreased tune to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work. In some embodiments, the methods herein decrease or inhibit reduction of brain volume. In some embodiments, brain volume is measured by percent brain volume change (PBVC). In some embodiments, the methods herein increase time to confirmed disease progression. In some embodiments, time to confirmed disease progression is increased by at least 10%, at least 20%, at least 30%, at least 40%, at least 50%, for example at least 20-60%. In some embodiments, the methods herein decrease abnormalities observed in whole Brain MTR histogram. In some embodiments, the accumulation of physical disability is measured by Kurtzke Expanded Disability Status Scale (EDSS) score. In some embodiments, the accumulation of physical disability is assessed by the time to confirmed disease progression as measured by Kurtzke Expanded Disability Status Scale (EDSS) score.

In some embodiments, the disease or disorder is a disease or disorder characterized by, or otherwise associated with, neuroinflammation. Treatment herein may provide cognitive benefits to subject's suffering from neurological and neurodegenerative diseases such as Alzheimer's disease and other dementia subtypes, Parkinson's disease, amyotrophc lateral sclerosis (ALS), and others where neuroinflammation is a hallmark of disease pathophysiology and progression. For example, emerging psychedelic research/clinical evidence indicates that psychedelics may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the methods of the present disclosure are used for the treatment of neurological and neurodegenerative disorders such as Alzheimer's disease, dementia subtypes, Parkinson's disease, and amyotrophc lateral sclerosis (ALS), where neuroinflammation is associated with disease pathogenesis. In some embodiments, the methods of the present disclosure are used for the treatment of Alzheimer's disease. In some embodiments, the methods of the present disclosure are used for the treatment of dementia. In some embodiments, the methods of the present disclosure are used for the treatment of Parkinson's disease. In some embodiments, the methods of the present disclosure are used for the treatment of amyotrophc lateral sclerosis (ALS). As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical formulations adapted for oral and/or extended-release dosing (e.g., subcutaneous) are appropriate for such treatment methods, with sub-psychedelic dosing being preferred. In some embodiments, treating according to the methods of the disclosure results in an improvement in cognition in subject's suffering from a neurological or neurodegenerative disease compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of a diary assessments, assessments by a clinical or caregiver, or clinical scales, described herein or known in the art.

Further, many of the behavioral issues associated with chronic and/or life-threatening illnesses, including neurodegenerative disorders such as Alzheimer's disease, may benefit from treatment with the compounds/salt forms disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. The methods described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder, including neurological and neurodegenerative diseases. In some embodiments, the methods provided herein reduce at least one sign or symptom of a neurological and/or neurodegenerative disease. In some embodiments, the methods provided herein reduce at least one sign or symptom of a neurological and/or neurodegenerative disease (e.g., depression, anxiety, and/or stress) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment, e.g., according to any one of the diary assessments, assessments by a clinical or caregiver, or clinical scales, described herein or known in the art.

In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is amyotrophc lateral sclerosis (ALS). In some embodiments, the methods herein are used for the treatment of depression, anxiety, and/or stress associated with amyotrophc lateral sclerosis (ALS). In some embodiments, the disease or disorder is cancer related depression and anxiety.

In some embodiments, the methods disclosed herein are used for treatment of brain injury, including traumatic brain injury (TBI). TBI is an injury to the brain caused by an external force, and can be classified based on severity, ranging from mild traumatic brain injury (mTBI/concussion) to severe traumatic brain injury. TBI can also be categorized by mechanism, as either a closed or penetrating head injury, or other features such as whether it is occurring in a specific location or over a widespread area. TBI can result in physical, cognitive, social, emotional and behavioral symptoms, which may be treated herein. Some of the imaging techniques used for diagnosis and recovery markers include computed tomography (CT) and magnetic resonance imaging (MRIs).

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the compounds and compositions disclosed herein are used for treatment of tic disorders, including Tourette's Syndrome, which is also variously referred to as Tourette Syndrome, Tourette's Disorder, Gilles de la Tourette syndrome (GTS), or simply Tourette's or TS. The tic disorder may also be a pediatric autoimmune disorder associated with streptococcal infection (PANDAS), a transient tic disorder, a chronic tic disorder, or a tic disorder not otherwise specified (NOS). Tic disorders are defined in the Diagnostic and Statistical Manual of Mental Disorders (DSM) based on type (motor or phonic) and duration of tics (sudden, rapid, nonrhythmic movements), or similarly by the World Health Organization (ICD-10 codes). Tics are involuntary or semi-voluntary, sudden, brief, intermittent, repetitive movements (motor) or sounds (phonic) that are classified as simple or complex. Simple tics, for example, eye blinking or facial grimacing, are relatively easy to camouflage and may go largely unnoticed. Complex tics, such as body contortions, self-injurious behavior, obscene gestures, or shouting of socially inappropriate word or phrases, can appear to be purposeful actions and are particularly distressing. Transient tic disorders are generally characterized by multiple motor and/or phonic tics that occur for at least four weeks but less than 12 months. Chronic tic disorders are generally characterized by either single or multiple motor or phonic tics, but not both, which are present for more than a year. Tourette's Syndrome is diagnosed when both motor and phonic tics are present (although not necessarily concurrently) for more than one year. Thus, Tourette's syndrome (TS) is a chronic neuropsychiatric disorder characterized by the presence of fluctuating motor and phonic tics. The typical age of onset is between five and seven years. Affected children may become the target of teasing by peers, which in turn can result in low self-esteem, social isolation, poor school performance, depression and anxiety. In addition to causing social embarrassment, sudden, forceful tics can be painful, and violent head and neck tics have been reported to cause secondary neurologic deficits, such as compressive cervical myelopathy. Tourette's Syndrome patients are also at increased risk for obsessive-compulsive disorder (OCD), depression, and attention-deficit-hyperactivity disorder (ADHD). Tic disorder NOS is diagnosed when tics are present but do not meet the criteria for any specific tic disorder. The methods of the present disclosure can also be used for the treatment of tics induced as a side effect of a medication; tics associated with autism; and Tourettism (the presence of Tourette-like symptoms in the absence of Tourette's Syndrome (e.g., as a result of another disease or condition, such as a sporadic, genetic, or neurodegenerative disorder)).

In some embodiments, the disease or disorder is a fluency disorder. As used herein, a “fluency disorder” is an interruption in the flow of speaking characterized by atypical rate, rhythm, and disfluencies (e.g., repetitions of sounds, syllables, words, and phrases; sound prolongations; and blocks), which may also be accompanied by excessive tension, speaking avoidance, struggle behaviors, and secondary mannerisms. People with fluency disorders also frequently experience psychological, emotional, social, and functional impacts as a result of their communication disorder. Childhood-onset fluency disorder (stuttering), the most common fluency disorder, is an interruption in the flow of speaking characterized by specific types of disfluencies, including (i) repetitions of sounds, syllables, and monosyllabic words; (ii) prolongations of consonants when it isn't for emphasis; and (iii) blocks (i.e., inaudible or silent fixation or inability to initiate sounds). These disfluencies can affect the rate and rhythm of speech and may be accompanied by negative reactions to speaking; avoidance behaviors (i.e., avoidance of sounds, words, people, or situations that involve speaking); escape behaviors, such as secondary mannerisms (e.g., eye blinking and head nodding or other movements of the extremities, body, or face); and physical tension. Children and adults who stutter also frequently experience psychological, emotional, social, and functional consequences from their stuttering, including anxiety (e.g., social anxiety), a sense of loss of control, and negative thoughts or feelings about themselves or about communication. Childhood-onset fluency disorder (stuttering) can co-occur with other disorders, such as attention-deficit/hyperactivity disorder, autism spectrum disorder, intellectual disability, language or learning disability, seizure disorders, anxiety disorders (e.g., social anxiety disorder), speech sound disorders, and other developmental disorders.

In some embodiments, the disease or disorder may include conditions of the autonomic nervous system (ANS).

In some embodiments, the disease or disorder may include pulmonary disorders (e.g., asthma and chronic obstructive pulmonary disorder (COPD).

In some embodiments, the disease or disorder may include cardiovascular disorders (e.g., atherosclerosis).

In some embodiments, the disclosure provides for the management of different kinds of pain, including but not limited to cancer pain, e.g., refractory cancer pain; neuropathic pain; postoperative pain; opioid-induced hyperalgesia and opioid-related tolerance; neurologic pain; postoperative/post-surgical pain; complex regional pain syndrome (CRPS); shock; limb amputation; severe chemical or thermal burn injury; sprains, ligament tears, fractures, wounds and other tissue injuries; dental surgery, procedures and maladies; labor and delivery; during physical therapy; radiation poisoning; acquired immunodeficiency syndrome (AIDS); epidural (or peridural) fibrosis; orthopedic pain; back pain; failed back surgery and failed laminectomy; sciatica; painful sickle cell crisis; arthritis; autoimmune disease; intractable bladder pain; pain associated with certain viruses, e.g., shingles pain or herpes pain; acute nausea, e.g., pain that may be causing the nausea or the abdominal pain that frequently accompanies sever nausea; migraine, e.g., with aura; and other conditions including depression (e.g., acute depression or chronic depression), depression along with pain, alcohol dependence, acute agitation, refractory asthma, acute asthma (e.g., unrelated pain conditions can induce asthma), epilepsy, acute brain injury and stroke, Alzheimer's disease and other disorders. The pain may be persistent or chronic pain that lasts for weeks to years, in some cases even though the injury or illness that caused the pain has healed or gone away, and in some cases despite previous medication and/or treatment. In addition, the disclosure includes the treatment/management of any combination of these types of pain or conditions.

In some embodiments, the pain treated/managed is acute breakthrough pain or pain related to wind-up that can occur in a chronic pain condition. In some embodiments of the disclosure, the pain treated/managed is cancer pain, e.g., refractory cancer pain. In some embodiments of the disclosure, the pain treated/managed is post-surgical pain. In some embodiments of the disclosure, the pain treated/managed is orthopedic pain. In some embodiments of the disclosure, the pain treated/managed is back pain. In some embodiments of the disclosure, the pain treated/managed is neuropathic pain. In some embodiments of the disclosure, the pain treated/managed is dental pain. In some embodiments of the disclosure, the condition treated/managed is depression. In some embodiments of the disclosure, the pain treated/managed is chronic pain in opioid-tolerant patients.

In some embodiments, the disease or disorder is arthritis. Types of arthritis include osteoarthritis, rheumatoid arthritis, childhood arthritis, fibromyalgia, gout, and lupus. In some embodiments, the disease or disorder is osteoarthritis. In some embodiments, the disease or disorder is rheumatoid arthritis. In some embodiments, the disease or disorder is childhood arthritis. In some embodiments, the disease or disorder is gout. In some embodiments, the disease or disorder is lupus. In some embodiments, the disease or disorder is fibromyalgia. Fibromyalgia is a disorder characterized by widespread musculoskeletal pain accompanied by fatigue, sleep, memory and mood issues. Fibromyalgia is believed to amplify painful sensations by affecting brain and spinal cord processes involving painful and nonpainful signaling. Symptoms often begin after an event, such as physical trauma, surgery, infection or significant psychological stress. In other cases, symptoms gradually accumulate over time with no single triggering event. Women are more likely to develop fibromyalgia than are men. Many people who have fibromyalgia also have tension headaches, temporomandibular joint (TMJ) disorders, irritable bowel syndrome, anxiety and depression.

In some embodiments, the disease or disorder is inflammatory bowel disease (IBD). IBD is a term for two conditions, Crohn's disease and ulcerative colitis, that are characterized by chronic inflammation of the gastrointestinal (GI) tract, with such prolonged inflammation resulting in damage to the GI tract. Subjects suffering from IBD may experience persistent diarrhea, abdominal pain, rectal bleeding/bloody stools, weight loss, and fatigue. IBD may be diagnosed, and treatment may be monitored, using one or more of endoscopy, colonoscopy, contrast radiography, MRI, computed tomography (CT), stool samples, and blood tests, known by those of ordinary skill in the art.

In some embodiments, the disease or disorder is a sleep disorder such as narcolepsy, insomnia, nightmare disorder, sleep apnea, central sleep apnea, obstructive sleep apnea, hypopnea, sleep-related hypoventilation, restless legs syndrome, and jet lag. In some embodiments, the disease or disorder is narcolepsy.

In some embodiments, the method is used to treat a disease or condition by modulating N-methyl-D-aspartic acid (NMDA) activity. In some embodiments, the disease or condition is selected from: levodopa-induced dyskinesia; dementia (e.g., Alzheimer's dementia), tinnitus, treatment resistant depression (TRD), major depressive disorder, melancholic depression, atypical depression, dysthymia, neuropathic pain, agitation resulting from or associated with Alzheimer's disease, pseudobulbar effect, autism, Bulbar function, generalized anxiety disorder, Alzheimer's disease, schizophrenia, diabetic neuropathy, acute pain, depression, bipolar depression, suicidality, neuropathic pain, or post-traumatic stress disorder (PTSD). In some embodiments, the disease or condition is a psychiatric or mental disorder (e.g., schizophrenia, mood disorder, substance induced psychosis, major depressive disorder (MDD), bipolar disorder, bipolar depression (BDep), post-traumatic stress disorder (PTSD), suicidal ideation, anxiety, obsessive compulsive disorder (OCD), and treatment-resistant depression (TRD)). In other embodiments, the disease or condition is a neurological disorder (e.g., Huntington's disease (HD), Alzheimer's disease (AD), or systemic lupus erythematosus (SLE)).

In some embodiments, the disclosure relates to a method of treating an ocular disease, such as uveitis, corneal disease, iritis, iridocyclitis, glaucoma, and cataracts, by administering ophthalmically the pharmaceutical formulation, for example, in the form of an eye drop formulation.

The administering physician can provide a method of treatment that is prophylactic or therapeutic by adjusting the amount and timing of any of the salt forms of the compounds described herein on the basis of observations of one or more symptoms of the disorder or condition being treated.

In some embodiments, the subject is a mammal. In some embodiments, the mammal is a human.

In some embodiments, the pharmaceutical formulations of the present disclosure may be used as a standalone therapy. In some embodiments, the pharmaceutical formulations of the present disclosure are administered without psychological support.

### Adjuvant Combination Therapy

In some embodiments, the pharmaceutical formulations of the present disclosure may be used as an adjuvant/combination therapy. In some embodiments, the subject with a disorder is administered a pharmaceutical formulation of the present disclosure and at least one additional therapy and/or therapeutic. In some embodiments, administration of an additional therapy and/or therapeutic is prior to administration of the pharmaceutical formulation of the present disclosure. In some embodiments, administration of an additional therapy and/or therapeutic is after administration of the pharmaceutical formulation of the present disclosure. In some embodiments, administration of an additional therapy and/or therapeutic is concurrent with administration of the pharmaceutical formulation of the present disclosure. In some embodiments, administration of an additional therapy and/or therapeutic is prior to, during, and/or after administration of the pharmaceutical formulation of the present disclosure. In some embodiments, administration of the pharmaceutical formulation of the present disclosure, as part of combination or adjunctive therapy, may be accompanied by psychotherapy before, during, and/or after each dose.

In the adjunctive or combination therapy described herein, the pharmaceutical formulation of the present disclosure may be administered using a different dosing schedule than the additional therapy and/or therapeutic. For example, the subject may be taking an antidepressant medication on a daily schedule (e.g., daily oral dosing), while the pharmaceutical formulation of the present disclosure may be administered using an intermittent dosing schedule as described herein, e.g., one dose in a treatment course or two doses administered three weeks apart (±3 days) in a treatment course. In some embodiments, adjunctive therapy or combination therapy provides detectable plasma levels of the psychopharmaceutical agent and the at least one additional therapeutic (e.g., antidepressant medication) at the same time. For example, a subject taking an antidepressant medication on a daily schedule (e.g., daily oral dosing) who is administered the pharmaceutical formulation of the present disclosure may have detectable plasma levels of each agent shortly after dosing of the pharmaceutical formulation of the present disclosure. In some embodiments, adjunctive therapy or combination therapy does not lead to detectable plasma levels of the psychopharmaceutical agent and the at least one additional therapeutic (e.g., antidepressant medication) at the same time.

In some embodiments, the pharmaceutical formulation comprises a tryptamine psychedelic (e.g., a pharmaceutically acceptable salt of a compound of Formula (I) through (III)) as the psychopharmaceutical agent, and the additional therapeutic is an antidepressant medication, an anticonvulsant, lisdexamfetamine dimesylate, an antipsychotic, an anxiolytic, an anti-inflammatory drug, a benzodiazepine, an N-methyl-D-aspartate (NMDA) receptor antagonist, an analgesic drug, a cardiovascular drug, an opioid antagonist, or combinations thereof.

In some embodiments, the additional therapeutic is an antidepressant medication. In some embodiments, the pharmaceutical formulation of the present disclosure is used in adjuvant therapy for patients already taking an antidepressant medication (e.g., SSRIs, SNRIs, etc.). For clarity, when referencing combination or adjunctive therapy involving an “antidepressant medication” (or simply an “antidepressant”) the psychopharmaceutical agent contained within the pharmaceutical formulation of the present disclosure is considered herein to be separate and distinct from the antidepressant medication, despite both potentially having antidepressant properties. Instead, “antidepressant medication” (or “antidepressant”) refers to non-psychedelic agents, with most regulator-approved antidepressant medications being monoamine antidepressant agents characterized by a mode of action which addresses one or more imbalance (usually a deficiency) of neurotransmitters serotonin, norepinephrine and dopamine. In some embodiments, the antidepressant medication is an approved pharmacological treatment for a depressive disorder in at least one jurisdiction, such as the United States of America, the European Union, the United Kingdom, Australia, New Zealand, and Japan. In some embodiments, an antidepressant medication indirectly affects a neurotransmitter receptor, e.g., via interactions affecting the reactivity of other molecules at a neurotransmitter receptor. In some embodiments, the antidepressant medication is an agonist. In some embodiments, the antidepressant medication is an antagonist. In some embodiments, the antidepressant medication is a tricyclic antidepressant (“TCA”), selective serotonin reuptake inhibitor (“SSRI”), serotonin reuptake inhibitor (“SRI”), serotonin and noradrenaline reuptake inhibitor (“SNRI”), dopamine reuptake inhibitor (“DRI”), noradrenaline reuptake inhibitor (“NRU”), dopamine, serotonin, and noradrenaline reuptake inhibitor (“DSNRI”), a monoamine oxidase inhibitor (“MAOI”), including a reversible inhibitor of monoamine oxidase type A (RIMA), or combinations thereof. In some embodiments, the antidepressant medication is a TCA. In some embodiments, the TCA is imipramine or clomipramine. In some embodiments, the antidepressant medication is a serotonin reuptake inhibitor (“SRI”). In some embodiments, the antidepressant medication is a selective serotonin reuptake inhibitor (SSRI). In some embodiments, the SSRI is citalopram, escitalopram, paroxetine, sertraline, fluvoxamine, fluoxetine, or combinations thereof. In some embodiments, the antidepressant medication is a serotonin and noradrenaline reuptake inhibitor (SNRI). In some embodiments, the SNRI is venlafaxine, desvenlafaxine, duloxetine, levomilnacipran, or combinations thereof. In some embodiments, the antidepressant effects are augmented with the use of pregabalin as an additional therapeutic. In some embodiments, the antidepressant medication acts (either directly or indirectly) at more than one type of neurotransmitter receptor. In some embodiments, the antidepressant medication is chosen from buproprion, citalopram, duloxetine, escitalopram, fluoxetine, fluvoxamine, milnacipran, mirtazapine, paroxetine, reboxetine, sertraline, venlafaxine, dextromethorphan, cariprazine, vortioxetine, vilazodone, and levomilnacipram. Antidepressant medications such as SSRIs are usually taken orally, typically in tablet form, and usually need to be taken daily. In some embodiments, the pharmaceutical formulation of the present disclosure is used as adjuvant therapy for patients taking a stable dose of antidepressant medication, such as patients on background antidepressant medication(s) including SSRIs and SNRIs. In some embodiments, the pharmaceutical formulation of the present disclosure is used as adjuvant therapy for patients taking a stable dose of antidepressant medication with an inadequate response to that antidepressant medication. In some embodiments, the subject is taking an antidepressant medication monotherapy. In some embodiments, the subject is taking a combination of antidepressant medications. In some embodiments, the subject is taking an antidepressant medication such as citalopram, escitalopram, paroxetine, sertraline, fluoxetine, venlafaxine, desvenlafaxine, duloxetine, levomilnacipran, buproprion, vortioxetine, vilazodone, or combinations thereof.

In some embodiments, the additional therapeutic is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapeutic is an anxiolytic. In some embodiments, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the additional therapeutic is an anti-inflammatory drug. In some embodiments, the anti-inflammatory drug is a nonsteroidal anti-inflammatory drugs (NSAIDS), steroid, acetaminophen (COX-3 inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic agent, 3-hydroxy-3-methylglutaryl coenzyme A inhibitor (statins), estrogen, androgen, antiplatelet agent, antidepressant, Helicobacter pylori inhibitors, proton pump inhibitor, thiazolidinedione, dual-action compounds, or combination thereof.

In some embodiments, the additional therapeutic is a benzodiazepine. In some embodiments, the benzodiazepine is diazepam or alprazolam.

In some embodiments, the additional therapeutic is a N-methyl-D-aspartate (NMDA) receptor antagonist. In some embodiments, the NMDA receptor antagonist is ketamine. In some embodiments, the NMDA receptor antagonist is nitrous oxide.

In some embodiments, the additional therapeutic is an analgesic. In some embodiments, the analgesic is acetaminophen. In some embodiments, the analgesic is a nonsteroidal anti-inflammatory drug (NSAID), such as aspirin, ibuprofen and naproxen. In some embodiments, the analgesic is a COX-2 inhibitor, such as rofecoxib, celecoxib, parecoxib, and etoricoxib. In some embodiments, the analgesic is an opioid.

In some embodiments, the additional therapeutic is a cardiovascular drug. Non-limiting examples of cardiovascular drugs include digoxin or (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribohexopyranosyl) oxy]-12,14-dihydroxy-card-20(22)-enolide, lisinopril, captopril, ramipril, trandolapril, benazepril, cilazapril, enalapril, moexipril, perindopril, quinapril, fludrocortisone, enalaprilate, quinapril, perindopril, apixaban, dabigatran, edoxaban, heparin, rivaroxaban, warfarin, aspirin, clopidogrel, dipyridamole, prasugrel, ticagrelor, azilsartan, candesartan, eprosartan, irbesartan, losartan, olmesartan, telmisartan, valsartanscaubitril, acebutolol, atenolol, betaxolol, bisoprolol, metoprolol, nadolol, propranolol, sotalol, amlodipine, diltiazem, felodipine, nifedipine, nimodipine, nisolidipine, verapamil, statins, nicotinic acids, diuretics, vasodilators, and combinations thereof.

In some embodiments, the additional therapeutic is an opioid antagonist. Non-limiting examples of opioid antagonists include naloxone, naltrexone, nalmefene, nalorphine, nalrphine dinicotinate, levallrphan, samidorphan, nalodeine, alvimopan, methylnaltrexone, naloxegol, 6-naltrexol, axelopran, bevenopran, methylsamidorphan, naldemedine, buprenorphine, decozine, butorphanol, levorphanol, nalbuphine, pentazocine, and phenazocine.

In some embodiments, the additional therapeutic is a serotonin receptor modulator. Non-limiting examples of serotonin receptor modulators include glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin (ACO-103), nelotanserin, lorcaserin, flibanserin, and roluperiodone.

In some embodiments, the subject is administered at least one therapy in addition to administration of the pharmaceutical formulation of the present disclosure. Non-limiting examples of therapies include transcranial magnetic stimulation, cognitive behavioral therapy, interpersonal psychotherapy or psychedelic-assisted psychotherapy (PAP), dialectical behavior therapy, mindfulness techniques, or acceptance, commitment therapy, or combinations thereof. In some embodiments, the subject is administered a six-domain framework for psychedelic-assisted psychotherapy referred to as EMBARK therapy.

In some embodiments, a method of treating a subject in need thereof comprises administering to the subject a therapeutically effective amount of the pharmaceutical formulation in a controlled environment, wherein the subject is provided with psychological support.

In some embodiments, a method of treating a subject in need thereof comprises at least one of the following:
(i) administering to the subject a therapeutically effective amount of the pharmaceutical formulation in a controlled environment, wherein the subject is provided with psychological support;
(ii) having the subject participate in one or more pre-administration psychological support session(s); and/or
(iii) having the subject participate in one or more post-administration psychological support session(s).

In some embodiments, after administration, the subject may not feel the effects of the drug for about 1 minute, about 5 minutes, about 10 minutes, about 15 minutes, about 30 minutes, about 45 minutes, about 60 minutes, or any range therebetween. This period after administration and before the onset of effects will be referred to herein as the initial stage of the treatment session. The time marked by the onset of the drug's effects will be referred to herein as the early stage of the treatment session.

In some embodiments, the subject will experience peak effects at about 0.5 hours, about 0.75 hours, about 1.0 hours, about 1.25 hours, about 1.5 hours, about 1.75 hours, about 2 hours after administration, or any range therebetween. The time period marked by the peak drug experience will be referred to herein as the peak stage of the treatment session.

In some embodiments, the effects may substantially wear off from about 0.5 hours, about 0.75 hours, about 1.0 hours, about 1.25 hours, about 1.5 hours, about 2 hours after administration, or any range therebetween. This time period will be referred to as the late stage of the treatment session.

In some embodiments, the subject's ability to reach a non-dual state (e.g., a mystical experience), or a sense of unity, boundlessness, ego-dissolution or transcendence correlates with positive clinical outcome. Each of these terms may be commonly defined as the breakdown of the usual relationship between self and other, whereby the subject might feel a oneness and increased sense of connectedness to the surrounding environment and/or the world at large. In some embodiments, low levels of emotional arousal-which could indicate avoidance, lack of involvement or intellectualization-might, in some embodiments, be correlated with little or no improvement in treatment outcomes.

Factors that may influence the subjective experience may include, for example, (i) dose, (ii) the mindset of the participant prior to the session, (iii) the setting of the session, (iv) the subject's ability to focus and stay with the experience, and/or (v) the subject's prior experience with psychedelics. These, and other factors, will be described in more detail below, along with ways to maximize therapeutic benefit of the treatment session.

### Pre-Administration Psychological Support Sessions (“Preparation Sessions”)

In some embodiments, the subject participates in at least one psychological support session before administration begins (“pre-administration psychological support session” or “preparation session”). In some embodiments, a pre-administration psychological support session may be held about 1 month prior to the administration. In some embodiments, a pre-administration psychological support session may be held about 2 weeks prior to the administration. In some embodiments, a pre-administration psychological support session may be held about 1 week prior to the administration. In some embodiments, a pre-administration psychological support session may be held about 3 days prior to the administration. In some embodiments, a pre-administration psychological support session may be held about 1 day prior to the administration. In some embodiments, a pre-administration psychological support session may be held on the same day as and prior to administration. In some embodiments, a pre-administration psychological support session may be held in any range in between about 1 month prior to the administration to the same day as administration.

In some embodiments, the subject may participate in one, two, three, four, five, six, seven, or eight pre-administration psychological support sessions. In some embodiments, the subject may participate in at least two pre-administration psychological support sessions. In some embodiments, the subject may participate in at least three pre-administration psychological support sessions. In some embodiments, the subject may participate in pre-administration psychological support sessions at least once per week, for at least two or three weeks prior to the administration session. In some embodiments, the subject may additionally participate in a pre-administration psychological support session the day before the administration session.

The pre-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the pre-administration psychological support session(s).

In some embodiments, the goals of the pre-administration session may include (i) establishing therapeutic alliance between subject and therapist; (ii) answering the subject's questions and addressing any concerns; and/or (iii) demonstrating and practicing the skills of self-directed inquiry and experiential processing. In some embodiments, the pre-administration psychological support sessions focus on discussion of possible psychedelic effects, and/or preparing subjects for the dosing session by practicing relevant therapeutic techniques to reduce avoidance and anxiety, eliciting relevant therapeutic goals, building rapport, and/or establishing therapeutic alliance. During the psychological support session, skills of self-directed inquiry and experiential processing may be demonstrated and/or practiced. In some embodiments, breathing exercises meant to promote calm and/or ease anxiety may be demonstrated and/or practiced. In some embodiments, the breathing exercise comprise instructing the subject to focus on their breath and/or sensations associated with the breath throughout the body. For example, the subject may be instructed to breathe in for a count of four, to hold their breath for a moment, and then to breathe out for a count of eight. In some embodiments, the therapist and subject may discuss the most helpful ways to support in case of emotional distress during the treatment session. In some embodiments, the subject is given access (e.g., online access) to materials concerning the safety and mechanism of action of the drug.

In some embodiments, the pre-administration psychological support sessions will serve to establish a therapeutic goal for the treatment session. In some embodiments, the subject suggests the therapeutic goal for herself or himself. In some embodiments, the therapist suggests the therapeutic goal to the subject. In some embodiments, the subject is reminded of the therapeutic goal during the pre-administration psychological support session.

In some embodiments, the therapists are trained to counsel the subject before, during, and/or after the treatment sessions. In some embodiments, the therapist will have mental health training. In some embodiments, the therapist will be a clinical psychologist, a psychiatrist, a social worker, a doctor or a nurse. In some embodiments, the therapist will meet the following criteria: (i) demonstrate independent clinical experience with direct subject care in areas that require counselling and psychotherapeutic skills; (ii) current unrestricted professional license and/or good professional standing with no history of suspension, professional misconduct or disciplinary actions; and/or (iii) high level of openness to learning new approaches and receiving feedback.

### Psychological Support During Treatment Sessions (“Medicine Sessions”)

During the treatment session, the subject may be supervised by one or more trained therapists. The therapist supervising the subject during the treatment session may be the same therapist from the subject's pre-administration psychological support session(s), or may be a different therapist. The therapist(s) may provide psychological support to the subject as necessary. As used herein, the term “psychological support” refers to any measure(s) taken by the therapist during the subject's treatment session to ensure the safety of the subject and maximize the clinical effectiveness of the treatment session. For example, the psychological support may be anything done by the therapist to (1) to ensure psychological safety of the subject; (2) to allow the subject's subjective experience to unfold naturally within the boundaries of the therapeutic intention set at the preparation; (3) to maintain participant's attention and awareness on the experience of the present moment thus allowing exposure and processing of the challenging emotional states and personal memories; and/or (4) to generate insights and solutions for the resolution of challenging personal situations, conflicts and traumatic experiences. In some embodiments, support can be in the form of therapeutic touch, verbal reassurance, guided imagery and/or relaxation or breathing exercises. In some embodiments, the support may comprise reminders, encouragement, or active guiding. Typically, only one technique is applied at a time to allow for minimal intervention and interference with the subject's unique process.

In some embodiments, the main therapeutic goals of the therapist during the treatment session are to (i) minimize extreme anxiety, and (ii) provide appropriate support that enables the skills and processes of self-directed inquiry and experiential processing. In some embodiments, the therapist demonstrates genuine presence, patience, curiosity, and/or openness during the treatment session. “Presence” refers to being totally available and present with the subject during all stages of the treatment session, and exuding calmness at all times. “Curiosity” refers to interest and willingness to understand the subject's experience, without making assumptions. “Patience” means that the therapist facilitates the participant taking as much time as needed to explore their experiences without controlling the natural urge to help or direct the experience. “Openness” is the ability of the therapist to remain cognitively and experientially open, including a capacity to be curious about how the subject's mind may uniquely choreograph the unfolding content of a session. This includes welcoming all emotions and expressions that might occur.

In some embodiments, the psychological support may comprise curious questioning. In this technique, brief, but detailed, questioning of subjects is used to help the subjects shift and sustain their attention towards different levels of cognition and emotions (“How does that make you feel?”). Due to the applicability across a range of mental states and within various settings, the technique of curious questioning can typically be used safely and consistently during the treatment session, regardless of the quality or intensity of the experience of each subject.

In some embodiments, the level of psychological support will vary during the various stages of the subject's treatment experience (e.g., the initial stage, the early stage, the peak stage, and the late stage). In some embodiments, the type of psychological support will vary during the various stages of the subject's treatment experience (e.g., the initial stage, the early stage, the peak stage, and the late stage). Because non-dual, ego-dissolution or “unitive” experiences have been shown to positively correlate with the magnitude and durability of the clinical response, the therapist will, in some embodiments, attend to such states with particular care.

In some embodiments, a subject may experience of a compromised sense of self during the subject's treatment experience. In some embodiments, this is interpreted from a psychoanalytic perspective as a disruption of ego-boundaries, which results in a blurring of the distinction between self-representation and object-representation, and precludes the synthesis of self-representations into a coherent whole. In some embodiments, non-dual, ego-dissolution or “unitive” experiences refer to an altered state of consciousness in which there is a reduction in the self-referential awareness that defines normal waking consciousness, resulting in a compromised sense of “self” and instead only a undivided background awareness, often characterized by a sense of unity or “oneness” that exceeds sensory or cognitive apprehension. In some embodiments, a non-dual experience is state of consciousness in which the subject-object dichotomy in normal waking consciousness is substituted for a unified background awareness that is centerless and undivided. In some embodiments, an ego dissolution experience is a spontaneously occurring state of consciousness where there is a reduction in the self-referential awareness that defines normal waking consciousness, resulting in a compromised sense of “self”. In some embodiments, a unitive experience is an experience characterized by a sense of unity or “oneness” that exceeds sensory or cognitive apprehension.

At the initial and early stage of the treatment session, psychological support may be used to reduce severe and/or prolonged anxiety. Anxiety prior to or during the onset of effects may not be uncommon, and the therapists may be specially trained to recognize and actively manage subjects through such periods of anxiety until the subject is comfortable enough to continue on their own. In some embodiments, therapists validate the subject's feelings of anxiety without providing interpretations of perceptual disturbances or guiding subjects towards a particular image or memory, other than encouraging them to stay relaxed and open to the emergent experiences. For example, in some embodiments, the therapist may help alleviate anxiety using a grounding exercise. In such an exercise, the subject may be encouraged to pay attention to the sounds around them or to sensations on their skin when touching the bed/couch, ground, or other objects.

At the initial and early stage of the treatment session, the therapist may encourage the subject to lie down, practice relaxation and breathing exercises, and/or listen to calming music. In some embodiments, the therapist may remind the subject of the intention for the treatment session. For example, the therapist may ask the subject “What does feeling better or recovery feel like?” or any number of similar questions. Such reminders prior to the onset of or at the onset of effects provide an implicit direction for the subjective experience during the treatment session. In some embodiments, the therapist may remind the subject that their primary task during this session is to simply collect new and interesting experiences which can then be discussed with the therapist after the session. The therapist may remind the participant of the purpose of the therapy and the role of experiential processing, namely allowing the participant to be open and curious to whatever arises and encountering thoughts and feelings previously unknown to them. In some embodiments, the therapist emphasizes that this process inherently requires letting go and a willing passivity to the psychedelic experience.

During the acute onset of action, the subject might experience perceptual changes in visual, auditory or olfactory modes, and a range of unusual physical sensations. These experiences could be anxiety-provoking. In some embodiments, the therapist may practice reassuring “arm holding”. This is where, upon the subject's request, a therapist will place his or her hand on the subject's wrist, arm, hand, or shoulder, as a way of helping the subject feel secure during this phase. This exercise may have been previously practiced during the pre-administration psychological support session.

In some embodiments, the therapist may encourage the subject to put on an eye mask, such as a Mindfold eyeshade. In some embodiments, the therapist encourages the subject to put on the eye mask before, during, or after the onset of the effects.

In some embodiments, the therapist may encourage the subject to put on headphones and listen to music. In some embodiments, the headphones reduce outside noise (e.g., “noise-cancelling” headphones). In some embodiments, the music is calming music such as instrumental (e.g., classical) music. In some embodiments, the music comprises nature sounds and/or the sound of moving water (e.g., ocean sounds). In some embodiments, the music comprises isochronic tones. In some embodiments, the music comprises moments of silence. In some embodiments, the music is emotionally evocative. In some embodiments, the music comprises a playlist which mirrors the pharmacodynamics of a typical high-dose treatment session: the initial stage, the early stage, the peak stage, and the late stage. In some embodiments, listening to music helps the subject to focus on their internal experience.

In case of prolonged anxiety or distress, therapists may, in some embodiments, actively guide participants through such experiences without interpreting or judging the experiences or giving advice. Once participants are comfortable, the therapist may encourage them to again engage in introspection.

During the peak and late stages of the treatment session, the therapist may encourage subjects to face and explore their experience, including the challenging ones. Therapists may direct subjects to participate self-directed inquiry and experiential processing to develop a different perspective on their personal challenges and conflicts, and to generate their own solutions. Such self-generated insights are not only therapeutic because of the emotional resolution, but also empowering to subjects.

As used herein, the term “self-directed inquiry” refers to directing attention to internal states. Subjects are encouraged to be curious about experiences in the present moment, including foreground and background thoughts, emotions, and physical sensations. During the preparation and integration stages, this inquiry might mean asking specific and detailed questions to help direct attention to internal states. However, during the period of drug action, inquiry might simply mean an attitude of openness to inner experiences.

As used herein, “experiential processing” refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the therapist will employ a transdiagnostic therapy. In some embodiments, the transdiagnostic therapy is a Method of Levels (MOL) therapy. In still further embodiments, the MOL therapy comprises Self-Directed Enquiry and Experiential Processing. Typically, MOL uses brief, but detailed, curious questioning to help subjects shift and sustain their attention towards different levels of cognition and emotions. The emphasis within MOL is on identifying and working with a subject's underlying distress as opposed to just their symptoms. Such MOL related methods and techniques can include: (1) Self-directed enquiry-directing attention to internal states. Participants are encouraged to be curious about experiences in the present moment, including foreground and background thoughts, emotions, and physical sensations; during the preparation and integration stages, such enquiry can mean asking specific and detailed questions to help direct attention to internal states, although for some embodiments, during the period of drug action, enquiry can refer to an attitude of openness to inner experiences; and (2) Experiential processing-sustained focus on the experience; which refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the psychological support comprises mindfulness-based therapy or cognitive behavioral therapy (CBT). In some embodiments, the psychological support is informed by a functional theory of human behavior called Perceptual Control Theory.

Occasionally, the subject will try to avoid emerging experiences or distract him/herself while trying to regain cognitive control over the unusual state of their mind. Such distractions may take different forms. For example, the subject might want to engage in a conversation or prematurely describe in detail their experience, visions or insights. When this occurs, the therapist may aim to remain as silent as possible, thereby enabling the subject and his/her inner experience to direct the course of the treatment session. In some embodiments, the therapist may use active listening skills paired with prompts to encourage the subject to continue focusing attention on present experiences, particularly if the participant engages the therapist in conversation. In another example, a subject might ask to go to the bathroom or have a drink of water. The sudden and urgent character of such requests might suggest that they are really trying to avoid emerging material. In such cases, the therapist may encourage the subject to stay with the experience by simply redirecting their attention. For example, the therapist may say something like, “We will take a bathroom break at the end of this piece of music” or “I will get you water in a little while. Why don't you put the eye shades back on and relax for a few minutes?” If the subject is trying to avoid a difficult experience, they might listen to the suggestion and relax.

In some embodiments, spontaneous movement such as shaking, stretching or dancing while engaging with the experience is accepted and often encouraged, unless the movement seems to be a way to distract oneself from the experience. In some embodiments, if the subject continues to move around a lot, reminders to periodically return to a lying down position and to actively focus inwards may be provided.

The therapist is not required to understand, support or even have an opinion about the nature or content of the subject's experiences, but the therapist may validate them and convey openness toward the subject's own view of them without dismissing or pathologizing any experience based on its unusual content. These experiences may provide the subject with a perspective that goes beyond identification with their personal narrative. In some embodiments, the therapist will validate one or more of the subject's experiences. In some embodiments, validation of the experiences simply means acknowledging the courage of opening up to the experience and the possibility that any experience will serve the intention of the session.

In some embodiments, a therapist provides psychological support for about 0.15 hours, about 0.25 hours, about 0.5 hours, about 0.75 hours, about 1.0 hours, about 1.25 hours, about 1.5 hours, about 2 hours, about 3 hours, or any range therebetween, or longer, immediately after administration. In some embodiments, the therapist uses guided imagery and/or breathing exercises to calm the subject and/or focus the subject's attention. In some embodiments, the therapist holds the hand, arm, or shoulder of the subject. In some embodiments, the therapist counsels the subject to do one or more of the following: (1) to accept feelings of anxiety, (2) to allow the experience to unfold naturally, (3) to avoid psychologically resisting the experience, (4) to relax, and/or (5) to explore the subject's own mental space.

In some embodiments, the therapist avoids initiating conversation with the subject, but responds if the subject initiates conversation. Typically, active intervention is kept to a minimum during the treatment experience. In some embodiments, the subject is encouraged to explore their own mental space, and simple guided imagery may be used to assist relaxation. “Guided imagery” refers to an exercise wherein the subject is asked to imagine a scene (e.g., “Invite a scene, perhaps a landscape, and tell me where you find yourself”; “Imagine a place that feels safe to you.”).

### Post-Administration Psychological Support Session (“Integration Session”)

In some embodiments, subjects may be encouraged to engage in post-administration integration sessions with their therapist. Integration is a process that involves processing, or embodying, a psychedelic experience within a therapeutic context. The process initially begins by the subject verbalizing and reflecting upon any experience from the treatment session, and discussing it openly with their therapist. Successful integration of a therapy experience accommodates for emotional changes and comprises of translating experiences into new insights, perspectives, and subsequently new behaviors that can be used to benefit the subject's quality of life. New perspectives might in turn influence the participant's current knowledge or values and lead to new ways of relating to cognitions, emotions, behaviors and physical experiences.

In some embodiments, the goals and supportive methods used by the therapist throughout integration sessions should remain consistent, regardless of the intensity or content of the subjective experience explored by the subject. That said, the methods of support used by the therapist should accommodate for the full range of experiences a subject might have faced.

The integration process is not one that should be limited to the sessions with the therapist, and is a process that will likely continue to unfold beyond the visits in clinic. The therapist might encourage the participant to use methods such as spending time in nature, exercise, or creative expression to help facilitate the process further. The subject might also be encouraged to discuss experiences with their friends, family, and/or support network. The role of the integration sessions is not to cover and work on every experience, but to empower the participant by building their capacity to experientially process information safely. This enables the participant to continue self-directed integration, even outside of study visits.

In some embodiments, the subject participates in at least one psychological support session after administration (“post-administration psychological support session” or “integration session”). In some embodiments, a post-administration psychological support session may be held on the same day as the treatment session, after the effects of the drug (e.g., a compound of Formula (I) through (III)) have substantially worn off. In some embodiments, a post-administration psychological support session may be held the day after the treatment session. In some embodiments, a post-administration psychological support session may be held two days after the treatment session. In some embodiments, a post-administration psychological support session may be held three days after the treatment session. In some embodiments, a post-administration psychological support session may be held about one week after the treatment session. In some embodiments, a post-administration psychological support session may be held about two weeks after the treatment session. In some embodiments, a post-administration psychological support session may be held about one month after the treatment session. In some embodiments, a post-administration psychological support session may be held about three months after the treatment session. In some embodiments, a post-administration psychological support session may be held about six months after the treatment session. In some embodiments, a post-administration psychological support session may be held about twelve months after the treatment session. In some embodiments, post-administration psychological support session may be held in a time range in between any of the preceding times.

In some embodiments, the subject may participate in one, two, three, four, five, six, seven, or eight post-administration psychological support sessions. In some embodiments, the subject may participate in at least two, or at least three post-administration psychological support sessions.

The post-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the post-administration psychological support session(s).

In some embodiments, the post-administration psychological support session may focus on integration of the treatment experience. Integration may involve processing a psychedelic experience in a therapeutic context. Integration may comprise psychological and somatic processing of the experience and a successful assimilation of insights into the subject's life for the purpose of growth, healing and/or well-being. During an integration session, a subject may be encouraged to talk about and reflect upon their experiences during the treatment session. In some embodiments, integration may comprise an external expression of the treatment experience, such as choice of words, tone of voice, gestures, and/or particular physical activities (yoga, exercise, bodywork, etc.). In some embodiments, integration comprises creatively expressing any insights or experiences gained during a treatment experience, for example through poetry, art, music/singing, dance, writing or drawing. In some embodiments, the subject may be encouraged to reflect on both the thoughts and the feelings that he or she underwent during the treatment session, as well as to express those ideas and emotions into a concrete form that can serve as a tool for continuing to remember and integrate those lessons into the future. In some embodiments, the subject may be encouraged to acknowledge and connect with the range of the emotional cognitive and physical experiences of the treatment session and relate them to current experiences in their life situation. This may be accomplished, for example, by discussing them initially with their therapist, and perhaps later with their family, friends, and support circle. Integration helps accommodate changes in emotional states as new insights are generated and integrated. When further explored through oscillating attention between foreground and background thoughts and emotions, such insights may lead to natural and effortless changes in perspectives or behaviors. In some embodiments, the integration process is not limited to initial integration meetings with the therapist, but continues to unfold spontaneously through a participant's own processing and actions in every day life.

In the case of a low-intensity experience, the integration process might focus on the mental content that emerged during the hours of relaxation and introspection. This might also include reactions to what might have been an unremarkable experience, such as feeling of disappointment, anger, relief, etc.

### EMBARK

In some embodiments, the psychological support administered to the subject is a six-domain framework for psychedelic-assisted psychotherapy (PAP) referred to as EMBARK, which is a transdiagnostic, trans-drug model. The six clinical domains of EMBARK are: (1) Existential-Spiritual, (2) Mindfulness, (3) Body Aware, (4) Affective-Cognitive, (5) Relational, and (6) Keeping Momentum.

(1) Existential-Spiritual. Psychedelic medicines are well known to catalyze profound encounters with mystical or spiritual content and existential concerns, such as mortality, alienation, or questions of life meaning. Several PAP clinical trials have found that participants often report profound experiences of an existential or spiritual nature that may hold enduring significance for them. The potency of participants' mystical experiences has been found to correlate with a range of treatment benefits, including reductions in symptoms of depression and treatment-resistant depression, increased motivation to stop problematic cocaine use, decreases in cancer-related depression and anxiety, greater success in nicotine cessation, and other positive changes in psychological functioning. Despite calls for further elucidation of specific therapeutic mechanisms in this domain, existential and spiritual elements of PAP warrant recognition as potential sources of treatment benefit. The current state of knowledge suggests that anti-depressive outcomes may be facilitated simply by having a mystical experience during a medicine session. However, providing support for a participant's post-medicine spiritual self-development may contribute additional benefit, as suggested by prior PAP research and non-psychedelic findings of negative correlations between spirituality and depressive symptoms. The role of EMBARK therapists by way of this domain is thus to create the conditions for mystical or spiritual phenomena to potentially arise and to support participants in using them as an impetus for spiritual growth. In the preparation phase, therapists assess for any intrinsic motivation a participant may have to bring existential-spiritual elements into their treatment and work with them in developing this motivation into their intentions for the medicine session. For the medicine session, therapists prepare the physical treatment space in a way that demonstrates respect for the subjective sense of sacredness that may arise for the participant and open the session with a brief, collaboratively designed ritual. If phenomena in this domain arise during the medicine session, therapists are invited to use supportive psychotherapy or evidence-based interventions of their preference during the integration phase to explore the participant's experience, support them in developing a sense of what actions it could motivate, and co-create plans for life changes and/or further spiritual self-development. Examples of approaches with evidentiary bases to use in this process include meaning-oriented psychotherapies, Logotherapy, or Spiritual Guidance, which is derived from the evidence-based approach of motivational interviewing (MI), despite not being an Evidence-Based Therapy (EBT) itself. Throughout all phases, therapists are taught to attend to their own biases and beliefs in order to avoid imposing their own understanding or interpretation on a participant's experience in this domain.

(2) Mindfulness. This domain refers to treatment events that result in the participant becoming more capable of recognizing symptomatic internal states and responding to them with a greater capacity for self-compassion and self-regulation. Mindfulness in EMBARK has significant conceptual overlap with the notion of “psychological flexibility” that the ACE Model derives from Acceptance and Commitment Therapy (ACT). In previous PAP trials, participants have often experienced various forms of disruption of their habitual self, a sense of “mental freedom,” or an increased feeling of sovereignty in how one relates to the workings of their own mind. Overall, the “M” domain represents a place in EMBARK for preparing the participant to attend to their thoughts and feelings with compassion during a medicine session and for working with increases in psychological flexibility and other metacognitive shifts during integration. Mindfulness has been found helpful in disrupting ruminative thought patterns, enabling more cognitive flexibility, and fostering a more compassionate stance toward oneself. To support these outcomes, EMBARK therapists begin a course of PAP treatment by teaching basic mindfulness skills to the participant. This teaching is only meant to ensure that the participant knows how to attend to their internal experience during the medicine session, which is when a more enduringly increased capacity for mindfulness may arise. During the integration phase, therapists help to anchor whatever aspect of mindfulness arose for the participant through the use of indication-specific mindfulness practices. Therapists may also help the participant integrate a new feeling of self-compassion or support them in using the momentary abatement of ruminative thoughts as an opportunity to develop mindfulness-based skills that may prevent a recurrence. Therapists can use their own mindfulness-based interventions within the bounds of the guidelines provided, or they can use suggested interventions drawn from Rumination-Focused Cognitive-Behavioral Therapy (RF-CBT) Mindfulness-Based Cognitive Therapy (MBCT) included in the treatment manual. At all times, therapists are taught to work with the participant in this domain within a trauma-informed approach to mindfulness and to avoid imposing one's own beliefs or biases onto the participant's experience.

(3) Body Aware. PAP participants have reported that embodied phenomena are a notable part of their experience of a medicine session. There are few EBTs that provide support in conceptualizing or responding to these phenomena, though innovative somatic psychotherapy approaches have offered suggestions. EMBARK therapists are prepared to respond to embodied treatment events using the most widely accepted elements of these novel somatic approaches, such as “pendulation,” or the process of helping a participant alternate between active, embodied engagement with trauma material and self-soothing. EMBARK also encourages integration of somatic elements from more established EBTs, such as somatic awareness training exercises and self-regulation skills from Mindfulness-Based Relapse Prevention (MBRP) or Dialectical Behavioral Therapy (DBT) to support therapeutic outcomes in this domain. The techniques used by EMBARK therapists in this domain do not require them to work with the body in an intensive, hands-on way and are thus not prohibitively far beyond their standard psychotherapeutic training. In preparation, therapists train the participant in basic somatic awareness so that they can attend to and thereby facilitate pro-therapeutic bodily phenomena during the medicine session. Once the medicine is administered, the therapists' role is to guide the participant back toward an awareness of their body when clinically indicated and help them remain within their zone of optimal arousal through the use of the self-soothing interventions or therapist-participant touch-based interventions. There remains a lack of knowledge or consensus around the efficacy, safety, or necessity of therapist-participant touch in PAP and thus EMBARK therapists are instructed to prioritize non-touch interventions and limit their touch-based interventions to basic supportive touch that minimizes points of contact between parties, like handholding or placing a hand on a participant's shoulder to convey support or offer grounding. More intensive forms of touch (e.g., full-body embraces) are omitted until further research establishes them as safe, effective interventions. Therapists also rigorously assess for the participant's consent to touch-based interventions during preparation and proactively give participants a chance to reject any form of touch during preparation, immediately before the provision of touch in the medicine session, and at any time during the touch, so as not to move beyond the participant's espoused level of comfort. The primary distinction between the Body aware and Mindfulness domains is found less in their associated interventions than in participants' subjective experiences of how benefits arise and the integration goals that best support these benefits. Body aware treatment goals follow from medicine session events that a participant locates in their body and involve integration practices that work with shifts in somatic awareness. Treatment goals in the Mindfulness domain involve benefits that adhere more closely to the core concepts of psychological flexibility and other metacognitive shifts and are sustained with practices that build upon a participant's revised relationship with the contents of their mind. In the treatment of depression such as MDD, somatic phenomena are hypothesized to contribute to therapeutic outcomes in two ways. Depressive symptoms are notable for their disturbance of embodiment (e.g., fatigue or energy loss, disruptions of sleep and appetite, weight gain or loss). It has been suggested that disruptions of one's lived experience of their body are the most fundamental and cross-culturally consistent MDD phenomena. As such, the experiences of enlivenment and sensory embodiment that some PAP participants describe may form the basis for a relationship with one's body that opposes the recurrence of depressive symptoms. If such an experience arises for a participant, therapists may work with them to build and strengthen this new relationship. Additionally, it is likely that treating depression will often involve working with trauma. The comorbidity and symptomology of MDD and trauma has been posited as evidence that many cases of MDD can be meaningfully thought of as a subtype of PTSD characterized by an internalizing response to trauma. Although classic psychedelics have not yet been used to address trauma in a completed clinical trial, it has been suggested that the disinhibiting effect of psilocybin on frontal-limbic neural circuits of emotional regulation are similar to those of MDMA, which has more established efficacy in treating trauma. Classic psychedelics may thus have facility in trauma treatment by way of similar disinhibitory mechanisms. It is likely that PAP treatment of depression such as MDD with a classic psychedelic will sometimes facilitate the appearance of trauma symptoms, possibly in the form of the somatic phenomena described earlier (nausea, shaking, etc.). When this occurs, a skillful therapeutic response may facilitate lasting resolution.

(4) Affective-Cognitive. During a medicine session, some PAP participants experience dramatic shifts in their emotions and cognition. Participants have often reported that they experience a degree of emotionality that is ordinarily unavailable to them, including feelings of bliss, love, despair, fear, and grief, often in the context of a healing catharsis. They may also find themselves confronting maladaptive self-beliefs with a directness they would normally avoid. Engaging non-defensively with these emotions and beliefs during a medicine session has become a widely accepted practice among PAP models. This practice mirrors that found in several non-psychedelic EBTs, such as ACT's emphasis on acceptance and Emotion-Focused Therapy's (EFT) emphasis on entering into maladaptive states in service of transformation. This domain serves as a space within EMBARK for incorporating approach-oriented practices for working with emotions and self-beliefs in way that best addresses the indication being studied. For example, the EMBARK approach to depression (e.g., MDD) in this domain begins with the notion that many depressed individuals have developed a habitual response to challenging feelings that entails dimming their awareness of them through a kind of automatic, unconscious avoidance and characteristic depressive experience of feeling numb and withdrawn. Participants in PAP medicine sessions have often experienced a greater facility in reconnecting with this avoided material and have implicated these experiences in their positive treatment outcomes. In the preparation phase, EMBARK therapists are tasked with helping participants understand the importance of adopting an approach orientation during the medicine session. They also help the participants develop one or more self-soothing techniques. During the medicine session, the therapists' role is to remind the participant to welcome challenging content if needed and to help them utilize the previously learned self-soothing techniques when necessary. To integrate these experiences, EMBARK therapists help participants use experiences of approaching challenging material as the basis for updating maladaptive core beliefs about oneself or the world and cultivating an enduring attitude of greater acceptance in their emotional life. All of these interventions are taught and employed in a way that is respectful of participant autonomy, the principles of trauma-informed care, and cultural differences in relating to and expressing one's emotions.

(5) Relational. Practitioner-participant interactions are often significant events in medicine sessions that have yet to be adequately characterized by other PAP models. These interactions may be home to amplified versions of dynamics that are typically found in any relational form of psychotherapy, including transferences, projection, or reenactments of traumatizing dynamics or events. As in talk therapy, the appearance of these phenomena represents an opportunity for clinical gain if handled skillfully or rupture and harm if handled poorly. EMBARK therapists are thus trained to work ethically and efficaciously in this domain through the didactic and experiential processes discussed later under the Ethically rigorous care cornerstone. In the treatment of depression such as MDD, for example, these relational events are framed as potential moments of relational repatterning that may reduce depressive symptoms. A core element of depression is social isolation, both actual and felt. This isolation may derive from patterns or beliefs learned in early life relationships, such as a sense that one is unacceptable, deserves to be alone, or may lose love if they express themselves freely in the presence of another person. The altered relational dynamics of a PAP medicine session may provide opportunities for relational repatterning that supplants these maladaptive beliefs. EMBARK therapists are also prepared to support relational benefits that may arise for the participant outside of their interactions with the therapists, such as an internally felt sense of social connectedness or emotional empathy or an autobiographical review process that examines past and present relationships in the participant's life. If these shifts in social cognition occur and are skillfully worked with in the integration phase, a depressed participant can be helped to use them as the grounds for a less isolative social life.

(6) Keeping Momentum. A course of PAP treatment is brief, and its most enduring benefits have been conceptualized as those that continue to unfold well beyond the final session. PAP participants often emerge from a medicine session with a sharp uptick in the sense of motivation, self-efficacy, and commitment to making pro-therapeutic changes to their behavior or life context. Some may also develop a clarified sense of their deeply held values, which may form the basis for beneficial post-treatment actions. At the neural level, it has been suggested that the plasticity brought about by many psychedelic medicines may signal the reopening of a social reward critical learning period for weeks after administration, suggesting that pro-therapeutic changes may take root during a time period that extends beyond the end of what is typically considered integration. The EMBARK approach recognizes this unique opportunity. Therapists are trained to support a participant's movement from the setting of intentions to the planning of concrete actions by attending to importance of post-treatment changes throughout all stages of PAP treatment. For depression (e.g., MDD), anxiety, or any other indication under study, these changes are expected to look very different for each participant. EMBARK therapists are trained to apply a broad lens to what helpful post-treatment change might look like. For some participants, the most supportive change may be at the level of personal behaviors, such as problem drinking or procrastination. For others, change may be warranted in their personal contexts, such as relationships or work environments. Some participants may find additional benefit in taking aim at collective concerns that have a bearing on their life, such as structural racism or exploitative work conditions, through participation in collective organizing. The EMBARK approach recognizes the potential of both individual and collective forms of change in the service of enhancing a participant's psychological, spiritual, and social wellbeing.

The EMBARK model adheres to the three-phase PAP treatment design that has been used in all clinical trials published to date. It involves non-drug preparation sessions prior to administration (Pre-Administration Psychological Support Sessions), medicine sessions in which the psychedelic medicine is administered (Psychological Support During Treatment Sessions), and non-drug integration sessions after the date of administration (Post-Administration Psychological Support Sessions).

For the sessions within each phase, therapists are given a set of general tasks, as well as domain-specific tasks for each of the six domains. For the preparation and medicine phases, these two sets of tasks are woven together into suggested agendas for each session, which therapists can apply with flexibility and responsiveness to the needs of a specific participant. In the integration phase, therapists and participants collaboratively choose which integration goals they will pursue based on their pertinence to what arose in the participant's medicine session. Each indication-specific EMBARK treatment manual provides guidance on which integration goals to consider with the participant based on the specific treatment events that arose during the medicine session. The way in which EMBARK's eclectic, six-domain approach comes together into a unified approach across the three phases of treatment is described below.

Preparation Sessions. All EMBARK protocols developed so far have included three preparation sessions leading up to each medicine session, though this may change if called for by the indication under study. The therapists' general aims for this phase have included building rapport and trust, learning about the participant's experience of their mental health challenges, explaining basic elements of PAP treatment and what to expect from the medicine session, providing preparatory instructions about diet and aftercare, and responding to participant questions about PAP treatment. Therapists are also given domain-specific tasks in the preparation phase, which may vary across protocols in response to the specific proposed mechanisms of change for the clinical indication under study. These tasks lay the groundwork for the participant to receive benefit within whichever of the six domains become important for them during the medicine and integration phases. Every participant is prepared for potential benefit across all six domains because EMBARK therapists make no determination before the medicine session about which domains may or may not become relevant later in treatment, despite any prediction or wish held by them or the participant during this phase. Even if a participant frames their disease or disorder in, for example, primarily spiritual terms and expresses a desire to address their symptoms within a spiritual framing, it is still very possible that treatment benefits will occur for them in other domains, in addition to or instead of the “E” domain, if they are properly prepared. During preparation, the participant's lived experience of their symptoms and their functional significance are assessed, but no attempt is made by therapists to use this information to focus the participant's treatment on a specific subset of psychedelic-induced phenomena during the medicine session. However, this information is taken into consideration later during the integration phase when deciding what integration goals would be most supportive. Since it is unknown during the preparation phase which domains and domain-associated mechanisms of change will be most relevant to the specific participant, therapists' role in this phase tends to be more standardized than it is in later phases. However, while preparation tasks are fixed in their intent, therapists are invited to bring in interventions from their preferred clinical orientation(s), as long as they conform to the guidelines that ensure that the intended utility of each task is conferred to the participant. For example, therapists tasked with teaching basic mindfulness skills to a participant may do so using mindfulness-based tools from ACT, DBT, other mindfulness-based EBTs, or a meditative spiritual tradition, provided that the tool meets the following criteria set forth in the EMBARK manual: (1) it invites the participant to cultivate a receptive, attentive state, (2) does not contain elements that could potentially clash with a participant's religious beliefs, (3) and abides by trauma-informed practices detailed in the manual. The manual also provides example interventions for therapists who do not have relevant expertise to bring in for any given task. Ethical considerations based on the four care cornerstones are woven throughout the guidelines set forth for therapist interventions. For example, the guidelines for the discussion of therapist-participant dynamics requires that it include an exploration of cultural dynamics, in line with the cornerstone of culturally competent care.

Medicine Sessions. At the outset of a medicine session, the therapists maintain an agnosticism about which benefits and which domains will ultimately become most salient for a participant, much as they did in the preparation phase. The pre-dosing therapist tasks thus serve a similar purpose to the tasks in the preparation phase in that they set the stage for phenomena in any domain to arise and bring the potential for benefit. These stage-setting tasks are woven together into a suggested pre-dosing agenda for the medicine phase that includes a collaborative ritual (E), a check-in about intentions (K), and a brief somatic awareness practice (B). The six EMBARK domains thus continue to serve therapists as a conceptual frame for laying the groundwork for a broad variety of pro-therapeutic outcomes. However, once a psychedelic medicine is administered, the therapists' role becomes more responsive to the specific situation and less about a domain-agnostic approach to preparation. At this point, the course of a participant's treatment starts to come into focus, and specific EMBARK domains present themselves as more pertinent to the participant's experience of the medicine. EMBARK's six-domain framework comes to serve therapists in a new way as a practical rubric for helping them to characterize in-session events and determine what domain-specific interventions these events might call for. For example, if they observe the participant experiencing what they identify as a somatic trauma process, they can recall the training they received in the Body aware domain, and apply the interventions associated with it. Support in identifying and responding to these events is provided in the EMBARK training program and in each indication-specific EMBARK manual.

The value of organizing the preparation work by domains becomes clear during this phase, as many of the responsive interventions applied in a particular domain will draw upon work that was conducted with the participant during the preparation phase in that same domain. For example, a reminder to “move toward” challenging emotional material will draw from preparatory psychoeducation provided in the Affective-cognitive domain, or a reminder to use a somatic self-soothing technique in response to intense somatic events will rely on what was taught during preparation in the Body aware domain. Therapists' incorporation of their own favored interventions during the preparation phase also ensures that they will be working squarely within their competence during this more unpredictable phase as well.

Integration Sessions. At this point, the therapists and participant are likely to have a strong sense of the most beneficial direction for a participant's continued therapeutic progress. During the first debrief session, all parties collaboratively determine what treatment goals might be best to work toward in integration. This process entails (1) debriefing and supporting the participant's sense of what transpired in the medicine session, (2) relating material that arose in the medicine session to their symptoms and treatment goals, (3) collaboratively identifying new attitudes, beliefs, behaviors, values, or other subjective shifts that may contribute to symptom reduction, and (4) planning post-treatment changes that may support and sustain this outcome. A set of suggested integration goals are provided in each indication-specific manual, along with guidelines and suggestions for working toward each of these goals. These goals are organized by domain to provide continuity with events that arose in the medicine session. Together, therapists and participants choose a subset of these goals, or develop their own, as long as they are based on a clear clinical rationale. This selection process is guided primarily by what transpired in the medicine session using a tool provided in each EMBARK manual. For instance, a participant who had an experience that they identify as spiritual may benefit from support in advancing their spiritual self-development or spiritual practices (E), or a participant who had a strong emotional opening might be best served by continued processing and reflection that may lead to revised core beliefs or a sustained movement away from emotional avoidance (A). However, the selection process is also informed by the participant's previously stated intentions for treatment and the functional meanings of their symptoms. For instance, if a depressed participant initially framed their suffering in terms of loneliness and set an intention of understanding their isolation, it might benefit them to consider goals in the Relational domain, even if nothing observable transpired in that domain during the medicine session. All integration goals are framed in terms of three possible spheres of change: individual behavior, personal context, and broader context. Individual behavior changes may include stopping a maladaptive behavior, changing an old behavior, or adopting a new practice. Personal context changes may include updates to social, vocational, or physical contexts that support treatment benefit, such as moving away from a social circle that encourages problem drinking or moving into a vocational field more congruent with one's revised personal values. The broader context refers to structural, cultural, or economic conditions that have real, mental health consequences for the individual. Participants who decide that change at this level would be supportive for them may benefit from taking collective action (e.g., community activism, labor organizing) that addresses broader conditions in a way that feels congruent with their revised values or sense of self and/or serve as a form of socialization or behavioral activation. This may have acute benefit for them while also contributing to the amelioration the conditions that had engendered or exacerbated their distress in the first place (e.g., exploitative work conditions). Participants and therapists determine together which of the three spheres might be appropriate foci for post-treatment changes that support participant wellbeing. The number of integration sessions is left to the discretion of the group employing EMBARK, though all EMBARK manuals written to date have included a standard of three integration sessions per medicine session. It is suggested that, whatever the intended number may be, therapists and participants both use their judgment to determine if additional sessions would be supportive of the participant's wellbeing and to undergo these sessions whenever possible. Otherwise, therapists working within the EMBARK approach are required to be prepared to make appropriate outside referrals if a participant requires ongoing therapeutic support. While EMBARK was designed to support short-term PAP interventions in clinical trial settings, aftercare may also be considered or needed to properly support the patient after their participation in the trial or other treatment settings.

### Psychological Support Provided Remotely

In some embodiments, psychological support may be provided remotely to a subject. For example, a therapist providing psychological support may not be in the same room, the same building, or in the same facility as a subject. Remote psychological support may be provided, for example by telephone (i.e., by voice call), by video call or video conference, by text, or by email.

In some embodiments, a pre-administration therapy session is conducted remotely. In some embodiments, a post-administration therapy session (e.g., an integration session) is conducted remotely.

In some embodiments, psychological support is provided remotely during the subject's treatment session. For example, in some embodiments, the subject is administered the drug in his or her own home, and a therapist provides psychological support by voice call, video call, text, email, etc., for a time period (e.g., about 0.15 hours, about 0.25 hours, about 0.5 hours, about 0.75 hours, about 1.0 hours, about 1.25 hours, about 1.5 hours, about 2 hours, about 3 hours, or any range therebetween, or longer) after the subject has taken the drug. In some embodiments, the subject is administered the drug in an administration facility as described herein, and the therapist provides psychological support to the subject a therapist provides psychological support by voice call, video call, text, email, etc., for a time period (e.g., about 0.15 hours, about 0.25 hours, about 0.5 hours, about 0.75 hours, about 1.0 hours, about 1.25 hours, about 1.5 hours, about 2 hours, about 3 hours, or any range therebetween, or longer) after the subject has taken the drug.

In some embodiments, remote psychological support is provided to the subject using a digital or electronic system. In some embodiments, the digital or electronic system may comprise one or more of the following features: 1) The digital or electronic system securely connects patients with one or more therapists or physicians for “virtual visits.” These virtual visits may be introductory or routine; 2) The digital or electronic system allows a subject to qualify, prequalify, or register for a clinical trial, or a psychological support session; 3) The digital or electronic system is configured to help therapists and/or physicians manage and interact with patients, for example, the electronic system may allow the therapist to share documents with subjects, keep notes about sessions, or schedule future sessions; 4) The digital or electronic system is configured to provide alerts for crisis intervention, for example, the digital or electronic system may allow the subject to contact the therapist if they are feeling anxiety or otherwise urgently need to talk to the therapist; 5) The digital or electronic system is configured to help prepare the subject for a visit with their therapist and/or physician, for example, the digital or electronic system may contain information regarding drug (e.g., psilocybin or a deuterated form thereof), the therapeutic protocol, etc.; 6) The digital or electronic system is configured to allow the therapist to provide psychological support during the subject's treatment session, for example, the system may comprise a video calling or chat feature; 7) The digital or electronic system is configured to allow the therapist to provide psychological support during a post-administration session (e.g., an integration session); 8) The digital or electronic system is configured to track the subject's adherence to the treatment regimen or goals; 9) The digital or electronic system is configured to assess one or more clinical endpoints in the subject, for example the system may comprise one or more questionnaires or exercises for the subject to complete, and the results may be made available to the subject's physician and/or therapist.

In some embodiments, the digital or electronic system is an “app” for use on a mobile phone or a computer. In some embodiments, the digital or electronic system is a website. In some embodiments, the digital or electronic system comprises a “chat” feature which allows communication between the subject and the therapist in real time. In some embodiments, the website comprises a video calling feature, which allows for the therapist to communicate with the subject using video communication. In some embodiments, the digital or electronic system is configured to allow a single therapist to provide psychological support to one or more subjects at or around the same time.

In some embodiments, psychological support sessions may be pre-recorded (e.g., audio or video recording) and provided to the subject for use at the subject's convenience via the digital or electronic system.

### Administration Facility, “Set and Setting”

As used herein, the term “set and setting” refers to the subject's mindset (“set”) and the physical and social environment (“setting”) in which the user has the treatment session. In some embodiments, the pharmaceutical formulation may be administered in a particular set and setting. In some embodiments, the set and setting is controlled, to the extent possible, to maximize therapeutic benefit of the treatment session.

In some embodiments, administration is performed in a facility specifically designed for treatment. Administration to the subject in a facility where the subject feels safe and comfortable may help ease anxiety in the subject, and may facilitate maximum clinical benefit. Administration may be performed, for example, in the subject's home or at a clinical facility.

In some embodiments, administration is performed in a facility (e.g., a room) with a substantially non-clinical appearance, for example, in a room that comprises soft furniture (e.g., plush couches, chairs, or pillows) and/or plants. In some embodiments, the room may be decorated using muted colors (e.g., greyed, dulled, or desaturated colors). In some embodiments, the light in the room is dimmed and/or light levels are kept or adjusted to be relatively low. In some embodiments, the room lighting is adjusted for intensity and/or color. In some embodiments, a virtual reality or augmented reality system (e.g., computer with visual/graphical and auditory outputs) is used. In some embodiments, the room comprises a sound system, for example a high-resolution sound system. In some embodiments, the sound system can allow for simultaneous ambient and earphone listening. In some embodiments, the subject may bring meaningful photographs or objects into the administration room.

In some embodiments, the room comprises a couch. In some embodiments, the room comprises a bed. In some embodiments the room comprises more than one couch or bed, such as 2, 3, 4, 5, 6, 7, 8, 9, or 10 couches or beds. In some embodiments, the subject sits on or lies in the couch or bed for approximately 1 hour, 2 hours, 3 hours, or any range therebetween, or a substantial fraction thereof, immediately after administration. In some embodiments, the subject listens to music for approximately 1 hour, 2 hours, 3 hours, or any range therebetween, or a substantial fraction thereof, immediately after administration. In some embodiments, the subject wears an eye mask for approximately 1 hour, 2 hours, 3 hours, or any range therebetween, or a substantial fraction thereof, immediately after administration. In some embodiments, the subject is provided with a weighted blanket.

In some embodiments, each subject is supervised by one therapist during the treatment session. In some embodiments, each subject is supervised by more than one therapist during the treatment session, such as two therapists, three therapists, four therapists, or five therapists. In some embodiments, one therapist may supervise multiple subjects, wherein each subject is participating in a treatment session. For example, one therapist may supervise two, three, four, five, six, seven, eight, nine, or ten subjects.

Embodiments of the disclosure include use of additional tools and/or technique(s) with dosage/administration, including various transcranial magnetic stimulation (TMS) methods and protocols, for example, prior or subsequent to one or more dosing(s), biofeedback devices, etc.

Some embodiments can be used with a digital health product or digital solution. Teachings of the disclosure include utilization of such digital health products and/or related digital biomarkers as diagnostic and/or prognostic tools for patient monitoring and management pre-treatment, during treatment, and/or post treatment. Digital biomarkers can include, but are not limited to number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like. For example, in one embodiment, a digital health product can be utilized to determine dosing amount and/or dosing frequency, indicator of a need for re-dosing, re-dosing amount, a warning or alert, as tracking of compliance, etc.

In some embodiments, methods of treatment can include providing a clearance time for a subject or patient, such one or more medications is not present or substantially cleared from the system of the subject/patient. For example, methods of treatment can be configured such that, upon administration, the subject is not taking other serotonergic medications such as: selective-serotonin reuptake inhibitors, selective norepinephrine reuptake inhibitors, tricyclic anti-depressants, monoamine oxidase inhibitors and/or antipsychotics. In some embodiment, the method of treatment includes treatment concurrently with one or more medications, including but not limited to selective-serotonin reuptake inhibitors, selective norepinephrine reuptake inhibitors, tricyclic antidepressants, and/or monoamine oxidase inhibitors. In some embodiments, the method includes treatment such that subjects or patients take concomitant compounds or medications, including but not limited to benzodiazepines, cannabidiol (CBD) and/or other cannabinoids (e.g., THC (tetrahydrocannabinol); THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); CBT (cannabicitran); and/or the like), magnesium, levomefolic acid, e.g., for a period of time prior to, just prior to, and/or at the same time as receiving the pharmaceutical formulation of the present disclosure. In some embodiments, the methods of the present disclosure allow the patient being treated to remain on any ongoing antidepressants (e.g., SSRIs) throughout the treatment course.

In some embodiments, the method includes treatment such that a subject has not taken one or more medications, particularly has not taken one or more serotonergic medications for at least 2 days, at least, 3 days, at least 4 days, at least 5 days, at least six days, at least 1 week, at least 2, 3, or 4 weeks before administration of the pharmaceutical formulation. In some embodiments, the method includes treatment such that a subject has taken one or more medications, particularly has taken one or more serotonergic medications for at least 2 days, at least, 3 days, at least 4 days, at least 5 days, at least six days, at least 1 week, at least 2, 3, or 4 weeks before administration of the pharmaceutical formulation.

Embodiments of the disclosure include methods utilizing a digital biomarker, for example, as a diagnostic and/or prognostic tool for patient management pre-, during and/or post treatment, wherein the digital biomarker is one or more biomarkers associated with executive function, cognitive control, working memory, processing speed, and/or emotional valence.

In some embodiments the digital biomarker is identified from patterns in smartphone use such as swipes, taps, and other touchscreen activities, and can be scientifically validated to provide measurements of subject status, such as cognition and mood, including, by way of non-limiting example, as disclosed in one or more of the following, each of which is herein expressly incorporated by reference for all purposes: US20170086727, US20170258382, US20170258383, US20170287348, U.S. Ser. No. 10/148,534, U.S. Pat. No. 9,737,759, and/or U.S. Ser. No. 10/231,651.

Biomarkers which may serve as a diagnostic and/or prognostic tool for patient management pre, during and/or post treatment may be identified using one or more of: number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like. In some embodiments, health components and/or connected biomonitors and/or smart devices/wearables can be utilized to collect information to be used in diagnostic and/or prognostic outputs. For example, in some embodiments, a heart rate monitor or similar device can collect a subject's data and heart rate variability (for example only, as disclosed in U.S. Pat. No. 10,058,253, the entirety of which is herein incorporated by reference) can be used to assess/determine a metric relating to the subject's current emotional state, relative change in emotional state, etc., which can be used in determining a new or follow-on treatment plan, adjusting a treatment plan, etc.

In accordance with a further aspect of the disclosure there is provided a method of assessing a subject pre, during and/or post treatment of a central nervous system disorder to determine whether to provide treatment or a further treatment comprising monitoring one or more biomarkers associated with executive function, cognitive control, working memory, processing speed, and emotional valence, and determining the treatment based on an outcome. The method can further comprise the step of administering the pharmaceutical formulation of the present disclosure, for a first or a subsequent time.

In some embodiments, the biomarker is identified from patterns in smartphone use such as swipes, taps, and other touchscreen activities, and are scientifically validated to provide measurements of cognition and mood. For example, in some instances, the pattern is identified using one or more of: number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like.

Embodiments include a method of assessing a subject pre, during and/or post treatment of a central nervous system disorder to determine whether to provide treatment or a further treatment with the pharmaceutical formulation of the present disclosure, comprising monitoring one or more biomarkers associated with executive function, cognitive control, working memory, processing speed, and emotional valence, and determining the treatment based on an outcome; the method can further comprise administering the pharmaceutical formulation of the present disclosure for a first or a subsequent time. In some embodiments, the disclosure provides for treating 2 or more subjects, the method comprising administering to each subject a therapeutically effective amount of the pharmaceutical formulation of the present disclosure, at the same time or substantially the same time (e.g., dosed within several minutes of each other, within 5, 10, 15, 20, 25, or 30 min of each other), wherein each subject is aware of the other subject also receiving treatment. In some embodiments, the subjects are in the same room. In some embodiments, the subjects are in different rooms.

In some embodiments, the disclosure provides a method of treating a subject, the method comprising administering to the subject a therapeutically effective amount of the pharmaceutical formulation of the present disclosure, and providing a virtual reality/immersive reality digital tool. In some embodiments, the light in the room is dimmed and/or light levels are kept or adjusted to be relatively low. In some embodiments, darkened glasses or eye shades are provided. In some embodiments, the room lighting is adjusted for intensity and/or color. In some embodiments, a virtual reality or augmented reality system (e.g., computer with visual/graphical and auditory outputs) is used.

### Subjects

In some embodiments, the subject is a male. In some embodiments, the subject is a female. In some embodiments, the female subject is pregnant or post-partum. In some embodiments, the subject is attempting to reduce or eliminate their use of a pharmaceutical agent, such as an antidepressant or an anti-epileptic drug. In some embodiments, the subject is attempting to reduce or eliminate their use of the pharmaceutical agent before becoming pregnant, having surgery or other medical procedure, or starting to use a different pharmaceutical agent.

The subject may be a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age.

The subject may have a chronic disease or a terminal disease. In some embodiments, the subject may have a life-altering disease or condition (such as the loss of a limb or onset of blindness).

The subject may have recently been diagnosed with a disease, disorder, or condition. For example, the subject may have been diagnosed within 1 month, within 3 months, within 6 months, or within 1 year. In some embodiments, the subject may have been living with a disease, disorder, or condition for an extended period time, such as at least 6 months, at least 1 year, at least 3 years, at least 5 years, or at least 10 years.

In some embodiments, the subject may be a cancer patient, such as a Stage 4 or terminal cancer patient. In some embodiments, the subject may have been determined to have a limited time to live, such as less than 1 year, less than 6 months, or less than 3 months.

The subject may have previously taken a psychedelic drug, or may have never previously taken a psychedelic drug. For example, the subject may or may not have previously taken psilocybin, a psilocybin mushroom (“magic mushroom”), LSD (lysergic acid diethylamide or acid), mescaline, or DMT (N,N-dimethyltryptamine).

In some embodiments, the subject may have previously taken one or more serotonergic antidepressants (e.g., selective serotonin reuptake inhibitors (SSRIs)). In some embodiments, the subject has never previously taken a serotonergic antidepressant. In some embodiments, the subject has not taken any serotonergic antidepressants for at least 2 weeks, at least 4 weeks, or at least 6 weeks prior to receiving treatment with the pharmaceutical formulation. In some embodiments, the subject treated with the methods herein is taking an antidepressant such as a serotonergic antidepressant as part of ongoing treatment. For example, the subject to be treated herein may be taking a stable chronic dose of antidepressant medication(s). In some embodiments, the subject treated with the methods herein is taking a sedative or hypnotic as part of ongoing treatment.

In some embodiments, the subject may have previously received electroconvulsive therapy (ECT). In some embodiments, the subject has not received any ECT for at least 2 weeks, at least 4 weeks, or at least 6 weeks prior to receiving treatment with the pharmaceutical formulation.

The subject may have a medical condition that prevents the subject from receiving a particular medical therapy (such as an SSRI or ECT). In some embodiments, the subject may have previously had an adverse reaction to a particular medical therapy (such as an SSRI or ECT). In some embodiments, a prior medical therapy (such as an SSRI or ECT) was not effective in treating a disease, disorder, or condition (e.g., GAD, MDD, etc.) in the subject.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### I. Synthetic Procedures

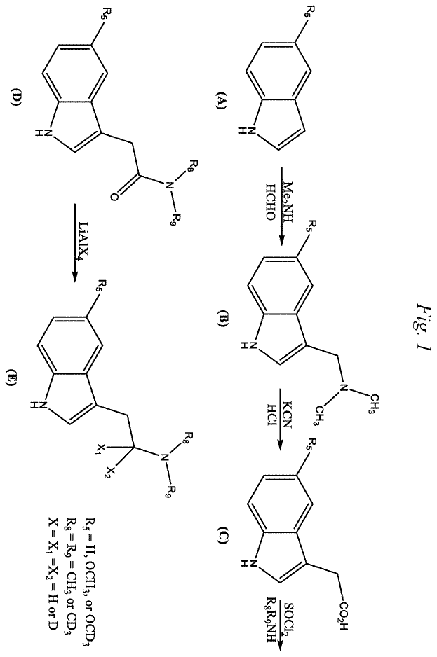

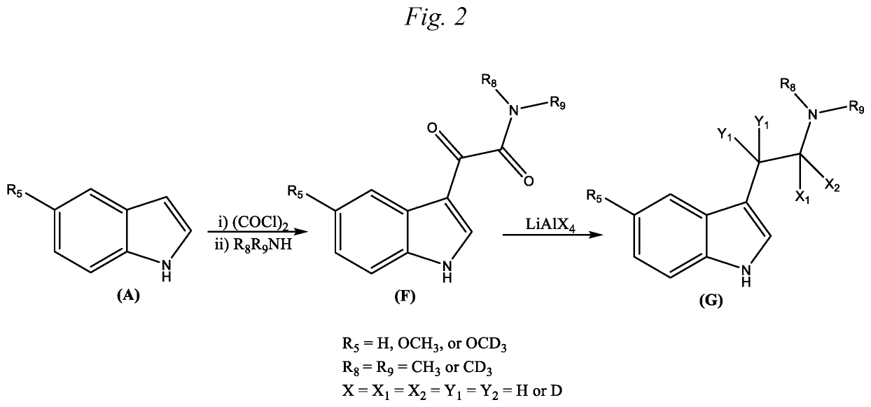

Synthetic procedures for accessing DMT, 5-MeO-DMT, deuterated analogs thereof, and salt forms thereof, are presented below and in FIGS. 1-2. See also PCT/EP2023/050702, which is incorporated herein by reference in its entirety.

### Salt forms of 2-(1H-indol-3-yl)-N,N-dimethylethan-1-amine-1,1-d₂ (I-2)

Synthesis of 2-(1H-indol-3-yl)-N,N-dimethylethan-1-amine-1,1-d₂ (I-2) was carried out according to FIG. 1. Indole (A) (R₅=H) was iminoformylated using formaldehyde/dimethylamine to produce intermediate (B), which was then converted to the 3-acetic acid intermediate (C) using potassium cyanide in HCl. Subsequent treatment with thionyl chloride and dimethylamine (R₈=R₉=CH₃) produced amide (D, R₈=R₉=CH₃), that was reduced by LiAlD₄ to yield compound I-2 (free base). The structure of the product was confirmed by ¹H NMR.

Various salt forms are prepared by crystallization of I-2 (free base) with stoichiometric (1.0 molar equivalent) quantities of the corresponding organic acid (fumaric acid, benzoic acid, salicylic acid, or succinic acid) from ethanol. The salt form identifier and salt type are provided in Table 3.

TABLE 3

| Salt form identifier | Salt type of compound |
|---|---|
| I-2a | Fumarate of I-2 |
| I-2b | Benzoate of I-2 |
| I-2c | Salicylate of I-2 |
| I-2d | Succinate of I-2 |

### Salt forms of 2-(1H-indol-3-yl)-N,N-bis(methyl-d₃)ethan-1-amine-1,1-d₂ (I-6)

Synthesis of 2-(1H-indol-3-yl)-N,N-bis(methyl-d₃)ethan-1-amine-1,1-d₂ (I-6) was carried out according to FIG. 1. Indole (A) (R₅=H) was iminoformylated using formaldehyde/dimethylamine to produce intermediate (B), which was then converted to the 3-acetic acid intermediate (C) using potassium cyanide in HCl. Subsequent treatment with thionyl chloride and dimethyl-d6-amine (R₈=R₉=CD₃) produced amide (D, R₈=R₉=CD₃), that was reduced by LiAlD₄ to yield compound I-6 (free base). The structure of the product was confirmed by ¹H NMR.

Various salt forms are prepared by crystallization of I-6 (free base) with stoichiometric (1.0 molar equivalent) quantities of the corresponding organic acid (fumaric acid, benzoic acid, salicylic acid, or succinic acid) from ethanol. The salt form identifier and salt type are provided in Table 4.

TABLE 4

| Salt form identifier | Salt type of compound |
|---|---|
| I-6a | Fumarate of I-6 |
| I-6b | Benzoate of I-6 |
| I-6c | Salicylate of I-6 |
| I-6d | Succinate of I-6 |

### Salt forms of 2-(1H-indol-3-yl)-N,N-dimethylethan-1-amine (I-1)

Synthesis of 2-(1H-indol-3-yl)-N,N-dimethylethan-1-amine (I-1) was carried out according to FIG. 2. Indole (A) (R₅=H) was acylated with oxalyl chloride and then reacted with dimethylamine to produce amide (F, R₈=R₉=CH₃). Subsequent reduction with LiAlH₄ yielded compound I-1 (free base). The structure of the product was confirmed by ¹H NMR.

Various salt forms of were prepared by crystallization of I-1 (free base) with stoichiometric (1.0 molar equivalent) quantities, or with sub-stoichiometric (0.5 molar equivalents) quantities in the case of hemi-salts, of the corresponding organic acid (fumaric acid, benzoic acid, salicylic acid, succinic acid, oxalic acid, or glycolic acid), from ethanol. The salt form identifier and salt type are provided in Table 5.

TABLE 5

| Salt form identifier | Salt type of compound |
|---|---|
| I-1a | Fumarate of I-1 |
| I-1b | Benzoate of I-1 |
| I-1c | Salicylate of I-1 |
| I-1d | Succinate of I-1 |
| I-1e | Oxalate of I-1 |
| I-1f | Glycolate of I-1 |
| I-1g | Hemi-oxalate of I-1 |
| I-1h | Hemi-fumarate of I-1^{a} |
| I-1i | Hemi-fumarate of I-1^{a} |

^{a}Two crystalline polymorphs of the hemi-fumarate salt were isolated

### Salt forms of 2-(1H-indol-3-yl)-N,N-bis(methyl-d₃)ethan-1-amine (I-4)

Synthesis of 2-(1H-indol-3-yl)-N,N-bis(methyl-d₃)ethan-1-amine (I-4) is carried out according to FIG. 2. Indole (A) (R₅=H) is acylated with oxalyl chloride and is then reacted with dimethyl-d6-amine to produce amide (F, R₈=R₉=CD₃). Subsequent reduction with LiAlH₄ yields compound I-4 (free base). The structure of the product will be confirmed by ¹H NMR.

Various salt forms are prepared by crystallization of I-4 (free base) with stoichiometric (1.0 molar equivalent) quantities of the corresponding organic acid (fumaric acid, benzoic acid, salicylic acid, or succinic acid) from ethanol. The salt form identifier and salt type are provided in Table 6.

TABLE 6

| Salt form identifier | Salt type of compound |
|---|---|
| I-4a | Fumarate of I-4 |
| I-4b | Benzoate of I-4 |
| I-4c | Salicylate of I-4 |
| I-4d | Succinate of I-4 |

### Salt forms of 2-(1H-indol-3-yl)-N,N-dimethylethan-1-amine-1,1,2,2-d₄ (I-5)

Synthesis of 2-(1H-indol-3-yl)-N,N-dimethylethan-1-amine-1,1,2,2-d₄ (I-5) was carried out according to FIG. 2. Indole (A) (R₅=H) was acylated with oxalyl chloride and then reacted with dimethylamine to produce amide (F, R₈=R₉=CH₃). Subsequent reduction with LiAlD₄ yielded compound I-5 (free base). The structure of the product was confirmed by ¹H NMR.

Various salt forms are prepared by crystallization of I-5 (free base) with stoichiometric (1.0 molar equivalent) quantities of the corresponding organic acid (fumaric acid, benzoic acid, salicylic acid, or succinic acid) from ethanol. The salt form identifier and salt type are provided in Table 7.

TABLE 7

| Salt form identifier | Salt type of compound |
|---|---|
| I-5a | Fumarate of I-5 |
| I-5b | Benzoate of I-5 |
| I-5c | Salicylate of I-5 |
| I-5d | Succinate of I-5 |

### Salt forms of 2-(1H-indol-3-yl)-N,N-bis(methyl-d₃)ethan-1-amine-1,1,2,2-d₄ (I-8)

Synthesis of 2-(1H-indol-3-yl)-N,N-bis(methyl-d₃)ethan-1-amine-1,1,2,2-d₄ (I-8) was carried out according to FIG. 2. Indole (A) (R₅=H) was acylated with oxalyl chloride and then reacted with dimethyl-d6-amine to produce amide F (R₈=R₉=CD₃). Amide F (R₉=R₉=CD₃) (7.04 g, 31.67 mmol) was charged to a flask under a nitrogen atmosphere. 2-MeTHF (140 ml, 20 vol) was then charged and stirred at room temperature for 15 minutes, though not all solid dissolved fully in this time. Lithium aluminum deuteride (LiAlD₄)(4.04 g, 96.23 mmol, 3.0 eq) was charged in portions over 45 minutes, maintaining the temperature of the reaction mixture <40° C. After complete addition, the reaction mixture was heated to 65° C. for 16 hours. The batch was cooled to room temperature and completion confirmed by HPLC analysis. A water/THF mixture (35 ml, 5 vol, 1:9) was added dropwise over 90 minutes, maintaining the temperature <30° C. 15% NaOH (aq) (3.5 ml, 0.5 vol) was then added dropwise, followed by further water (10.5 ml, 1.5 vol). This mixture was stirred at room temperature for 30 minutes then filtered and the solids washed with 2-MeTHF (3×70 ml, 3×10 vol), slurrying them each time before deliquoring. The liquors were concentrated to dryness, leaving compound I-8 (free base) as a viscous orange-red oil. The structure of the product was confirmed by ¹H NMR. Compound I-8 (free base) was then taken up in ethanol (49 ml, 7 vol), and this I-8 (free base) ethanol solution was used in the salt formation experiments provided below. The salt form identifier and salt type are provided in Table 8.

TABLE 8

| Salt form identifier | Salt type of compound |
|---|---|
| I-8a | Fumarate of I-8 |
| I-8b | Benzoate of I-8 |
| I-8c | Salicylate of I-8 |
| I-8d | Succinate of I-8 |

I-8a: A flask was charged with 15 ml of I-8 (free base) ethanol solution which was heated to reflux, and a single charge of fumaric acid (1.07 g, 1.05 eq) added. After complete dissolution, this was allowed to cool to room temperature, then cooled further to 0° C. and filtered, with additional ethanol (3×5 ml, 3×2 vol) used to rinse out the flask and wash the cake. I-8a was isolated as a crystalline solid with a yield of 1.480 g (4.71 mmol, 44.6%, 97.0% by LC, ¹H NMR confirmed identity as 1:1 fumarate salt).

I-8b: A flask was charged with 18 ml of I-8 (free base) ethanol solution which was heated to reflux, and then benzoic acid (4.04 g, 3.15 eq) was added in one charge. After ensuring all solid had dissolved, the solution was cooled in an ice bath and stirred for an additional 60 minutes at this temperature then filtered and further ice cold ethanol (2×2 vol) used to rinse out the flask and wash the cake. 2.272 g (7.09 mmol) of I-8b was obtained. Due to residual impurities observed in the HPLC analysis of this compound, it was slurried in ethanol (5 ml, 2 vol) for 16 hours. It was then cooled to 0° C. and filtered, with further ethanol (5 ml, 2 vol) used to rinse out the flask and wash the solids. However, the solids were amorphous and did not have a crystalline form. They were therefore dissolved in ethanol (15 ml, 7.5 vol) at reflux, cooled to 0° C. and filtered, using ethanol (5 ml, 2 vol) to wash them. The isolated I-8b was thus obtained as a white crystalline solid in a yield of 1.534 g (4.79 mmol, 45.4%, 91.1% by LC, ¹H NMR confirmed identity as 1:1 benzoate salt).

I-8c: A flask was charged with 18 ml of I-8 (free base) ethanol solution which was heated to reflux, and further ethanol (11.7 ml, total 12 vol) and salicylic acid (1.52 g, 1.05 eq) was added as a single charge. Once fully dissolved, the solution was cooled to 0° C. The resulting solids were filtered and washed with ice cold ethanol (2×2 vol), providing I-8c with a yield of 2.860 g (8.50 mmol). Due to residual impurities observed in the HPLC analysis of this compound, it was slurried in ethanol (5 ml, 2 vol) for 16 hours. It was then cooled to 0° C. and filtered, with further ethanol (5 ml, 2 vol) used to rinse out the flask and wash the solids. However, the solids were amorphous and did not have a crystalline form. They were therefore dissolved in ethanol (60 ml, 30 vol) at reflux, cooled to 0° C. and filtered, using ethanol (5 ml, 2 vol) to wash them. The isolated I-8c was thus obtained as a white crystalline solid in a yield of 2.311 g (6.87 mmol, 65.1%, 91.5% by LC, ¹H NMR confirmed identity as 1:1 salicylate salt).

I-8d: I-8d is prepared analogously by crystallization of I-8 (free base) with a stoichiometric (1.0 molar equivalent) quantity of succinic acid from ethanol.

### II. Formulations and Release Testing—Tryptamines

### Materials and Methods

Fumarate salt of 2-(1H-indol-3-yl)-N,N-bis(methyl-d₃)ethan-1-amine-1,1,2,2-d₄ (DMT-d₁₀ fumarate; 1:1)(I-8a) was prepared as described above. Hemi-fumarate salt of 2-(5-methoxy-1H-indol-3-yl)-N,N-bis(methyl-d₃)ethan-1-amine-1,1,2,2-d₄ (5-MeO-DMT-d₁₀ hemi-fumarate; 2:1) was made analogously to I-8a, except starting from Indole (A) (R₅=OMe) and using 0.5 eq of fumaric acid, by Sterling Pharma Solutions (HPLC purity: 99.85%; LCMS (M+H)⁺: 229.37). All water used was deionized water. Sodium chloride was ACS reagent grade, >99.0% (available from Sigma Aldrich). The release modifier used is shown in Table 9 in terms of the molecular weight range (and in parentheses the weight average molecular weight, Mw), and the commercial supplier.

TABLE 9

| | | Molecular | |
|---|---|---|---|
| | | Weight Range | |
| Release | Product | (Weight average | Commercial |
| Modifier | name | in parentheses) | supplier |
| Sodium | HA5K | <5 kDa | Lifecore |
| hyaluronate | | (Mw = 4.6 kDa) | |
| | HA60K | 66-99 kDa | Biomedical, Inc |
| | | (Mw = 70 kDa) | |
| | HA500K | 301-475 kDa | |
| | | (Mw = 381 kDa) | |
| | HA700K | 500-<750 kDa | |
| | | (Mw = 622 kDa) | |
| | HA1M | 750-1,000 kDa | |
| | | (Mw = 830 kDa) | |
| | HA15M | >1,000-1,800 kDa | |
| | | (Mw = 1,670 kDa) | |
| Sodium | Aqualon ™ | (Mw = 250 kDa) | Ashland |
| Carboxymethyl | 7MF PH | | |
| cellulose | | | |

pH. pH was measured by Accumet™ Research Model AR10 pH meter, available from Fisher Scientific.

Osmolality. Osmolality was measured using Advanced® Micro-Osmometer, Model 3320, from Advanced Instruments.

Filtration was performed using a Syringe filter unit, 0.22 μm, PVDF, 33 mm filter, available from Sigma-Aldrich.

Dialysis—Drug Release Test. A 200 mL Erlenmeyer flask containing 100 mL of release medium (0.9% w/v NaCl) was equipped with a magnetic stir bar and placed into a water bath to maintain a release medium temperature of 37° C. The stirring rate was set to 400 rpm. A 1-2 mL volume of test item (formulation or control) was introduced into a Pur-A-Lyzer™ Maxi dialysis tube (pore size: 6-8 kDa molecular weight cutoff (MWCO), available from Sigma Aldrich). The dialysis tube was then immersed into the 100 mL of release medium inside the Erlenmeyer flask and a timer was started. Dissolution medium (0.5 mL) was withdrawn from the Erlenmeyer flask at 10, 20, 30, 45, 60, 90, and 120 minutes for assays, and an equal volume of fresh release medium was used to replace the withdrawn dissolution medium. The time-sampled dissolution medium was assayed by ultra performance liquid chromatography (UPLC) to analyze the drug release of the test item. The percent drug release was then plotted versus time (minutes), and also modelled as a first order kinetic plot. From these plots, the time in minutes required for 25% of drug release (t_{25%}) and 50% of drug release (t_{50%}) were calculated.

Ultra Performance Liquid Chromatography (UPLC). Analysis was performed on an Agilent 1100/1200 Quaternary Pump 1 system equipped with a d₁₀ de array detector UV Diode array 220-269 nm. The UPLC method parameters used for the analysis are presented in Table 10.

TABLE 10

| Sample Preparation: | 0.2-0.5 mg · mL⁻¹ in water | | |
|---|---|---|---|
| Column: | Waters T3 4.6 × 150 mm, 3 μm | | |
| Column Temperature (° C.): | 25 | | |
| Injection (μL): | 5 μl | | |
| Detection: Wavelength, | UV Diode array 220 nm | | |
| Bandwidth (nm): | Monitor/system check 290 nm | | |
| Phase A: | Water + 0.1% H₃PO₄ | | |
| Phase B: | ACN + 0.1% H₃PO₄ | | |
| Flow Rate (mL · min⁻¹) | 1.000 ml/min | | |
| Stop Time | 18.00 min | | |
| | Time | % | % |
| | (min) | Phase A | Phase B |
| Timetable: | 0 | 90 | 10 |
| | 1.0 | 90 | 10 |
| | 8.0 | 70 | 30 |
| | 8.10 | 10 | 90 |
| | 13.0 | 10 | 90 |
| | 13.10 | 90 | 10 |
| | 18.00 | 90 | 10 |

### Formulations and Release Profiles

Formulations and their preparative methods are presented below. The physiochemical properties of the formulations such as pH and osmolality were determined where indicated. Formulations are presented in terms of the parameters A, B, and C shown in Table 11. The ratio of A:B and the ratio of C:A may also be indicated, rounded to the closest whole number. The ratio of A:B was calculated by dividing parameter A in mg/mL (nominal) by parameter B in % w/v (nominal). The ratio of C:A was calculated by dividing the Mw value in kDa by the parameter A in mg/mL (nominal). Both the ratio of A:B and C:A are written below without the consequent “1” and the ratio symbol (:), for example a ratio of 50:1 is simply represented as 50.

TABLE 11

| Parameter | Description |
|---|---|
| A | Free base concentration of psychopharmaceutical |
| | agent (mg/mL, nominal) |
| B | Release modifier concentration |
| | (% w/v, nominal) |
| C | Molecular weight of release modifier |
| | (Mw in kDa) |

The release profiles of the formulations are also evaluated by the Dialysis—Drug Release Test, including the time in minutes required for 25% of drug release (t_{25%}) and 50% of drug release (t_{50%}), and the respective percent changes in these values compared to the control (made without release modifier).

### Formulations 1-6

To investigate the role of different concentrations of sodium hyaluronate (0.1, 0.2, 0.3, 0.4, 0.5, and 0.75% w/v, nominal) in the formulations, six different sodium hyaluronate solutions were made by dissolving a corresponding amount of sodium hyaluronate (750-1,000 kDa) (1.0 mg, 2.0 mg, 3.0 mg, 4.0 mg, 5.0 mg, and 7.5 mg, respectively), in water (0.7 mL), followed by incubation at 37° C. and stirring. The sodium hyaluronate solutions were then filtered through a 0.22 μm PVDF filter. Solutions of DMT-d₁₀ fumarate were prepared by dissolving 15.85 mg of DMT-d₁₀ fumarate (10 mg free base equivalence of DMT-d₁₀) in water (0.3 mL), followed by incubation at 37° C. and stirring. Each sodium hyaluronate solution was then mixed with the solution of DMT-d₁₀ fumarate, and the resulting formulations were vigorously stirred to ensure complete mixing. A control formulation (10 mg/mL free base equivalence of DMT-d₁₀, nominal) without release modifier was prepared by diluting the solution of DMT-d₁₀ fumarate with water (0.7 mL). Formulations are shown in Table 12.

TABLE 12

| | A) Free base conc. | B) Sodium |
|---|---|---|
| | of DMT-d₁₀ | hyaluronate conc. |
| | (mg/mL, nominal) | (% w/v, nominal) |
| Control | 10 | — |

C) Sodium hyaluronate = 750-1,000 kDa (Mw = 830 kDa)

| | | |
|---|---|---|
| Formulation 1 | 10 | 0.1% |
| Formulation 2 | 10 | 0.2% |
| Formulation 3 | 10 | 0.3% |
| Formulation 4 | 10 | 0.4% |
| Formulation 5 | 10 | 0.5% |
| Formulation 6 | 10 | 0.75% |

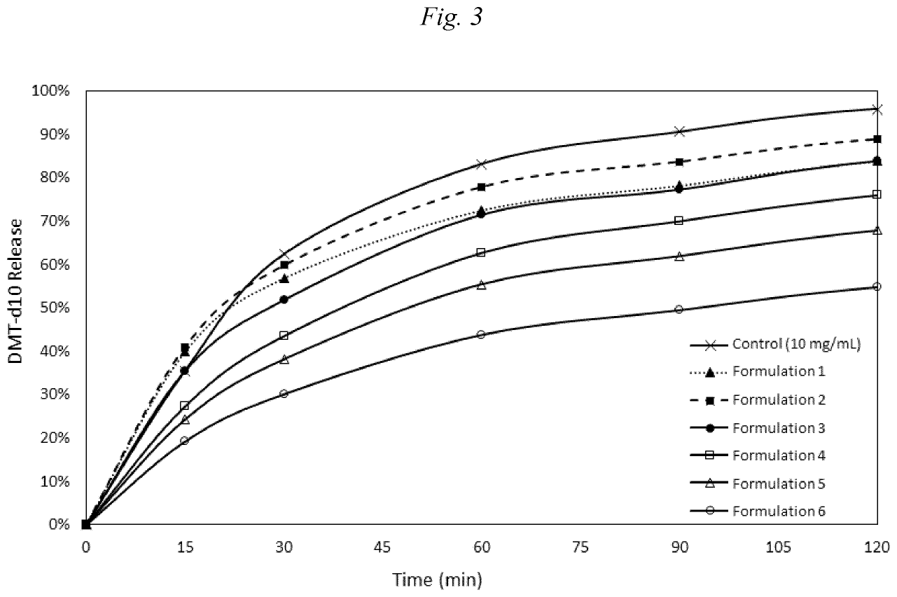

Each formulation was subjected to the Dialysis—Drug Release Test. FIG. 3 shows the drug release is highly controlled by the concentration of sodium hyaluronate in the formulation, with the control formulation providing the fastest release, and increasing sodium hyaluronate concentrations providing increasingly slower drug release, with the slowest release provided by the highest sodium hyaluronate content (Formulation 6). To increase osmolality for improved clinical tolerance, these formulations will also be remade with an appropriate saline vehicle instead of water without effecting the release profile. Nonetheless, these formulations support the notion that sodium hyaluronate serves as a robust polymer matrix for tunable and controlled-release of drug.

### Formulations 7-9

To investigate the effects of drug concentration, formulations were prepared using a fixed sodium hyaluronate concentration (1% w/v, nominal) at 10 mg/mL, 25 mg/mL, and 50 mg/mL free base equivalence of DMT-d₁₀ (nominal). Solutions of DMT-d₁₀ fumarate were prepared by dissolving 15.85 mg of DMT-d₁₀ fumarate (10 mg free base equivalence of DMT-d₁₀), 39.62 mg of DMT-d₁₀ fumarate (25 mg free base equivalence of DMT-d₁₀), or 79.25 mg of DMT-d₁₀ fumarate (50 mg free base equivalence of DMT-d₁₀) in water (0.3 mL), followed by incubation at 37° C. and stirring. Sodium hyaluronate solutions were prepared using 10 mg of sodium hyaluronate (750-1,000 kDa) and water (0.7 mL), followed by incubation at 37° C. and stirring. The sodium hyaluronate solutions were then filtered through a 0.22 μm PVDF filter. Each solution of DMT-d₁₀ fumarate was then mixed with a sodium hyaluronate solution, and the resulting formulations were vigorously stirred to ensure complete mixing. Corresponding control formulations (10, 25, or 50 mg/mL free base equivalence of DMT-d₁₀, nominal) without release modifier were prepared by diluting each solution of DMT-d₁₀ fumarate with water (0.7 mL). Formulations are shown in Table 13.

TABLE 13

| | |
|---|---|
| A) Free base conc. | B) Sodium |
| of DMT-d₁₀ | hyaluronate conc. |
| (mg/mL, nominal) | (% w/v, nominal) |

C) Sodium hyaluronate = 750-1,000 kDa (Mw = 830 kDa)

| | | |
|---|---|---|
| Control | 10 | — |
| Formulation 7 | 10 | 1% |
| Control | 25 | — |
| Formulation 8 | 25 | 1% |
| Control | 50 | — |
| Formulation 9 | 50 | 1% |

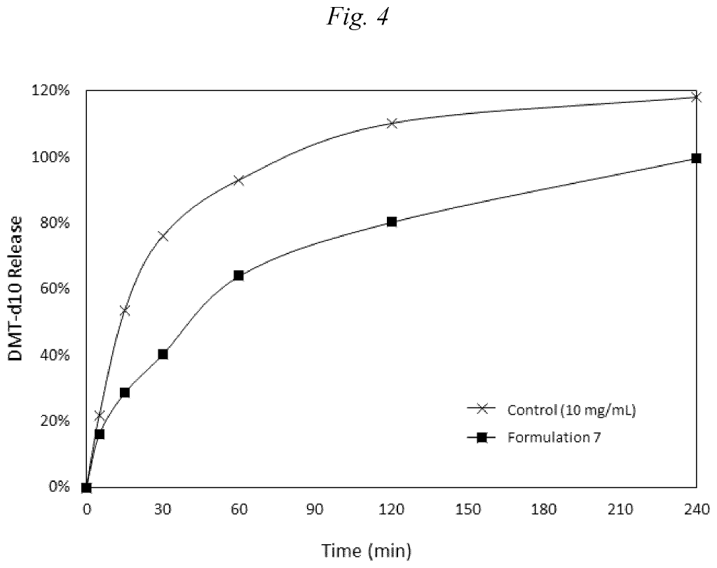

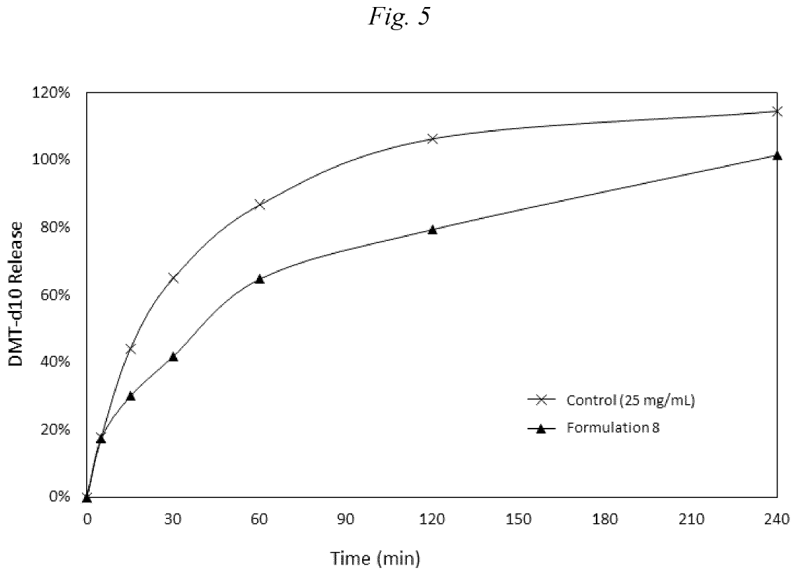

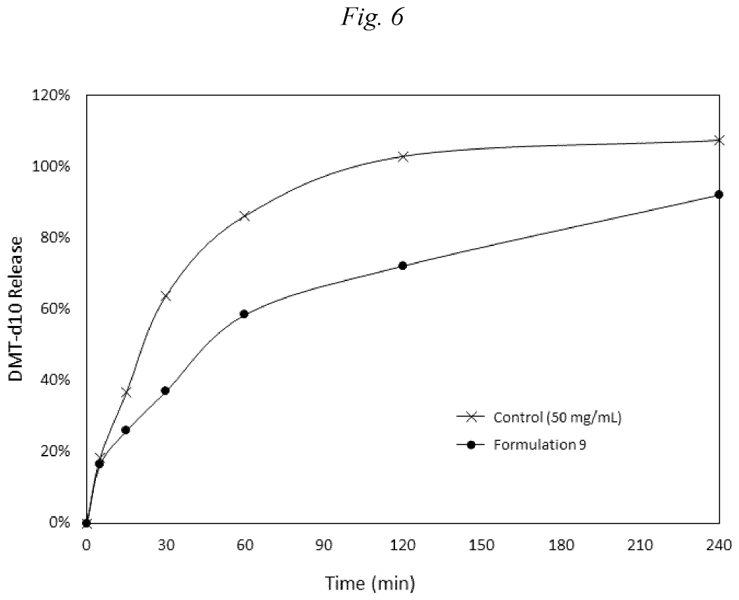

Each formulation was subjected to the Dialysis—Drug Release Test. FIGS. 4-6 show the drug release of Formulations 7, 8, and 9, respectively, and that each formulation provided a desirable controlled-release profile at the tested drug loading.

### Formulations 10-15

To investigate the role of sodium hyaluronate molecular weight, different formulations were prepared with varying molecular weights of sodium hyaluronate using a fixed sodium hyaluronate concentration (0.5% w/v, nominal) and free base concentration of DMT-d₁₀ (25 mg/mL, nominal). Stock solutions were prepared as follows:
Stock (a). 79.25 mg of DMT-d₁₀ fumarate (50 mg free base equivalence of DMT-d₁₀) was dissolved in 0.5% (w/v) saline (0.75 mL). The pH was then adjusted to 6.0±0.1 using 1M NaOH solution (0.25 mL). The pH and osmolality were measured.
Stock (b). 10 mg of sodium hyaluronate was dissolved in 0.5% (w/v) saline (1.0 mL), followed by incubation at 37° C. and intermittent vortexing until the solution became clear. The osmolality was then measured.

The stock solutions (a) and (b) were then mixed in a 1:1 volume ratio to form the formulations, after which the osmolality was measured. A control formulation (25 mg/mL free base equivalence of DMT-d₁₀, nominal) without release modifier was prepared by diluting stock solution (a) with 1.0 mL of 0.5% (w/v) saline. Formulations are shown in Table 14.

TABLE 14

| | A) Free base | | | |
|---|---|---|---|---|
| | conc. of | B) Sodium | | |
| | DMT-d₁₀ | hyaluronate | | |
| | (mg/mL, | conc. (% w/v, | | Osmolality |
| | nominal) | nominal) | pH | (mOsm/kg) |
| Control | 25 | — | — | — |

C) Sodium hyaluronate = <5 kDa (Mw = 4.6 kDa)

| | | | | |
|---|---|---|---|---|
| Stock (a) | 50 | — | 6.10 | 696.33 |
| Stock (b) | — | 1% | — | 202.34 |
| Formulation 10 | 25 | 0.5% | — | 490.00 |

C) Sodium hyaluronate = 66-99 kDa (Mw = 70 kDa)

| | | | | |
|---|---|---|---|---|
| Stock (a) | 50 | — | 6.10 | 696.33 |
| Stock (b) | — | 1% | — | 202.34 |
| Formulation 11 | 25 | 0.5% | — | 482.00 |

C) Sodium hyaluronate = 301-475 kDa (Mw = 381 kDa)

| | | | | |
|---|---|---|---|---|
| Stock (a) | 50 | — | 6.10 | 696.33 |
| Stock (b) | — | 1% | — | 197.00 |
| Formulation 12 | 25 | 0.5% | — | 495.00 |

C) Sodium hyaluronate = 500-<750 kDa (Mw = 622 kDa)

| | | | | |
|---|---|---|---|---|
| Stock (a) | 50 | — | 6.10 | 696.33 |
| Stock (b) | — | 1% | — | 202.34 |
| Formulation 13 | 25 | 0.5% | — | 462.66 |

C) Sodium hyaluronate = 750-1,000 kDa (Mw = 830 kDa)

| | | | | |
|---|---|---|---|---|
| Stock (a) | 50 | — | 6.10 | 696.33 |
| Stock (b) | — | 1% | — | 202.34 |
| Formulation 14 | 25 | 0.5% | — | 470.22 |

C) Sodium hyaluronate = >1,000-1,800 kDa (Mw = 1,670 kDa)

| | | | | |
|---|---|---|---|---|
| Stock (a) | 50 | — | 6.10 | 696.33 |
| Stock (b) | — | 1% | — | 208.80 |
| Formulation 15 | 25 | 0.5% | — | 475.60 |

The osmolality of all formulations remained within an acceptable range for injection, and the pH adjustment of stock (a) (solution of DMT-d₁₀ fumarate) was well tolerated with respect to stability of the final formulations.

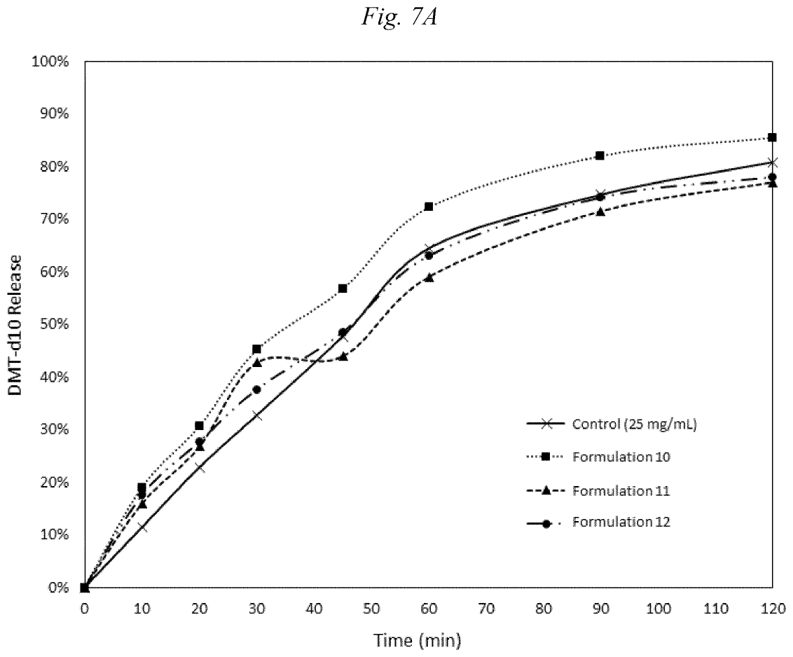

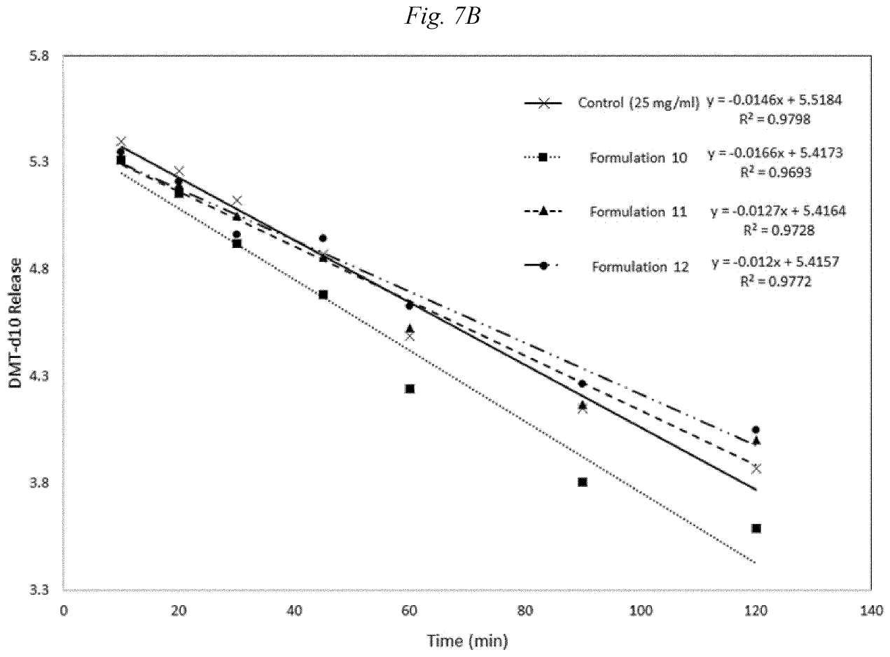

Each formulation was subjected to the Dialysis—Drug Release Test. As shown in FIGS. 7A and 7B, and Table 1S, Formulations 10-12 made from low molecular weight sodium hyaluronate, <5 kDa, 66-99 kDa, and 301-475 kDa, respectively, failed to provide meaningful controlled-release effects and instead provided a release similar to control (no release modifier).

TABLE 15

| | | | T_{25%}, minutes | | T_{50%}, minutes | |
|---|---|---|---|---|---|---|
| | | | (% change | | (% change | |
| | A:B | C:A | from control) | | from control) | |
| Control | — | — | 19.7 | | 47.5 | |
| Formulation 10 | 50 | <1 | 17.3 | (−12%) | 41.8 | (−12%) |
| (<5 kDa) | | | | | | |
| Formulation 11 | 50 | 3 | 24.0 | (+22%) | 57.8 | (+22%) |
| (66-99 kDa) | | | | | | |
| Formulation 12 | 50 | 15 | 22.7 | (+15%) | 54.6 | (+15%) |
| (301-475 kDa) | | | | | | |

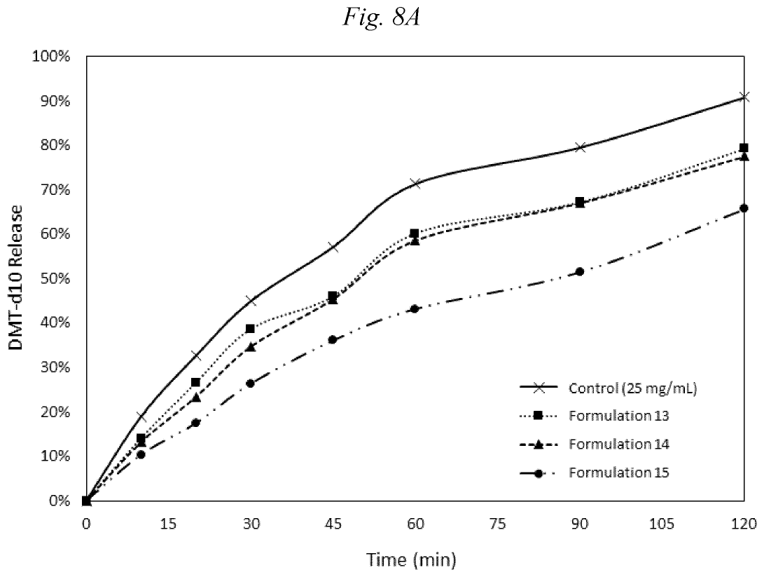

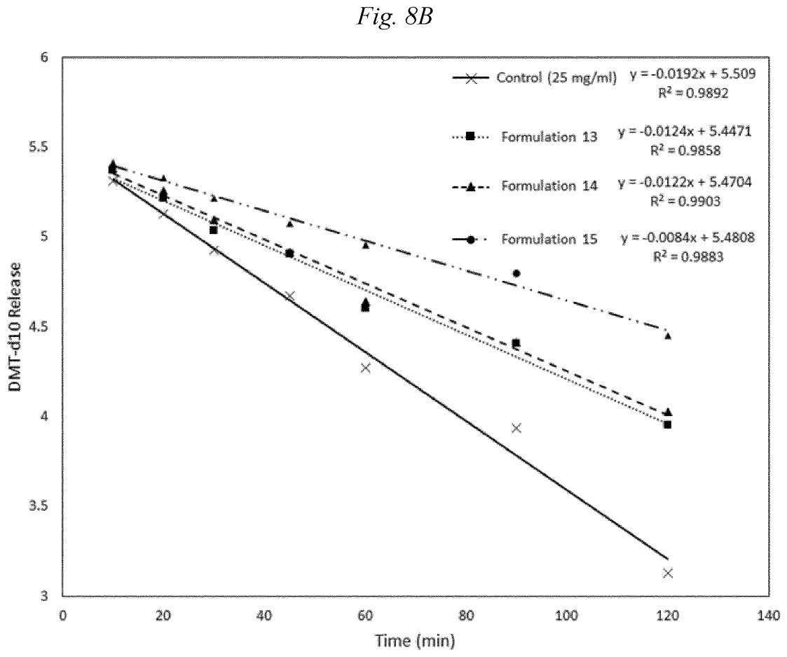

To the contrary, Formulations 13-15 made from higher molecular weight sodium hyaluronate, 500-<750 kDa, 750-1,000 kDa, and >1,000-1,800 kDa, respectively, provided a desirable controlled-release profile, with the highest molecular weight (Formulation 15) providing a nearly 130% increase at each of T₂₅% and T_{50%} (FIGS. 8A and 8B, and Table 16).

TABLE 16

| | | | T_{25%}, minutes | | T_{50%}, minutes | |
|---|---|---|---|---|---|---|
| | | | (% change | | (% change | |
| | A:B | C:A | from control) | | from control) | |
| Control | — | — | 15.0 | | 36.1 | |
| Formulation 13 | 50 | 25 | 23.2 | (+55%) | 55.9 | (+55%) |
| (500-<750 kDa) | | | | | | |
| Formulation 14 | 50 | 33 | 23.6 | (+57%) | 56.8 | (+57%) |
| (750-1,000 kDa) | | | | | | |
| Formulation 15 | 50 | 67 | 34.2 | (+128%) | 82.5 | (+129%) |
| (>1,000-1,800 kDa) | | | | | | |

These data indicate that a sodium hyaluronate molecular weight cutoff of at least 500 kDa seems to exist for establishing controlled-release characteristics.

### Formulations 16-18

To investigate the role of sodium hyaluronate molecular weight at high drug loadings, different formulations were prepared with varying molecular weights of sodium hyaluronate using a fixed sodium hyaluronate concentration (0.5% w/v, nominal) and free base concentration of DMT-d₁₀ (70 mg/mL, nominal) in 0.9% (w/v) saline with no pH adjustments. Stock solutions were prepared as follows:
Stock (a). 221.9 mg of DMT-d₁₀ fumarate (140 mg free base equivalence of DMT-d₁₀) was dissolved in 0.9% (w/v) saline (1.0 mL). The pH and osmolality were measured.
Stock (b). 10 mg of sodium hyaluronate was dissolved in 0.9% (w/v) saline (1.0 mL), followed by incubation at 37° C. and intermittent vortexing until the solution became clear. The osmolality was then measured.

The stock solutions (a) and (b) were then mixed in a 1:1 volume ratio to form the formulations, after which the osmolality was measured. A control formulation (70 mg free base equivalence of DMT-d₁₀, nominal) without release modifier was prepared by diluting stock solution (a) with 1.0 mL of 0.9% (w/v) saline. Formulations are shown in Table 17.

TABLE 17

| | A) Free base | | | |
|---|---|---|---|---|
| | conc. of | B) Sodium | | |
| | DMT-d₁₀ | hyaluronate | | |
| | (mg/mL, | conc. (% w/v, | | Osmolality |
| | nominal) | nominal) | pH | (mOsm/kg) |
| Control | 70 | — | — | — |

C) Sodium hyaluronate = 500-<750 kDa (Mw = 622 kDa)

| | | | | |
|---|---|---|---|---|
| Stock (a) | 140 | — | 2.87 | 633.00 |
| Stock (b) | — | 1% | — | 202.45 |
| Formulation 16 | 70 | 0.5% | — | 665.00 |

C) Sodium hyaluronate = 750-1,000 kDa (Mw = 830 kDa)

| | | | | |
|---|---|---|---|---|
| Stock (a) | 140 | — | 2.87 | 633.00 |
| Stock (b) | — | 1% | — | 202.34 |
| Formulation 17 | 70 | 0.5% | — | 660.00 |

C) Sodium hyaluronate = >1,000-1,800 kDa (Mw = 1,670 kDa)

| | | | | |
|---|---|---|---|---|
| Stock (a) | 140 | — | 2.87 | 633.00 |
| Stock (b) | — | 1% | — | 208.34 |
| Formulation 18 | 70 | 0.5% | — | 705.00 |

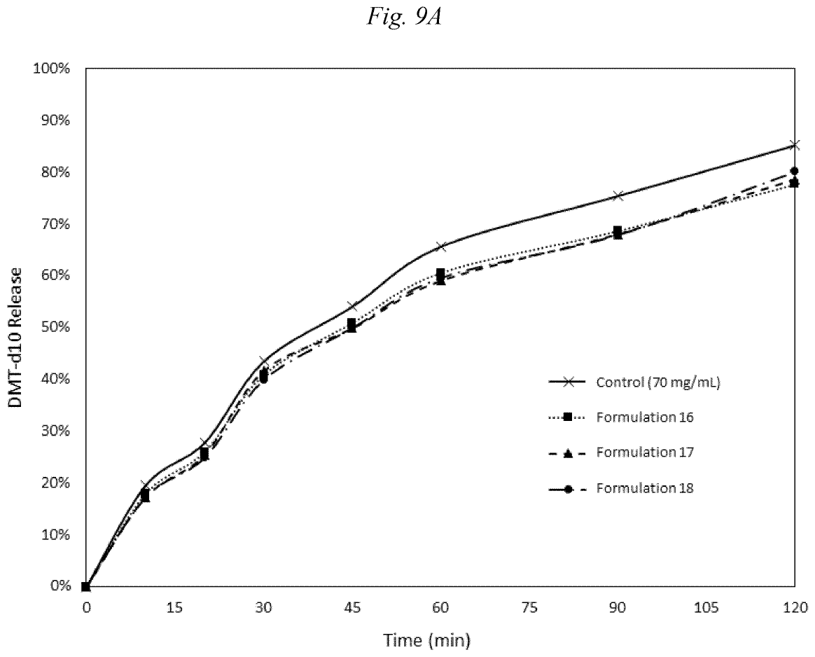

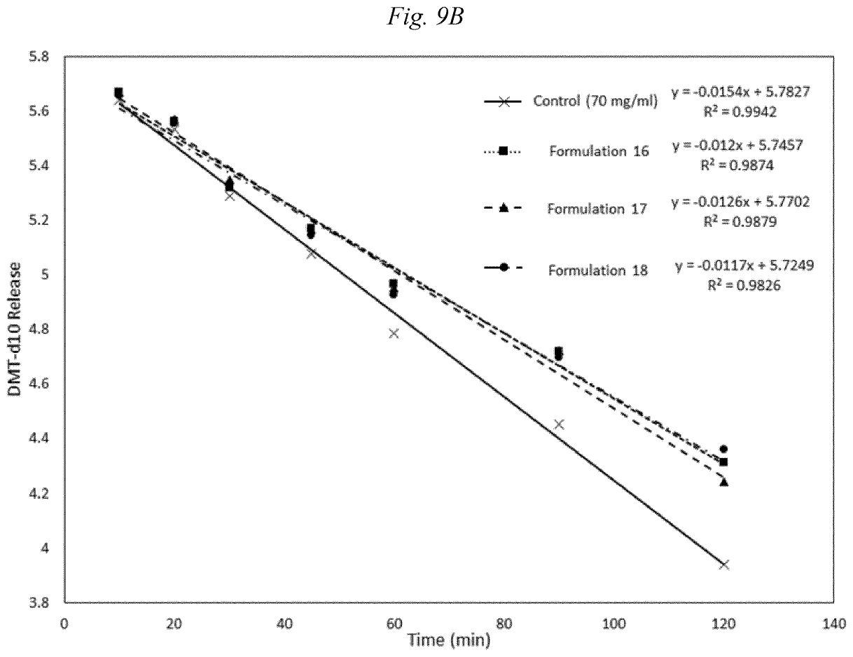

Each formulation was subjected to the Dialysis—Drug Release Test. As shown in FIGS. 9A and 9B, and Table 18, Formulations 16-18 made from high drug loading in high molecular weight sodium hyaluronate (500-<750 kDa, 750-1,000 kDa, and >1,000-1,800 kDa) provided a release that was marginally slower than control (no release modifier), with percent change of T_{50%} values compared to control of +32%, +28%, and +24%, respectively. This is indicative of not enough sodium hyaluronate being used to compensate for the high drug content.

TABLE 18

| | | | T_{25%}, minutes | | T_{50%}, minutes | |
|---|---|---|---|---|---|---|
| | | | (% change | | (% change | |
| | A:B | C:A | from control) | | from control) | |
| Control | — | — | 18.7 | (—) | 45.0 | (—) |
| Formulation 16 | 140 | 9 | 24.6 | (+32%) | 59.2 | (+32%) |
| (500-<750 kDa) | | | | | | |
| Formulation 17 | 140 | 12 | 24.0 | (+28%) | 57.8 | (+28%) |
| (750-1,000 kDa) | | | | | | |
| Formulation 18 | 140 | 24 | 22.8 | (+22%) | 55.8 | (+24%) |
| (>1,000-1,800 kDa) | | | | | | |

However, the physiochemical properties of Formulations 16-18 were not within acceptable ranges; the osmolality of all formulations exceeded the acceptable upper limit of 600 mOsm/kg, and the pH values were very acidic. As a result, a precipitation event was observed in all formulations within hours of storage under refrigerated conditions.

### Formulations 19-21

To investigate the release profile with 2-(5-methoxy-1H-indol-3-yl)-N,N-bis(methyl-d₃)ethan-1-amine-1,1,2,2-d₄ (5-MeO-DMT-d₁₀), different formulations were prepared with varying molecular weights of sodium hyaluronate using a fixed sodium hyaluronate concentration (0.5% w/v, nominal) and free base concentration of 5-MeO-DMT-d₁₀ (25 mg/mL, nominal) in 0.3% (w/v) saline with no pH adjustments. Stock solutions were prepared as follows:
Stock (a). 62.7 mg of 5-MeO-DMT-d₁₀ hemi-fumarate (50 mg free base equivalence of 5-MeO-DMT-d₁₀) was dissolved in 0.3% (w/v) saline (1.0 mL). The pH and osmolality were measured.
Stock (b). 10 mg of sodium hyaluronate was dissolved in 0.3% (w/v) saline (1.0 mL), followed by incubation at 37° C. and intermittent vortexing until the solution became clear. The pH and osmolality was then measured.

The stock solutions (a) and (b) were then mixed in a 1:1 volume ratio to form the formulations, after which the pH and osmolality was measured. A control formulation (25 mg free base equivalence of 5-MeO-DMT-d₁₀, nominal) without release modifier was prepared by diluting stock solution (a) with 1.0 mL of 0.3% (w/v) saline. Formulations are shown in Table 19.

TABLE 19

| | A) Free base | | | |
|---|---|---|---|---|
| | conc. of | | | |
| | 5-MeO- | B) Sodium | | |
| | DMT-d₁₀ | hyaluronate | | |
| | (mg/mL, | conc. (% w/v, | | Osmolality |
| | nominal) | nominal) | pH | (mOsm/kg) |
| Control | 25 | — | — | — |

C) Sodium hyaluronate = 500-<750 kDa (Mw = 622 kDa)

| | | | | |
|---|---|---|---|---|
| Stock (a) | 50 | — | 5.77 | 199.00 |
| Stock (b) | — | 1% | 7.01 | 124.01 |
| Formulation 19 | 25 | 0.5% | 5.76 | 217.76 |

C) Sodium hyaluronate = 750-1,000 kDa (Mw = 830 kDa)

| | | | | |
|---|---|---|---|---|
| Stock (a) | 50 | — | 5.77 | 199.00 |
| Stock (b) | — | 1% | 7.12 | 127.87 |
| Formulation 20 | 25 | 0.5% | 5.79 | 218.34 |

C) Sodium hyaluronate = >1,000-1,800 kDa (Mw = 1,670 kDa)

| | | | | |
|---|---|---|---|---|
| Stock (a) | 50 | — | 5.77 | 199.00 |
| Stock (b) | — | 1% | 7.09 | 129.04 |
| Formulation 21 | 25 | 0.5% | 5.76 | 218.23 |

All formulations were prepared with 0.3% (w/v) saline and had an osmolality within an acceptable range for injection. The formulations also had an acceptable pH (about 5.7) without any pH adjustments, and there was no observed instability.

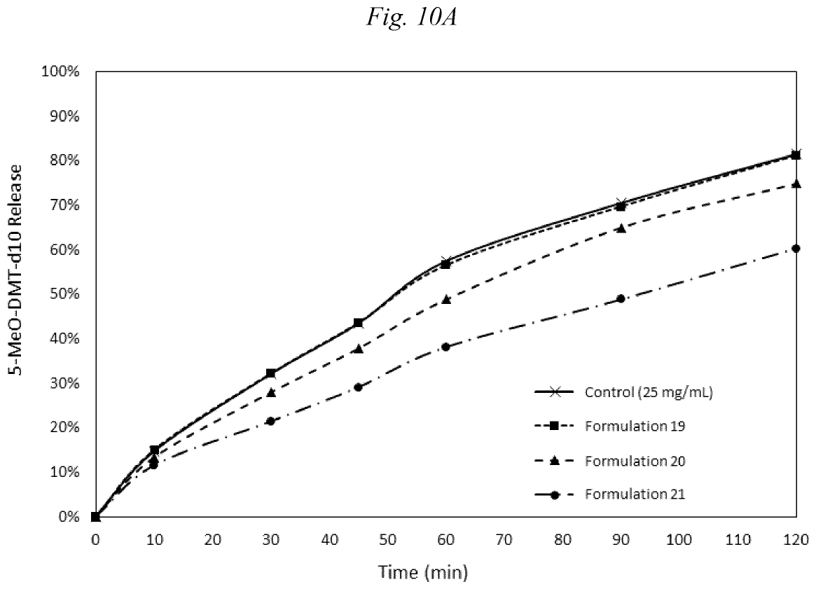

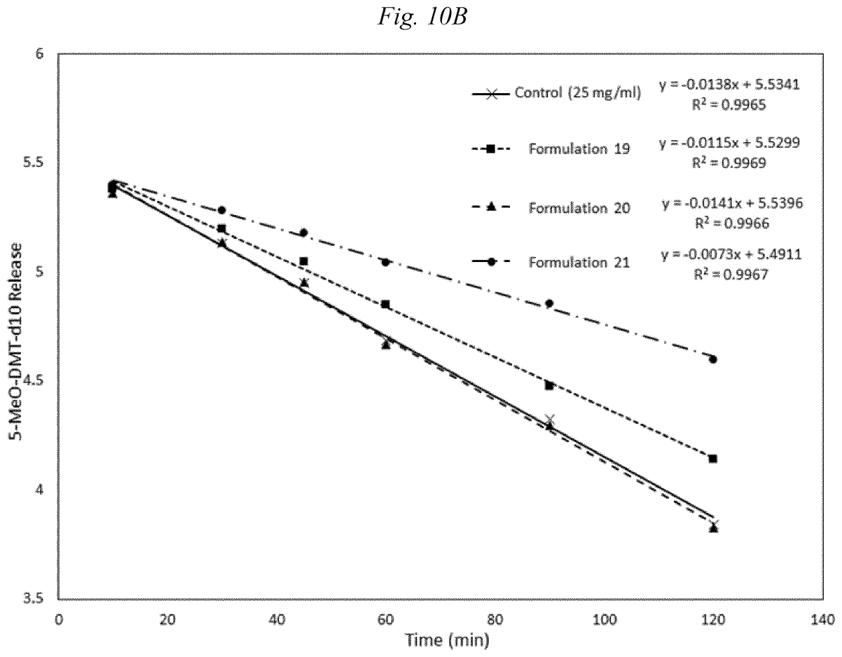

Each formulation was subjected to the Dialysis—Drug Release Test. As shown in FIGS. 10A and 10B, and Table 20, Formulation 19 made using 5-MeO-DMT-d₁₀ hemi-fumarate in the lowest molecular weight sodium hyaluronate tested (500-<750 kDa) gave a release profile which was practically no different than control (no release modifier), and Formulation 20 made with sodium hyaluronate 750-1,000 kDa fared somewhat better. Formulation 21 made with sodium hyaluronate >1,000-1,800 kDa was much more effective in providing a controlled-release, with percent change of T_{25%} and T_{50%} values compared to control of +93% each.

TABLE 20

| | | | T_{25%}, minutes | | T_{50%}, minutes | |
|---|---|---|---|---|---|---|
| | | | (% change | | (% change | |
| | A:B | C:A | from control) | | from control) | |
| Control | — | — | 20.4 | (—) | 49.2 | (—) |
| Formulation 19 | 50 | 25 | 20.8 | (+2%) | 50.2 | (+2%) |
| (500-<750 kDa) | | | | | | |
| Formulation 20 | 50 | 33 | 25.0 | (+23%) | 60.3 | (+23%) |
| (750-1,000 kDa) | | | | | | |
| Formulation 21 | 50 | 67 | 39.4 | (+93%) | 95.0 | (+93%) |
| (>1,000-1,800 kDa) | | | | | | |

### Formulations 22-27

To investigate the release modifier sodium carboxymethyl cellulose and the role of different concentrations thereof (0.1, 0.2, 0.3, 0.4, 0.5, and 0.75% w/v, nominal) in the formulations, six different sodium carboxymethyl cellulose solutions were made by dissolving a corresponding amount of sodium carboxymethyl cellulose (Aqualon™ 7MF PH; Mw=250 kDa) (1.0 mg, 2.0 mg, 3.0 mg, 4.0 mg, 5.0 mg, and 7.5 mg, respectively), in water (0.7 mL), followed by incubation at 37° C. and stirring. The sodium carboxymethyl cellulose solutions were then filtered through a 0.22 μm PVDF filter. Solutions of DMT-d₁₀ fumarate were prepared by dissolving 15.85 mg of DMT-d₁₀ fumarate (10 mg free base equivalence of DMT-d₁₀) in water (0.3 mL), followed by incubation at 37° C. and stirring. Each sodium carboxymethyl cellulose solution was then mixed with the solution of DMT-d₁₀ fumarate, and the resulting formulations were vigorously stirred to ensure complete mixing. A control formulation (10 mg/mL free base equivalence of DMT-d₁₀, nominal) without release modifier was prepared by diluting the solution of DMT-d₁₀ fumarate with water (0.7 mL). Formulations are shown in Table 21.

TABLE 21

| | A) Free base | B) Sodium carboxymethyl |
|---|---|---|
| | conc. of DMT-d₁₀ | cellulose conc. |
| | (mg/mL, nominal) | (% w/v, nominal) |
| Control | 10 | — |

C) Sodium carboxymethyl cellulose (Mw = 250 kDa)

| | | |
|---|---|---|
| Formulation 22 | 10 | 0.1% |
| Formulation 23 | 10 | 0.2% |
| Formulation 24 | 10 | 0.3% |
| Formulation 25 | 10 | 0.4% |
| Formulation 26 | 10 | 0.5% |
| Formulation 27 | 10 | 0.75% |

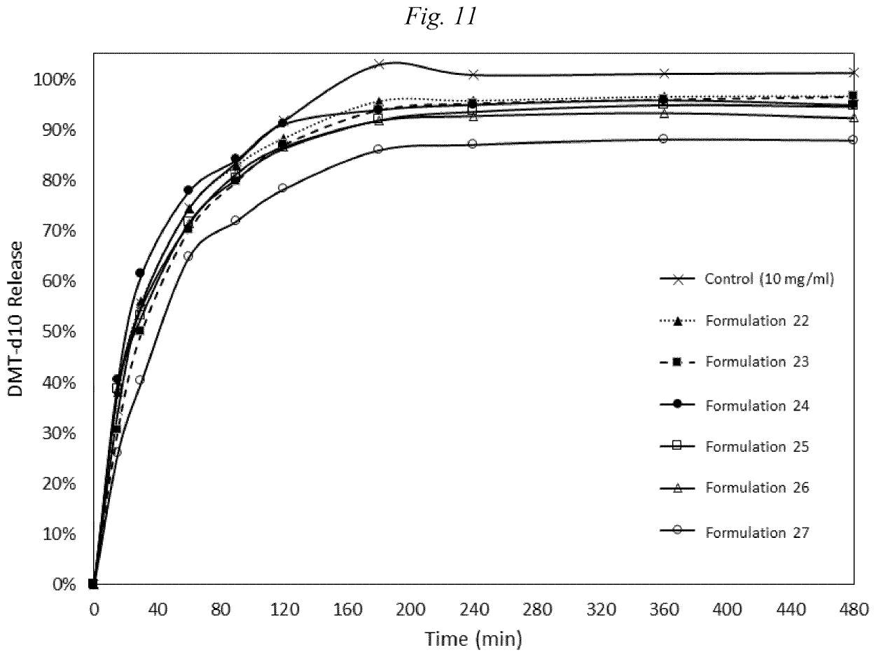

Each formulation was subjected to the Dialysis—Drug Release Test. FIG. 11 shows that the release properties from sodium carboxymethyl cellulose formulations was markedly different from sodium hyaluronate-based formulations. Here, the fastest release profile was observed for control and no prominent delayed release effects were achieved with 0.1, 0.2, 0.3, 0.4, or 0.5% w/v (nominal) sodium carboxymethyl cellulose (Formulations 22-26). A delayed effect was only seen with sodium carboxymethyl cellulose loadings higher than 0.5% w/v (nominal), such as 0.75% w/v (nominal) (Formulation 27), which was modest compared to the effects at the same concentration of sodium hyaluronate. These results indicate that sodium carboxymethyl cellulose can serve as a polymer matrix for controlled-release of drug, but that the workable concentration range is narrow and the release is not highly tunable.

### III. Animal Studies—Tryptamines

The pharmacokinetics of 2-(1H-indol-3-yl)-N,N-bis(methyl-d₃)ethan-1-amine-1,1,2,2-d₄ (DMT-d₁₀) were investigated in dogs following subcutaneous (SC) administration of DMT-d₁₀. Three studies were conducted to establish whether: 1) SC administration produced quantifiable plasma concentrations of DMT-d₁₀; 2) hyaluronate salt modified the release of DMT-d₁₀ after SC administration; 3) hyaluronate salt aqueous concentration altered the absorption profile of DMT-d₁₀; and 4) an increase in dose concentration produced an extemporaneous release (‘dumping’) of DMT-d₁₀ from the formulation.

The following studies were conducted at the same Contract Research Organization (CRO). Detailed animal care, test materials, bioanalytical and pharmacokinetic analysis are described in part A and pertain to parts B and C also.

### A: Proof of Concept—Pharmacokinetics (PK) of DMT-d₁₀ in Male Beagle Dogs Following Subcutaneous Administration in Saline or Sodium Hyaluronate

The objective of this study was to determine if a 1% (w/v) sodium hyaluronate formulation could modify the SC release of DMT-d₁₀ compared to 0.9% (w/v) saline formulation.

Animals. Three, non-naïve, male Beagle dogs aged ca 2-5 years and weighing 10-15 kg at dosing were supplied by Charles River for use on this study and were bred for use in scientific procedures. The animal work in this study was conducted under the UK Home Office Project License No. PPL PP9376768, Pharmacokinetics of Pharmaceuticals. This was a cross-over design study to reduce the number of dogs in use and as well as decrease interindividual variability.

Housing. Animals were uniquely identified by microchip and tattoo in this study. During pre-trial and on-study periods, the animals were group housed in caging appropriate to the species. The animals were housed singly for up to 4 h per day and during this time had access to their daily ration of diet.

IPS 5007 diet was provided as a daily ration throughout the study. A 350 g ration of feed per dog was provided once daily, except during designated procedures. On the morning of dosing (pre-dose), the animals were deprived from their daily food allowance until 2 h post dose. Mains quality tap water was available ad libitum. Details of diet and water are maintained at the test facility.

Prior to acceptance for use on study, animals were subject to an examination and the results found to be satisfactory. All animals were weighed prior to each dose administration and the bodyweights recorded. Animals were checked regularly throughout the duration of the study. Any clinical signs were closely monitored and recorded.

Holding and study areas had automatic control of light cycles and temperature. Light hours were 07:00-19:00 h. Ranges of temperature and humidity measured during the study were 15-24° C. and 40-70%, respectively.

Materials. DMT-d₁₀ fumarate (I-8a), Batch No. ABN-1177 was supplied by Quotient Sciences and was stored frozen and protected from light and moisture in a freezer set to maintain a temperature of ≤−20° C. at the Test Facility. DMT fumarate (I-1a) Batch No. 0000040525 was supplied by Biosynth. Table 22 shows the material characteristics.

TABLE 22

| | | | MW Free | | |
|---|---|---|---|---|---|
| | | | Base | MW Salt | Correction |
| Material | Batch No. | Purity (%) | (g/mol) | (g/mol) | Factor |
| DMT-d₁₀ | ABN-1177 | 99.3 | 198.34 | 314.4 | 1.585 |
| fumarate | | | | | |
| DMT | 0000040525 | 99.0 | 188.27 | 304.34 | 1.617 |
| fumarate | | | | | |

Sodium hyaluronate was Hyatrue® HA-EP1.8 (molecular weight range of 900-1,400 kDa), available from Bloomage Freda Biopharm Co. Ltd.

Test Items. DMT-d₁₀ fumarate was formulated as a solution in 0.9% (w/v) saline, pH 5.5-6 (control) or in 1% (w/v, nominal) sodium hyaluronate in water for injection (WFI) (Formulation 28) at a free base concentration of 0.4 mg/mL (nominal) for SC injection. Each test item was prepared on the morning of dose administration and stored at ambient temperature, protected from light, until dosing.

Control (0.9% w/v saline): On the day prior to dosing, DMT-d₁₀ fumarate was removed from storage (frozen, in a freezer set to maintain ≤−20° C., protected from light) and allowed to equilibrate to room temperature. Using a precision analytical balance, 9.7 mg of DMT-d₁₀ fumarate was weighed into a 20 mL headspace vial which was pre-calibrated to a final volume of 15 mL with a magnetic stir bar inside. The weighed API was returned to storage. On the morning of dosing, the weighed API was removed from storage and allowed to equilibrate to room temperature. 13.55 mL of vehicle (0.9% w/v saline) was added to the vial (equivalent to 90% of final volume) and set to magnetically stir for ca 35 minutes. Using a calibrated pH meter, the solution pH was measured. The initial reading showed the pH as 3.90, this was then adjusted using 0.1M NaOH to a final pH of 5.97. The solution was made to final volume of 15 mL, using the pre-calibrated line, with 0.9% w/v saline. The final pH was recorded as 5.53. The solution was then filtered using a 0.22 μM PVDF syringe filter in the laminar air flow cabinet (LAFC). The visual appearance of the solution was recorded as a clear, colorless, homogenous solution. The final control solution had a free base concentration of 0.41 mg/mL DMT-d₁₀. The control solution was protected from light at all times.

Formulation 28: On the morning of dosing, DMT-d₁₀ fumarate was removed from storage (frozen, in a freezer set to maintain ≤−20° C., protected from light) and allowed to equilibrate to room temperature. Using a precision analytical balance, 11.02 mg of DMT-d₁₀ fumarate was weighed into a 20 mL headspace vial containing a magnetic stir bar. 3.5 mL of WFI was added to the vial and set to stir, making a stock solution with a free base concentration of 2 mg/mL (nominal) DMT-d₁₀. Once homogenous, 2 mL of the stock solution was dispensed into a vial containing 8 mL of 1.25% (w/v, nominal) sodium hyaluronate and vortex mixed until homogenous. The formulation thus had a free base concentration of 0.4 mg/mL (nominal) DMT-d₁₀ and a sodium hyaluronate concentration of 1% w/v (nominal).

Subcutaneous injections. The target dose was 0.1 mg/kg, free base at a dose concentration of 0.4 mg/mL. Appropriate volumes of dose were calculated per each animal based on real animal bodyweights and a target dose volume of 0.25 mL/kg. The volumes were pulled into plastic Troge 2.5 mL syringes. Animals were identified by microchip and tattoo. Animals were dosed via SC injection into the back of the neck over ca 30 seconds using a 21G (AGANI) needle. Animals were restrained appropriately throughout and returned to their pre-trial housing post dose. Three male Beagle dogs received a SC administration of control. After a 7-day washout period, the same animals were administered Formulation 28 as shown in Table 23.

TABLE 23
Experimental Design-Dose Formulation

| | | | Dose | | Dose | Dose |
|---|---|---|---|---|---|---|
| Animal | | Route of | Levelª | Release | Concentration^{b} | Volume |
| ID | Test Item | Administration | (mg/kg) | modifier | (mg/mL) | (mL/kg) |
| 068M | Control | SC | 0.1 | — | 0.4 | 0.25 |
| 069M | | | | | | |
| 070M | | | | | | |

7 Day Washout

| | | | | | | |
|---|---|---|---|---|---|---|
| 068M | Formulation | SC | 0.1 | 1% w/v | 0.4 | 0.25 |
| 069M | 28 | | | (nominal) | | |
| 070M | | | | sodium | | |
| | | | | hyaluronate | | |

^{a}calculated as free base
^{b}nominal free base concentration

Sample collection. Blood samples (ca 1 mL) were collected from the jugular vein by venipuncture into tubes containing K₂EDTA anticoagulant at the following time-points: Predose, 0.083 (5 min), 0.25 (15 min), 0.5 (30 min), 0.75 (45 min), 1, 2, 4 and 8 hr post dose. Immediately following blood sample collection, the tubes were inverted to ensure mixing with the anticoagulant and immediately placed on wet ice. As soon as practically possible, but no later than 30 minutes from collection, samples were centrifuged in a refrigerated (ca +4° C.) centrifuge at approximately 1500 g for 10 minutes. The resultant plasma was decanted into appropriately labelled polypropylene tubes in 96-well plate format and stored in a freezer set to maintain a temperature of ≤−65° C. until analysis. The samples were protected from light.

Bioanalysis. Plasma samples were analyzed for DMT-d₁₀ using an established LC-MS/MS assay. Pharmacokinetic parameters were determined from the DMT-d₁₀ plasma concentration-time profiles using commercially available software (Phoenix® WinNonlin®).

### Results.

All dose administrations of DMT-d₁₀ were performed without incident. No adverse reactions to the administration were observed in any of the animals dosed. Mild, transient clinical signs were recorded post-dose.

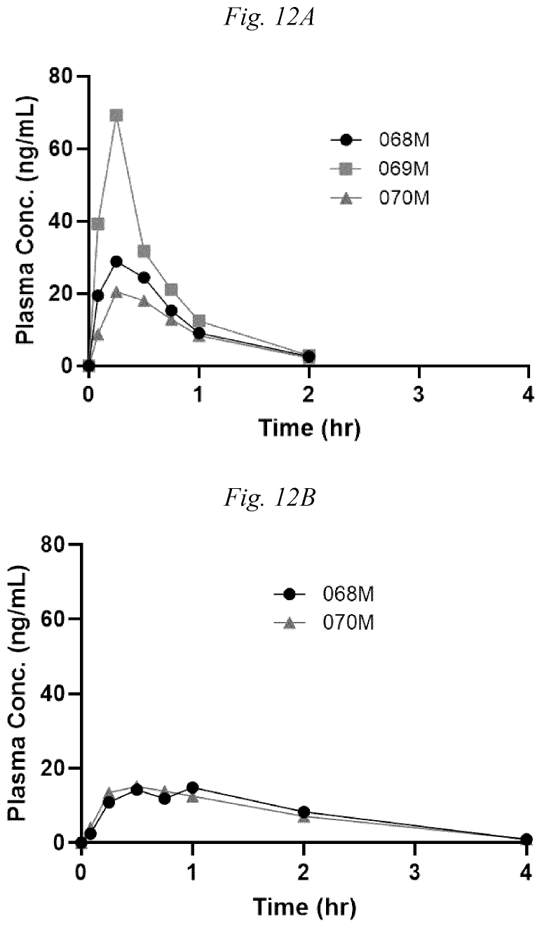

The pharmacokinetic parameters for DMT-d₁₀ formulated in either 0.9% w/v saline (control) or 1% w/v (nominal) sodium hyaluronate (Formulation 28) are presented. DMT-d₁₀ was administered SC at a free base equivalent dose of 0.1 mg/kg. Plasma DMT-d₁₀ concentrations were quantifiable up to 2 hours in all animals following SC administration of control (FIG. 12A), and up to 4 hours following SC administration of Formulation 28 (FIG. 12B).

As shown in Table 24 and FIGS. 12A-12B, after SC administration of DMT-d₁₀ at 0.1 mg/kg the pharmacokinetic profiles were dependent on formulation. The use of 1% w/v (nominal) sodium hyaluronate (n=2) increased the median Tₘₐₓ from 0.25 to 0.75 hr with subsequent reduction of mean Cₘₐₓ from 39.5 to 15.0 ng/mL, control (n=3) to Formulation 28 (n=2), respectively. The total exposure of DMT-d₁₀, AUC₀₋ₜ (31.1 h*ng/mL) from Formulation 28 was similar to that observed with control (30.6 h*ng/mL) indicating that sodium hyaluronate did not affect SC bioavailability. The elimination t_{1/2} after Formulation 28 was 0.836 hr (n=1); after control t_{1/2} was 0.479 hr (n=3), though these data are based on limited numbers.

TABLE 24
DMT-d₁₀ PK Profile after SC Administration of Control or Formulation 28.

| | Doseª | | Cₘₐₓ | Tₘₐₓ^{d} | AUC_{inf} | t_{1/2} | MRT_{inf} |
|---|---|---|---|---|---|---|---|
| Test Item | (mg/kg) | Stats^{b} | (ng/ml) | (hr) | (hr*ng/ml) | (hr) | (hr) |
| Control | 0.1 | mean | 39.5 | 0.25 | 30.6 | 0.479 | 0.746 |
| | | sd | 26.1 | (0.25-0.25) | 11.6 | 0.0384 | 0.108 |
| Formulation | 0.1 | mean | 15.0^{c} | 0.75^{d} | 31.1^{e} | 0.836^{e} | 1.42^{e} |
| 28 | | sd | NC | (0.5-1.0) | NC | NC | NC |

^{a}DMT-d₁₀ free base
^{b}n = 3 unless otherwise noted
^{c}n = 2; therefore, standard deviations were not calculated
^{d}median (minimum-maximum)
^{e}n = 1

B: Dose Proportionality—Pharmacokinetics (PK) of DMT-d₁₀ in Male Beagle Dogs Following Subcutaneous Administration Formulated in 1% w/v Sodium Hyaluronate at Increasing Dose Levels of DMT-d₁₀

The objective of this study was to determine if a SC administration of DMT-d₁₀ formulated in 1% w/v (nominal) sodium hyaluronate yields a dose proportional increase in plasma DMT-d₁₀ concentrations. This study was conducted using the same CRO, male Beagle dogs, housing, and materials as described above in part A. All conditions described in part A pertain to this study with the exceptions noted below.

Test Items. DMT-d₁₀ fumarate was formulated in 1% (w/v, nominal) sodium hyaluronate in water for injection (WFI) at a nominal free base concentration of 2 mg/mL (Formulation 29) or 4 mg/mL (Formulation 30) for SC injection. Each test item was prepared on the morning of dose administration and stored at ambient temperature, protected from light, until dosing.

Formulation 29: On the morning of dosing, DMT-d₁₀ fumarate was removed from storage (frozen, in a freezer set to maintain ≤−20° C., protected from light) and allowed to equilibrate to room temperature. Using a precision analytical balance, 110.7 mg of DMT-d₁₀ fumarate was weighed into a 20 mL headspace vial containing a magnetic stir bar. 6.914 mL of WFI was added to the vial and set to stir, making a stock solution with a free base concentration of 10.1 mg/mL (nominal) DMT-d₁₀. The stock solution had a density of 1.002 g/cm³. Once homogenous, 2.796 mL of the stock solution was dispensed into a vial containing 11.029 mL of 1.25% (w/v, nominal) sodium hyaluronate and vortex mixed until homogenous. The formulation thus had a free base concentration of 2.04 mg/mL (nominal) DMT-d₁₀ and a sodium hyaluronate concentration of 1% w/v (nominal). The visual appearance of the formulation was recorded as a clear, colorless, homogenous solution.

Formulation 30: On the morning of dosing, DMT-d₁₀ fumarate was removed from storage (frozen, in a freezer set to maintain ≤−20° C., protected from light) and allowed to equilibrate to room temperature. Using a precision analytical balance, 222.2 mg of DMT-d₁₀ fumarate was weighed into a 20 mL headspace vial containing a magnetic stir bar. 6.816 mL of WFI was added to the vial and set to stir, making a stock solution with a free base concentration of 20.6 mg/mL (nominal) DMT-d₁₀. The stock solution had a density of 1.006 g/cm³. Once homogenous, 2.830 mL of the stock solution was dispensed into a vial containing 11.208 mL of 1.25% (w/v, nominal) sodium hyaluronate and vortex mixed until homogenous. The formulation thus had a free base concentration of 4.15 mg/mL (nominal) DMT-d₁₀ and a sodium hyaluronate concentration of 1% w/v (nominal). The visual appearance of the formulation was recorded as a clear, colorless, homogenous solution.

Subcutaneous injections. The target dose was 0.5 mg/kg free base at a dose concentration of 2 mg/mL, or 1.0 mg/kg free base at a dose concentration of 4 mg/mL. Appropriate volumes of dose were calculated per each animal based on real animal bodyweights and a target dose volume of 0.25 mL/kg. The volumes were pulled into plastic Troge 2.5 mL syringes. Animals were identified by microchip and tattoo. Animals were dosed via SC injection into the back of the neck over ca 30 seconds using a 21G (AGANI) needle. Animals were restrained appropriately throughout and returned to their pre-trial housing post dose. The same animals were used with a 7-day washout period in between administrations as shown in Table 25.

TABLE 25
Experimental Design-Dose proportionality

| | | | Dose | | Dose | Dose |
|---|---|---|---|---|---|---|
| Animal | | Route of | Level | Release | Concentration^{b} | Volume |
| ID | Test Item | Administration | (mg/kg)^{a} | modifier | (mg/mL) | (mL/kg) |
| 068M | Formulation | SC | 0.1^{c} | 1% w/v | 0.4 | 0.25 |
| 069M | 28 | | | (nominal) | | |
| 070M | | | | sodium | | |
| | | | | hyaluronate | | |

7 Day Washout

| | | | | | | |
|---|---|---|---|---|---|---|
| 068M | Formulation | SC | 0.5 | 1% w/v | 2 | 0.25 |
| 069M | 29 | | | (nominal) | | |
| 070M | | | | sodium | | |
| | | | | hyaluronate | | |
| 068M | Formulation | SC | 1.0 | 1% w/v | 4 | 0.25 |
| 069M | 30 | | | (nominal) | | |
| 070M | | | | sodium | | |
| | | | | hyaluronate | | |

^{a}calculated as free base
^{b}nominal free base concentration
^{c}The 0.1 mg/kg dose with Formulation 28 was administered in part A. It is provided here for ease of comparing the PK parameters

### Results.

All dose administrations of DMT-d₁₀ were performed without incident. No adverse reactions to the administration were observed in any of the animals dosed. Mild, transient clinical signs were recorded post-dose.

The pharmacokinetic parameters for DMT-d₁₀ formulated in Formulations 28-30 SC at 0.1, 0.5 and 1 mg/kg are presented. The 0.1 mg/kg data (Formulation 28) were calculated in part A (see Table 24) and are repeated here to determine dose proportionality.

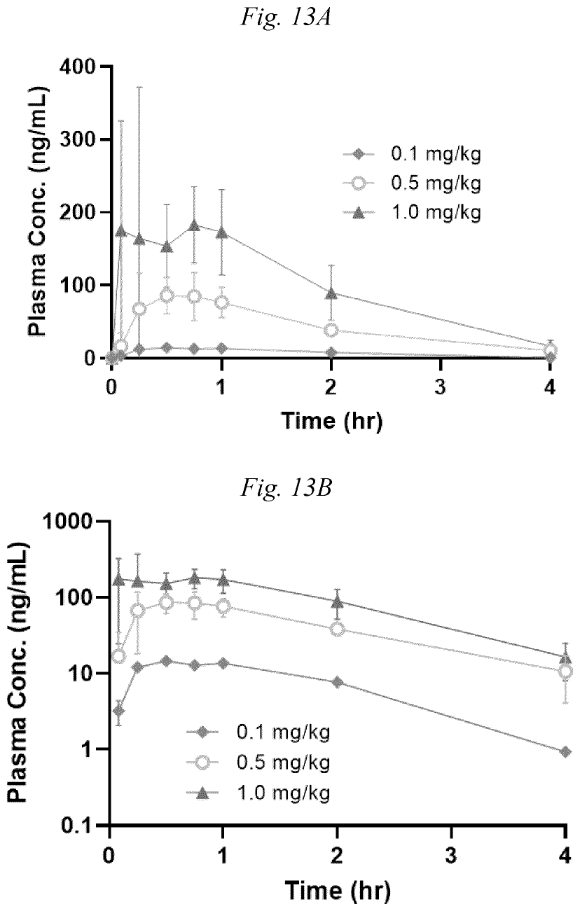

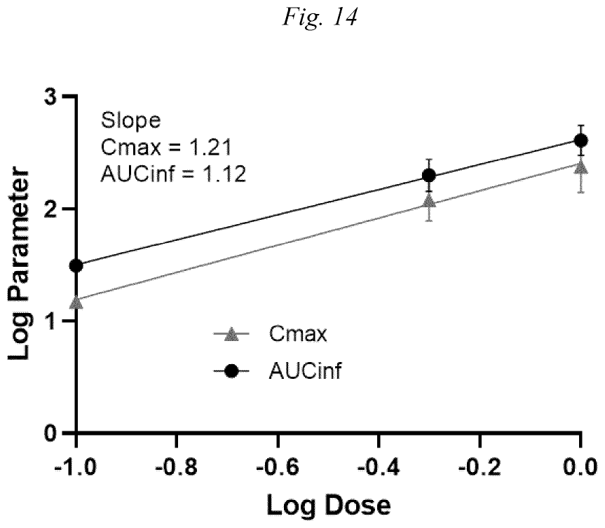

Plasma DMT-d₁₀ concentrations were quantifiable up to 4 hours in all animals following SC administration. Median Tₘₐₓ ranged from 0.5 to 0.75 hr suggesting similar absorption profiles after SC administration for the lower dose levels (FIGS. 13A-13B). However, the interpretation of the absorption kinetics may be impacted by the sparse samples collected during the phase and the interindividual variability at the high dose (1 mg/kg; Table 26). Independent of dose, elimination (t_{1/2}) of DMT-d₁₀ from the systemic circulation was consistent, approximately 0.9 hr, as was the length of time (MRT_{inf}) that DMT-d₁₀ presided in the circulation about 1.5 hr. As shown in FIG. 14, Cₘₐₓ and AUC_{inf} increased in a dose proportional manner. Taken together, these data indicate that a SC injection of DMT-d₁₀ formulated in 1% w/v (nominal) sodium hyaluronate behaves in a first order manner, resulting in dose independent absorption and elimination from the systemic circulation.

TABLE 26
DMT-d₁₀ PK Profile after SC Administration of Formulations 28-30

| | Doseª | | Cₘₐₓ | Tₘₐₓ^{c} | AUC_{inf} | t_{1/2} | MRT_{inf} |
|---|---|---|---|---|---|---|---|
| Test Item | (mg/kg) | Stats | (ng/ml) | (hr) | (hr*ng/ml) | (hr) | (hr) |
| Formulation 28 | 0.1 | mean | 15.0^{b} | 0.75 | 31.1^{d} | 0.836^{d} | 1.42^{d} |
| | | sd | NC | (0.5-1.0) | NC | NC | NC |
| Formulation 29 | 0.5 | mean | 127 | 0.5 | 204 | 0.939 | 1.61 |
| | | sd | 51.7 | (0.25-2.0) | 65.9 | 0.331 | 0.235 |
| Formulation 30 | 1.0 | mean | 261 | 0.75 | 421 | 0.875 | 1.50 |
| | | sd | 134 | (0.25-0.75) | 122 | 0.158 | 0.398 |

^{a}DMT-d₁₀ free base
^{b}n = 2; therefore, standard deviations were not calculated (NC)
^{c}median (minimum-maximum)
^{d}n = 1

C: Formulation Stability—Pharmacokinetics (PK) of DMT-d₁₀ in Male Beagle Dogs Following Subcutaneous Administration at 1 mg/kg Formulated in Increasing Sodium Hyaluronate Concentrations

The objective of this study was to determine how increasing concentrations of sodium hyaluronate affect the release of DMT-d₁₀ into systemic circulation after SC administration. This study was conducted using the same CRO, housing, and materials as described above in part A. All conditions described in part A pertain to this study with the exceptions noted below.

Test Items. DMT-d₁₀ fumarate was formulated in 0.1% (w/v, nominal) sodium hyaluronate in water for injection (WFI) at a nominal free base concentration of 2 mg/mL (Formulation 31), 0.25% (w/v, nominal) sodium hyaluronate in water for injection (WFI) at a nominal free base concentration of 4 mg/mL (Formulation 32), or 0.5% (w/v, nominal) sodium hyaluronate in water for injection (WFI) at a nominal free base concentration of 4 mg/mL (Formulation 33) for SC injection. Each test item was prepared on the morning of dose administration and stored at ambient temperature, protected from light, until dosing.

Formulation 31: On the morning of dosing, DMT-d₁₀ fumarate was removed from storage (frozen, in a freezer set to maintain ≤−20° C., protected from light) and allowed to equilibrate to room temperature. Using a precision analytical balance, 58.32 mg of DMT-d₁₀ fumarate was weighed into a 20 mL headspace vial containing a magnetic stir bar. 16.9 mL of WFI was added to the vial and set to stir, making a stock solution with a free base concentration of 2.18 mg/mL (nominal) DMT-d₁₀. The stock solution had a density of 0.999 g/cm³. Once homogenous, 12.9 mL of the stock solution was dispensed into a vial containing 1.14 mL of 1.25% (w/v, nominal) sodium hyaluronate and vortex mixed until homogenous. The formulation thus had a free base concentration of 2.00 mg/mL (nominal) DMT-d₁₀ and a sodium hyaluronate concentration of 0.1% w/v (nominal). The visual appearance of the formulation was recorded as a clear, colorless, homogenous solution and the pH was 3.90.

Formulation 32: On the morning of dosing, DMT-d₁₀ fumarate was removed from storage (frozen, in a freezer set to maintain ≤−20° C., protected from light) and allowed to equilibrate to room temperature. Using a precision analytical balance, 120.4 mg of DMT-d₁₀ fumarate was weighed into a 20 mL headspace vial containing a magnetic stir bar. 14.9 mL of WFI was added to the vial and set to stir, making a stock solution with a free base concentration of 5.10 mg/mL (nominal) DMT-d₁₀. The stock solution had a density of 1.000 g/cm³. Once homogenous, 11.2 mL of the stock solution was dispensed into a vial containing 2.80 mL of 1.25% (w/v, nominal) sodium hyaluronate and vortex mixed until homogenous. The formulation thus had a free base concentration of 4.08 mg/mL (nominal) DMT-d₁₀ and a sodium hyaluronate concentration of 0.25% w/v (nominal). The visual appearance of the formulation was recorded as a clear, colorless, homogenous solution and the pH was 3.59.

Formulation 33: On the morning of dosing, DMT-d₁₀ fumarate was removed from storage (frozen, in a freezer set to maintain ≤−20° C., protected from light) and allowed to equilibrate to room temperature. Using a precision analytical balance, 130.92 mg of DMT-d₁₀ fumarate was weighed into a 20 mL headspace vial containing a magnetic stir bar. 12.4 mL of WFI was added to the vial and set to stir, making a stock solution with a free base concentration of 6.66 mg/mL (nominal) DMT-d₁₀. The stock solution had a density of 1.001 g/cm³. Once homogenous, 8.39 mL of the stock solution was dispensed into a vial containing 5.55 mL of 1.25% (w/v, nominal) sodium hyaluronate and vortex mixed until homogenous. The formulation thus had a free base concentration of 4.01 mg/mL (nominal) DMT-d₁₀ and a sodium hyaluronate concentration of 0.50% w/v (nominal). The visual appearance of the formulation was recorded as a clear, colorless, homogenous solution and the pH was 3.76.

Subcutaneous injections. The target dose was 0.5 mg/kg free base at a dose concentration of 2 mg/mL, or 1.0 mg/kg free base at a dose concentration of 4 mg/mL. Appropriate volumes of dose were calculated per each animal based on real animal bodyweights and a target dose volume of 0.25 mL/kg. The volumes were pulled into plastic Troge 2.5 mL syringes. Animals were identified by microchip and tattoo. Animals were dosed via SC injection into the back of the neck over ca 30 seconds using a 21G (AGANI) needle. Animals were restrained appropriately throughout and returned to their pre-trial housing post dose. The same animals were used with a 7-day washout period in between administrations as shown in Table 27.

TABLE 27
Experimental Design-Formulation Stability

| | | | Dose | | Dose | Dose |
|---|---|---|---|---|---|---|
| Animal | | Route of | Level | Release | Concentration^{b} | Volume |
| ID | Test Item | Administration | (mg/kg)ª | Modifier | (mg/mL) | (mL/kg) |
| 027M | Formulation | SC | 0.5 | 0.1% w/v | 2 | 0.25 |
| 028M | 31 | | | (nominal) | | |
| 029M | | | | sodium | | |
| | | | | hyaluronate | | |

7 Day Washout

| | | | | | | |
|---|---|---|---|---|---|---|
| 027M | Formulation | SC | 1.0 | 0.25% w/v | 4 | 0.25 |
| 028M | 32 | | | (nominal) | | |
| 029M | | | | sodium | | |
| | | | | hyaluronate | | |
| 027M | Formulation | SC | 1.0 | 0.5% w/v | 4 | 0.25 |
| 028M | 33 | | | (nominal) | | |
| 029M | | | | sodium | | |
| | | | | hyaluronate | | |
| 068M | Formulation | SC | 1.0^{c} | 1% w/v | 4 | 0.25 |
| 069M | 30 | | | (nominal) | | |
| 070M | | | | sodium | | |
| | | | | hyaluronate | | |

^{a}calculated as free base
^{b}nominal free base concentration
^{c}The 1 mg/kg dose formulated in Formulation 30 was administered in part B. It is provided here for ease of comparing the PK parameters

### Results.

All dose administrations of DMT-d₁₀ were performed without incident. No adverse reactions to the administration were observed in any of the animals dosed. Mild, transient clinical signs were recorded post-dose.

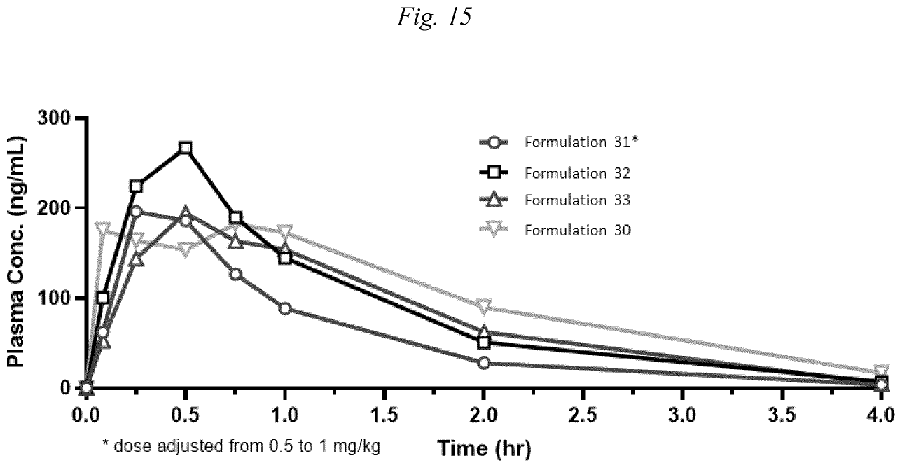

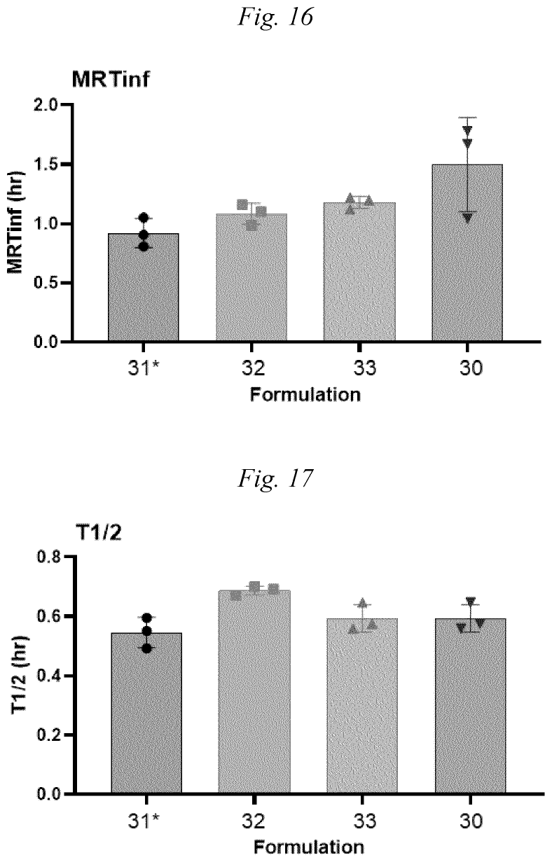

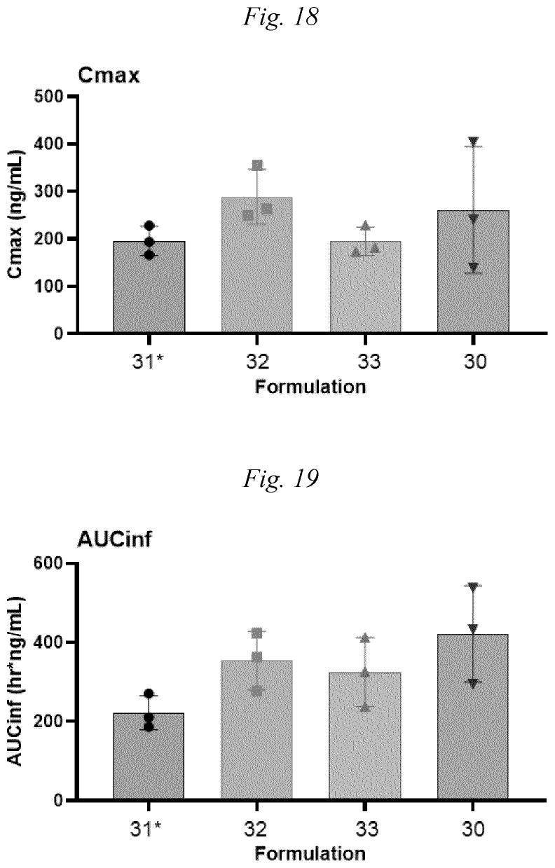

DMT-d₁₀ plasma concentration-time profiles formulated in 0.10% (Formulation 31), 0.25% (Formulation 32), 0.5% (Formulation 33), and 100 (Formulation 30) w/v (nominal) sodium hyaluronate at a SC dose of 1 mg/kg are presented in FIG. 15 (Formulation 31 dosed at 0.5 mg/kg free base was dose adjusted to 1 mg/kg). Corresponding pharmacokinetic parameters for DMT-d₁₀ are presented in Table 28. The PK parameters for Formulation 30 and control were calculated in part B and A, respectively, and are shown here for ease of comparison. Plasma DMT-d₁₀ concentrations were quantifiable up to 4 hours in all animals following SC administration. Median Tₘₐₓ increased as sodium hyaluronate concentration increased 3-fold from 0.25 hr at 0.1% w/v (nominal) sodium hyaluronate (Formulation 31) to 0.75 hr at 1% w/v (nominal) sodium hyaluronate (Formulation 30) (FIGS. 16-19). MRT_{inf}, a parameter describing the duration of DMT-d₁₀ systemic exposure, incrementally increased with higher sodium hyaluronate concentrations, i.e., 0.921 hr at 0.1% w/v (nominal) sodium hyaluronate (Formulation 31) to 1.50 hr at 1% w/v (nominal) sodium hyaluronate (Formulation 30). The incremental increase in MRT_{inf} can be seen graphically in FIG. 16.

However, over the same formulations, t_{1/2}, Cₘₐₓ and AUC_{inf} values were not changed (FIGS. 17-19, respectively). The data suggest that an increase in the sodium hyaluronate in the formulation can modulate the release of DMT-d₁₀ from the subcutaneous space (increases Tₘₐₓ and MRT_{inf}) without affecting the Cₘₐₓ and AUC_{inf} or the elimination of DMT-d₁₀ from the circulation.

TABLE 28
DMT-d₁₀ PK Profile after SC Administration of Formulations 30-33

| | Doseª | | Cₘₐₓ | Tₘₐₓ^{d} | AUC_{inf} | t_{1/2} | MRT_{inf} |
|---|---|---|---|---|---|---|---|
| Test Item | (mg/kg) | Stats | (ng/ml) | (hr) | (hr*ng/ml) | (hr) | (hr) |
| Formulation 31 | 1.0^{b} | mean | 196 | 0.25 | 222 | 0.546 | 0.921 |
| | | sd | 30.9 | (0.25-0.25) | 42.8 | 0.0519 | 0.122 |
| Formulation 32 | 1.0 | mean | 289 | 0.5 | 354 | 0.688 | 1.08 |
| | | sd | 57.6 | (0.25-0.5) | 73.5 | 0.0152 | 0.0874 |
| Formulation 33 | 1.0 | mean | 195 | 0.5 | 324 | 0.594 | 1.18 |
| | | sd | 30.1 | (0.5-0.5) | 87.7 | 0.0466 | 0.0522 |
| Formulation 30 | 1.0 | mean | 261 | 0.75 | 421 | 0.875 | 1.50 |
| | | sd | 134 | (0.25-0.75) | 122 | 0.158 | 0.398 |
| Controle | 1.0^{c} | mean | 395 | 0.25 | 306 | 0.479 | 0.746 |
| | | sd | 261 | (0.25-0.25) | 116 | 0.0384 | 0.108 |

^{a}DMT-d₁₀ free base
^{b}dose adjusted from 0.5 to 1.0 mg/kg
^{c}dose adjusted from 0.1 to 1.0 mg/kg
^{d}median (minimum-maximum)
^{e}Administered in part A. It is provided here for ease of comparing the PK parameters

D: Dose Concentration and Dose Volume—Pharmacokinetics of DMT-d₁₀ in Male Beagle Dogs Following Subcutaneous Administration at 1 mg/kg Formulated in Hyaluronic Acid Concentrations at Higher Dosing Solution Concentrations and Lower Dosing Volume

The objective of this study was to determine the effect of increasing the dosing solution concentration of DMT-d₁₀ and decreasing the dose volume on the SC release of DMT-d₁₀ into the systemic circulation. This study was conducted using the same CRO, housing, and materials as described above in part A. All conditions described in part A pertain to this study with the exceptions noted below.

Test Items. DMT-d₁₀ fumarate was formulated as a solution in 0.9% (w/v) saline, pH 5.5-6, at a free base concentration of 4 mg/mL (nominal) (control) or in 0.4% (w/v, nominal) sodium hyaluronate in 0.3% (w/v) saline at a free base concentration of 20 mg/mL (nominal) (Formulation 34) for SC injection. Each test item was prepared on the morning of dose administration and stored at ambient temperature, protected from light, until dosing.

Control (0.9% w/v saline): On the morning of dosing, DMT-d₁₀ fumarate was removed from storage (frozen, in a freezer set to maintain ≤−20° C., protected from light) and allowed to equilibrate to room temperature. Using a precision analytical balance, 88.8 mg of DMT-d₁₀ fumarate was weighed into a suitably sized sterile glass container, pre-calibrated to the final volume required. 90% final volume of vehicle (0.9% w/v saline) was added to the vial and set to magnetically stir. The pH was then measured using a calibrated pH meter and was found to be 3.18 initially. The pH was adjusted using 1M NaOH and 2M HCl to achieve a final pH of 5.51. The control solution was then made to final volume using a pre-calibrated line on the glass vial with 0.9% (w/v) saline. Sterile filtration using a 0.22 M filter was then carried out. The control solution had a free base concentration of 4 mg/mL (nominal) DMT-d₁₀.

Formulation 34: A 0.3% (w/v) saline solution was prepared by adding 3.35 mL of 0.9% (w/v) to a sterile glass vial with 6.65 mL of WFI, followed by magnetically mixing. Once homogeneous, the density was measured to be 1.000 g/cm³. On the morning of dosing, DMT-d₁₀ fumarate was removed from storage (frozen, in a freezer set to maintain ≤−20° C., protected from light) and allowed to equilibrate to room temperature. Using a precision analytical balance, 311.1 mg of DMT-d₁₀ fumarate was weighed into a suitably sized sterile glass container. 2.839 mL of the previously prepared 0.3% (w/v) solution (equivalent to 60% of the final stock volume) was added to the container and set to stir at a temperature of 37° C. at 300 rpm. The visual appearance was recorded as a clear/yellowish homogenous solution at this stage. The pH was measured using a calibrated pH meter and recorded to be 3.24. The pH was then adjusted to 5.98 by adding 1M NaOH until a final volume of 5 mL using a volumetric flask, and was set to stir magnetically. The density was measured to be 1.012 g/cm³. Sterile filtration using a 0.22 μM PVDF filter was then carried out. The stock solution had a free base concentration of 40 mg/mL (nominal) DMT-d₁₀.

A 0.8% (w/v, nominal) sodium hyaluronate solution was prepared by precisely mixing 3.193 mL of 1.25% (w/v, nominal) sodium hyaluronate and 1.799 mL of 0.8% (w/v) saline in a glass container until homogeneous. The density of the resultant 0.8% (w/v, nominal) sodium hyaluronate solution was recorded to be 1.002 g/cm³. 2.032 mL of the DMT-d₁₀ fumarate stock solution and 1.988 mL of the 0.8% (w/v, nominal) sodium hyaluronate solution were added to a new sterile vial and magnetically stirred until homogeneous. The formulation thus had a free base concentration of 20 mg/mL (nominal) DMT-d₁₀ and a sodium hyaluronate concentration of 0.40% w/v (nominal). The final visual appearance of the formulation was recorded as a clear, colorless, homogenous solution.

Subcutaneous injections. The target dose was 1 mg/kg free base at a dose concentration of 20 mg/mL (nominal), or 1.0 mg/kg free base at a dose concentration of 4 mg/mL (nominal). Appropriate volumes of dose were calculated per each animal based on real animal bodyweights and a target dose volume of 0.05 mL/kg or 0.25 mL/kg. The volumes were pulled into plastic Troge 2.5 mL syringes. Animals were identified by microchip and tattoo. Animals were dosed via SC injection into the back of the neck over ca 30 seconds using a 21G (AGANI) needle. Animals were restrained appropriately throughout and returned to their pre-trial housing post dose. The same animals were used with a 7-day washout period in between administrations as shown in Table 29.

TABLE 29
Experimental Design-Effect of Dose Solution Concentration and Dose Volume on
the PK Profile of DMT-d₁₀

| | | | Dose | | Dose | Dose |
|---|---|---|---|---|---|---|
| Animal | | Route of | Levelª | Release | Concentration^{b} | Volume |
| ID | Test Item | Administration | (mg/kg) | modifier | (mg/mL) | (mL/kg) |
| 006M | Formulation | SC | 1.0 | 0.4% w/v | 20 | 0.05 |
| 027M | 34 | | | (nominal) | | |
| 028M | | | | sodium | | |
| | | | | hyaluronate | | |

7 Day Washout

| | | | | | | |
|---|---|---|---|---|---|---|
| 006M | Control | SC | 1.0 | — | 4 | 0.25 |
| 027M | | | | | | |
| 028M | | | | | | |

^{a}calculated as free base
^{b}nominal free base concentration

Sample collection. Blood samples (ca 1 mL) were collected from the jugular vein by venipuncture into tubes containing K₂EDTA anticoagulant at the following time-points: Predose, 0.033 (2 min), 0.083 (5 min), 0.25 (15 min), 0.5 (30 min), 0.75 (45 min), 1, 2, and 4 hr post dose.

### Results.

All dose administrations of DMT-d₁₀ were performed without incident. No adverse reactions to the administration were observed in any of the animals dosed. Mild, transient clinical signs were recorded post-dose.

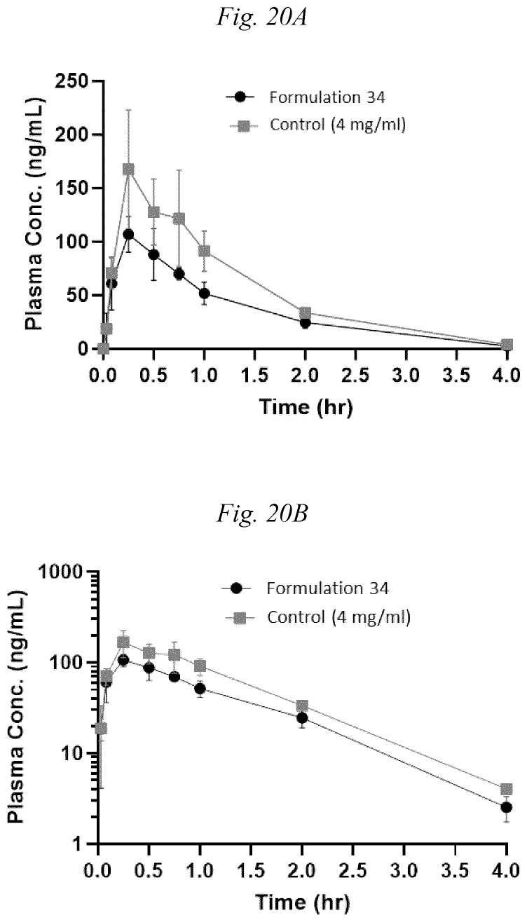

DMT-d₁₀ was formulated in 0.4% w/v (nominal) sodium hyaluronate containing 0.3% w/v NaCl at a SC dose solution concentration of 20 mg/mL (nominal) (Formulation 34) and delivered as a dose volume of 0.05 mL/kg or in 0.9% w/v NaCl at a SC dose solution concentration of 4 mg/mL (nominal) (control) and delivered as a dose volume of 0.25 mL/kg, both at a free base dose of 1 mg/kg. The DMT-d₁₀ plasma concentration-time concentrations are presented in FIGS. 20A and 20B. Corresponding pharmacokinetic parameters for DMT-d₁₀ are presented in Table 30. Plasma DMT-d₁₀ concentrations were quantifiable up to 4 hours in all animals following SC administration. Median Tₘₐₓ values were 0.25 hr for both Formulation 34 and control. The t_{1/2} (0.682 and 0.667 hr) and MRT_{inf} (1.11 and 1.10 hr) of Formulation 34 and control, respectively, were similar for both formulations. Cₘₐₓ and AUC_{inf} decreased by about 35% for Formulation 34 compared to control.

In summary, both formulations have solubility limits ≥20 mg/mL. The high dose solution concentration affords a lower SC injection volume (0.5 mL for a 10 kg dog) thereby diminishing possible discomfort and/or local tissue damage associated with the injection site. The resulting plasma concentration-time profiles were similar with the exception that the apparent bioavailability DMT-d₁₀ when administered in Formulation 34 was 65% compared to control.

TABLE 30
DMT-d₁₀ PK Profile after SC Administration of Control or Formulation 34.

| | Doseª | | Cₘₐₓ | Tₘₐₓ^{d} | AUC_{inf} | t_{1/2} | MRT_{inf} |
|---|---|---|---|---|---|---|---|
| Test Item | (mg/kg) | Stats | (ng/mL) | (hr) | (hr*ng/mL) | (hr) | (hr) |
| Formulation 34 | 1.0^{b} | mean | 112 | 0.25 | 144 | 0.682 | 1.11 |
| | | sd | 15.5 | (0.25-0.5) | 10.7 | 0.0415 | 0.087 |
| Control | 1.0^{c} | mean | 169 | 0.25 | 221 | 0.667 | 1.10 |
| | | sd | 54.4 | (0.25-0.5) | 39.2 | 0.0337 | 0.0696 |

^{a}DMT-d₁₀ free base
^{b}dose solution concentration at 20 mg/mL (nominal); dose volume administration at 0.05 mL/kg
^{c}dose solution concentration at 4 mg/mL (nominal); dose volume administration at 0.25 mL/kg
^{d}median (minimum-maximum)

E: Deuteration Effect—Pharmacokinetics of DMT-d₁₀ and DMT Co-Dosed in Male Beagle Dogs Following Subcutaneous Administration at 0.5 mg/kg/analyte (total dose: 1 mg/kg) Formulated in Sodium Hyaluronate

The objective of this study was to determine the effect of deuteration on the PK profile of DMT after a SC administration at 0.5 mg/kg/analyte formulated in 0.5% w/v (nominal) sodium hyaluronate in water for injection (WFI). This study was conducted using the same CRO, housing, and materials as described above in part A. All conditions described in part A pertain to this study with the exceptions noted below.

Test Items. DMT fumarate and DMT-d₁₀ fumarate were formulated together as a solution in 0.5% (w/v, nominal) sodium hyaluronate in water for injection (WFI) at a free base concentration of 10 mg/mL/analyte (nominal); total free base concentration of 20 mg/mL (nominal) (Formulation 35) for SC injection. Each test item was prepared on the morning of dose administration and stored at ambient temperature, protected from light, until dosing.

Formulation 35: On the morning of dosing, DMT-d₁₀ fumarate and DMT fumarate were removed from storage (frozen, in a freezer set to maintain ≤−20° C., protected from light) and allowed to equilibrate to room temperature. Using a precision analytical balance, 80.1 mg of DMT-d₁₀ fumarate and 82.3 mg of DMT fumarate were weighed into a suitably sized sterile glass container. 2.853 mL of WFI was added to the container and magnetically stirred. Once homogeneous, the stock solution was sterile filtered using a 0.22 μM PVDF filter. The stock solution had a free base concentration of 17.7 mg/mL (nominal) DMT-d₁₀ and a free base concentration 17.8 mg/mL DMT (combined free base concentration of DMT-d₁₀+DMT of 35.5 mg/mL). 1.832 mL of the stock solution was added to a new sterile glass vial. 1.203 mL of 1.25% (w/v, nominal) sodium hyaluronate solution was added to the vial and vortex mixed to homogenize. The formulation thus had a free base concentration of 10.7 mg/mL (nominal) DMT-d₁₀ and a free base concentration of 10.8 mg/mL (nominal) DMT (combined free base concentration of DMT-d₁₀+DMT of 21.5 mg/mL) and a sodium hyaluronate concentration of 0.5% w/v (nominal).

Subcutaneous injections. DMT+DMT-d₁₀ were co-dosed (via Formulation 35) at 0.5 mg/kg/analyte, total dose of 1.0 mg/kg (free base), each at a dose concentration of 10 mg/mL (nominal), total dose concentration of 20 mg/mL (nominal) and a dose volume of 0.05 mL/kg calculated per each animal based on real animal bodyweights. The volumes were pulled into plastic Troge 2.5 mL syringes. Animals were identified by microchip and tattoo. Animals were dosed via SC injection into the back of the neck over ca 30 seconds using a 21G (AGANI) needle. Animals were restrained appropriately throughout and returned to their pre-trial housing post dose. The experimental design is shown in Table 31.

TABLE 31
Experimental Design-Effect of Deuteration on the PK Profile of DMT in Sodium
Hyaluronate Formulations

| | | | Dose | | Dose | Dose |
|---|---|---|---|---|---|---|
| Animal | | Route of | Level^{a, b} | Release | Concentration^{c, d} | Volume |
| ID | Test Item | Administration | (mg/kg) | modifier | (mg/mL) | (mL/kg) |
| 006M | Formulation | SC | 0.5 | 0.5% w/v | 10 | 0.05 |
| 027M | 35 | | | (nominal) | | |
| 028M | (DMT + | | | sodium | | |
| | DMT-d₁₀) | | | hyaluronate | | |

^{a}calculated as free base
^{b}DMT and DMT-d₁₀ were co-dosed at 0.5 mg/kg/analyte: total dose of 1.0 mg/kg
^{c}nominal free base concentration
^{d}DMT and DMT-d₁₀ were co-dosed at a dose concentration of 10 mg/ml/analyte; total dose concentration of 20 mg/mL

Sample collection. Blood samples (ca 1 mL) were collected from the jugular vein by venipuncture into tubes containing K₂EDTA anticoagulant at the following time-points: Predose, 0.033 (2 min), 0.083 (5 min), 0.25 (15 min), 0.5 (30 min), 0.75 (45 min), 1, 2, and 4 hr post dose.

### Results.

All dose administrations were performed without incident. No adverse reactions to the administration were observed in any of the animals dosed. Mild, transient clinical signs were recorded post-dose.

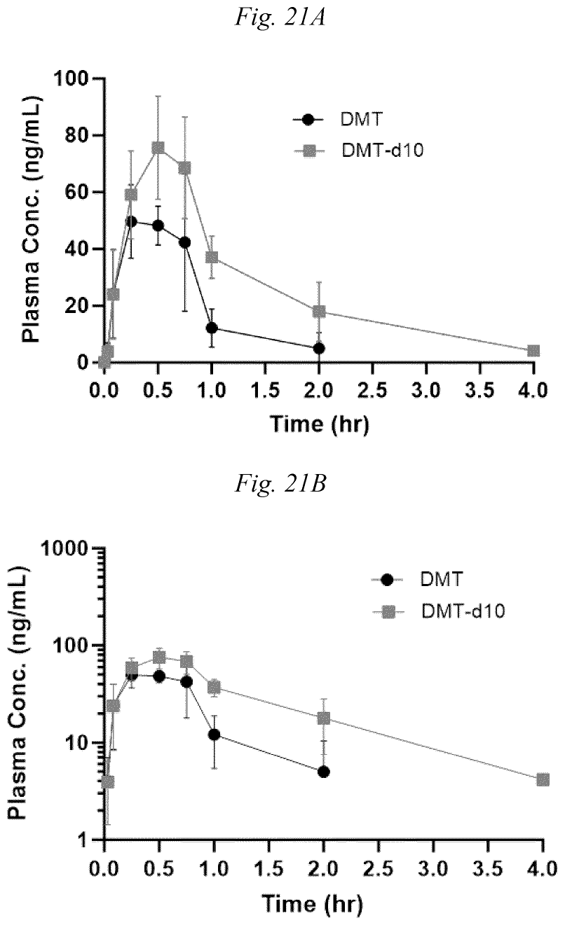

DMT and DMT-d₁₀ were co-dosed via Formulation 35 in 0.5% w/v (nominal) sodium hyaluronate in water for injection (WFI) at a SC dose solution concentration of 10 mg/mL/analyte (total: 20 mg/mL) (nominal) and dose volume of 0.05 mL/kg (0.5 mL for a 10 kg dog). DMT and DMT-d₁₀ plasma concentration-time concentrations are presented in FIGS. 21A and 21B. Corresponding pharmacokinetic parameters for DMT and DMT-d₁₀ are presented in Table 32. Plasma concentrations were quantifiable to 2 hr for DMT and 4 hr for DMT-d₁₀ following SC administration. Median Tₘₐₓ values were 0.5 hr for DMT and DMT-d₁₀. Deuteration of DMT, however, decreased apparent clearance (Cl/F) by 2.2× (10400 to 4650 mL/hr/kg) and decreased in the apparent volume of distribution (V_{z}/F) by 1.6× (7590 to 4729 mL/kg) leading to increased Cₘₐₓ, AUC_{inf}, t_{1/2} and MRT_{inf} by 1.3×, 2.2×, 1.4× and 1.4×, respectively.

These data indicate that deuteration has a significant impact on elimination from the systemic circulation as manifested in the 2.2× decrease in apparent systemic clearance.

TABLE 32
Effect of Deuteration on the PK Profile of DMT in Sodium Hyaluronate Formulation 35

| | Doseª | | Cₘₐₓ | Tₘₐₓ^{b} | AUC_{inf} | t_{1/2} | MRT_{inf} | Vz/F | Cl/F |
|---|---|---|---|---|---|---|---|---|---|
| Analyte | (mg/kg) | Stats | (ng/mL) | (hr) | (hr*ng/mL) | (hr) | (hr) | (mL/kg) | (mL/hr/kg) |
| DMT | 0.5 | mean | 58.1 | 0.5 | 48.7 | 0.533 | 0.867 | 7590 | 10400 |
| | | sd | 15.6 | (0.25-0.75) | NA | NA | NA | NA | NA |
| DMT-d₁₀ | 0.5 | mean | 75.6 | 0.5 | 109 | 0.722 | 1.23 | 4720 | 4650 |
| | | sd | 18.2 | (0.5-0.5) | NA | NA | NA | NA | NA |

^{a}DMT or DMT-d₁₀ free base
^{b}median (minimum-maximum)
NA: not applicable;
n = 2 therefore standard deviations (sd) were not calculated

### F: Clinical Observation Summary in Male Beagle Dogs After SC Administration of DMT or DMT-d₁₀ Formulated in Sodium Hyaluronate

As detailed in Table 33, clinical observations were dose and formulation dependent. At 1% w/v (nominal) sodium hyaluronate, the pharmacodynamic behaviors began to emerge at 1 mg/kg to include salivation and vocalization. With a decrease in sodium hyaluronate from 1% to 0.25%, 0.4%, and 0.5% w/v (nominal), at 1 mg/kg, a cluster of pharmacological effects included licking, salivation, panting, vocalization, and wide eyes (dilated pupils) occurred. In some cases, animals appeared calm. Categorized as mild to moderate clinical symptoms began to appear 10 min postdose with resolution over 1 hour.

TABLE 33
Clinical Observation Summary in Male Beagle Dogs After SC Administration of
Formulations 28-35 formulated in Sodium Hyaluronate.

| | Doseª | Release | | | | | |
|---|---|---|---|---|---|---|---|
| Test Item | (mg/kg) | Modifier | Licking | Salivation | Panting | Vocalization | Wide eyes |
| Formulation | 0.1 | 1% w/v | N/O | N/O | N/O | N/O | N/O |
| 28 | | (nominal) | | | | | |
| | | sodium | | | | | |
| | | hyaluronate | | | | | |
| Formulation | 0.5 | 1% w/v | N/O | N/O | N/O | N/O | N/O |
| 29 | | (nominal) | | | | | |
| | | sodium | | | | | |
| | | hyaluronate | | | | | |
| Formulation | 1.0 | 1% w/v | N/O | 30 min post | N/O | 30 min post | N/O |
| 30 | | (nominal) | | dose (mild) | | dose (mild) | |
| | | sodium | | | | | |
| | | hyaluronate | | | | | |
| Formulation | 0.5 | 0.1 w/v | N/O | N/O | N/O | N/O | N/O |
| 31 | | | | | | | |
| Formulation | 1.0 | 0.25% w/v | 10 min | 10 min post | 10 min post | \|30 min post | 30 min |
| 32 | | (nominal) | post dose | dose (mild) | dose (mild) | dose | post dose |
| | | sodium | (mild) | through 45 | through 45 | | |
| | | hyaluronate | | min | min | | |
| Formulation | 1.0 | 0.5% w/v | 15 min | N/O | 15 min post | 30 min post | 30 min |
| 33 | | (nominal) | post dose | | dose (mild) | dose (mild) | post dose |
| | | sodium | (mild) | | | | (mild) |
| | | hyaluronate | | | | | |
| Formulation | 1.0 | 0.4% w/v | 15 min | N/O | 15 min post | N/O | N/O |
| 34 | | (nominal) | post dose | | dose (mild) | | |
| | | sodium | (mild) | | through 1 | | |
| | | hyaluronate | through 1 | | hour | | |
| | | | hour | | | | |
| Formulation | 0.5^{b} | 0.5% w/v | 15 min | N/O | 15 min post | N/O | N/O |
| 35 | | (nominal) | post dose | | dose (mild) | | |
| (DMT + | | sodium | (mild) | | through 45 | | |
| DMT-d₁₀) | | hyaluronate | through 45 | | min | | |
| | | | min | | | | |

^{a}DMT or DMT-d₁₀ free base
^{b}DMT and DMT-d₁₀ were co-dosed at 0.5 mg/kg/analyte: total dose of 1.0 mg/kg
N/O = Not Observed

### IV. Formulations and Release Testing—Dissociatives

### Materials and Methods

Ketamine (±) HCl was purchased from Patheon Pharma Services. Sodium chloride was ACS reagent grade, >99.0% (available from Sigma Aldrich). The release modifier used was sodium hyaluronate HA15M (molecular weight range of >1,000-1,800 kDa; Mw=1,670 kDa; available from Lifecore Biomedical, Inc.). Formulations were prepared using water for injection (WFI).

Filtration was performed using a Syringe filter unit, 0.22 μm, PVDF, 33 mm filter, available from Sigma-Aldrich.

The Dialysis Drug Release Test and UPLC were performed as previously described in section II.

### Formulations and Release Profiles

Formulations and their preparative methods are presented below. Formulations are presented in terms of the parameters A, B, and C as shown previously in Table 11.

### Formulations 36-38

To investigate the role of different concentrations of sodium hyaluronate (0.5, 0.75, and 1% w/v, nominal) at fixed molecular weight on the release of ketamine (12.5 mg/mL free base equivalence, nominal), three different stock sodium hyaluronate solutions were made at 1, 1.5, and 2% w/v (nominal) concentrations by dissolving 10 mg, 15 mg, and 20 mg of sodium hyaluronate (>1,000-1,800 kDa), respectively, in a volume of WFI to reach 1 mL, followed by incubation at 37° C. and stirring. The stock sodium hyaluronate solutions were then filtered through a 0.22 μm PVDF filter. A stock solution of ketamine HCl (25 mg/mL free base equivalence of ketamine, nominal) was prepared by dissolving 115 mg of ketamine HCl in 4 mL of WFI, followed by incubation at 37° C. and stirring. The solutions were used as is and no pH adjustments were made. Each stock sodium hyaluronate solution was then mixed in a 1:1 volume ratio with the stock solution of ketamine HCl, and the resulting formulations were vigorously stirred to ensure complete mixing. A control formulation (12.5 mg/mL free base equivalence of ketamine, nominal) without release modifier was prepared by mixing stock solution of ketamine HCl with WFI in a 1:1 volume ratio. Formulations are shown in Table 34.

TABLE 34

| | A) Free base conc. | B) Sodium |
|---|---|---|
| | of ketamine | hyaluronate conc. |
| | (mg/mL, nominal) | (% w/v, nominal) |
| Control | 12.5 | — |

C) Sodium hyaluronate = >1,000-1,800 kDa (Mw = 1,670 kDa)

| | | |
|---|---|---|
| Formulation 36 | 12.5 | 0.5% |
| Formulation 37 | 12.5 | 0.75% |
| Formulation 38 | 12.5 | 1% |

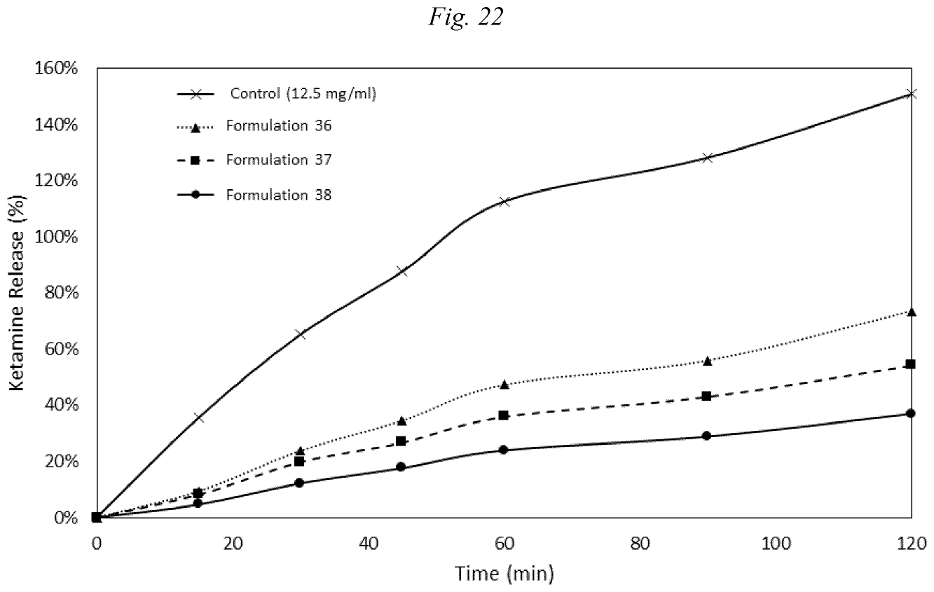

Each formulation was subjected to the Dialysis—Drug Release Test. FIG. 22 shows the drug release is highly controlled by the concentration of sodium hyaluronate in the formulation, with the control formulation providing the fastest release, and increasing sodium hyaluronate concentrations providing increasingly slower drug release, with the slowest release provided by the highest sodium hyaluronate content (Formulation 38). These formulations support the notion that sodium hyaluronate serves as a robust polymer matrix for tunable and controlled-release of drug.

The preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. An injectable pharmaceutical formulation, comprising:
a psychopharmaceutical agent;
a hyaluronate salt; and
an aqueous vehicle;
wherein the psychopharmaceutical agent is a pharmaceutically acceptable salt of a compound of Formula (I) or a stereoisomer, solvate, or prodrug thereof, 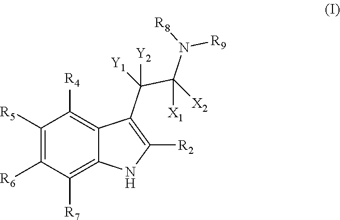
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, unsubstituted or substituted alkoxy, unsubstituted or substituted alkylthio, and unsubstituted or substituted acyloxy;
R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl; and
R₈ and R₉ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl,
or alternatively R₈ and R₉ together with the nitrogen atom attached thereto are optionally joined to form an unsubstituted or substituted heterocycloalkyl.

2. The injectable pharmaceutical formulation of claim 1, wherein the compound has a structure of Formula (II), or a stereoisomer, solvate, or prodrug thereof 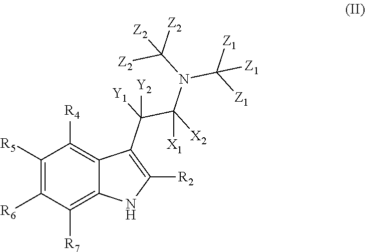
wherein:
X₁ and X₂ are independently hydrogen or deuterium,
Y₁ and Y₂ are independently hydrogen or deuterium,
each Z₁ is independently hydrogen or deuterium,
each Z₂ is independently hydrogen or deuterium, and
R₂, R₄, R₅, R₆, and R₇ are independently hydrogen or deuterium.

3. The injectable pharmaceutical formulation of claim 1, wherein the psychopharmaceutical agent is a pharmaceutically acceptable salt of at least one compound selected from the group consisting of 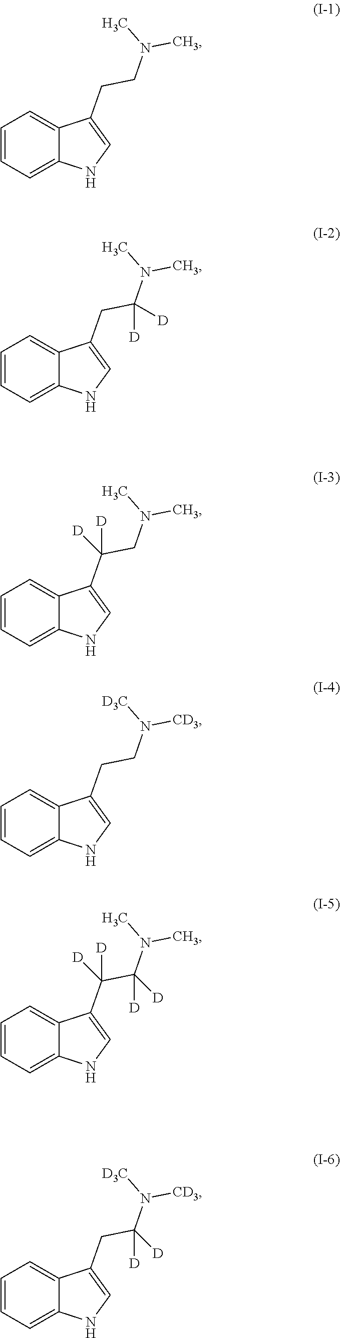 [SMILES: CN(C)CCc1c[nH]c2ccccc12 ; [2H]C([2H])(Cc1c[nH]c2ccccc12)N(C)C ; [2H]C([2H])(CN(C)C)c1c[nH]c2ccccc12 ; [2H]C([2H])([2H])N(CCc1c[nH]c2ccccc12)C([2H])([2H])[2H] ; [2H]C([2H])(c1c[nH]c2ccccc12)C([2H])([2H])N(C)C ; [2H]C([2H])([2H])N(C([2H])([2H])[2H])C([2H])([2H])Cc1c[nH]c2ccccc12] 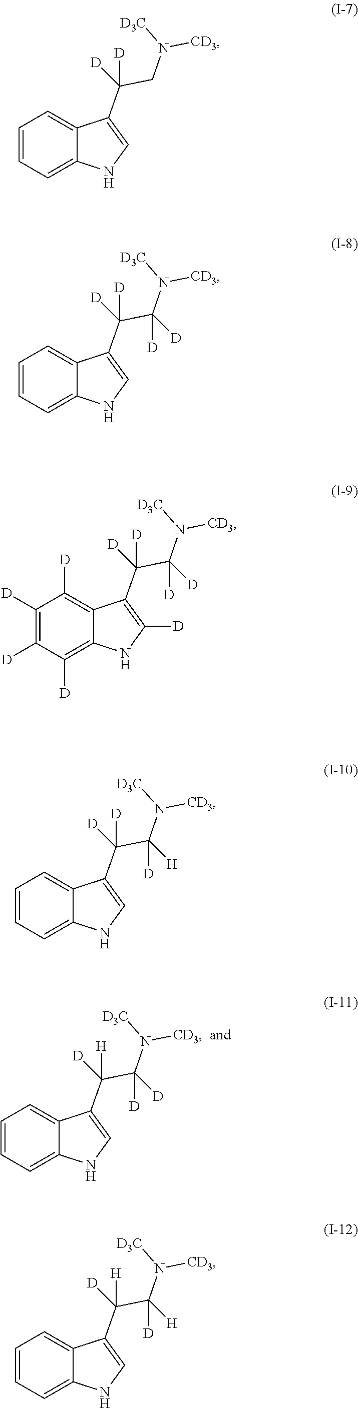
or a stereoisomer, solvate, or prodrug thereof.

4. The injectable pharmaceutical formulation of claim 1, wherein the psychopharmaceutical agent is a pharmaceutically acceptable salt of 2-(1H-indol-3-yl)-N,N-bis(methyl-d₃)ethan-1-amine-1,1,2,2-d₄ (I-8).

5-7. (canceled)

8. The injectable pharmaceutical formulation of claim 1, wherein the psychopharmaceutical agent is an active salt mixture comprising: (i) from 60% to 99% by weight of a pharmaceutically acceptable salt of 2-(1H-indol-3-yl)-N,N-bis(methyl-d₃)ethan-1-amine-1,1,2,2-d₄ (I-8), based on a total weight of the active salt mixture; and (ii) from 1% to 40% by weight, in sum, of a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), based on a total weight of the active salt mixture.

9. The injectable pharmaceutical formulation of claim 1, wherein the compound has a structure of Formula (III), or a stereoisomer, solvate, or prodrug thereof 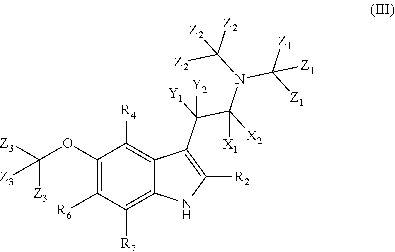
wherein:
X₁ and X₂ are independently hydrogen or deuterium,
Y₁ and Y₂ are independently hydrogen or deuterium,
each Z₁ is independently hydrogen or deuterium,
each Z₂ is independently hydrogen or deuterium,
each Z₃ is independently hydrogen or deuterium, and
R₂, R₄, R₆, and R₇ are independently hydrogen or deuterium.

10. (canceled)

11. The injectable pharmaceutical formulation of claim 1, wherein the psychopharmaceutical agent is a pharmaceutically acceptable salt of 2-(5-methoxy-1H-indol-3-yl)-N,N-bis(methyl-d₃)ethan-1-amine-1,1,2,2-d₄ (I-20).

12. (canceled)

13. The injectable pharmaceutical formulation of claim 1, wherein the pharmaceutically acceptable salt is a fumarate, a benzoate, a salicylate, a succinate, an oxalate, a glycolate, a hemi-oxalate, or a hemi-fumarate salt.

14. (canceled)

15. The injectable pharmaceutical formulation of claim 1, wherein a concentration of the psychopharmaceutical agent by weight (in terms of free base equivalence) per total volume of the pharmaceutical formulation is about 10 mg/mL to about 50 mg/mL.

16. The injectable pharmaceutical formulation of claim 1, wherein the hyaluronate salt is sodium hyaluronate.

17. The injectable pharmaceutical formulation of claim 1, wherein the hyaluronate salt has a weight average molecular weight of about 500 kDa to about 2,000 kDa.

18. (canceled)

19. The injectable pharmaceutical formulation of claim 1, wherein a concentration of the hyaluronate salt by weight per total volume of the pharmaceutical formulation is from about 0.1% to about 2% (w/v).

20-22. (canceled)

23. The injectable pharmaceutical formulation of claim 1, which has: (i) a pH of about 3 to about 7, (ii) an osmolality of about 150 mOsm/kg to about 600 mOsm/kg, or both (i) and (ii).

24-28. (canceled)

29. A kit suitable for preparing the injectable pharmaceutical formulation of claim 1, the kit comprising:
(a1) a first solution comprising the psychopharmaceutical agent and the aqueous vehicle; and
(b1) a second solution comprising the hyaluronate salt and the aqueous vehicle.

30. A method of treating a central nervous system (CNS) disorder and/or psychological disorder in a subject in need thereof, comprising administering to the subject a therapeutically effective amount of the injectable pharmaceutical formulation of claim 1.

31. The method of claim 30, wherein the CNS disorder and/or psychological disorder is a substance use disorder, an anxiety disorder, or a depressive disorder.

32. The method of claim 31, wherein the CNS disorder and/or psychological disorder is alcohol use disorder, major depressive disorder (MDD), or treatment-resistant depression (TRD).

33. (canceled)

34. The method of claim 30, wherein the CNS disorder and/or psychological disorder is generalized anxiety disorder (GAD), optionally wherein the generalized anxiety disorder is comorbid with depression.

35-38. (canceled)

39. The method of claim 30, wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

40. (canceled)

41. The method of claim 30, wherein the injectable pharmaceutical formulation is administered via subcutaneous injection.

42-48. (canceled)
